(19) United States
(12) Patent Application Publication
(10) Pub. No.: US 2012/0095387 A1
(43) Pub. Date: Apr. 19, 2012

(54) METHODS FOR DETECTION, DIAGNOSIS AND SELECTIVE ERADICATION OF NEOPLASMS IN VIVO USING MULTIDOMAIN BIOTAGS

(76) Inventors: Marek Malecki, Pomona, CA (US); Raf Malecki, San Francisco, CA (US)

(21) Appl. No.: 13/168,950

(22) Filed: Jun. 25, 2011

Related U.S. Application Data
(60) Provisional application No. 61/358,880, filed on Jun. 25, 2010.
(60) Provisional application No. 61/358,883, filed on Jun. 25, 2010.

(51) Int. Cl. A61M 37/00

(52) U.S. Cl. 604/20

(57) ABSTRACT

A method for treating cancer in a subject is provided, the method comprising administering to the subject an effective dose of a multidomain biotag that targets one or more cancer cells; and exposing the subject to one or more rounds of radiation. The one or more rounds of radiation kills the one or more cancer cells targeted by the biotag, but, in general, do not kill healthy cells or kills a negligible number of healthy cells.

### CROSS-REFERENCE TO RELATED APPLICATIONS

This application claims the benefit of U.S. Provisional Patent Application Nos. 61/358,880 and 61/358,883, both filed Jun. 25, 2010, which are incorporated by reference herein in their entirety.

### BACKGROUND

Many cancers are diagnosed in later stages of the disease because of low sensitivity of existing diagnostic procedures and processes. More than 1.5 million people will be newly diagnosed this year (Jemal et al. 2010), almost 600,000 people will die of cancer in the USA in 2010 and millions harbor early-stage cancer without knowing it. It is the number one killer for people under 80. These tragic statistics are largely a result of late diagnoses and inefficient therapies that have deleterious side effects. Bleak survival statistics exist for many types of cancer. Among them are breast cancer, ovarian cancer and brain cancer.

In 2010, the National Cancer Institute estimated that over 200,000 women will be diagnosed with over 40,000 women will die of breast cancer in the United States alone. Among more than 21,000 women that were diagnosed with ovarian cancer in a single year, 13,850 also died that year. The 5 year survival for women diagnosed with stage I ovarian cancer reaches 90%, but for women diagnosed with stage IV ovarian cancer that has metastasized to distant organs, the 5 year survival falls below 5% (Jemal et al. 2009). Another difficulty in dealing with ovarian cancer is that systemic therapies, including radiation and chemotherapy, affect not only the cancer cells but also affects the patient's ova. Thus, conventional therapies carry the risks of inducing mutations in the genomes, which may lead to infertility or congenital diseases in offspring. Currently, there is no screening program for women highly susceptible to acquire ovarian cancer, nor is there a method to detect metastasizing cancer cells in their blood or lymph. Instead, diagnosis, prognosis, and planning of therapy for ovarian cancer is based upon the fine needle or intrasurgical biopsy, followed by histopathology, immunocytochemsitry, and cytogenetics, which are stressful for the patients, time consuming (while the tumors progress), and expensive (often making it not affordable). Further, iatrogenic effects of any oncological surgery intervention include massive dissemination of cancer cells into blood and lymph circulation, with each of them being a potential source of multiple metastases.

Brain tumors may serve as another tragic example, where the initial symptoms are so non-specific that they remain unreported by patients, undetected during the routine lab tests, and very hard to identify during the physical examinations. Diagnosis is based upon image guided or stereotactic biopsy or open brain surgery involving resection of the tumor and histopathological examination of the removed tissue. Since cancer cells spread between functioning neurons, surgical removal of cancer cells includes removal of healthy cells as well. Therefore, immediate iatrogenic effects may include impaired brain functions. Moreover, dissemination of brain cancer cells by means of cerebrospinal and other fluids fluids leads to formation of metastases.

Prostate and lung cancer also have bleak survival statistics for patents with metastatic disease. Nearly 100% of patients diagnosed with stage 1 prostate cancer survive 5 years. However, as soon as the prostate cancer reaches stage III, the 5 year survival drops to 50%. The 5 year survival rate for stage 1 lung cancer patients is 50%, but stage IV patients have a 95% mortality rate over 5 years. Therefore, monitoring metastasis cancers progress is an important element of the oncological care. Upon early detection of metastasis, physicians may be able to provide better more effective treatments before cancers become too advanced for effective treatment.

While many of the metastasizing cancer cells are eliminated by the immune system's natural killer cells (NKC), it only takes one metastatic cell that is not eliminated to give a rise to a malignant, metastatic tumor remaining undetected until it is too late. While the current paradigm suggests that dissemination of cancer cells occurs at its very advanced stages, recent data suggests that cancer cells disseminate immediately upon the onset of the disease. Therefore, they are present in the circulation, posing a threat of establishing metastases (Podsypanina et al. 2008; Weinberg 2008). Successful diagnosis of neoplasms using diagnostic procedures and processes are contingent upon detecting qualitative and/or quantitative changes of cell surface molecules and/or their mutations that are over-expressed and/or distinctly present on neoplastic cells compared to quiescent cells. It is further contingent upon detection a very small number of these molecules as early as possible.

Current methods for diagnosing a malignant tumor require more than one screening procedure. Screening efforts aim to obtain the highest sensitivity in detecting the smallest tumors at the earliest stages. This is so that smaller primary foci and/or metastases do not go undetected and untreated, allowing a small tumor to progress to advanced stages where it can invade neighboring tissues (i.e., Stage III) and metastasize to distant organs (i.e., Stage IV). However, the sensitivity of the screening methods should not present health risks or undermine the current status of the patient's well being.

Presently, the first screening procedure involves detection of a tumor. Many cancer tumors, such as breast cancer are detected by self- or clinical examination. However, such tumors are typically detected after the tumor reaches a volume of 1 ml or 1 cc, when it contains approximately 10⁹ cells. Routine screening by mammography is more sensitive and allows detection of a tumor before it becomes palpable, but only after they reach an inch in diameter. MRI, PET and SPECT can reveal even smaller tumors than can be detected by mammograms contingent upon breast size and density. However, these imaging methods present significant disadvantages. Contrast agents for MRI are toxic and radionuclides delivered for SPECT or PET examination are sources of ionizing radiation. Because of the scans' relatively poor resolution, ovarian cancer often requires several follow up scans with CT or MRI, while undertaking all precautions to protect possible pregnancies, to reveal fine anatomy of developing tumors (Shin et al. 2011). Additionally, all of these diagnostic techniques require dedicated facilities, expensive equipment, well trained staff, and financial coverage.

Mammograms also present disadvantages. As a screening standard for breast cancers, mammography is routinely performed with x-ray. However, the x-ray doses delivered to the tissues during radiological examinations put patients at risk of causing mutations, which may lead to cancer. This is particularly dangerous for women with mutations in the DNA repair genes such as BRCA1,2. Thus, many screening methods induce genetic mutations and put the patient at risk for developing cancer from the very screening procedure designed to detect cancer. Additionally, the current screening methods may induce mutations in the genomes within reproductive organs leading to congenital diseases in newborns.

Detection of a tumor by clinical and/or radiological examinations does not provide the basis for the final diagnosis, for predicting prognosis, for establishing therapy regiments, or for monitoring an outcome. A second screening procedure is required for diagnosis. These procedures most often require immunohistopathological (IHP) examinations of the patients' cancer tissues, acquired by surgical fine needle and/or ex vivo biopsies. IHP examinations allow for the detection of cancer specific molecules using antibodies and/or probes to define the molecular diagnosis.

For example, increased levels of gene expression products for the EGF Receptor HER2 have been shown to be associated with high risks of invasion, metastasis, and recurrence. However, this does not always correspond with the gene amplification and/or levels of transcripts and/or gene expression products. Therefore, detection of gene expression products is the most reliable method to determine cancer malignancy. Moreover, although the ratios between HER2 and EGFR have been shown to differ in various cancers, increased levels of expression for HER2 were detected in 20 fold only in 30% of women with breast cancer (Slamon et al. 1987). Heterodimerization of the EGFR members (also known as the ErbB family) complicates the matter even further (Holbro et al. 2003). Diagnosis based on these relationships demands evaluation of all the members of the EGFR family and determination of the ratios between them. These relationships are also important for establishing any the targeted cancer therapy.

As a sensitive diagnostic standard, PET may also be performed as a diagnostic step. However, PET scans require introducing into the patients' bodies radioactive compounds such as 18FDG, which by themselves may cause mutations. Furthermore, they do not provide anatomical information about where the probe is localized, information concerning gene expression, or immunohistopathological diagnosis. PET scans also have a very poor spatial resolution. Hence there have been attempts to combine PET with CT. This combination multiplies significantly the dose of ionizing radiation, which is far beyond that sufficient for DNA breaking thus introducing mutations in the patients DNA in somatic or germ cells.

These problems reinforce the preference of surgical biopsies followed by histo- and immunopathological evaluations for cancer diagnosis. However, these evaluations are traumatic experiences for patients both physically, and psychologically. Additionally, the biopsies select only a small portion of the tumor under examination, which can lead to mis-diagnosis—especially when the large heterogeneity of cancer cell types that contribute to tumor growth is considered. Therefore, histopathological diagnosis is limited to the results from a very small selection of material, and does not provide the malignancy status for the entire tumor. Finally, it is a very physically traumatic, psychologically draining, time consuming, and expensive process.

With respect to treatment, surgery, radiation therapy and chemotherapy are the main methods of cancer therapy. Immunotherapy has recently become more prevalent. Success of all of these therapies is contingent upon detecting cancer at the earliest stages. As soon as cancer becomes invasive and metastatic, the tiny lines of invading cells or small foci of metastasizing cells may escape detection (“Indian lines”), thus become sources of relapses. These small populations of metastasizing cells require the use of toxic, systemic therapy. Such therapies expose both metastasizing cancer cells and healthy cells to the toxic therapy. One consequence of this type of therapy results in weakening or failure of the immune system, rendering a cancer patient helpless against infection and weakened against the cancer.

Further, when cancer becomes invasive and metastatic, the tiny lines of invading cells or small foci of metastasizing cells may escape detection, thus become sources of relapses. These small populations of metastasizing cells often require the use of toxic, systemic therapy. Like radiation therapy, such therapies expose both metastasizing cancer cells and healthy cells to the toxic therapy. One consequence of this type of therapy results in weakening or failure of the immune system, rendering a cancer patient helpless against infection.

Therefore, it would be advantageous to develop selective therapies for the treatment of cancer, that selectively targets tumor or metastatic cells while leaving healthy cells intact. Such therapies should minimize the risks involved in current treatment methods.

### SUMMARY

In one embodiment, a method for treating cancer in a subject is provided, the method comprising administering to the subject an effective dose of multidomain biotags or oncotags (molecules specifically targeting cancer cells only), that target one or more cancer cells; and exposing the subject to one or more rounds of radiation. The one or more rounds of radiation kills the one or more cancer cells targeted by the biotag or oncotag, but, in general, does not kill healthy cells or kills a negligible number of healthy cells.

In another embodiment, a method for treating cancer in a subject is provided, the method comprising administering to the subject an effective dose of a multidomain biotag or oncotag that targets one or more cancer cells; establishing a vascular access in the subject; connecting the vascular access to an anti-coagulation coated tube (e.g., a heparinized tube) to establish an extracorporeal circulation of a bodily fluid; and exposing the extracorporeal circulation to one or more doses of radiation, killing biotag or oncotag-targeted cancer cells.

In some embodiments, the multidomain biotag or oncotag used in the method for treating cancer comprises one or more binding domains; an internalization domain; an endosomal escape domain; a lysosomal escape domain; and a reporter domain. In some aspects, the reporter domain is a metal binding domain (MBD) that is chelated to a a metal nanoparticle tag.

In one embodiment, at least one of the one or more target binding domains is a cancer biomarker binding domain. In one embodiment, the cancer biomarker is ErbB 1-4, TfR or a mutant thereof. In another embodiment, at least one of the one or more target binding domains is a cancer cell specific anti-ROS blocker. In another embodiment, the molecular probe has at least two target binding domains, the at least two target binding domains comprising a cancer biomarker binding domain and a cancer cell specific anti-ROS blocker. In one embodiment, the one or more binding domain is a single chain variable fragment (scFv) or single domain variable fragment (sdFv).

In some embodiments, a biotag or oncotag has an internalization domain, which is a signal that causes the nanoprobe to enter or to be internalized by the targeted cancer cell. In one embodiment, the internalization domain may include, but is not limited to the following sequences: YHWYGYTPQNVI (SEQ ID NO:19); NPVVGYIGERPQYRDL (SEQ ID NO:20); or ICRRARGDNPDDRCT (SEQ ID NO:21).

In some embodiments, a biotag or oncotag has an endosomal escape domain and a lysosomal escape domain, which are signals that cause the internalized biotag to escape from endosomal and lysosomal pathways. Internalization followed by escape from the endosomal and lysosomal pathways results allows the biotag or oncotag to avoid degradation and recycling of its components by such pathways and also permanently tags the target cancer cell. The trapped biotags accumulated in the cytoplasm or nucleoplasm of target cancer cells act as a reporter or diagnostic payload for diagnosis and as a therapeutic payload for treatment. In one embodiment, the endosomal escape domain may include, but is not limited to the following sequences: GIGAVLKVLTTGLPALISWIKRKRQQ (SEQ ID NO:22); GRKKRRQRRRPPQ (SEQ ID NO:23); or GLFGAIAGFIENGWEGMIDGWYG (SEQ ID NO:24). The lysosomal escape domain may include, but is not limited to the following sequences: CHK6HC (SEQ ID NO:25); or H5WYG (SEQ ID NO:26)

According to embodiments of the disclosure, a molecular probe designed with a target binding domain having an MBD may be tagged with a metal nanoparticle tag to form a biotag to be used in conjunction with the methods described herein. In one embodiment, the MBD may include, but is not limited to the following sequences:

(SEQ ID NO: 27)
(Gly-)ₙ-Cys;
(SEQ ID NO: 28)
(Gly-Arg-)ₙ-Cys;
(SEQ ID NO: 29)
(Gly-Lys-)ₙ-Cys;
(SEQ ID NO: 30)
(Gly-Asp-Gly-Arg)ₙ-Cys;
(SEQ ID NO: 31)
(Gly-Glu-Gly_Arg)ₙ-Cys;
(SEQ ID NO: 32)
(Gly-Asp-Gly-Lys)ₙ-Cys;
(SEQ ID NO: 33)
(Gly-Glu-Gly-Lys)ₙ-Cys;
(SEQ ID NO: 34)
MAP16-B; (Glu-Glu-Glu-Glu-Glu)ₙ;
(SEQ ID NO: 35)
(Glu-Glu-Glu-Glu-Glu-Glu)ₙ;
(SEQ ID NO: 36)
(Asp-Asp-Asp-Asp-Asp)ₙ;
(SEQ ID NO: 37)
(Asp-Asp-Asp-Asp-Asp-Asp)ₙ;
(SEQ ID NO: 38)
Phe-His-Cys-Pro-Tyr-Asp-Leu-Cys-His-Ile-Leu;
(SEQ ID NO: 39)
(Gly-Asp-Gly-Arg)ₙ-(His)5,6;
(SEQ ID NO: 40)
(Gly-Glu-Gly_Arg)ₙ-(His)5,6;
(SEQ ID NO: 41)
(Gly-Asp-Gly-Lys)ₙ-(His)5,6;
(SEQ ID NO: 42)
(Gly-Glu-Gly-Lys)ₙ-(His)5,6;
(SEQ ID NO: 43)
(Gly-Arg-)ₙ-(His)5,6;
or
(SEQ ID NO: 44)
(Gly-Lys-v-(His)5,6.

In one embodiment, the metal nanoparticle tag is a noble metal. In another embodiment, the noble metal is Au, Pd, Pt, Ag. In another embodiment, the metal nanoparticle tag is a superparamagnetic, heavy, or fluorescent element. In another embodiment, the superparamagnetic, heavy, or fluorescent element is Gd, Eu, Fe, Ni, Co, Tb, Cu, F. In another embodiment, the nanoparticle tag is a core-shell nanoparticle, the core shell nanoparticle comprising an inner superparamagnetic metal core and an outer noble metal shell.

In some embodiments, the method for treating cancer further comprises administering an effective dose of a cancer cell specific ROS blocker. In one aspect, the cancer-cell specific anti-ROS blocker is part of the multidomain biotag or oncotag.

In some embodiments, the one or more targeted cancer cells are circulating tumor cells or metastatic circulating cancer cells (including but not limited to ovarian, brain, breast, lung, testicular cancer cells). In other embodiments, the targeted cancer cells are primary hematological neoplasm cells.

In some embodiments, the bodily fluid of the extracorporeal circulation is may be blood, lymph, interstitial, or cerebrospinal fluid.

In some embodiments, the one or more rounds of radiation is x-ray radiation. In other embodiments, the one or more rounds of radiation is AC electromagnetic (including but not limited to long, short, and visible wavelengths) radiation.

### BRIEF DESCRIPTION OF THE DRAWINGS

FIG. 1 is a representative CT image of wells labeled with an antiERBB scFv tagged with gold nanoparticles. Each well contained a different cell line. Upper row (from left to right): 1—AU565 from ATCC as CRL235; 2—UACC812; 3—MDA-MB453; 4—basal level control; 5—UACC893 (20× gene amp). Lower row: 6—normal breast culture cells; 7-8—connective and epithelial tissue normal control cells; 9—SKBR3 from ATCC as HTB30; 10—CRL2338 from ATCC with designation HCC1954. Differences in the brightness between the different wells are directly proportional to the differences in the levels of gene expression products present on the named cancer cells. Thus, the differences in the brightness between the different walls are also proportional to the levels of gene expression. The more malignant the cancer cell line, the brighter the wells are on a CT image.

FIG. 2 is a representative blot illustrating the expression level of an antiERBB scFv tagged with gold nanoparticles. Lane 1—CRL2338, Lane 2—MDA453; Lane 3—SKBR3 from ATCC as HTB30; Lane 4—UACC893, Lane 5—MCF7. Darkness of the bands shown in each lane corresponds to the quantity of the biomarker present in the same number of cells. Each cell line type was seeded at the same density. Accordingly, each well contained the same number of cells. The cells were electrophoresed, electroblotted, and labeled with an scFv*Au. The more malignant the cancer cell line, the darker the bands are on the blot.

FIG. 3 is a representative electroblot gel of lysed SKBR3 cells that shows all proteins that contribute to cellular structure (left) stained with silver, and a single band after labeling with the antiHER2 scFv tagged with gold nanoparticles (right), which illustrates the specificity of the Au*biotag. No other molecules were labeled on each lane, which indicates that the scFv has a high specificity toward the targeted biomarker.

FIG. 4 is a gated elemental spectrum generated from energy dispersive x-ray spectroscopy (EDX SI or EDS) in field emission scanning electron microscopy showing the elemental composition scFv probes tagged with gold.

FIG. 5 is a field emission scanning transmission electron image showing that Au*biotags undergoing rapid internalization into SKBR3 cells followed by escape from the endocytotic pathway.

FIG. 6 is an computed tomography x-ray image showing various levels of HER2 gene expression in SKBR3 cells. The cultured SKBR3 cells were labeled with biotags targeting HER2 and chelating Au nanoparticles for 1 h at 37 degC and then rinsed off in PBS and spun into the pellets. The concentration of biotag was adjusted to 1M followed by a sequence of 10 fold dilutions. Equal volumes (400 microliters) of the cancer cells labeled with biotags at different concentrations were dispensed into separate wells of a multiwell plate: 1 mM (well 7), 2^{nd} 10 mM (well 5), 3^{rd} 100 mM (well 3), and 4^{th} 1M (well 1). The plate was imaged at the standard x-ray mammography settings.

FIG. 7 are images of a nude mouse injected with Au*biotags in diffuse light (A) and imaged by Raman fluorescence (B). The Au*biotag was restricted to the positive tumor as shown in (B). After injection with transgenes blocking antioxidant enzymes (cocktail of antiCatalase, antiSOD, and antiGPX), the mouse was exposed to x-ray radiation to kill targeted cancer cells. Rapid, selective cancer cell death was detected using a biotag that targets the apoptosis marker, phosphatidylserine (C).

FIG. 8 is a graph illustrating antibody dose (scFv or IgG) versus the plasma half-life to illustrate the rapid clearance rate for scFv fragments that are not internalized by target cells. The rapid clearance illustrates an important characteristic of an scFv fragment used alone versus used as part of the biotags developed in the embodiments described herein. In contrast to scFv fragments that are not internalized, the biotags or oncotags bind cells expressing a selected biomarker, are internalized and escaped from endocytotic/lysosmal pathways to become permanent tags of cancer cells. Therefore, the half-life of the biotag does not limit its ability to be used as an imaging probe, but rather enhances the signal to noise ratio as all non-internalized scFv, sdFv, CDR, or SDR are cleared from the body, thus reducing or eliminating the background noise. On the other hand, the increased half life shown by the IgG justifies their use of larger antibodies or functional fragments thereof, but that increases their non-specific absorption by reticulo-endothelial system causing permanent noise, increases immunogenicity with the risk of anaphylactic shock in repeated applications, increases nephrotoxicity, increases non-specific binding due to presence of Fc.

FIG. 9 is a scanning electron image showing an ovarian cancer cell metastasizing onto endothelium. Human endothelium was grown upon the basement membrane model as previously described (Malecki et al. 1989). Ovarian cancer cells supplemented with human blood were laid over human endothelium and incubated for 1 hour at 37 degrees C. Thereafter, the endothelium with metastasizing cancer cells was washed, rapidly frozen, freeze-substituted, critical point dried, and impregnated with fast neutral atom beam. The cells were imaged with JEOL 840.

FIG. 10 is a representative gel illustrating that the scFV antiHER2 construct contains three non-overlapping target domains. Coding sequences of DNA for antiHER2 DNA were amplified by PCR, cloned under CMV promoter, and expressed in human myelomas. The secreted scFv were tested on blots as shown in FIG. 3. Non-overlapping clones were determined and their DNA amplified and run on 1% agarose gel (lanes 2-4). Clean bands are validated with the marker on the lane 1 (the 1^{st} from the left).

FIG. 11 illustrates highly specific labeling of scFv targeting four EGF receptors. Ovarian cancer cell lysates were labeled with four scFv targeting EGF Receptors 1-4 (clockwise from upper left; ERBB1 (A), ERBB2 (B), ERBB3 (C), and ERBB4 (D)). after transfer unto PVDF membranes with the specific scFv antiERBB 1-4 tagged with Au.

FIG. 12 is an energy dispersive x-ray photograph (FIG. 12A) and spectrum (FIG. 12B) collected from ovarian cancer cells, which were present in the blood, spun down at low g onto the silicate carrier (no interference from carbon counts), and washed with buffer to remove all scFv from the cell surfaces and background. The strong and clean signal indicates presence of cancer cells, loaded with scFv tagged with gold due to specific labeling of these cells with oncotag, which was internalized to make this cancer cell permanently tagged. The biotags and oncotags were entirely biocompatible, so that the cancer cells could be cultured for months (the amount of biotags present in each passage was reduced).

FIG. 13 is an example of a field emission, energy filtering transmission electron microscopy (FE EF TEM) picture showing internalization and endosomal escape of antiHER2 scFv*Au. The ovarian cancer cell HER2 receptors were labeled with scFv*Au. After thorough rinsing they were rapidly cryo-immobilized, freeze-substituted, embedded, and ultrathin-sectioned. They were viewed in the Philips 400 TEM with Gatan post-column energy filter. The lower, centered, endosome is filled with scFv*Au represented by black dots. Above it, there is an endosome containing some of the scFv*Au, but many of the scFv*Au have been depleted. To the left from both, there is a trail of scFv*Au escaping from the endocytotic compartments. Upon escaping from endocytotic and lysosomal pathways, these scFv*Au, are not recycled to the surface, but retained in the cytoplasm, thus establishing a permanent biotag for this cancer cell.

FIG. 14 illustrates ovarian cancer cells labeled with antiHER2*Gd superparamagnetic scFv. Ovarian cancer cells TOV-112D CRL-11731 were labeled with antiHER2 scFv chelated with clusters of Gd atoms and imaged in Hitachi 3400 SEM with EDXSI. Secondary electron emission shows the cell surface ultrastructure (A). X-ray radiation at the specific for Gd atoms energy determines presence of scFv (B). Gated elemental spectrum for scFv tagged with Gd extracted from a pixel acquired with the beam parked (C). Horizontal field width 65 microns.

FIG. 15 is an immunoblot of ovarian cancer cell (TOV-112D CRL-11731 and CRL-117320V-90 (lanes 1-2)) and breast cancer cell (CRL-2340 HCC2157). The cell lysates were electrotransferred onto PVDF membrane and labeled with anti HER2/neu scFv without (left) and with (right) chelating Gd or Eu atoms. Intentionally the space below and above the bands are not cut off to show absence of any non-specific binding. Only specific bands are present. Chelation did not change the specificity of scFv antibodies.

FIG. 16 illustrates isolation and separation of the SKBR3 ovarian cancer cells. 10,000 cells were mixed with full human blood from a healthy volunteer. The biotag was injected and the sample incubated for 15 min at 37 degC. The biotag was an antiHER2 sdFv chelated with superparamagnetic core-shell iron oxide-gold nanoparticles (FeAu*biotag). The sample was placed in magnetic field. Inverting a tube containing a sample that includes cells labeled with the FeAu*biotag within a magnetic field, results in the labeled cells to be attracted and retained against the magnets, while the unlabeled cells did fall away.

FIG. 17 is an energy dispersive x-ray spectrum collected from SKBR3 cancer cells which were present in the blood. The cancer cells were labeled with antiHER2-sdFv tagged with superparamagnetic core-shell iron oxide-gold nanoparticles (antiHER2*FeAu (core-shell)) superparamagnetic sdFv and isolated with the magnet, while all the blood leftovers were washed away with PBS. The intense peaks of Fe and Au indicate presence of the superparamagnetic sdFv internalized and escaped into the cytoplasm, while creating a permanent magnetically detected reporter for these cancer cells. These tags may also have therapeutic applications, when generating heat upon exposure to high frequency magnetic radiation. Since only cancer cells are labeled with the superparamagnetic nanoparticles, then only cancer cells are killed by magnetic radiation induced heat.

FIG. 18 is a representative CT phantom slice of cultured SKBR3 cells labeled with antiHER2 biotag. Cells were plated at volumes of 200 μl (2), 100 μl (3), 50 μl (4) and 25 μl (5), then was placed within the Aquilion clinical CT operated at 120 kV. Stacks of 2 mm slices were acquired. Signal intensity was measured by Haunsfield units.

FIG. 19 is an integrated energy dispersive x-ray spectrum (A) and composition (B) of biotags harboring nanoparticles of different metals analyzed using Noran software. The integrated spectrum (A) shows energy peaks for Au, Pd and Cu. The individual biotags were gated for the specific element (B).

FIG. 20 is an electron microscopy image of biotags harboring Au crystal that were was frozen and freeze-dried onto a carrier film supported by a 1000 mesh grid. The image was taken by a Phillips FESTEM. The image shows the atomic lattice of polycrystal, which validates the biotag's composition.

FIG. 21 is an electron microscopy image of biotags harboring core-shell ((Fe₃O₄/Fe₂O₃—Au) nanoparticles that were frozen and freeze-dried onto a carrier film supported by a 1000 mesh grid. The image was taken with the in-column energy filter on EFTEM Zeiss at zero loss (B) and with the energy spectrum filtered for Fe (A) and acquired on Fuji film. The image reveals Fe cores and Au shells, which validates the composition of biotags harboring superparamagnetic nanoparticles.

FIG. 22 is an electron microscopy image of a biopsied tumor tissue sensitized with biotags and antioxidative enzymes (antiCatalase (antiCAT), antiSuperoxide Dismutase (antiSOD), antiGlutathione Peroxidase (antiGPX) and exposed to radiation. (B) shows the collapse of chromatin against the nuclear membrane, a hallmark of apoptosis.

FIG. 23 is an electron microscopy image of a cancer cell undergoing apoptosis after treatment with core shell ((Fe₃O₄/Fe₂O₃—Au))*BioTags while in circulating blood. The cell illustrates “membrane blebs,” which are a sign of apoptosis.

FIG. 24 is an electron microscopy image of a cancer cell sensitized with Au*biotags while in circulating blood undergoing apoptosis after treatment with x-rays. The cell illustrates “membrane blebs,” which are a sign of apoptosis.

FIG. 25 shows a set of instruments used for ex vivo eradication of cancer cells from an extracorporeal circulation. (A) shows an X-ray radiation source, (B) shows a peristaltic pump, and (C) shows an extracorporeal circuit comprising heparinized tubes, through which a subject's blood, lymph or cerebrospinal fluid (CSF) is circulated while being exposed to the X-ray radiation.

FIG. 26 illustrates the progression of cancer during the diagnostic processes. On day 1 (A), the biotags described herein may be used to diagnose a developing tumor, even before the tumor is detectable by conventional diagnostic methods. In contrast, a cancerous tumor is typically not detected for at least 11 days after the first visit (B), allowing the volume of the cancerous tumor to grow to over 2,000 mL larger than on the first visit.

FIG. 27 is a schematic diagram of a biotag having a reporter (A), a reporter binding domain (B), four functional domains (C1-C4), a biotag or oncotag biomarker binding domain (D). The binding domain (D) targets a target biomarker (E) on a tumor or cancer cell according to embodiments described herein.

FIG. 28 illustrates expression of epidermal growth factor receptor variant III mutant (EGFRvIII) on the immunblot: (a) the cultured cells human glioma (U87) expressing EGFRwt (as the negative control), but not the mutant EGFRvIII, (b) the cultured cells of human glioma expressing the mutated gene EGFRvIII (as a positive control), (c) immunoblot of the patient with the clinical diagnosis of the brain tumor not expressing EGFRvIII (EGFRvIII negative); (d) immunoblot of the patient with the clinical diagnosis of the brain tumor expressing EGFRvIII (EGFRvIII positive) from CSF of the patients (representative of the EGFRvIII positive brain cancer cells); (e) EGFRvIII negative cells from CSF of the patient diagnosed with Other Neurological Diseases (OND) (e.g., Multiple Sclerosis or Brain Stroke).

FIG. 29 illustrates differences in the relaxation times measured within NMR, which were induced by labeling with superparamagnetic s*scFv_{EGFRvIII} of the cells from CSF of the patients diagnosed with brain cancers (Glioblastoma, Anaplastic astrocytoma, and Anaplastic oligodendroglioma) and identified as EGFR positive (BC EGFRvIII+) or EGFRvIII negative (BC EGFRvIII−), as well diagnosed with Other Neurological Diseases being all EGFRvIII negative (OND EGFRvIII−).

FIG. 30 illustrates expression of EGFRvIII on the immunoblot: (a) the cultured cells human OCC expressing EGFRwt considered to as the negative control (OCC EGFRvIII−), but not the mutant EGFRvIII showing no signs of labeling with s*scFv_{EGFRvIII}; (b) the cultured cells of human ovarian carcinoma cells expressing the mutated transgene EGFRvIII, as a positive control (OCC EGFRvIII+)), (c) immunoblot of the patient with the clinical diagnosis of the ovarian cancer not expressing EGFRvIII (OC EGFRvIII−); (d) immunoblot of the patient with the clinical diagnosis EGFRvIII positive from PF of the patients (representative of the EGFRvIII positive cancer cells (OC EGFRvIII+); (e) EGFRvIII negative cells from PF of the patient diagnosed with other diseases (OD EGFRvIII−) abdominal cavity.

FIG. 31 illustrates differences in the relaxation times measured within NMR in milliseconds (ms), which were induced by labeling with s*scFv_{EGFRvIII} of the cells from peritoneal washings, peritoneal effusions, or peritoneal fluid of patients, who were diagnosed as: ovarian cancer EGFR positive (OC EGFRvIII+), ovarian cancer EGFRvIII negative (OC.EGFRvIII−), and other diseases being all EGFRvIII negative (OD EGFRvIII−).

FIG. 32 is a model of an EGFRvIII scFv according to the embodiments described herein,

### DETAILED DESCRIPTION

Methods for treating cancer or a malignant tumor using multidomain biotags (also known as oncotags, bionanoprobes, nanotags, and nanoprobes) that target cancer cells are provided herein. Methods for treating cancer ex vivo using multidomain biotags ( ) that target cancer cells are provided herein. In some embodiments, the methods provided herein may be used to treat metastatic, metastasizing, and/or dormant cancer by killing metastatic, metastasizing, and/or dormant cancer cells present in a bodily fluid such as blood, lymph or cerebrospinal fluid (CSF). In other embodiments, the methods may be used to treat primary hematological cancer. In one embodiment, the biotags, which may also be used as a diagnostic tool, act as a radiosensitizer to render targeted cells more sensitive to radiation therapy, as compared to non-targeted, healthy cells. The ability of the biotags to act as a cancer specific targeted radiosensitizer is a significant improvement over current radiation treatment methods, because with the use of the biotags, radiation treatment may be used at a dose that is not lethal to non-labeled healthy cells, but is lethal to the labeled cancer cells. The dose is currently one of the main factors limiting the effectiveness of the applied dose, while also producing undesirable side effects. Therefore the usual dose of approximately 20 Gy may be distributed into multiple sessions with the single doses dependent of the patients' overall health status. Therefore, the methods for treating cancer or a malignant tumor as provided herein present a significant improvement over current therapeutic methods as they give rise to radiation treatment that is more effective, requiring far fewer visits, and is far less expensive as compared to current treatments with far fewer side effects.

In one embodiment, the method for treating cancer or a malignant tumor in a subject may comprise (1) administering an effective dose of a multidomain biotag that targets one or more cancer cells of the subject, and (2) exposing the subject to one or more rounds of radiation, the radiation killing the one or more cancer cells targeted by the biotag in vivo, but, generally, not killing non-targeted healthy cells or only killing a negligible number of cells. In another embodiment, the method for treating cancer or a malignant tumor in a subject may comprise (1) administering to the subject an effective dose of a multidomain biotag that targets one or more cancer cells; (2) establishing a vascular access in the subject; (3) connecting the vascular access to an anti-coagulation coated tube (e.g., a heparinized tube) to establish an extracorporeal circulation of a bodily fluid; and (4) exposing the extracorporeal circulation to one or more doses of radiation, killing biotag-targeted cancer cells ex vivo. The radiation may be x-ray radiation or alternating electromagnetic radiation. Optionally, the method for treating cancer or a malignant tumor includes administering an effective dose of anti-ROS enzyme blockers. The anti-ROS enzyme blockers may be administered as a transgene or alternatively, may be part of the multidomain biotag that targets the cancer cells or of an independent multidomain biotag, described further below.

In another embodiment, the method for treating cancer or a malignant tumor may follow detection and/or diagnosis of said cancer or tumor using a multidomain biotag. Therefore, a method for diagnosing cancer or a malignant tumor is also provided herein. The diagnostic method may comprise (1) administering an effective dose of a targeted contrast to the subject; (2) exposing the subject to a diagnostic imaging technique such as x-ray, CT, Raman, fluorescence and MRI; (3) detecting a population of cells expressing the cancer biomarker; and (4) quantifying the expression of the cancer biomarker in the population of cells; wherein an increased expression of biomarker indicates that the population of cells is a malignant tumor and the subject has cancer. Alternatively, the diagnostic method may comprise (1) obtaining a blood, lymph, cerebrospinal fluid, urine, or other bodily fluid sample from a subject; (2) exposing the sample in step (1) to a biotag that targets a cancer biomarker; (3) detecting the cancer cells by means of nuclear magnetic resonance (NMR), surface Plasmon resonance (SPR), flow cytometry (FCM), fluorometry, spectrometry, etc (4) isolating the cells from the sample that bind to the biotag for further cultures, imaging, genomic and proteomic analysis, or for use in personalized medicine.

The methods provided herein can be used to treat any type of cancer that has metastasized, wherein the cancer cells are present in one or more bodily fluids, such as blood, lymph or cerebrospinal fluid (CSF). Alternatively, the methods can also be used to treat a primary hematologic neoplasm. A primary hematologic neoplasm includes any type of blood, lymph or bone marrow-associated cancer, including, but not limited to leukemia (e.g., acute lymphoblastic leukemia (ALL), acute or chronic myelogenous leukemia (AML, CML), chronic lymphocytic leukemia (CLL) and acute monocytic leukemia (AML)), Hodgkin's lymphoma, non-Hodgkin's lymphomas (e.g., chronic lymphocytic leukemia (CLL), Diffuse large B-cell lymphoma (DLBCL), follicular lymphoma (FL), mantle cell lymphoma (MCL), marginal zone lymphoma (MZL), Burkitt's lymphoma (BL), hairy cell leukemia, post-transplant lymphoproliferative disorder (PTLD), Waldenström's macroglobulinemia/lymphoplasmacytic lymphoma, hepatosplenic-T cell lymphoma, and cutaneous T cell lymphoma, including Sezary's syndrome)

According to the embodiments described herein, the methods provided herein may comprise delivering a diagnostic or therapeutic payload to one or more cancer cells. The term “payload,” as used herein, relates to chemical moieties which are to be delivered, for example, into the cytoplasm of a living cell, or into the nucleus of a living cell. In some embodiments, the payload may have therapeutic value, for example, as a biologically active agent or therapeutic, or as a species which gives rise, directly or indirectly, to a biologically active agent or therapeutic, which is useful in therapy or treatment. In other embodiments, the payload may have diagnostic value, for example, as a detectable label or as a species which gives rise, directly or indirectly, to a detectable label. In other embodiments, the payload may have both therapeutic value and diagnostic value (e.g., a labeled drug, e.g., a peptide having a radioactive-iodine-labeled tyrosine residue). The payload may have other value, as an alternative to, or in addition to diagnostic and/or therapeutic value. Examples of therapeutic or diagnostic payloads include, but are not limited to, drugs, prodrugs, chemotherapeutics, radiotherapeutics, peptides, proteins, antibodies and functional fragments thereof (described below), enzymes, transcription factors, signaling protins, antisense peptides, zinc fingers, peptide vaccines, nucleotides, oligonucleotides, plasmids, nucleic acids, fluorophores, chromophores, isotope-enriched species, paramagnetic or other metallic species, radioactive species, scintillents and phosphors, and chelating agents.

In some embodiments, a diagnostic or therapeutic payload comprises one or more payload moieties. In other embodiments, a diagnostic or therapeutic payload comprises a plurality of payload moieties that serve the same or similar function or may serve more than one independent functions. For example, the one or more payload moieties may be homogenous (that is, only one type of payload moiety is present, e.g., a single drug, fluorophore, etc.). Thus, in one embodiment, the plurality of payload moieties are identical. Alternatively, the payload may be heterogeneous (that is, more than one type of payload moiety is present. Thus, in one embodiment, the plurality of payload moieties are of two types.

In one embodiment, the therapeutic or diagnostic payload may be delivered into the cytoplasm or nucleus of the target cell by a mechanism which involves binding a surface molecule, endocytosis and subsequent endosomal and lysosomal escape. In other embodiments, the therapeutic or diagnostic payload may be delivered into the cytoplasm or nucleus of the target cell by lipid bilayer disruption or any other suitable method.

According to the embodiments described herein, the one or more payload moieties may be part of a multi-domain biotag as described below. Examples of payload moieties that are part of such biotags include, but are not limited to, target binding domains, internalization domains, lysosomal escape domains, endosomal escape domains, and nanoparticles.

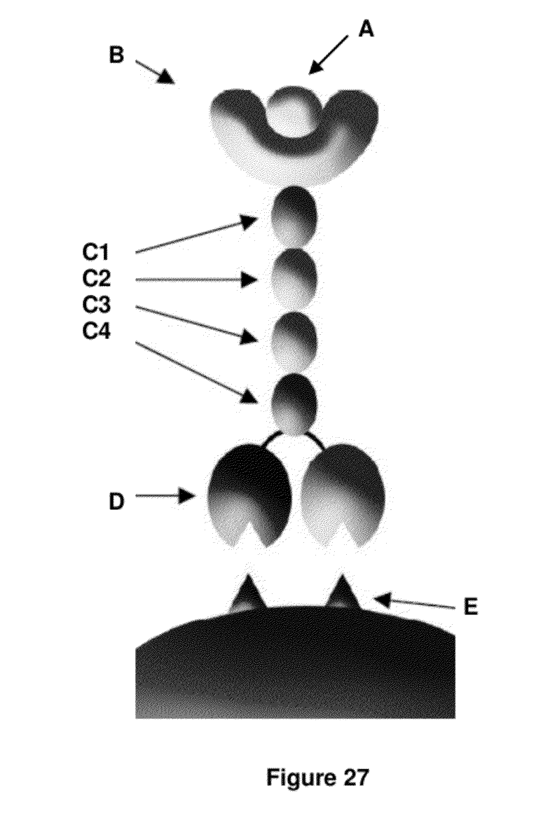

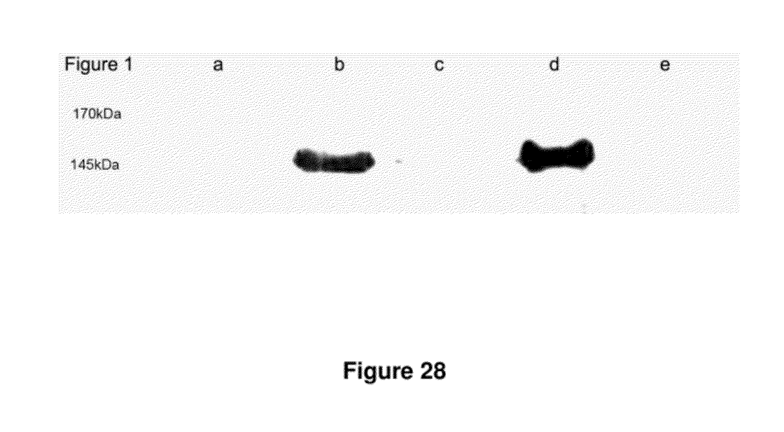

In some embodiments, the biotags may include a plurality of domains, including a receptor or biomarker binding domain (“binding domain”) for binding target cancer cells, one or more additional functional domains that are responsible for the internalization and permanent tagging of the cancer cells and a reporter (e.g., a metal nanoparticle tag) to allow for detection of a biotag's presence. In some embodiments, the biomarker binding domain and the one or more functional domains, form a molecular probe portion of the biotags described herein. In some aspects, the molecular probe portion may also include a reporter binding domain to provide a binding site for the reporter (FIG. 27).

Biomarker Binding Domain

A biotag biomarker binding domain (also referred to as a biotag target binding domain) that may be used in accordance with the disclosure may be any suitable substance that targets a cancer biomarker on a tumor cell or cancer cell. A biomarker may serve to detect any physiologic or pathologic process. In some embodiments, the biomarker is a cancer biomarker. Cancer biomarkers are factors or/and molecules that are present, absent, overexpressed or underexpressed in cancer cells as compared to normal cells. Examples of cancer biomarkers that may be targeted by the biotag biomarker binding domains described herein include, but are not limited to, α-Fetoprotein (AFP), CA125/MUC16, ErbB2/HER2, Estrogen Receptor-α (ERα/NR3A1), Estrogen Receptor-β (ERβ/NR3A2), Kallikrein 3 or Prostate Specific Antigen (PSA), Progesterone R/NR3C3, Carcinoembryonic Antigen (CEA), Prostate Specific Membrane Antigen (PSMA), Fibroblast Growth Factor Receptor (FGFR), Insulin Like Receptor (ILR), recepteur d′ origine nantais (RON) receptor, Vascular Endothelial Growth Factor Receptor (VEGFR), Transferrin Receptor (TfR) and any associated variants or mutants. In one embodiment, the cancer biomarker is targeted by the biotag. In some embodiments, the cancer biomarker may be one or more of the Epidermal Growth Factors Receptors 1-4 (ErbB 1-4) and related variant or mutants thereof, TfR and related variant or mutants thereof or a combination thereof.

Tumors express high levels of growth factors and their receptors, and many types of malignant cells appear to exhibit autocrine or paracrine-stimulated growth. Among the best studied growth factor receptor systems has been the EGF receptor family, ErbB 1-4 (also known as type I receptor tyrosine kinases or EGFR tyrosine kinase receptors) (Mendolsohn & Baselga 2000). This family is comprised of four homologous receptors: the epidermal growth factor receptor ErbB1 (also known as EGFr or HER1), ErbB2 (also known as HER2/neu), ErbB3 (also known as HER3) and ErbB4 (also known as HER4). These receptors are composed of an extracellular binding domain, a transmembrane lipophilic segment and an intracellular protein tyrosine kinase domain with a regulatory carboxyl terminal segment. ErbB3, however, is different from the other members in that it has a deficient tyrosine kinase domain.

The EGF receptor family (ErbB1-4) also includes naturally occurring mutant forms thereof as well as variants thereof, such as EGFRvIII. Variants of the EGF receptor family also include deletional, substitutional and insertional variants, for example those described in Lynch et al. (New England Journal of Medicine 2004, 350; 2129), Paez et al. (Science 2004, 304; 1497) and Pao et al. PNAS 2004, 101:13306). EGFRvIII is expressed at various stages of ovarian cancer reaching 75% of the patients diagnosed with the grade I ovarian carcinomas, but 92% of the patients with grade III ((Moscatello et al. 1995; Lassus et al. 2006; de Graeff et al. 2008; Steffensen et al. 2008). It results in a constitutively active kinase, but with the truncated, extracellular domain. EGFRvIII is also expressed in brain cancer and is responsible for activation of c-jun N-terminal kinase (Malden et al. 1988; Yamazaki et al. 1988; Sugawa et al. 1990; Ekstrand et al. 1990; de Palazzo et al. 1990; Wong et al. 1992; Antonyak et al. 1998).

Expression of ErbB 1-4 receptors and their ligands is detected in cancer cells and it is suggested that all four of the ErbB 1-4 receptors and variants or mutants thereof, such as EGFRvIII, play a role in many human cancers, including lung cancer, breast cancer, stomach cancer, colon cancer, esophageal cancer, liver cancer, pancreatic cancer, prostate cancer, renal cancer, bladder cancer, ovarian cancer, testicular cancer, brain cancer and head and neck cancer (Normanno et al., 2003, Jemal et al. 2010).

For example, Her2/neu is an oncogene amplified and overexpressed in ovarian and breast cancer cells (Di Fiore et al 1988, Berger et al 1988, Guerin et al 1988, van de Vijver et al 1988, Slamon et al 1989, Nielsen et al 2007). The level of its expression is associated with cancer malignancy (Berchuck et al 1990, King et al 1992, Zagouri et al 2007, Robert & Favret 2007). The ovarian or breast cancer cells may have approximately 1.5×10⁶ HER2/neu receptors expressed on their surface, which is quantitatively similar to the number expressed on A471 cells (having approximately 2M receptors). Healthy cells in these organs may have approximately 2×10⁴ HER2/neu receptors on their surfaces, which is approximately 5% of the number found on cancer cells. The overexpression of HER2/neu receptors on ovarian and breast cancer cells leads to a great increase in the stimulation of signal transduction pathways which accelerates cell cycles and increases cell proliferation (King et al 1988, Lahusen et al 2007). Her2/neu positive cancers are recognized as some of the most invasive cancers often having very poor prognosis. Therefore, having the ability to detect the level of gene expression of ErbB1-4 and related mutants and variants, including HER2/neu receptors and their distribution, may be of great diagnostic and prognostic value. Furthermore, because overexpression of ErbB1-4 typically indicates a more aggressive clinical behavior, Her2/neu and the other EGF receptor family members are currently a target for antibody-guided, receptor-targeted therapies (Hudziak et al 1989, Jorgensen et al 2007, Park et al 2007, Allen et al 2007).

Transferrin receptor (TfR) is a carrier protein for transferrin that is needed form the import of iron into a cell and is regulated in response to intracellular iron concentration. TfR imports iron by internalizing the transferrin-iron complex through receptor-mediated endocytosis. In addition, TfR is broadly expressed in human tumors and plays a significant role in cell proliferation and survival. Iron is essential for functioning of ribonucleotide reductase, which is needed for production of nucleotides needed in proliferating cells. Expression of the trasferrin receptor is correlated with cell proliferation and it has been suggested that this accounts for the high proportion of tumors that stain positively with transferrin receptor antibodies and limited staining of normal tissues. Because increased expression of TfR correlates with cell proliferation, higher levels of TfR also indicate a more aggressive clinical behavior of tumor cells. Thus, the ability to detect the level of gene expression of TfR is also of great diagnostic and prognostic value.

In some embodiments, the biomarker binding domain substance may be a natural ligand or a synthetic molecule capable of targeting a selected cancer biomarker, such as those biomarkers described above. In one embodiment, the binding domain may be an antibody or functional fragment thereof. An antibody or functional antibody fragment thereof refers to an immunoglobulin (Ig) molecule that specifically binds to, or is immunologically reactive with a particular target antigen, and includes both polyclonal and monoclonal antibodies and/or their natural or synthetic derived and/or de novo fragments. The term antibody includes genetically engineered or otherwise modified forms of immunoglobulins, such as chimeric antibodies, humanized antibodies, heteroconjugate antibodies (e.g., bispecific antibodies, diabodies, triabodies, tetrabodies, affibodies and minibodies). The term functional antibody fragment includes antigen binding fragments of antibodies, including, but not limited to, Fab′, F(ab′)₂, Fab, Fv, rIgG, sdFv, scFv, CDR, and SDR fragments. The term scFv refers to a single chain variable fragment (Fv) antibody in which a variable domain of the heavy chain is joined to a light chain by a linker to form one chain. A single domain fragment (sdFv) refers to a single monomeric variable antibody domain, e.g. a single variable heavy chain or a single variable light chain. In some aspects, a CDR region may be modified at one or more specificity determining residues (SDRs) to optimize binding to the target biomarker, thereby forming an SDR modified CDR. The antibodies and functional fragments thereof as described herein may additionally include recombinant (e.g., “rIgG”) or synthetic (e.g., “sIgG”) antibodies and functional fragments thereof.

While any antibody or functional fragment thereof may be suitable for use as a binding domain, a preferred embodiment for a binding domain is an scFv, sdFv, CDR, or SDR fragment or other small antibody functional fragment to reduce steric hindrance and sensitivity, as demonstrated and described in Malecki et al., 2002, which is incorporated herein in its entirety as if fully set forth herein. Thus, in some embodiments, the binding domain may include, but is not limited to, one or more complementarity determining regions (CDRs), a variable heavy chain (VH) fragment, a variable light chain (VL) fragment, a single domain fragment (sdFv), an scFv, CDR, SDR, or a combination thereof. In some aspects, a CDR region may be modified at one or more specificity determining residues (SDRs) to optimize binding to the target biomarker, thereby forming an SDR modified CDR. Other small substances may also be suitable for use as a binding domain, including, but not limited to, a nucleic acid, an aptamer, a small molecule, a peptide, a protein, a fusion protein, a chimeric protein or a peptibody. Any scFv, sdFv, CDR, SDR modified CDR or other molecule that may be used in accordance with the embodiments described herein may be a derivative of a natural antibody or a biomolecule generated by in vitro evolution or synthesized with the assistance of computer molecular modeling and/or engineering.

In some embodiments, the binding domain may include one or more complementary determining regions (CDRs) selected from SEQ ID NO:81-242, as shown in Table 1 below. The sequences shown in Table 1 are heavy chain CDR1, CDR2 and CDR3 sequences (i.e., H1, H2, H3 shown therein) and light chain CDR1, CDR2 and CDR3 sequences (i.e., light chains, L1, L2, L3 shown therein) specific to human EGFR (“anti-huEGFR”), human EGFRvIII (“anti-huEGFRvIII”), and human TfR (“anti-huTfR”). The binding domain may be a single CDR region, two or more conjugated CDR regions, or more than two conjugated CDR regions.

TABLE 1
CDR Sequences.

| | | | Translation |
|---|---|---|---|
| | | | (amino |
| Receptor | | Exemplar Sequence (nucleic | acid sequence) |
| Target | Consensus Sequence (5′→3′) | acid sequence) (5′→3′) | (5′→3′) |
| anti-huEGFR | Ggnttywsnttywsnacntayggnatgcaytrr | ggctttagctttagcacctatggcatgcattaa | GFSFSTYGMH (SEQ ID |
| H1ₐ | (SEQ ID NO: 81) | (SEQ ID NO: 135) | NO: 189) |
| anti-huEGFR | gtnathtgggaygayggnwsntayaartayttyg | gtgatttgggatgatggcagctataaatattttg | VIWDDGSYKYFGDSV |
| H2ₐ | gngaywsngtntrr (SEQ ID NO: 82) | gcgatagcgtgtaa (SEQ ID NO: 136) | (SEQ ID NO: 190) |
| anti-huEGFR | gtnathtgggaygayggnwsntayaartayttyg | gtgatttgggatgatggcagctataaatattttg | DAITMVRGVMKEYFDY |
| H3ₐ | gngaywsngtntrr (SEQ ID NO: 83) | gcgatagcgtgtaa (SEQ ID NO: 137) | (SEQ ID NO: 191) |
| anti-huEGFR | ggnttyacntaywsnacntayggnatgcaytrr | ggctttacctatagcacctatggcatgcattaa | GFTYSTYGMH (SEQ ID |
| H1_{b} | (SEQ ID NO: 84) | (SEQ ID NO: 138) | NO: 192) |
| anti-huEGFR | gtnathtgggargayggnwsntayaartaytay | gtgatttgggaagatggcagctataaatattat | VIWEDGSYKYYGDSV |
| H2_{b} | ggngaywsngtntrr (SEQ ID NO: 85) | ggcgatagcgtgtaa (SEQ ID NO: 139) | (SEQ ID NO: 193) |
| anti-huEGFR | gayggnathwsnatggtnmgngcngtnatgm | gatggcattagcatggtgcgcgcggtgatgcg | DGISMVRAVMRDYFDF |
| H3_{b} | gngaytayttygayttytrr | cgattattttgatttttaa | (SEQ ID NO: 194) |
| | (SEQ ID NO: 86) | (SEQ ID NO: 140) | |
| anti-huEGFR | ggnttyacnttywsnacnttygcnatgcaytrr | ggctttacctttagcacctttgcgatgcattaa | GFTFSTFAMH (SEQ ID |
| H1_{b} | (SEQ ID NO: 87) | (SEQ ID NO: 141) | NO: 195) |
| anti-huEGFR | gtnathtgggaygayggnwsntayaarttytayg | gtgatttgggatgatggcagctataaattttatg | VIWDDGSYKFYAESV |
| H2_{b} | cngarwsngtntrr (SEQ ID NO: 88) | cggaaagcgtgtaa (SEQ ID NO: 142) | (SEQ ID NO: 196) |
| anti-huEGFR | gayggnathacnatggtnmgnggngtnatgm | gatggcattaccatggtgcgcggcgtgatgcg | DGITMVRGVMRDYFDF |
| H3_{b} | gngaytayttygayttytrr | cgattattttgatttttaa | (SEQ ID NO: 197) |
| | (SEQ ID NO: 89) | (SEQ ID NO: 143) | |
| anti-huEGFR | mgngcnwsncargayathwsnwsngcnytn | cgcgcgagccaggatattagcagcgcgctg | RASQDISSALV (SEQ |
| L1ₐ | gtntrr (SEQ ID NO: 90) | gtgtaa (SEQ ID NO: 144) | ID NO: 198) |
| anti-huEGFR | gaygcnwsnwsnytngartrr (SEQ ID | gatgcgagcagcctggaataa (SEQ ID | DASSLE (SEQ ID |
| L2ₐ | NO: 91) | NO: 145) | NO: 199) |
| anti-huEGFR | carcarttyaaywsntayccnytnacntrr | cagcagtttaacagctatccgctgacctaa | QQFNSYPLT (SEQ ID |
| L3ₐ | (SEQ ID NO: 92) | (SEQ ID NO: 146) | NO: 200) |
| anti-huEGFR | mgngcnwsncargarathwsnwsngcnytny | cgcgcgagccaggaaattagcagcgcgctg | RASQEISSALL (SEQ ID |
| L1_{b} | tntrr (SEQ ID NO: 93) | ctgtaa (SEQ ID NO: 147) | NO: 201) |
| anti-huEGFR | gargcnwsnwsnytngaracntrr (SEQ ID | gaagcgagcagcctggaaacctaa (SEQ | EASSLET (SEQ ID |
| L2_{b} | NO: 94) | ID NO: 148) | NO: 202) |
| anti-huEGFR | caraayttyaaywsntayccnytnwsntrr | cagaactttaacagctatccgctgagctaa | QNFNSYPLS(SEQ ID |
| L3_{b} | (SEQ ID NO: 95) | (SEQ ID NO: 149) | NO: 203) |
| anti-huEGFR | mgngcnwsncargayathacnwsngcnytny | cgcgcgagccaggatattaccacgcgctgc | RASQDITSALL (SEQ ID |
| L1_{c} | tntrr (SEQ ID NO: 96) | tgtaa (SEQ ID NO: 150) | NO: 204) |
| anti-huEGFR | gaygcnwsnwsnytngarwsn (SEQ ID | gatgcgagcagcctggaaagc (SEQ ID | DASSLES(SEQlD |
| L2_{c} | NO: 97) | NO: 151) | NO: 205) |
| anti-huEGFR | aaycarttycarwsntayccnytnwsn (SEQ | aaccagtttcagagctatccgctgagc | NQFQSYPLS (SEQ ID |
| L3_{c} | ID NO: 98) | (SEQ ID NO: 152) | NO: 206) |
| anti- | ggnttywsnttymgnaarttyggnatgwsntrr | ggctttagctttcgcaaatttggcatgagctaa | GFSFRKFGMS (SEQ ID |
| huEGFRvIII | (SEQ ID NO: 99) | (SEQ ID NO: 153) | NO: 207) |
| H1ₐ | | | |
| anti- | wsnathwsnacnggnggntayaaywsntayt | agcattagcaccggcggctataacagctatta | SISTGGYNSYYSDNV |
| huEGFRvIII | aywsngayaaygtntrr | tagcgataacgtgtaa (SEQ ID | (SEQ ID NO: 208) |
| H2ₐ | (SEQ ID NO: 100) | NO: 154) | |
| anti- | ggnttywsnwsnacnwsntaygcnatggayta | ggctttagcagcaccagctatgcgatggattat | GFSSTSYAMDY (SEQ |
| huEGFRvIII | yln (SEQ ID NO: 101) | taa (SEQ ID NO: 155) | ID NO: 209) |
| H3ₐ | | | |
| anti- | ggnttyacnttyaaraarttyggnatgwsntrr | ggctttacctttaaaaaatttggcatgagctaa | GFTFKKFGMS (SEQ ID |
| huEGFRvIII | (SEQ ID NO: 102) | (SEQ ID NO: 156) | NO: 210) |
| H1_{b} | | | |
| anti- | wsnathwsnacnggnggnttyaayacntayta | agcattagcaccggcggctttaacacctattat | SISTGGFNTYYSDNV |
| huEGFRvIII | ywsngayaaygtntrr (SEQ ID NO: 103) | agcgataacgtgtaa (SEQ ID NO: 157) | (SEQ ID NO: 211) |
| H2_{b} | | | |
| anti- | ggntaywsnwsnacnwsnttyggnatggayta | ggctatagcagcaccagctttggcatggattat | GYSSTSFGMDY (SEQ |
| huEGFRvIII | ytrr (SEQ ID NO: 104) | taa (SEQ ID NO: 158) | ID NO: 212) |
| H3_{b} | | | |
| anti- | ggntaywsnttymgnaarttyggnatgwsntrr | ggctatagctttcgcaaatttggcatgagctaa | GYSFRKFGMS (SEQ ID |
| huEGFRvIII | (SEQ ID NO: 105) | (SEQ ID NO: 159) | NO: 213) |
| H1_{c} | | | |
| anti- | wsnathwsnacnggnggntaycaracntayta | agcattagcaccggcggctatcagacctatta | SISTGGYQTYYSDNV |
| huEGFRvIII | ywsngayaaygtntrr (SEQ ID NO: 106) | tagcgataacgtgtaa (SEQ ID | (SEQ ID NO: 214) |
| H2_{c} | | NO: 160) | |
| anti- | ggntaywsnwsnacnwsntaygcnatggaytt | ggctatagcagcaccagctatgcgatggatttt | GYSSTSYAMDF (SEQ |
| huEGFRvIII | ytrr (SEQ ID NO: 107) | taa(SEQ ID NO: 161) | ID NO: 215) |
| H3_{c} | | | |
| anti- | mgngcnwsncarwsngtncaywsngayggn | cgcgcgagccagagcgtgcatagcgatggc | RASQSVHSDGNTYMQ |
| huEGFRvIII | aayacntayatgcartrr | aacacctatatgcagtaa (SEQ ID | (SEQ ID NO: 216) |
| L1ₐ | (SEQ ID NO: 108) | NO: 162) | |
| anti- | gcngcnwsnaaymgnttywsntrr (SEQ ID | gcggcgagcaaccgctttagctaa (SEQ | AASNRFS (SEQ ID |
| huEGFRvIII | NO: 109) | ID NO: 163) | NO: 217) |
| L2ₐ | | | |
| anti- | carcarggnacncarytnccnmgnacntrr | cagcagggcacccagctgccgcgcacctaa | QQGTQLPRT (SEQ ID |
| huEGFRvIII | (SEQ ID NO: 110) | (SEQ ID NO: 164) | NO: 218) |
| L3ₐ | | | |
| anti- | mgnwsnwsncarwsngtncaywsngaygg | Cgcagcagccagagcgtgcatagcgatgg | RSSQSVHSDGNSYLS |
| huEGFRvIII | naaywsntayytnwsntrr (SEQ ID | caacagctatctgagctaa (SEQ ID | (SEQ ID NO: 219) |
| L1_{b} | NO: 111) | NO: 165) | |
| anti- | ggngcnwsnaayaarttywsntrr (SEQ ID | ggcgcgagcaacaaatttagctaa (SEQ | GASNKFS (SEQ ID |
| huEGFRvIII | NO: 112) | ID NO: 166) | NO: 220) |
| L2_{b} | | | |
| anti- | carcarggnacncarytnccnmgnacntrr | Cagcagggcacccagctgccgcgcaccta | QQGTQLPRT (SEQ ID |
| huEGFRvIII | (SEQ ID NO: 113) | a (SEQ ID NO: 167) | NO: 221) |
| L3_{b} | | | |
| anti- | aarwsncarwsnytngtncaywsngayggna | aaaagccagagcctggtgcatagcgatggc | KSQSLVHSDGNSYLS |
| huEGFRvIII | aywsntayytnwsntrr | aacagctatctgagtaa (SEQ ID | (SEQ ID NO: 222) |
| L1_{c} | (SEQ ID NO: l14) | NO: 168) | |
| anti- | mgnathwsnaaymgnttywsntrr (SEQ ID | cgcattagcaaccgctttagctaa (SEQ ID | RISNRFS (SEQ ID |
| huEGFRvIII | NO: 115) | NO: 169) | NO: 223) |
| L2_{c} | | | |
| anti- | carcarggnacncarytnccnmgnacntrr | cagcagggcacccagctgccgcgcacctaa | QQGTQLPRT (SEQ ID |
| huEGFRvIII | (SEQ ID NO: 116) | (SEQ ID NO: 170) | NO: 224) |
| L3_{c} | | | |
| anti-huTfR | ggntaywsntaywsnwsntaytggatgtrr | ggctatagctatagcagctattggatgtaa | GYSYSSYWM (SEQ ID |
| H1ₐ | (SEQ ID NO: 117) | (SEQ ID NO: 171) | NO: 225) |
| anti-huTfR | gcnathgayccnmgnaaywsngayacnatht | gcgattgatccgcgcaacagcgataccattta | AIDPRNSDTIYNPQF |
| H2ₐ | ayaayccncarttytrr (SEQ ID NO: 118) | taacccgcagttttaa (SEQ ID NO: 172) | (SEQ ID NO: 226) |
| anti-huTfR | ytntaytayttygaywsntrr | ctgtattattatgatagctaa | LYYYDS |
| H3ₐ | (SEQ ID NO: 119) | (SEQ ID NO: 173) | (SEQ ID NO: 227) |
| anti-huTfR | ggntayacnathwsnwsntaytggatgtrr | ggctataccattagcagctattggatgtaa | GYTISSYWM |
| H1_{b} | (SEQ ID NO: 120) | (SEQ ID NO: 174) | (SEQ ID NO: 228) |
| anti-huTfR | gcngcngayccnmgnaaywsngayacnatht | gcggcggatccgcgcaacagcgataccattt | AADPRNSDTIYQPQY |
| H2_{b} | aycarccncartaytrr | atcagccgcagtattaa | (SEQ ID NO: 229) |
| | (SEQ ID NO: 121) | (SEQ ID NO: 175) | |
| anti-huTfR | ytntaytayttygaywsntrr | ctgtattattttgatagctaa (SEQ ID | LYYFDS |
| H3_{b} | (SEQ ID NO: 122) | NO: 176) | (SEQ ID NO: 230) |
| anti-huTfR | ggntayacngcnacnacntaytggatgtrr | ggctataccgcgaccacctattggatgtaa | GYTATTYWM |
| H1_{c} | (SEQ ID NO: 123) | (SEQ ID NO: 177) | (SEQ ID NO: 231) |
| anti-huTfR | atgathcayccnwsngaywsngargtnmgnyt | atgattcatccgagcgatagcgaagtgcgcct | MIHPSDSEVRLNQ |
| H2_{c} | naaycartrr | gaaccagtaa | (SEQ ID NO: 232) |
| | (SEQ ID NO: 124) | SEQ ID NO: 178) | |
| anti-huTfR | ytntaytayttygarwsntrr | ctgtattattttgaaagctaa | LYYFES |
| H3_{c} | (SEQ ID NO: 125) | (SEQ ID NO: 179) | (SEQ ID NO: 233) |
| anti-huTfR | gayathaayaaytaygtntgytrr | gatattaacaactatgtgtgctaa | DINNYVC |
| L1ₐ | (SEQ ID NO: 126) | (SEQ ID NO: 180) | (SEQ ID NO: 234) |
| anti-huTfR | aargcnaaymgnytngtngaytrr | aaagcgaaccgcctggtggattaa | KANRLVD |
| L2ₐ | (SEQ ID NO: 127) | (SEQ ID NO: 181) | (SEQ ID NO: 235) |
| anti-huTfR | ytncartaygaygarttyccntayacntrr | ctgcagtatgatgaatttccgtatacctaa | LQYDEFPYT |
| L3ₐ | (SEQ ID NO: 128) | (SEQ ID NO: 182) | (SEQ ID NO: 236) |
| anti-huTfR | garathaayaaytayytntgytrr | gaaattaacaactatctgtgctaa | EINNYLC |
| L1_{b} | (SEQ ID NO: 129) | (SEQ ID NO: 183) | (SEQ ID NO: 237) |
| anti-huTfR | mgngcnaayaarytngtngaytrr | cgcgcgaacaaactggtggattaa | RANKLVD |
| L2_{b} | (SEQ ID NO: 130) | (SEQ ID NO: 184) | (SEQ ID NO: 238) |
| anti-huTfR | ytncartaygaygayttyccntayacntrr | ctgcagtatgatgattttccgtatacctaa | LQYDDFPYT |
| L3_{b} | (SEQ ID NO: 131) | (SEQ ID NO: 185) | (SEQ ID NO: 239) |
| anti-huTfR | gayathaaycarttyytntgytrr | gatattaaccagtttctgtgctaa | DINQFLC |
| L1_{c} | (SEQ ID NO: 132) | (SEQ ID NO: 186) | (SEQ ID NO: 240) |
| anti-huTfR | mgngcnaaymgnytngtngaytrr | cgcgcgaaccgcctggtggattaa | RANRLVD |
| L2_{c} | (SEQ ID NO: 133) | (SEQ ID NO: 187) | (SEQ ID NO: 241) |
| anti-huTfR | gtncartaygaygarttyccntaywsntrr | gtgcagtatgatgaatttccgtatagctaa | VQYDEFPYS |
| L3_{c} | (SEQ ID NO: 134) | (SEQ ID NO: 188) | (SEQ ID NO: 242) |

*The consensus sequences are degeneracy sequences which follow the standard IUPAC symbols for DNA (R = A or G; Y = C or T; M = A or C; W = A or T; S = C or G; B = C, G or T; D = A, G or T; H = A, C or T; V = A, C or G; and N is any nucleotide (A, C G or T)).

In some embodiments, the binding domain is an scFv. In such an embodiment, the scFv includes one variable heavy chain fragment (V_{H}) joined to a variable light chain fragment (V_{L}) by a short peptide linker, which is usually between approximately about 5 to about 30 amino acids, as known in the art. The linker is usually rich in glycine for flexibility as well as serine or threonine for solubility, and can either connect the N-terminus of the V_{H} with the C-terminus of the V_{L}, or vice versa. An scFv that may be used according to the embodiments described herein may include a V_{H} sequence and a V_{H} sequence selected from SEQ ID NO:244-297, as shown in Table 2 below. The sequences shown in Table 2 are V_{H} sequences (i.e., heavy chains, HC₁, HC₂, HC₃ shown therein) and V_{L} sequences (i.e., light chains, LC₁, LC₂, LC₃ shown therein) specific to human EGFR (“anti-huEGFR”), human EGFRvIII (“anti-huEGFRvIII”), and human TfR (“anti-huTfR”). In other embodiments, the binding domain is a single domain fragment, or an sdFv or a CDR or a SDR. In such embodiments, an sdFv that may be used according to the embodiment described herein may include a single V_{H} sequence or a single V_{H} sequence selected from SEQ ID NO:244-297, as shown in Table 2 below. In some embodiments, the sdFv is SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296 or SEQ ID NO:297.

V_{H} (“HC”) and V_{L} (“LC”) sequences

| | | | Translation |
|---|---|---|---|
| | | | (amino acid |
| Receptor | | Exemplar Sequence (nucleic acid | sequence |
| Target | Consensus Sequence (5′→3′) | sequence) (5′→3′) | (5′→3′) |
| anti- | Cargtncarytngtngaywsnggngcnggngtngt | caggtgcagctggtggatagcggcgcgggcgtggt | QVOLVDSGAGVV |
| huEGFR | ncarccnggnmgnwsnytnmgngtnwsntgygc | gcagccgggccgcagcctgcgcgtgagctgcgcg | QPGRSLRVSCAA |
| HC₁ | ngcnwsnggnttywsnttywsnacntayggnatg | gcgagcggctttagctnagcacctatggcatgcattg | SGFSFSTYGMHW |
| | caytgggtnmgncarggnccnggnaarggnytng | ggtgcgccagggcccgggcaaaggcctggaatgg | VRQGPGKGLEWV |
| | artgggtngcngtnathtgggaygayggnwsntay | gtggcggtgatttgggatgatggcagctataaatatttt | AVIWDDGSYKYF |
| | aartayttyggngaywsngtnmgnggnmgntaya | ggcgatagcgtgcgcggccgctataccattagcaa | GDSVRGRYTISKE |
| | cnathwsnaargarcarwsnaargtnacnytnttyg | agaacagagcaaagtgaccctgtttgtgcagatgaa | QSKVTLFVQMNS |
| | tncaratgaaywsnytnaargcngaygaracngcn | cagcctgaaagcggatgaaaccgcgggcttttattg | LKADETAGFYCAR |
| | ggnttytaytgygcnmgngaygcnathacnatggt | cgcgcgcgatgcgattaccatggtgcgcggcgtgat | DAITMVRGVMKE |
| | nmgnggngtnatgaargartayttygaytaytgggg | gaaagaatattttgattattggggccagggcaccctg | YFDYWGQGTLVT |
| | ncarggnacnytngtnacngtntrr (SEQ ID | gtgaccgtgtaa (SEQ ID NO: 262) | V (SEQ ID NO: |
| | NO: 244) | | 280) |
| anti- | cargtncarytngtngaracnggngcnggngtngtn | caggtgcagctggtggaaaccggcgcgggcgtggt | 5′QVQLVETGAGV |
| huEGFR | carccnggnmgnwsnytnaargtnwsntgygcn | gcagccgggccgcagcctgaaagtgagctgcgcg | VQPGRSLKVSCA |
| HC₂ | gcnwsnggnttyacntaywsnacntayggnatgc | gcgagcggctttacctatagcacctatggcatgcatt | ASGFTYSTYGMH |
| | aytgggtnmgncargcnccnggnmgnggnytng | gggtgcgccaggcgccgggccgcggcctggaatg | WVRQAPGRGLE |
| | artgggtngcngtnathtgggargayggnwsntay | ggtggcggtgatttgggaagatggcagctataaatat | WVAVIWEDGSYK |
| | aartaytayggngaywsngtnaarggnmgnttyac | tatggcgatagcgtgaaaggccgctttaccgcgagc | YYGDGVKGRFTA |
| | ngcnwsnmgngayaaywsnmgnaayacnytnt | cgcgataacagccgcaacaccctgtatctgaacatg | SRDNSRNTLYLN |
| | ayytnaayatgaaywsnytnaargcngaygayws | aacagcctgaaagcggatgatagcgcggtgtattatt | MNSLKADDSAVY |
| | ngcngtntaytaytgygcnmgngayggnathwsn | gcgcgcgcgatggcattagcatggtgcgcgcggtg | YCARDGISMVRA |
| | atggtnmgngcngtnatgmgngaytayttygaytty | atgcgcgattattttgattttttggggccagggcaccc | VMRDYFDFWGQ |
| | tggggncarggnacnytngtnacngtntrr (SEQ | tggtgaccgtgtaa (SEQ ID NO: 263) | GTLVTV (SEQ ID |
| | ID NO: 245) | | NO: 281) |
| anti- | cargtncarytngtngaywsnggnggnggngtnyt | caggtgcagctggtggatagcggcggcggcgtgct | QVQLVDSGGGVL |
| huEGFR | ncarccnggnmgnwsnytnaarytnwsntgygcn | gcagccgggccgcagcctgaaactgagctgcgcg | QPGRSLKLSCAA |
| HC₃ | gcnwsnggnttyacnttywsnacnttygcnatgca | gcgagcggctttacctttagcacctttgcgatgcattg | SGFTFSTFAMHW |
| | ytgggtnmgncargcnccngcnaarggnytngart | ggtgcgccaggcgccggcgaaaggcctggaatgg | VRQAPAKGLEWV |
| | gggtngcngtnathtgggaygayggnwsntayaar | gtggcggtgatttgggatgatggcagctataaattttat | AVIWDDGSYKFYA |
| | ttytaygcngarwsngtnmgnggnmgnttyacng | gcggaaagcgtgcgcggccgctttaccggcacccg | ESVRGRFTGTRD |
| | gnacnmgngayaaywsnaargtnacnytnttyyt | cgataacagcaaagtgaccctgtttctgcagatgca | NSKVTLFLQMQSL |
| | ncaratgcarwsnytnmgngcngargayacngcn | gagcctgcgcgcggaagataccgcggtgttttattgc | RAEDTAVFYCAR |
| | gtnttytaytgygcnmgngayggnathacnatggtn | gcgcgcgatggcattaccatggtgcgcggcgtgatg | DGITMVRGVMRD |
| | mgnggngtnatgmgngaytayttygayttytgggg | cgcgattattttgatttttggggccagggcaccctggtg | YFDFWGQGTLVT |
| | ncarggnacnytngtnacngtntrr (SEQ ID | accgtgtaa (SEQ ID NO: 264) | V (SEQ ID NO: |
| | NO: 246) | | 282) |
| anti- | gcnathcarytnacnaaywsnccnwsnwsnytn | gcgattcagctgaccaacagcccgagcagcctgag | AIQLTNSPSSLSA |
| huEGFR | wsngcnwsngtnggngaymgngtnacnathws | cgcgagcgtgggcgatcgcgtgaccattagctgccg | SVGDRVTISCRAS |
| LC₁ | ntgymgngcnwsncargayathwsnwsngcnyt | cgcgagccaggatattagcagcgcgctggtgtggta | QDISSALVWYQQ |
| | ngtntggtaycarcaraarccngcnmgngcnccna | tcagcagaaaccggcgcgcgcgccgaaactggtg | KPARAPKLVIYDA |
| | arytngtnathtaygaygcnwsnwsnytngarwsn | atttatgatgcgagcagcctggaaagcggcgtgccg | SSLESGVPTKFTG |
| | ggngtnccnacnaarttyacnggnacngaywsng | accaaatttaccggcaccgatagcggcaccgatttta | TDSGTDFSLTISSL |
| | gnacngayttywsnytnacnathwsnwsnytnca | gcctgaccattagcagcctgcagccggatgattttgc | QPDDFATFYCQQ |
| | rccngaygayttygcnacnttytaytgycarcart | gaccttttattgccagcagtttaacagctatccgctgac | FNSYPLTFGGGTK |
| | tyaaywsntayccnytnacnttyggnggnggnacn | ctttggcggcggcaccaaagtgtaa (SEQ ID | V (SEQ ID NO: |
| | aargtntrr (SEQ ID NO: 247) | NO: 265) | 283) |
| anti- | gcnathcargtnacncarwsnccnacnwsnytnw | gcgattcaggtgacccagagcccgaccagcctgag | AIQVTOQPTSLSA |
| huEGFR | sngcnacngtnggngaymgngtnwsnathacnt | cgcgaccgtgggcgatcgcgtgagcattacctgccg | TVGDRVSITCRAS |
| LC₂ | gymgngcnwsncargarathwsnwsngcnytny | cgcgagccaggaaattagcagcgcgctgctgtggt | QEISSALLWYQQK |
| | tntggtaycarcaraarccnggnaargcnccnmgn | atcagcagaaaccgggcaaagcgccgcgcctgct | PGKAPRLLIYEAS |
| | ytnytnathtaygargcnwsnwsnytngaracngg | gatttatgaagcgagcagcctggaaaccggcgtgc | SLETGVPSKFTGS |
| | ngtnccnwsnaarttyacnggnwsngaracnggn | cgagcaaatttaccggcagcgaaaccggcagcga | ETGSDFTRTISSV |
| | wsngayttyacnmgnacnathwsnwsngtncar | ttttacccgcaccattagcagcgtgcagccggaagat | QPEDAYTYFCQN |
| | ccngargaygcntayacntayttytgycaraayttya | gcgtatacctatttttgccagaactttaacagctatc | FNSYPLSFGGGT |
| | aywsntayccnytnwsnttyggnggnggnacnaa | cgctgagctttggcggcggcaccaaagtgtaa (SEQ | KV (SEQ ID |
| | rgtntrr (SEQ ID NO: 248) | ID NO: 266) | NO: 284) |
| anti- | gcnathcarytnacncarwsnccnwsnacnytna | gcgattcagctgacccagagcccgagcaccctgac | AIQLTQSPSTLTA |
| huEGFR | cngcnwsngtnggngaymgngtnacnathacnt | cgcgagcgtgggcgatcgcgtgaccattacctgccg | SVGDRVTITCRAS |
| LC₃ | gymgngcnwsncargayathacnwsngcnytny | cgcgagccaggatattaccagcgcgctgctgtggta | QDITSALLWYQQR |
| | tntggtaycarcarmgnccngcnaargcnccnaar | tcagcagcgcccggcgaaagcgccgaaagtgctg | PAKAPKVLIYDAS |
| | gtnytnathtaygaygcnwsnwsnytngarwsng | atttatgatgcgagcagcctggaaagcggcgtgccg | SLESGVPSRFSG |
| | gngtnccnwsnmgnttywsnggnwsngaywsn | agccgctttagcggcagcgatagcggcagcgaata | SDSGSEYTLTISS |
| | ggnwsngartayacnytnacnathwsnwsngtna | taccctgaccattagcagcgtgaacccggatgattat | VNPDDYATYYCN |
| | ayccngaygaytaygcnacntaytaytgyaaycart | gcgacctattattgcaaccagtttcagagctatccgct | QFQSYPLSFGGG |
| | tycarwsntayccnytnwsnttyggnggnggnacn | gagctttggcggcggcaccaaagtgtaa (SEQ | TKV (SEQ ID |
| | aargtntrr (SEQ ID NO: 249) | ID NO: 267) | NO: 285) |
| anti- | cargtnaarytncarcarwsnggnggnggnytncc | caggtgaaactgcagcagagcggcggcggcctgc | QVKLQQSGGGLP |
| huEGFRvIII | naargtngcnggnwsnytnaarytnwsntgygtna | cgaaagtggcgggcagcctgaaactgagctgcgtg | KVAGSLKLSCVTS |
| HC₁ | cnwsnggnttywsnttymgnaarttyggnatgwsn | accagcggctttagctttcgcaaatttggcatgagctg | GFSFRKFGMSWV |
| | tgggtnmgncaracnwsngayaarmgnytngart | ggtgcgccagaccagcgataaacgcctggaatgg | RQTSDKRLEWIG |
| | ggathggnwsnathwsnacnggnggntayaayw | attggcagcattagcaccggcggctataacagctatt | SISTGGYNSYYSD |
| | sntaytaywsngayaaygtnaarggnmgnttyac | atagcgataacgtgaaaggccgctttaccattagcc | NVKGRFTISRENA |
| | nathwsnmgngaraaygcnaaraayacnytntay | gcgaaaacgcgaaaaacaccctgtatctgaacatg | KNTLYLNMSSLKS |
| | ytnaayatgwsnwsnytnaarwsngargayacng | agcagcctgaaaagcgaagataccgcgctgtattat | EDTALYYCARGFS |
| | cnytntaytaytgygcnmgnggnttywsnwsnac | tgcgcgcgcggctttagcagcaccagctatgcgatg | STSYAMDYWGQ |
| | nwsntaygcnatggaytaytggggncarggnacn | gattattggggccagggcaccaccgtgaccgtgtaa | GTTVTV (SEQ ID |
| | acngtnacngtntrr (SEQ ID NO: 250) | (SEQ ID NO: 268) | NO: 286) |
| anti- | cargtnaargtncaraaywsnggnggnggnytngt | caggtgaaagtgcagaacagcggcggcggcctgg | QVKVQNSGGGLV |
| huEGFRvIII | naarccnggngcnwsnytnaarytnwsntgygtn | tgaaaccgggcgcgagcctgaaactgagctgcgtg | KPGASLKLSCVTS |
| HC₂ | acnwsnggnttyacnttyaaraarttyggnatgwsn | accagcggctttacctttaaaaaatttggcatgagctg | GFTFKKFGMSWV |
| | tgggtnaarcaracnwsngayaaraarytngartgg | ggtgaaacagaccagcgataaaaaactggaatgg | KQTSDKKLEWVA |
| | gtngcnwsnathwsnacnggnggnttyaayacnt | gtggcgagcattagcaccggcggctttaacacctatt | SISTGGFNTYYSD |
| | aytaywsngayaaygtnaarggnmgnttyacnat | atagcgataacgtgaaaggccgctttaccattagcc | NVKGRFTISRENG |
| | hwsnmgngaraayggnaaraayacnytntaygtn | gcgaaaacggcaaaaacaccctgtatgtgcagatg | KNTLYVQMSSLKS |
| | caratgwsnwsnytnaarwsngargayacngcny | agcagcctgaaaagcgaagataccgcgctgtattat | EDTALYYCTRGYS |
| | tntaytaytgyacnmgnggntaywsnwsnacnw | tgcacccgcggctatagcagcaccagctttggcatg | STSFGMDYWGQ |
| | snttyggnatggaytaytggggncarggnacnacn | gattattggggccagggcaccaccgtgtaa (SEQ | GTTV (SEQ ID |
| | gtntrr (SEQ ID NO: 251) | ID NO: 269) | NO: 287) |
| anti- | cargtnaarytncarcarwsnggngcnggnytngt | caggtgaaactgcagcagagcggcgcgggcctgg | QVKLQQSGAGLV |
| huEGFRvIII | naarccnggngcnwsnytnaarytnwsntgygtn | tgaaaccgggcgcgagcctgaaactgagctgcgtg | KPGASLKLSCVTS |
| HC₃ | acnwsnggntaywsnttymgnaarttyggnatgw | accagcggctatagctttcgcaaantggcatgagct | GYSFRKFGMSWV |
| | sntgggtnmgncarwsnacngayaarmgnytng | gggtgcgccagagcaccgataaacgcctggaatg | RQSTDKRLEWVA |
| | artgggtngcnwsnathwsnacnggnggntayca | ggtggcgagcattagcaccggcggctatcagacct | SISTGGYQTYYSD |
| | racntaytaywsngayaaygtnaarggnmgnttya | attatagcgataacgtgaaaggccgctttaccattag | NVKGRFTISRENA |
| | cnathwsnmgngaraaygcnaaraayacnytnta | ccgcgaaaacgcgaaaaacaccctgtatctgcaga | KNTLYLQMSSLKS |
| | yytncaratgwsnwsnytnaarwsngargayacn | tgagcagcctgaaaagcgaagataccgcgctgtatt | EDTALYYCTRGYS |
| | gcnytntaytaytgyacnmgnggntaywsnwsna | attgcacccgcggctatagcagcaccagctatgcga | STSYAMDFWGQG |
| | cnwsntaygcnatggayttytggggncarggnacn | tggatttttggggccagggcaccaccgtgaccagct | TTVTS (SEQ ID |
| | acngtnacnwsntrr (SEQ ID NO: 252) | aa (SEQ ID NO: 270) | NO: 288) |
| anti- | gayathgtnatgacncaracnccnwsnacnttyws | gatattgtgatgacccagaccccgagcacctttagcg | DIVMTQTPSTFSA |
| huEGFRvIII | ngcnacngtnggngaraargtnacnathacntgy | cgaccgtgggcgaaaaagtgaccanacctgccgc | TVGEKVTITCRAS |
| LC₁ | mgngcnwsncarwsngtncaywsngayggnaa | gcgagccagagcgtgcatagcgatggcaacacct | QSVHSDGNTYMQ |
| | yacntayatgcartggtaycarcaraatwsnggnm | atatgcagtggtatcagcagaaaagcggccgcggc | WYQQKSGRGPKF |
| | gnggnccnaarttyytnathtaygcngcnwsnaay | ccgaaatttctgatttatgcggcgagcaaccgctttag | LIYAASNRFSGVP |
| | mgnttywsnggngtnccngayaarwsnggnwsn | cggcgtgccggataaaagcggcagcggcggcgg | DKSGSGGGTDFT |
| | ggnggnggnacngayttyacnytnwsnggnatha | caccgattttaccctgagcggcattaacaccctgcag | LSGINTLQSEDFA |
| | ayacnytncarwsngargayttygcnacntaytayt | agcgaagattttgcgacctattattgccagcagggca | TYYCQQGTQLPR |
| | gycarcarggnacncarytnccnmgnacnttyggn | cccagctgccgcgcacctttggccagggcaccaaa | TFGQGTKVEATR |
| | carggnacnaargtngargcnacnmgnacntrr | gtggaagcgacccgcacctaa (SEQ ID | T (SEQ ID NO: |
| | (SEQ ID NO: 253) | NO: 271) | 289) |
| anti- | gayathgtnatgacncarwsnccnacnwsnttyw | gatattgtgatgacccagagcccgaccagctttagc | DIVMTQSPTSFSA |
| huEGFRvIII | sngcnacngtnggngaraargtnacnathwsntgy | gcgaccgtgggcgaaaaagtgaccattagctgccg | TVGEKVTISCRSS |
| LC₂ | mgnwsnwsncarwsngtncaywsngayggna | cagcagccagagcgtgcatagcgatggcaacagc | QSVHSDGNSYLS |
| | aywsntayytnwsntggtaycarcaraarwsnggn | tatctgagctggtatcagcagaaaagcggcaaagg | WYQQKSGKGPRF |
| | aarggnccnmgnttyytnathtayggngcnwsna | cccgcgctttctgatttatggcgcgagcaacaaattta | LIYGASNKFSGVP |
| | ayaarttywsnggngtnccngayaarwsnggnws | gcggcgtgccggataaaagcggcagcggcgcgg | DKSGSGAGTDYT |
| | nggngcnggnacngaytayacnytnwsnggnat | gcaccgattataccctgagcggcattaacaccgtgc | LSGINTVQSEDFA |
| | haayacngtncarwsngargayttygcnacntayt | agagcgaagattttgcgacctattattgccagcaggg | TYYCQQGTQLPR |
| | aytgycarcarggnacncarytnccnmgnacntty | cacccagctgccgcgcacctttggccagggcacca | TFGQGTKVEATG |
| | ggncarggnacnaargtngargcnacnggngcntr | aagtggaagcgaccggcgcgtaa (SEQ ID | A (SEQ ID NO: |
| | r (SEQ ID NO: 254) | NO: 272) | 290) |
| anti- | gayathgtnatgacnaaywsnccnacnwsnttya | gatattgtgatgaccaacagcccgaccagctttaccg | DIVMTNSPTSFTA |
| huEGFRvIII | cngcnacngtnggngaraargtnacnwsnathws | cgaccgtgggcgaaaaagtgaccagcattagctgc | TVGEKVTSISCKS |
| LC₃ | ntgyaarwsncarwsnytngtncaywsngayggn | aaaagccagagcctggtgcatagcgatggcaaca | QSLVHSDGNSYL |
| | aaywsntayytnwsntggytncaycarmgnwsn | gctatctgagctggctgcatcagcgcagcggccgcg | SWLHQRSGRAPR |
| | ggnmgngcnccnmgnttyytnathtaymgnath | cgccgcgctttctgatttatcgcattagcaaccgcttta | FLIYRISNRFSGVP |
| | wsnaaymgnttywsnggngtnccngaygartayg | gcggcgtgccggatgaatatggcagcggcgcggg | DEYGSGAGTDYT |
| | gnwsnggngcnggnacngaytayacnytnwsng | caccgattataccctgagcggcattaacaccattcag | LSGINTIQSEDFAS |
| | gnathaayacnathcarwsngargayttygcnws | agcgaagattttgcgagctattattgccagcagggca | YYCQQGTQLPRT |
| | ntaytaytgycarcarggnacncarytnccnmgna | cccagctgccgcgcacctttggccagggcaccaaa | FGQGTKVEATGA |
| | cnttyggncarggnacnaargtngargcnacnggn | gtggaagcgaccggcgcgtaa (SEQ ID | (SEQ ID NO: |
| | gcntrr (SEQ ID NO: 255) | NO: 273) | 291) |
| anti- | gargtncarytncarcarwsnggnacnytnytngcn | gaagtgcagctgcagcagagcggcaccctgctggc | EVQLQQSGTLLAK |
| huTfR HC₁ | aarccnggngcnwsngtnaaratgwsntgyaarg | gaaaccgggcgcgagcgtgaaaatgagctgcaaa | PGASVKMSCKAS |
| | cnwsnggntaywsntaywsnwsntaytggatgca | gcgagcggctatagctatagcagctattggatgcatt | GYSYSSYWMHWI |
| | ytggathaarcarmgnccnggncarggnytngart | ggattaaacagcgcccgggccagggcctggaatg | KQRPGQGLEWIG |
| | ggathggngcnathgayccnmgnaaywsngay | gattggcgcgattgatccgcgcaacagcgataccat | AIDPRNSDTIYNP |
| | acnathtayaayccnaayttyaarcayaargcnaar | ttataacccgaactttaaacataaagcgaaactgag | NFKHKAKLSAVTS |
| | ytnwsngcngtnacnwsnacnwsnacngcntay | cgcggtgaccagcaccagcaccgcgtatatggaag | TSTAYMEVNSLTN |
| | atggargtnaaywsnytnacnaaygargaywsng | tgaacagcctgaccaacgaagatagcgcggtgtatt | EDSAVYYCTPLYY |
| | cngtntaytaytgyacnccnytntaytaytaygayw | attgcaccccgctgtattattatgatagctggggccag | YDSWGQGTTLTV |
| | sntggggncarggnacnacnytnacngtnwsnws | ggcaccaccctgaccgtgagcagctaa (SEQ ID | SS (SEQ ID |
| | ntrr (SEQ ID NO: 256) | NO: 274) | NO: 292) |
| anti- | gargtncarytncarcarwsnggnacngtnytngcn | gaagtgcagctgcagcagagcggcaccgtgctgg | EVQLQQSGTVLA |
| huTfR HC₂ | aarccngcngcnwsnatgmgnatgwsntgyaar | cgaaaccggcggcgagcatgcgcatgagctgcaa | KPAASMRMSCKA |
| | gcnwsnggntayacnathwsnwsntaytggatgc | agcgagcggctataccattagcagctattggatgcat | SGYTISSYWMHWI |
| | aytggathaarcarmgnccnggncarggnytnga | tggattaaacagcgcccgggccagggcctggattg | KQRPGQGLDWIV |
| | ytggathgtnggnathgayccnmgnaaywsnga | gattgggcattgatccgcgcaacagcgataccgc | GIDPRNSDTAYNP |
| | yacngcntayaayccncarttyaarcayaargcna | gtataacccgcagtttaaacataaagcgaaactgac | QFKHKAKLTAVTS |
| | arytnacngcngtnacnwsnwsnwsnacngcnt | cgcggtgaccagcagcagcaccgcgtatatggaac | SSTAYMELNSLTN |
| | ayatggarytnaaywsnytnacnaaygaygayws | tgaacagcctgaccaacgatgatagcgcggtgtatt | DDSAVYYCTPLYY |
| | ngcngtntaytaytgyacnccnytntaytayttygay | attgcaccccgctgtattattttgatagctggggccag | FDSWGQGTTLTV |
| | wsntggggncarggnacnacnytnacngtnwsn | ggcaccaccctgaccgtgagcagctaa (SEQ ID | SS (SEQ ID |
| | wsntrr (SEQ ID NO: 257) | NO: 275) | NO: 293) |
| anti- | gargtncarytncarcarwsnggnacnytnytngcn | gaagtgcagctgcagcagagcggcaccctgctggc | EVQLQQSGTLLA |
| huTfR HC₃ | mgnccnggnathacngtnaaratgwsntgyaarg | gcgcccgggcattaccgtgaaaatgagctgcaaag | RPGITVKMSCKAS |
| | cnwsnggntayacngcnacnacntaytggatgca | cgagcggctataccgcgaccacctattggatgcattg | GYTATTYWMHWI |
| | ytggathaarcarmgnccnggncarggnytngary | gattaaacagcgcccgggccagggcctggaactg | KQRPGQGLELIVA |
| | tnathgtngcngcngayccnmgnaaywsngaya | attgtggcggcggatccgcgcaacagcgataccatt | ADPRNSDTIYQPQ |
| | cnathtaycarccncartayaarcayaarggnaary | tatcagccgcagtataaacataaaggcaaactgac | YKHKGKLTAVTST |
| | tnacngcngtnacnwsnacnacnwsnathtayat | cgcggtgaccagcaccaccagcatttatatggatctg | TSIYMDLNSLTNE |
| | ggayytnaaywsnytnacnaaygargaywsngc | aacagcctgaccaacgaagatagcgcggtgtattat | DSAVYYCTPLYYF |
| | ngtntaytaytgyacnccnytntaytayttygarws | tgcaccccgctgtattattttgaaagctggggccagg | ESWGQGTTLTVS |
| | ntggggncarggnacnacnytnacngtnwsnwsntr | gcaccaccctgaccgtgagcagctaa (SEQ ID | S (SEQ ID NO: |
| | r(SEQ ID NO: 258) | NO: 276) | 294) |
| anti- | gayathmgnatgwsncarwsnccnacnwsnatg | gatattcgcatgagccagagcccgaccagcatgtat | DIRMSQSPTSMY |
| huTfR LC₁ | taygcnwsnytnggngarmgngtnacntayacnt | gcgagcctgggcgaacgcgtgacctatacctgccg | ASLGERVTYTCR |
| | gymgngcnwsncargayathaayaaytaygtntg | cgcgagccaggatattaacaactatgtgtgctggtttc | ASQDINNYVCWF |
| | ytggttycarcaraarccnggnaarwsnccnaarw | agcagaaaccgggcaaaagcccgaaaagcctga | QQKPGKSPKSLIY |
| | snytnathtayaargcnaaymgnytngtngayggn | tttataaagcgaaccgcctggtggatggcgtgccga | KANRLVDGVPSR |
| | gtnccnwsnmgntaywsnggnwsnggnwsng | gccgctatagcggcagcggcagcggccaggaata | YSGSGSGQEYSL |
| | gncargartaywsnytnacnathwsnwsnytngar | tagcctgaccattagcagcctggaatatgaagatatg | TISSLEYEDMGIYY |
| | taygargayatgggnathtaytaytgyytncartty | ggcatttattattgcctgcagtttgatgaatttccgt | CLQFDEFPYTFG |
| | gaygarttyccntayacnttyggnggnggnacnaar | atacctttggcggcggcaccaaactggaataa (SEQ | GGTKLEIK (SEQ |
| | ytngartrr(SEQ ID NO: 259) | ID NO: 277) | ID NO: 295) |
| anti- | gayathaaratgacncarwsnccnwsnwsnatgt | gatattaaaatgacccagagcccgagcagcatgtat | DIKMTQSPSSMYA |
| huTfR LC₂ | aygcnwsngtnggngaymgngtnacnttyacntg | gcgagcgtgggcgatcgcgtgacctttacctgcaaa | SVGDRVTFTCKA |
| | yaargcnwsncargarathaayaaytayytntgytg | gcgagccaggaaattaacaactatctgtgctggtttc | SQEINNYLCWFQ |
| | gttycarcarmgnccnggnaaracnccnmgnacn | agcagcgcccgggcaaaaccccgcgcaccctgat | QRPGKTPRTLIYR |
| | ytnathtaymgngcnaayaarytngtngayggngt | ttatcgcgcgaacaaactggtggatggcgtgccgag | ANKLVDGVPSRF |
| | nccnwsnmgnttywsnggnwsnggnwsngcnc | ccgctttagcggcagcggcagcgcgcaggattata | SGSGSAQDYSLTI |
| | argaytaywsnytnacnathwsnwsnytngartay | gcctgaccattagcagcctggaatatgaagatatgg | SSLEYEDMGIYYC |
| | gargayatgggnathtaytaytgyytncartaygayg | gcatttattattgcctgcagtatgatgattttccgta | LQYDDFPYTFGG |
| | ayttyccntayacnttyggnggnggnacnaarytng | tacctttggcggcggcaccaaactggaaattcgctaa | GTKLEIR (SEQ ID |
| | arathmgntrr (SEQ ID NO: 260) | (SEQ ID NO: 278) | NO: 296) |
| anti- | gaygcnaaratgacnaaywsnccnwsnwsnatg | gatgcgaaaatgaccaacagcccgagcagcatgt | DAKMTNSPSSMY |
| huTfR LC₃ | taygcnwsnytnggngarmgngtnacnttyacntg | atgcgagcctgggcgaacgcgtgacctttacctgca | ASLGERVTFTCKA |
| | yaargcnwsncargayathaaycarttyytntgytg | aagcgagccaggatattaaccagtttctgtgctggttt | SQDINQFLCWFQ |
| | gttycarcaraarccnggnaaracnccnaaracnyt | cagcagaaaccgggcaaaaccccgaaaaccctg | QKPGKTPKTLIYR |
| | nathtaymgngcnaaymgnytngtngayggngt | atttatcgcgcgaaccgcctggtggatggcgtgccg | ANRLVDGVPSRF |
| | nccnwsnmgnttywsnggnacnggnwsnggnc | agccgctttagcggcaccggcagcggccaggattat | SGTGSGQDYSLTI |
| | argaytaywsnytnacnathwsnwsnytngartty | agcctgaccattagcagcctggaatttgaagatatgg | SSLEFEDMGIYYC |
| | gargayatgggnathtaytaytgygtncartaygay | gcatttattattgcgtgcagtatgatgaatttccgtat | VQYDEFPYSFGG |
| | garttyccntaywsnttyggnggnggnacnaarytn | agctttggcggcggcaccaaactggaaattaaataa | GTKLEIK (SEQ ID |
| | garathaartrr (SEQ ID NO: 261) | (SEQ ID NO: 279) | NO: 297) |

*The consensus sequences are degeneracy sequences which follow the standard IUPAC symbols for DNA (R = A or G; Y = C or T; M = A or C; W = A or T; S = C or G; B = C, G or T; D = A, G or T; H = A, C or T; V = A, C or G; and N is any nucleotide (A, C G or T)).

According to the embodiments described herein, the biotag biomarker binding domains described herein may target one or more tumor cells that are benign or malignant. The one or more tumor cells may be part of an intact primary or metastatic tumor or may be circulating tumor cells (single or clustered) found in a physiological fluid, e.g., blood, serum, plasma, urine, prostate fluid, tears, mucus ascites fluid, oral fluid, saliva, semen, seminal fluid, mucus, stool, sputum, cerebrospinal fluid (CSF), bone marrow, lymph, and fetal fluid. Circulating tumor cells (CTCs) are cells of epithelial origin that are present in the circulation of a patient's physiological fluids with various solid tumors or malignancies. They are derived from clones of the primary tumor and are malignant. (See Fehm et al. Clin Cancer Res. 8:2073-84, 2002, which is hereby incorporated by reference in its entirety as if fully set forth herein). CTCs may be considered an independent diagnostic for cancer occurrence, and in addition to standard diagnostics, may be used as a screening test for persons at high risks of cancer (e.g., BRCA1,2 mutants) before clinical symptoms, progression of carcinomas in several cancers, including, but not limited to, breast cancer, brain cancer (e.g., glioma), colorectal cancer, melanoma, ovarian cancer, prostate cancer, thyroid cancer, lung cancer, colorectal, testicular, and gastric cancer. CTCs may also be detectable before the primary tumor is established, thus allowing early stage diagnosis. Therefore, they may provide an important tool for early stage diagnosis. They may also decrease in number in response cancer therapy, so the ability to quantitate the number of CTCs allows for monitoring the effectiveness of a given therapeutic regimen. They can also be used as a tool for monitoring for recurrence in patients with no measurable disease. Further, CTCs may be used to predict progression-free survival and overall survival, as the presence or number of CTCs in patients with metastatic carcinoma has been shown to be correlated with both progression-free and overall survival (see e.g., Cristofanilli et al. J Clin Oncol 23(1):1420-1430, 2005; Cristofanilli et al., NEJM 351(8):781-791, 2004).

Antioxidant-Enzymes and Markers of Apoptosis and/or Necrosis. In some embodiments, a biotag target binding domain may be an antioxidant enzyme blocker, (also known as an anti-ROS enzyme blocker, or anantioxidative enzyme inhibitor). Cellular exposure to certain types of stresses induces the generation of reactive oxygen species (ROS), and excessive ROS generation induces DNA injury, which may induce cell death or carcinogenesis. The intrinsic antioxidant enzyme system is a defense mechanism that protects cells against oxidative injury. This system is composed of 3 types of protein, superoxide dismutase (SOD), which converts superoxide to hydrogen peroxide, and catalase and glutathione peroxidase (GPx), which convert hydrogen peroxide to water. There are two types of SOD, namely, manganese (Mn)-SOD, which exist mainly in mitochondria, and copper, zinc (Cu, Zn)-SOD, which exists mainly in the cytoplasm. This system converts two toxic radicals, namely, superoxide and hydrogen peroxide into water. Thus, in some embodiments, a biotag may include one or more anti-ROS enzyme blocker that target SOD, GPx, caspase or a combination thereof. They inhibit antioxidant.enzymes, what not only causes rapid increases in ROS by itself, but also disables biotag (aka oncotags) sensitized cancer cells to neutralize increased number of ROS upon radiation therapy.

In some embodiments, an anti-ROS enzyme blocker that targets an antioxidant enzyme (e.g., SOD, GPx or caspase) inhibits/blocks the antioxidant enzyme's activity, thereby increasing a cell's exposure to ROS and inflicting oxidative stress within the target cell. Therefore, when a biotag that includes such a binding domain, excessive ROS buildup increases the induction of apoptosis or necrosis in the target cell. In some embodiments, an anti-ROS enzyme blocker is a cancer-specific anti-ROS enzyme blocker that specifically targets cancer cells, resulting in the induction of apoptosis and/or necrosis of the cancer cells, but not in healthy cells.

In some embodiments, induction of apoptosis and/or necrosis of cells resulting from treatment with a biotag having an anti-ROS enzyme blocker can be detected using a marker for cell death. Such a marker may target substances that are indicative of apoptosis, necrosis, or both. In one embodiment, the cell death marker is, but is not limited to, phophatidylserine, which is usually found on the cytosolic side of cell membranes. Upon induction of apoptotic cell death, phophatidylserine becomes exposed on the surface of the cell. Phosphatidylserine is also a marker for necrosis. The phophatidylserine may be detected using a metal nanoparticle-associated biotag as described herein that includes a target binding domain that targets phophatidylserine.

Functional Domains

According to the embodiments described herein, biotags described herein may include one or more functional domains. Such functional domains may include, but are not limited to, an internalization domain, an endosomal escape domain, a lysosomal escape domain and a nuclear localization signal domain.

Rapid internalization of the biotags upon binding a targeted biomarker receptor (e.g., ErbB1-4, EGFRvIII or TfR) leads to their rapid clearance and synthesis of the new receptors followed by their trafficking to the cell surfaces. These processes lead to constant import of the biotags into the cells. When two cells, one expressing 3M cells on its cell surface and the second one expressing 30K receptors on its surface, are exposed to the same concentration of biotags tagged with gold, the first one will generate a minimum or approximately 100× stronger signal for imaging than the latter. With the refresh rate of about 1000 per hour, the total account for the imported biotags into the cells reaches 0.2-0.4×10²³ or 0.2-0.4M. This catapults the concentration of the gold atoms tagging biotags to molar (M) range, which is well within the detection threshold (DT) and with a signal to noise ratio to 100/1. This calculation accounts for average recycling of the receptors, during which time, the biotags pass through the endosomal recycling pathway, and subsequently escape from these pathway to saturate cell cytoplasm with gold atoms. In addition, the cancer cells have much higher metabolism and proliferation rate. Therefore, in-take of biotags in these cells is much higher than in healthy cells (except inflammatory or regenerating cells).

Presence of endosomal escape signals on the biotags results in their escape from the endosome and lysosome pathways, while entering the cytoplasm. They remain retained there or if nuclear localization signal is included, then they are retained in the cancer cell nuclei. With almost entire clearance of the scFv from blood within one hour, the residual signal from the presence of the biotags in the circulation is minimal or absent, while the signal from the biotags tagged with nanogold or superparamagnetic particles retained within the cells remain unchanged. This catapults the signal to noise ratio far within the detection range of SPR, Raman, X-ray, CT, MRI and NMR.

Thus, in some embodiments, a biotag has an internalization domain, which is a signal that causes the nanoprobe to enter or to be internalized into the labeled cancer cell. In one embodiment, the internalization domain may include, but is not limited to the following sequences: YHWYGYTPQNVI (SEQ ID NO:19); NPVVGYIGERPQYRDL (SEQ ID NO:20); or ICRRARGDNPDDRCT (SEQ ID NO:21).

In some embodiments, a biotag also has an endosomal escape domain and a lysosomal escape domain, which are signals that cause the internalized biotag to escape from endocytotic and lysosomal pathways, resulting in permanently tagging the target cancer cell with the biotag, acting as a reporter. In one embodiment, the endosomal escape domain may include, but is not limited to the following sequences: GIGAVLKVLTTGLPALISWIKRKRQQ (SEQ ID NO:22); GRKKRRQRRRPPQ (SEQ ID NO:23); or GLFGAIAGFIENGWEGMIDGWYG (SEQ ID NO:24). The lysosomal escape domain may include, but is not limited to the following oligopeptide sequences: CHK6HC (SEQ ID NO:25); or H5WYG (SEQ ID NO:26). In some embodiments, a biotag has a nuclear localization sequence (PKKKRKV from SV 40 Large T antigen or KR[PAATKKAGQA]KKKK from nucleoplasmin as described in Malecki 1996), which is the signal guiding the entry of the biotag into the cell nucleus.

The biotag domains may be associated with each other by any suitable method of conjugation or connection (or association), known in the art. According to some embodiments, the biotag domains may be connected using known methods of linking proteins, peptides, antibodies and functional fragments thereof, metals, atoms and molecules. In one aspect, the domains may be designed with overlapping complementary strands so that they may be joined together. In one aspect, the biotag domains are joined by site-specific conjugation using a suitable linkage or bond. In another aspect, the biotag domains may be joined by a bifunctional linker, the design of which would be known by one skilled in the art. Site-specific conjugation is more likely to preserve the binding activity of an antibody or functional antibody fragment. Alternatively, other linkages or bonds used to connect the biotag domains may include, but is not limited to, a covalent bond, a non-covalent bond, a chemical bond, an electrostatic bond, an intermolecular force, an ionic bond, a hydrogen bond, van der Waal forces, a dipole-dipole interaction, metallic bonds, a sulfide linkage, a hydrazone linkage, a hydrazine linkage, an ester linkage, an amido linkage, and amino linkage, an imino linkage, a thiosemicabazone linkage, a semicarbazone linkage, an oxime linkage and a carbon-carbon linkage. In another aspect the domains may be fused-in-frame, the DNA coding sequences by overlap extension, or may otherwise be formed by a single recombinant protein.

Reporters and Reporter Binding Domains

In some embodiments, the biotags described herein include a reporter to allow said biotags to be detected when internalized by the target cell. Thus, a biotag that includes a reporter may deliver a diagnostic payload to the cell. In some embodiments, the diagnostic payload may be delivered by combination with a contrast for use with diagnostic imaging techniques such as radiography, computed tomography (CT), magnetic resonance imaging (MRI), ultrasonography (USG), fluoroscopy, and Raman spectroscopy as described below. Alternatively, the biotags may be modified to accept radionuclides for use with diagnostic imaging techniques such as positron emission tomography (PET), single photon emission computed tomography (SPECT) and gamma scintigraphy.

According to the embodiments described herein, the reporter may be any suitable diagnostic agent. A “diagnostic agent” is an atom, molecule, or compound that is useful in diagnosing, detecting or visualizing a disease. According to the embodiments described herein, diagnostic agents may include, but are not limited to, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions). In addition, it should be noted that some nanoparticles, for example quantum dots and metal nanoparticles, e.g., noble metal, superparamagnetic metal, and core-shell nanoparticles (described below) may also be suitable for use as a detection agent.

Radioactive substances that may be used as diagnostic agents in accordance with the embodiments of the disclosure include, but are not limited to, ¹⁸F, ³²P, ³³P, ⁴⁵Ti, ⁴⁷Sc, ⁵²Fe, 59Fe, ⁶²Cu, ⁶⁴Cu, ⁶⁷Cu, ⁶⁷Cu, ⁶⁸Ga, ⁷⁵Sc, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as diagnostic agents in accordance with the embodiments of the disclosure include, but are not limited to, ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, Ru, and Lu.

Contrast agents that may be used as diagnostic agents in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof. Targeted contrast agents that may be used according to the embodiments described herein are described in further detail below.

Bioluminescent and fluorescent compounds or molecules and dyes that may be used as diagnostic agents in accordance with the embodiments of the disclosure include, but are not limited to, fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, fluorescent metals including, but not limited to Eu, Tb, Ru, fluorescent amino acids (e.g., Tyrosine), or combination thereof. According to embodiments described herein, a fluorescent reporter may be used to sort cells targeted by the biotags described herein using fluorescent flow cytometry methods known in the art including, but not limited to, fluorescence-activated cell sorting (FACS).

Enzymes that may be used as diagnostic agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the biotags described herein include a reporter binding domain to provide a binding site for the reporter. For example, when the reporter or diagnostic agent is a metal (e.g., a noble metal or superparamagnetic metal) or paramagnetic ion, the biotag may include a metal binding domain. In such case, the reporter or diagnostic agent may be reacted with a reagent having a long tail with one or more chelating groups attached to the long tail for binding these ions. The long tail may be a polymer such as a polylysine, polysaccharide, or other derivatized or derivatizable chain having pendant groups to which may be bound to a chelating group for binding the ions. Examples of chelating groups that may be used according to the disclosure include, but are not limited to, ethylenediaminetetraacetic acid (EDTA), EGTA, diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the antibody or functional antibody fragment by a group which enables formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium, gadolinium, iodine, and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al—¹⁸F complex, to a targeting molecule for use in PET analysis.

According to some embodiments of the disclosure, a biotag designed with a metal binding domain (MBD) may be tagged with a metal nanoparticle tag. In one embodiment, the MBD may include, but is not limited to the following sequences: (Gly-)ₙ-Cys (SEQ ID NO:27); (Gly-Arg-)ₙ-Cys (SEQ ID NO:28); (Gly-Lys-)ₙ-Cys (SEQ ID NO:29); (Gly-Asp-Gly-Arg)ₙ-Cys (SEQ ID NO:30); (Gly-Glu-Gly-Arg)ₙ-Cys (SEQ ID NO:31); (Gly-Asp-Gly-Lys)ₙ-Cys (SEQ ID NO:32); (Gly-Glu-Gly-Lys)ₙ-Cys (SEQ ID NO:33); MAP16-B; (Glu-Glu-Glu-Glu-Glu)ₙ (SEQ ID NO:34); (Glu-Glu-Glu-Glu-Glu-Glu)ₙ (SEQ ID NO:35); (Asp-Asp-Asp-Asp-Asp)ₙ (SEQ ID NO:36); (Asp-Asp-Asp-Asp-Asp-Asp)ₙ (SEQ ID NO:37); Phe-His-Cys-Pro-Tyr-Asp-Leu-Cys-His-Ile-Leu (SEQ ID NO:38); (Gly-Asp-Gly-Arg)ₙ-(His)5,6 (SEQ ID NO:39); (Gly-Glu-Gly-Arg)ₙ-(His)5,6 (SEQ ID NO:40); (Gly-Asp-Gly-Lys)ₙ-(His)5,6 (SEQ ID NO:41); (Gly-Glu-Gly-Lys)ₙ-(His)5,6 (SEQ ID NO:42); (Gly-Arg-)ₙ-(His)5,6 (SEQ ID NO:43); or (Gly-Lys-v-(His)5,6 (SEQ ID NO:44). Moreover, the amino acid dendrimers including but not limited to MAP, may have functional domains modified to link with scFv, sdFv, CDR, SDR, while chelating metals through functionalized DOTA, DTPA, TETA, etc

The metal nanoparticle tag allows for visualization and/or quantification of the biotag using diagnostic imaging techniques such as radiography, computed tomography (CT), magnetic resonance imaging (MRI), Raman, gamma scintigraphy, PET and SPECT as described below. Additionally, the metal nanoparticle tag may act as a radiosensitizer to render the targeted cells more sensitive to radiation therapy as compared to healthy, non-targeted cells (Brun et al. 2009). The metal nanoparticle tags may be formed from a single suitable solid metal or a related metal compound (e.g., Fe₃O₄, γ-Fe₂O₃, ferritin), a combination of two or more suitable metals or related metal compounds (e.g., Fe₃O₄, γ-Fe₂O₃, ferritin) or a combination thereof. In some embodiments, the metal nanoparticle tag may comprise a nanoparticle derived from a noble metal, including, but not limited to, Gold (Au), Platinum (Pt), Palladium (Pd) and Silver (Ag). In other embodiments, the metal nanoparticle tag may comprise a superparamagnetic metal, including, but not limited to, Europium (Eu), Gadolinium (Gd), Iron (Fe), Nickel (Ni), Cobalt (Co) or a related metal compound (e.g., Fe₃O₄, γ-Fe₂O₃, ferritin). In other embodiments, the metal nanoparticle tag may comprise a fluorescent metal, including, but not limited to Eu, Tb. The superparamagnetic, heavy, or fluorescent metal tag can be made as chelated nanoclusters or as core-shell nanoparticles, which have a superparamagnetic core that is sealed inside a noble-metal layer (or “core-shell”). This shell eliminates the risk of biotoxicity through transmetallation, while assuring the biocompatibility of internalized biotags (aka oncotags).

According to embodiments of the disclosure, a molecular probe designed with a target binding domain having an MBD may be tagged with a metal nanoparticle tag to form a biotag to be used in conjunction with the methods described herein. In one embodiment, the MBD may include, but is not limited to the following sequences:

(SEQ ID NO: 27)
(Gly-)ₙ-Cys;
(SEQ ID NO: 28)
(Gly-Arg-)ₙ-Cys;
(SEQ ID NO: 29)
(Gly-Lys-)ₙ-Cys;
(SEQ ID NO: 30)
(Gly-Asp-Gly-Arg)ₙ-Cys;
(SEQ ID NO: 31)
(Gly-Glu-Gly_Arg)ₙ-Cys;
(SEQ ID NO: 32)
(Gly-Asp-Gly-Lys)ₙ-Cys;
(SEQ ID NO: 33)
(Gly-Glu-Gly-Lys)ₙ-Cys;
(SEQ ID NO: 34)
MAP16-B; (Glu-Glu-Glu-Glu-Glu)ₙ;
(SEQ ID NO: 35)
(Glu-Glu-Glu-Glu-Glu-Glu)ₙ;
(SEQ ID NO: 36)
(Asp-Asp-Asp-Asp-Asp)ₙ;
(SEQ ID NO: 37)
(Asp-Asp-Asp-Asp-Asp-Asp)ₙ;
(SEQ ID NO: 38)
Phe-His-Cys-Pro-Tyr-Asp-Leu-Cys-His-Ile-Leu;
(SEQ ID NO: 39)
(Gly-Asp-Gly-Arg)ₙ-(His)5,6;
(SEQ ID NO: 40)
(Gly-Glu-Gly_Arg)ₙ-(His)5,6;
(SEQ ID NO: 41)
(Gly-Asp-Gly-Lys)ₙ-(His)5,6;
(SEQ ID NO: 42)
(Gly-Glu-Gly-Lys)ₙ-(His)5,6;
(SEQ ID NO: 43)
(Gly-Arg-)ₙ-(His)5,6;
or
(SEQ ID NO: 44)
(Gly-Lys-v-(His)5,6.

In some embodiments, the biotags described herein may be used to deliver a diagnostic payload by combining them with a contrast for use with diagnostic imaging techniques such as x-ray radiography, computed tomography (CT), magnetic resonance imaging (MRI), fluoroscopy, and Raman as described below. Alternatively, the biotags may be modified to accept radionuclides for use with diagnostic imaging techniques such as positron emission tomography (PET), single photon emission computed tomography (SPECT) and gamma scintigraphy. In other embodiments, the biotags may be used as a radiosensitizer to deliver a therapeutic payload, by converting x-ray or electromagnetic radiation into precise treatment tools to kill cells that have internalized the biotag while leaving healthy cells that are free of biotags untouched. In other embodiments, a biotag may be designed to deliver a combination of diagnostic and therapeutic payloads, to include one or more diagnostic payloads, and one or more therapeutic payload. In other embodiments, the biotags or oncotags may be used for ex vivo diagnosis of cancer by detecting the cancer cells in physiological fluids drawn in the laboratory and their, in vitro by means of flow cytometry (FCM), spectroscopy, nuclear magnetic resonance (NMR), surface Plasmon resonance (SPR), radio-scintillation.

The metal nanoparticle tag allows for visualization and/or quantification of the biotag using diagnostic imaging techniques such as radiography, computed tomography (CT), magnetic resonance imaging (MRI), Raman, gamma scintigraphy, PET and SPECT as described below. Additionally, the metal nanoparticle tag may act as a radiosensitizer to render the targeted cells more sensitive to radiation therapy as compared to healthy, non-targeted cells (Brun et al. 2009). The metal nanoparticle tags may be formed from a single suitable solid metal or from a combination of two or more suitable metals. In some embodiments, the metal nanoparticle tag may comprise a nanoparticle derived from a noble metal, including, but not limited to, Gold (Au), Platinum (Pt), Palladium (Pd) and Silver (Ag). In other embodiments, the metal nanoparticle tag may comprise a superparamagnetic metal, including, but not limited to, Europium (Eu), Gadolinium (Gd), Iron (Fe), Nickel (Ni) or Cobalt (Co). The superparamagnetic metal tag can be made as chelated nanoclusters or as core-shell nanoparticles, which have a superparamagnetic core that is sealed inside a noble-metal layer (or “core-shell”).

Therapeutic Agents

In another embodiment, the biotag may include or be further conjugated to a therapeutic agent. A “therapeutic agent” as used herein is an atom, molecule, or compound that is useful in the treatment of cancer or other conditions associated with a cancer biomarkers as described herein. Examples of therapeutic agents that may be associated with the biotag include, but are not limited to, drugs, chemotherapeutic agents, therapeutic antibodies and antibody fragments, toxins, radioisotopes, enzymes (e.g., enzymes to cleave prodrugs to a cytotoxic agent at the site of the tumor), nucleases, hormones, immunomodulators, antisense oligonucleotides, chelators, boron compounds, photoactive agents and dyes. As described above, the metal nanoparticle tag may act as a therapeutic agent, acting as a radiosensitizer to render the targeted cells more sensitive to radiation therapy as compared to healthy, non-targeted cells.

Chemotherapeutic agents are often cytotoxic or cytostatic in nature and may include alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as therapeutic agents in accordance with the embodiments of the disclosure include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte-colony stimulating factor, granulocyte macrophage-colony stimulating factor, hexamethylmelamine, hydrocortisone hydroxyurea, ibritumomab, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

Therapeutic antibodies and functional fragments thereof, that may be used as therapeutic agents in accordance with the embodiments of the disclosure include, but are not limited to, alemtuzumab, bevacizumab, cetuximab, edrecolomab, gemtuzumab, ibritumomab tiuxetan, panitumumab, rituximab, tositumomab, and trastuzumab

Toxins that may be used as diagnostic agents in accordance with the embodiments of the disclosure include, but are not limited to, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

Radioisotopes that may be used as therapeutic agents in accordance with the embodiments of the disclosure include, but are not limited to, ³²P, ⁸⁹Sr, ⁹⁰Y. ^{99m}Tc, ⁹⁹Mo, ¹³¹I, ¹⁵³Sm, ¹⁷⁷Lu, ¹⁸⁶Re, ²¹³Bi, ²²³Ra and ²²⁵AC.

In another embodiment, the biotags described herein may include or be conjugated to a nanoparticle. The term “nanoparticle” refers to a microscopic particle whose size is measured in nanometers, e.g., a particle with at least one dimension less than about 100 nm. Nanoparticles are particularly useful as detectable substances because they are small enough to scatter visible light rather than absorb it. For example, gold nanoparticles possess significant visible light extinction properties and appear deep red to black in solution. As a result, compositions comprising PSCA-specific antibody or fragments conjugated to nanoparticles can be used for the in vivo imaging of tumors or cancerous cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles, when conjugated to a PSMA antibody or functional antibody fragment, can be used as imaging agents for the in vivo detection of tumor cells as described above. Alternatively, nanoparticles can be used in therapeutic applications as drug carriers that, when conjugated to a biotag described herein, deliver chemotherapeutic agents, hormonal therapeutic agents, radiotherapeutic agents, toxins, or any other cytotoxic or anti-cancer agent known in the art to the target cancer cells.

The biotags described herein have at least the following advantageous properties. First, they label cancer cells permanently, unlike monoclonal or polyclonal antibody-based (or functional fragments thereof) probes. Antibody-based probes bind to the outside of the cells, in a non-permanent fashion. Such probes bind and unbind their targed according to their on/off characteristics, which are dependent on the physiological environment conditions. This property can result in false negative results (i.e., patients will leave the hospital having undetected cancer). To the contrary, because biotags are internalized and escape from the lysosomal and endosomal processes, the cells are permanently tagged, resulting a more sensitive and accurate diagnostic and treatment outcome. Second, they generate a stable signal that does not fade like fluorochromes. Third, they are highly specific to cancer cells and do not result in non-specific binding (e.g., binding to receptors for the Fc portion of an IgG (FcR)) that is common in antibody based probes. Fourth, they can label multiple domains of one cancer cell receptor due to their small size, thereby enhancing the signal and detecting mutations, unlike 155 kDa mono- or polyclonal antibodies, which prevent multiple labels to reach the target due to steric hindrance or large magnetic, optical, or colloidal beads, which may be phagocytosed by macrophages and raise false positive results. Fifth, they are able to permanently label and act as a radiosensitizer in cancer cells, resulting in sensitization of cancer cells only. Because only the cancer cells are sensitized, the effective dose of radiation is much lower than what is generally used in current treatment regimens (Brun et al. 2009). This property is a significant improvement over current radiation treatment methods, because radiation treatment may be used at a dose that is not lethal to non-labeled healthy cells, but is lethal to the labeled cancer cells—resulting in a treatment that is at least equally effective to current treatments with far fewer side effects.

Targeted Contrast Compositions

One problem with designing new contrast agents for molecular imaging has been the lack of methods that provide information concerning contrast agents and their cell surface distribution and subcellular trafficking at the supramolecular level directly in situ. The introduction of Electron Energy Loss Spectroscopid Imaging (EELSI) and Energy Dispersive X-Ray Analysis Spectroscopic Imaging (EDXSI) provided sensitive methods of molecular detection in situ. (Malecki 1995, Malecki et al 2001). In EELSI and EDXSI, genetically engineered antibodies tagged with atoms of selected exogenous elements can be localized within three-dimensional architecture of cells and cell organelles with atomic accuracy. In combination with rapid cryo-immobilization (Malecki 1992), which “freezes” within nanoseconds living processes in their living configuration, information obtained from these imaging methods is similar to endogenous processes. Therefore, the methods developed herein are advantageous because they exploit the molecular mechanisms governing bio-distribution and bio-compatibility. The targeted contrast described herein provides a similarly sensitive method for detecting such information in vivo.

According to some embodiments, a targeted contrast composition is provided comprising a contrast agent and the biotags described herein. The targeted contrast composition may be used with diagnostic imaging techniques such as X-ray, computed tomography (CT), fluoroscopy, Raman, MRI, PET, SPECT, USG, SPR, FCM, scintigraphy, and NMR to provide a more accurate localization and diagnosis of malignant tumors in a subject's body in vivo.

A contrast agent is a substance that is used to enhance the contrast of structures or fluid within the body in diagnostic imaging techniques. Contrast agents are commonly used to enhance the visibility of blood vessels, respiratory system, and the urinary and gastrointestinal tract. In some embodiments described herein, a targeted contrast composition may be used to enhance visibility of tumor cells that express a cancer biomarker. In one embodiment, the cancer biomarkers are ErbB1-4 and TfR (including their wild types and mutants).

Examples of contrast agents include, but are not limited to, barium, water, water soluble iodine, iodine mixed with water or oil, sterile saline, air occurring naturally or introduced into the body and paramagnetic substances. The type of contrast agent used can be classified, generally, based on the type of imaging technique used. Such techniques may include, but are not limited to, X-ray, fluorescence or magnetic resonance or may be based on injection of radionuclides. However, the injection of radionuclides introduces sources of ionizing radiation into the patients' bodies to provide a signal to show the distribution of the radionuclides while exposing patients to the risks of mutations, but without providing any anatomical information.

Targeted contrast compositions for x-ray-based diagnostic imaging and therapy. Iodine (I) and barium (Ba) are the most common types of contrast agents for enhancing x-ray based imaging methods such as radiography and CT. Various iodinated contrast media exist, with variations occurring between the osmolarity, viscosity and absolute iodine content of different agents. For example, contrast agents for x-ray based diagnostic imaging are based on tri-iodobenzene with substituents added for water solubility. Diatrizoate, an ionic corm, was introduced in 1954, but the high osmolality of this compound (1.57 osm/kg for a 300 mg/ml solution) was found to be the source of chemotoxicity. In the 1970s, a non-ionic form, iohexyl, lowered osmolality (0.67 osm/kg), and is still widely used today under the names Omnipaque® and Exypaque®. Because osmolality was still excessive, a dimeric form was introduced, iodixanol (Acupaque® and Visipaque®; 0.5 osm/kg). Intravascular agents based on other mid-Z to high-Z elements have not been successful due to toxicity, performance or cost. The low molecular weights of the iodine agents (diatrizoate, 613; iohexyl, 821; iodixanol, 1550) effect rapid renal clearance and vascular permeation, necessitating short imaging times. Therefore, intra-arterial catheterization is commonly needed, but carries the risks of arterial puncture, dislodgement of plaque, stroke, myocardial infarction, anaphylactic shock and renal failure. A further shortcoming of the available contrast agents is that, even when conjugated with antibodies or other targeting moieties, they fail to deliver iodine to desired sites at detectable concentrations.

Several other experimental X-ray based contrast materials are used as blood pool agents, including standard iodine agents encapsulated in liposomes, a dysprosium-DTPA-dextran polymer, polymeric iodine-containing PEG-based micelles, perfluoroctyl bromide, derivatized polylysine linked to iodine, and iodine linked to a polycarboxylate core (P743, MW=12.9 kDa). Iron nanoparticles have also been used successfully as magnetic resonance imaging (MRI) contrast agents. Nevertheless, none of these contrast agents were targeted to specifically bind to any biomarker or other biologic target.

In one embodiment, the metal nanoparticle tag associated with the nanoparticles used herein is gold. With a higher atomic number (Au, 79 vs. I, 53), and a higher absorption coefficient (at 100 keV: gold=5.16 cm²/g; iodine=1.94 cm²/g; soft tissue=0.169 cm²/g; and bone=0.186 cm²/g), gold provides about 2.7 times greater contrast per unit weight than iodine. Imaging gold at 80-100 keV reduces interference from bone absorption and takes advantage of lower soft tissue absorption which reduces patient radiation dose. Further, the higher molecular weight of noble metal nanoparticles permits much longer blood retention, so that useful imaging may be obtained after intravenous injection, likely obviating the need for invasive arterial catheterization for diagnostic triage. Other noble metals have similar advantages over iodine. According to some embodiments, molecular imaging with gold is possible because each nanoparticle bound to a targeting agent such as a biotag described above would deliver approximately 100-250,000,000 gold atoms to a cognate receptor, thereby significantly increasing the signal.

Targeted contrast compositions for magnetic resonance based diagnostic imaging and therapy. The most commonly used compounds for contrast enhancement for magnetic resonance imaging are gadolinium (Gd) based. Other superparamagnetic metals such as Eu, Fe, Ni and Co are also suitable for use with in vivo or in vitro MRI or in other in vitro methods such as nuclear magnetic resonance (NMR). Magnetic resonance based contrast agents alter the relaxation times of tissues and body cavities where they are present. In particular, the agents shorten the T1 or T2 relaxation time of protons located nearby. A reduction of T1 relaxation time results in a hypersignal, while a reduced T2 relaxation time reduces the signal. Such registered contrast differences between various tissue compartments that are generated by local differences in relaxivities of water protons between those compartments translate into varying degrees of brightness of the image details on the MRI scanner's screen or changes in the recordings of relaxation times in the NMR instruments. Therefore, it is not the strength of the resonance signal itself, but rather the relative differences in signal intensity between various structures and/or in the signal to noise ratios that result in successful visualization of the analyzed features.

Superparamagnetic metal atoms affect water proton relaxivity in their very immediate vicinity. Pico- to nano-molar concentrations of Gd are currently considered to be the thresholds for inducing such a change in relaxivity of water that it will be detected in NMR or MRI (Sherry et al 2009, Flacke et al 2001). If chelated into a biotag target binding domain as described herein (e.g., an scFv, sdFv, CDR or SDR modified CDR targeting ErbB 1-4, TfR, and their associated variants or mutants), these atoms indirectly report the presence of molecules that were targeted by the biotags. Previous attempts to introduce paramagnetic properties were made by randomly attaching reporters such as Gd chelates, dendrimers, or Fe nanoparticles to monoclonal IgG antibodies (Curtet et al. 1985, Mendonca et al. 1986, Linger et al. 1986, Weissleder 1991, Unger et al. 1999, Kobayashi et al. 2003). However, three main factors have contributed to the failure of these attempts. First, random incorporation of reporters into IgG molecules leads to compromised specificity of antibodies upon their denaturation, resulting in low specific binding signal and high background due to non-specific binding. Second, the significant size of the IgG antibodies including the reporters as well as the changes in their properties due to the reporter incorporation led to steric hindrance and repulsion forces. Third, none of the IgG antibodies were internalized by the target cell, but were instead bound to extracellular receptors and retained an equilibrium between bound and free antibodies. An entirely different approach to improving labeling effectiveness by genetically engineering heterospecific, polyfunctional molecules is used herein. As described above, the biotags described herein are engineered to contain multiple highly specific, yet separate domains that are assigned to their functions. Such domains, as described above, may include: a binding domain (e.g., an scFv, sdFv, CDR or SDR modified CDR), a metal atom chelating domains (also known as metal binding domains, or MBDs), an internalization domain, an endosomal escape domain, and a lysosomal escape domain, which comprise one or more signaling sequences. Upon incorporation of a superparamagnetic metal nanoparticle tag, these biotags gain superparamagnetic properties without adversely affecting their targeting functions.

Administration of targeted contrast composition. In some embodiments, the biotags can be used for detection and quantification in vivo and in vitro (described below) of one or more cancer biomarkers. In one embodiment, a targeting contrast agent comprising an imaging contrast agent composition and a quantity of biotags as described above may be used for detection and quantification of one or more cancer biomarkers in vivo. Such detection and quantification can be used to diagnose the malignancy and/or the aggressiveness of a tumor. When used in conjunction with a contrast for detection of cancer biomarkers using a diagnostic imaging technique, the biotags provide a method for evaluation of cancer cell malignancy based upon the level of gene expression products of one or more cancer biomarkers for ErbB 1-4 TfR or their associated variants or mutants, revealing a pinpointed localization of cancer cells in tumors that express a cancer biomarker within a subject's body, and choosing, monitoring and/or effecting a course of cancer therapy by highlighting these ErbB1-4, TfR or associated variant or mutant biomarkers in vivo using CT scanning.

In one embodiment, a biotag used for detection and diagnosis of cancer malignancy may be produced via genetic and chemical engineering of biotags targeting ErbB 1-4, TfR or associated variants or mutants tagged with metal nanoparticle tags. In one embodiment, the biotag includes an scFv, sdFv, CDR or SDR modified CDR binding domain and the metal nanoparticle tag is a gold nanoparticle tag. The gold-tagged biotag (Au*biotag), or other noble metal-tagged biotag minimizes the chance of toxicity and may be used for determining levels of gene expression of ErbB 1-4, TfR or associated variants or mutants, which is indicative of cancer malignancy. When used as part of a targeted contrast composition, the gold-tagged biotag may be a safe method for detection and diagnosis of cancers. According to some embodiments, the cancer cells labeled with the biotag may be detected in vivo and/or in vitro with CT, EDX, and with surface plasmon resonance (SPR) fluorescence, or Raman with greater sensitivity under significantly lower doses of radiation than currently used in oncological radiology. In other embodiments, the cancer cells labeled with the biotag may also be detected with magnetic resonance imaging (MRI) and with NMR in vivo and/or in vitro. MRI offers good spatial resolution as compared to other in vivo imaging modalities currently available, and also provides a topographic reference for the location of the biotags within the anatomy of the human body. Changing relaxivities by retained superparamagnetic biotags in vivo in some body locations or in vitro in a physiological fluid sample indicates the presence of cancer biomarkers or clusters of cancer biomarkers.

Use of Biotags and Targeted Contrast Compositions for Diagnosing and Treating Cancer In Vivo

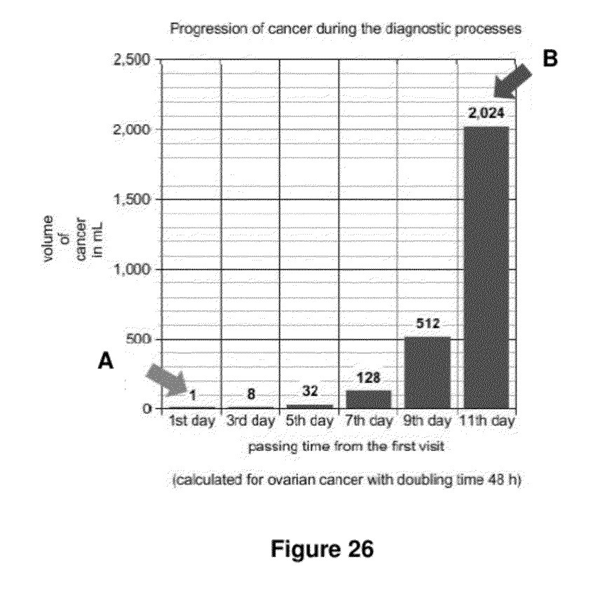

In some embodiments, methods for use of the biotags described above, with or without a contrast agent, during a diagnostic imaging technique are provided for localization of tumors, detection or diagnosis of a cancer, diagnosis of a tumor's aggressiveness, and determining a prognosis of cancer. Cancers and tumor types that may be detected, diagnosed, localized or prognosticated according to the methods described herein include but are not limited to bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, uterine cancer and all subtypes related to any of the above cancers. The methods described herein may be used as an early screening tool, as it allows an efficient way to detect cancerous cells significantly earlier and at significantly less advanced stages as conventional diagnostic processes used in the clinic (FIG. 26).

In some embodiments, the methods described herein include administering an effective dose of a biotag, such as the biotags described above and in the Examples below, to a subject having cancer or suspected of having cancer. The subject may be a human patient or any other mammal that may be diagnosed with cancer, such as mice, rats, rabbits, dogs, cats, or other domesticated or wild animals. The biotags described herein can be administered in an effective dose to a subject with or without a contrast agent, as described in detail above. An effective dose of a biotag with or without a contrast agent for purposes herein is determined by such considerations as are known in the art. For example, an effective amount of the biotag is that amount necessary to deliver a sufficient amount of the biotag to the cytoplasm of target cancer cells to visualize and induce target cancer cell death upon radiation. One of skill in the art can readily determine appropriate single dose sizes for systemic administration based on the size of the patient and the route of administration. An effective dose of the biotag, with a contrast agent, can be selected according to techniques known to those skilled in the art such that a sufficient contrast enhancing effect is obtained. The dose of the contrast agent to be administered can be selected according to techniques known to those skilled in the art such that a sufficient contrast enhancing effect is obtained.

The targeted contrast agents can be administered by any suitable route depending on the type of procedure and anatomical orientation of the tissue being examined. Suitable administration routes include, but are not limited to, intravascular (arterial or venous) administration by catheter, intravenous injection, rectal administration, subcutaneous administration, intrathecal administration, intracisternal administration, intra-cerebrospinal fluid administration, intraperitoneal space administration, intrapleural space administration, oral administration and administration via inhalation.

According to some embodiments, the methods for localization of tumors, detection or diagnosis of a cancer, diagnosis of a tumor's aggressiveness, and determining a prognosis of cancer also include exposing the subject to a diagnostic imaging technique to visualize the any cells targeted by the biotag after administration. The diagnostic imaging technique may be any suitable technique for detecting the biotag, including, but not limited to, radiography, computed tomography (CT), magnetic resonance imaging (MRI), ultrasonography (USG), Raman spectroscopy, positron emission tomography (PET), single photon emission computed tomography (SPECT) and gamma scintigraphy. The diagnostic imaging technique allows a population of cells expressing a cancer biomarker targeted by the biotag to be detected according to the methods described herein. Further, the diagnostic imaging technique may be performed with stationary instruments, hand-held instruments, or both.

An increased expression of the targeted cancer biomarker as determined by the methods described herein may be indicative of various results. In a subject suspected of having cancer, an increased expression of the cancer biomarker indicates that the subject has cancer. Higher quantitative levels may also indicate more aggressive cancer. In a subject that has been previously diagnosed with cancer, an increased expression of the cancer biomarker may indicate a poor prognosis (i.e., a lower cancer-free survival or overall survival), or that a particular treatment regimen is not effective and should be changed.

The methods described herein allow practitioners such as radiologists and oncologists to detect a tumor with a very low radiation dose—much lower than currently used, and the methods allow a practitioner to diagnose tumor malignancy and agressiveness based upon determination of the number of expressed biomarker receptors with high sensitivity. In addition, the biotags described herein are significantly more sensitive in detecting much smaller number of cells than any previous detection method. Therefore, they may be used to detect cancer occurrences at much earlier stages, resulting in saving lives, reducing trauma and reducing healthcare costs.

In some embodiments, methods for use of a targeted contrast composition during a diagnostic imaging technique are provided for localization or tumors, diagnosis of malignant cancer, diagnosis of a tumor's aggressiveness, and prognosis of malignant cancer. The methods described herein allow practitioners such as radiologists and oncologists to detect a tumor with a very low radiation dose—much lower than currently used, and the methods allow a practitioner to diagnose tumor malignancy and aggressiveness based upon determination of the number of expressed biomarker receptors with high sensitivity. In addition, the biotags described herein are significantly more sensitive in detecting a much smaller number of cells than any previous detection method. Therefore, they may be used to detect cancer occurrences at much earlier stages, resulting in saving lives, reducing trauma and reducing healthcare costs.

In some embodiments, the biotags can be used for detection and quantification in vivo and in vitro (described below) of one or more cancer cell targets. In one embodiment, a targeting contrast agent comprising an imaging contrast agent composition and a quantity of biotags as described above may be used for detection and quantification of one or more cancer biomarkers in vivo. Such detection and quantification can be used to diagnose the malignancy and/or the aggressiveness of a tumor. When used in conjunction with a contrast for detection of cancer biomarkers using a diagnostic imaging technique, the biotags provide a method for evaluation of cancer cell malignancy based upon the level of gene expression products of one or more cancer biomarkers for ERBB 1-4, revealing a pinpointed localization of cancer cells in tumors that express a cancer biomarker within a subject's body, and choosing, monitoring and/or effecting a course of cancer therapy by highlighting these ERBB1-4 biomarkers in vivo using CT scanning.

As described above, a biotag used for detection and diagnosis of cancer malignancy may be produced via genetic and chemical engineering of molecular probes targeting ERBB tagged with a metal nanoparticle tag. In one embodiment, the molecular probe includes an ERBB 1-4 scFv or sdFv target binding domain and the metal nanoparticle tag is a gold nanoparticle tag. The gold-tagged biotag (Au*biotag), or other noble metal-tagged biotag eliminates the chance of toxicity and may be used for determining levels of gene expression of ERBB 1-4, which is indicative of cancer malignancy. When used as part of a targeted contrast composition, the gold-tagged biotag may be a safe method for detection and diagnosis of cancers. According to some embodiments, the cancer cells labeled with the biotag may be detected with CT with greater sensitivity under significantly lower doses of radiation than currently used in oncological radiology.

In other embodiments, the cancer cells labeled with the biotag may also be detected with magnetic resonance imaging (MRI). MRI offers good spatial resolution as compared to other in vivo imaging modalities currently available, and also provides a topographic reference for the location of the biotags within the anatomy of the human body.

Quantitative analysis of each of the receptor gene expression products, their ratios, and total concentration allow physicians to broadcast rational prognosis and plan targeted therapy. Moreover, as discussed above, by determining the location of the receptor gene expression products on cancer cells, the biotag can serve as a targeted radio-sensitizer for delivering radiation therapy with great precision. For example, in some embodiments, targeted delivery of such biotags having noble metal or superparamagnetic nanoparticle tags, can be followed by exposure to x-ray or electromagnetic radiation, respectively, killing the cancer cells but, generally, not killing healthy cells or killing a negligible amount of healthy cells.

In addition to their effectiveness as diagnostic agents, the biotags described herein are also effective as a therapeutic agent. As described above, biotags target cancer cells only and become permanently incorporated into targeted cancer cells. According to some embodiments, when biotags are tagged with a heavy metal (Au, Pt, Ag, Pd) core, the effects of x-ray radiation are amplified (Zhao et al. 2009) and the dose of radiation needed for effectiveness is lower. Therefore, multiple rounds of radiation to the area where the cancer tumor is detected using diagnostic biotags, is safe for healthy cells, but lethal for cancer cells filled with the biotags.

According to other embodiments, when biotags are tagged with a superparamagnetic core-shell nanoparticle (containing any combination of Fe, Ni, Co, Au, Pd, Pt, Ag) as the core, substantial heat is generated in cells harboring biotags upon irradiation with electromagnetic radiation but not in non-labeled cells (Balivada et al. 2010). Therefore, several rounds of electromagnetic radiation is safe for healthy cells, but lethal for cancer cells filled with biotags.

The biotags can be administered in an effective dose to a subject with or without a contrast agent. An effective dose of a biotag with or without a contrast agent for purposes herein is determined by such considerations as are known in the art. For example, an effective amount of the biotag is that amount necessary to deliver a sufficient amount of the biotag to the cytoplasm of target cancer cells to visualize and induce target cancer cell death upon radiation. One of skill in the art can readily determine appropriate single dose sizes for systemic administration based on the size of the patient and the route of administration.

An effective dose of the biotag, with a contrast agent, can be selected according to techniques known to those skilled in the art such that a sufficient contrast enhancing effect is obtained. The targeted contrast agents can be administered by any suitable route depending on the type of procedure and anatomical orientation of the tissue being examined. Suitable administration routes include intravascular (arterial or venous) administration by catheter, intravenous injection, rectal administration, subcutaneous administration, intrathecal administration, intracisternal administration, oral administration and administration via inhalation.

Use of Biotags for Detecting Cancer In Vitro and Treatment of Cancer Ex Vivo

In some embodiments, methods for the use of the biotags described above are provided for detecting circulating or disseminated tumor cells (CTC and DTC, respectively), diagnosing cancer, diagnosis of a tumor's aggressiveness and determining a prognosis of cancer. In some embodiments, the methods described herein include incubating a physiological fluid sample from a subject having cancer or suspected of having cancer with a biotag described herein for targeting a cancer biomarker, wherein the biotag binds cells in the sample expressing the cancer biomarker. “Physiological fluid” or “biological fluid” refers to a fluid from a subject and includes blood, serum, plasma, urine, prostate fluid, tears, mucus ascites fluid, oral fluid, saliva, semen, seminal fluid, mucus, stool, sputum, cerebrospinal fluid (CSF), bone marrow, lymph, and fetal fluid.

One of the earliest markers in the progressing cancer by invasion and/or formation of metastases is presence of cancer cells in blood, lymph, CSF, urine, or feces of patients susceptible, suspected of, and/or diagnosed with cancer. Detection of cancer cells in these samples can only be accomplished by distinguishing cancer cells from all other normal blood or lymph cells. This is not an easy task, as evidenced by the fact that even after separating 4-6 million red blood cells in one microliter of blood, there are approximately 10,000 white blood cells that remain (Anderson et al 2009). This means that 1 liter of blood may contain 4-10×10⁹ making a total of 5×10¹⁰ (50 billion cells) to search through.

Most efforts in oncology are devoted to studying the genomic and proteomic mechanisms of cancerogenesis, which is associated with almost 90% of funds directed to developing new methods of therapy. However, in the clinical practice the first step before undertaking any therapy is to make a diagnosis. Thereafter, an important element of care for cancer patients is to prevent tumors from metastasizing, and if they do escape, use focused therapy by surgery or radiation to capture the metastasis at the earliest stage. One important element to this approach is detection of the cancer cells in blood, lymph, peritoneal, pleural, cerebrospinal fluids-based diagnosis their pathology, and testing the most effective therapy to destroy the metastasizing cells.

In some embodiments, the biotags described herein may be used to detect cancer cells in the blood, lymph, peritoneal fluid, pleural fluid, cerebrospinal fluid or other physiological fluid of a subject who is suspected of having cancer. Thus, the biotags may be used to diagnose a subject who has not yet been diagnosed with cancer. Detection of cancer cells in this manner may also be used to confirm an ongoing metastasis of a primary tumor in a subject who has already been diagnosed with a malignant tumor but metastases were or were not yet discovered. All of these scenarios may have profound impact on choices of planned therapies.

In one embodiment, detection of cancer cells in blood, lymph, peritoneal fluid, pleural fluid, cerebrospinal fluid or other physiological fluid may include one or more of the following steps. The first step towards detection of metastasizing cancer cells is to identify a cancer specific biomarker. In one embodiment, the cancer specific biomarkers are ErbB 1-4, TfR or their related mutants. The second step is to develop a specific biotag, such as those biomarkers described herein, to a cancer biomarker that binds to the cancer biomarker with unique specificity and high affinity though its biotag biomarker binding domain. The biomarker binding domain may be an antibody or functional fragment thereof, which are described above. In one embodiment, the binding domain is an scFv, sdFv, CDR or SDR modified CDR. The third step involves development of a tag to function as a specific reporter, which provides a signaling presence and visualization of the location of the biotag bound to the cancer biomarker on the cancer cell. In some embodiments, the reporter is any diagnostic agent described above. In some embodiments, the tag is a metal nanoparticle tag or a fluorescent agent tag. Such metal nanoparticle tag may be a noble metal or superparamagnetic metal as described above. The fourth step involves exposing a physiological fluid (e.g., blood, lymph, peritoneal fluid, pleural fluid, cerebrospinal fluid or other physiological fluid) sample to the biotag, detecting it, and then isolating of the cancer cells bound by the biotag for further analysis. Isolation of the cells may be accomplished based on the type of reporter associated with the biotag. For metals such as noble metals, a weight or mass gradient may be performed to separate the heavier tagged cells. For superparamagnetic metals, the isolation may be accomplished by a magnetic separation using a magnet. For biotags having a fluorescent reporter, isolation may be performed by a cytometry method such as FACS. These steps result in the detection of metastasizing cells in the blood, lymph, peritoneal fluid, pleural fluid, cerebrospinal fluid or other physiological fluid sample obtained from a patient. The isolated cancer cells may be used for testing resistance to various cancer therapies.

In vitro detection of biotags. Detection of a proportional increase in the number of ErbB 1-4 and Transferrin (Tf) receptors per cell results in a proportional change in relaxation times or relaxivity via biotags attached to the cancer cells studied with NMR and MRI. Consequently, this results in a proportional increase in relaxivity of the surrounding water leading to a proportional increase in the signal strength (as measured by brightness or shortening T) recorded with magnetic resonance receivers. Prior to this disclosure, none of the commercially available probes met the criteria outlined above with toxicity of various probes revealed in the most recent long-term studies being of significant concern (Deo et al 2007, Reilly 2009). The embodiments described herein are a solution to the problems mentioned above. The biotags described herein are advantageous for several reasons, some of which are as follows. First, unlike monoclonal and polyclonal antibodies or their fragment-based probes which disassociate from their receptor or antigen according to their on/off characteristics that depend on the physiological environment conditions, the biotags are internalized, thereby labeling cancer cells permanently. This prevents false negative results (i.e., patients will leave the hospital carrying undetected cancer). Second, the biotags generate a stable signal. Third, the biotags are highly specific to cancer cells, unlike monoclonal and polyclonal antibodies or antibody fragment-based probes that may result in non-specific binding (e.g., FcR binding). Fourth, the biotags can label multiple domains of a single cancer cell receptor due to their small size, thereby enhancing the signal and detecting mutations. In contrast, monoclonal and polyclonal antibodies are approximately 155 kDa, which prevents multiple labels from reaching the target due to steric hindrance and prevents large magnetic, optical, or colloidal beads from forming, which may be phagocytosed by macrophages generating false positive results. Fifth, the biotags bypass cellular degradation and recycling pathways, making them a long term or permanent tag.

Therefore, in some embodiments, the studies described herein enable the use of superparamagnetic or noble metal tagged biotags that target ErbB1-4, TfR or related variants or mutants for the detection of cells disseminating from the primary tumor and/or metastasizing cancer cells. The biotags also allow for evaluation of differences in levels of gene expression products in cells in vitro and in vivo. In one embodiment, the studies described herein that label cell receptors with superparamagnetic biotag scFvs, sdFvs, CDRs or SDR modified CDRs resulted in a dramatic shortening of the T1 relaxation time. This shortening of T1 was proportional to the number of superparamagnetic atoms (e.g., Gd or Eu) harbored by scFvs, sdFvs, CDRs or SDR modified CDRs and anchored to the cell surface receptors. The significant differences between the number of the receptors on surfaces of cancer and normal cells correlated to the significant differences in the signal intensity between these cells. These studies may also be extrapolated to in vivo studies involving targeted contrast compositions as described above.

In addition to their effectiveness as diagnostic agents, the biotags described herein are also effective as a therapeutic agent. As described above, biotags target cancer cells only and become permanently incorporated into targeted cancer cells. According to some embodiments, when biotags are tagged with a heavy metal (Au, Pt, Ag, Pd) core, the effects of X-ray radiation are amplified (Zhao et al. 2009) and the dose of radiation needed for effectiveness is lower. Therefore, multiple rounds of radiation to the area where the cancer tumor is detected using diagnostic biotags, is safe for healthy cells, but reach lethal doses for cancer cells filled with the biotags.

According to other embodiments, when biotags are tagged with a superparamagnetic core-shell nanoparticle (containing any combination of Fe, Ni, Co, Au, Pd, Pt, Ag) as the core, substantial heat is generated in cells harboring biotags upon irradiation with electromagnetic radiation but not in non-labeled cells (Balivada et al. 2010). Therefore, several rounds of electromagnetic radiation is safe for healthy cells, but reach lethal doses for cancer cells filled with biotags.

In other embodiments, the biotags described herein may be used to treat metastatic cancer or primary hematologic neoplasms by killing cancer cells present in a bodily fluid (e.g., blood, lymph or cerebrospinal fluid). Such cancer cells are targeted by administering to a subject an effective dose of a biotag that targets a cancer cell biomarker as described above. The biotag then binds and is internalized by the cancer cells, permanently labeling them with metal nanoparticles that act as radiosensitizers in those cells. After administering the biotag, the cells may be exposed to one or more treatments to specifically target and kill the cancer cells.

In one embodiment, the treatment may be an extracorporeal procedure. An extracorporeal procedure is a procedure in which blood is taken from a patient's circulation to have a process applied to it, ex vivo, before it is returned to the circulation. The apparatus carrying the blood outside of the body is known as the extracorporeal circuit, and diversion of a subject's blood flow through such a circuit that is continuous with the normal in vivo body circulation is known as an extracorporeal circulation.

In some embodiments, the extracorporeal procedure is an extracorporeal radiotherapy procedure. Thus, in some embodiments, an extracorporeal circulation may be established and exposed to the extracorporeal radiotherapy procedure. The extracorporeal radiotherapy procedure may be one or more doses of radiation (e.g., X-ray therapy or electromagnetic therapy) directed to the bodily fluid that flows through the extracorporeal circuit, killing the cells labeled with the biotag.

In one embodiment, to establish the extracorporeal circulation, a vascular access is established in a subject. A vascular access is a site on the subject's body from which blood is removed and returned, and may include, but is not limited to, an arteriovenous (AV) fistula, an AV graft, or a venous catheter. Once a vascular access is established, it may be connected to an anti-coagulation coated tube (e.g., a heparinized tube) to establish the extracorporeal circulation.

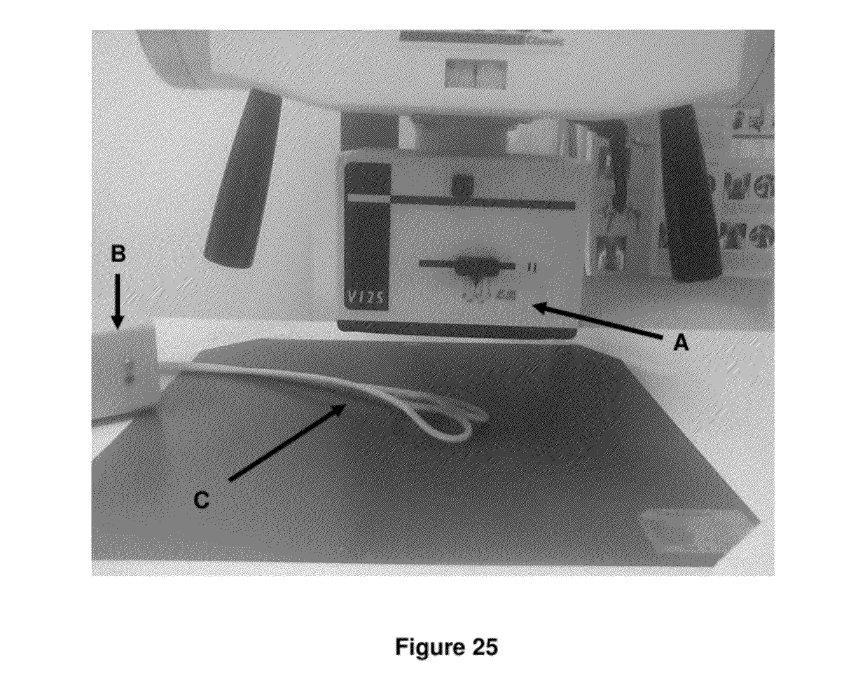

The extracorporeal radiotherapy procedure may be carried out using a set of instruments that include a radiation source (e.g., X-ray radiation or electromagnetic radiation), a pump to keep the extracorporeal circulation flowing (e.g. peristaltic pump) and an extracorporeal circuit (e.g., heparinized or other anti-coagulation coated tubes). An example of these instruments is shown in FIG. 25.

Current methods of treatment using radiation or chemotherapy often result in death of both cancer cells and other cells found in the blood or other bodily fluid, thereby requiring a blood or bone marrow transfusion to replenish the blood cells lost during treatment. The methods of treatment described herein results in efficient eradication of metastasizing cancer cells or primary hematologic neoplasm cancer cells from a cancer patient's blood, without the deleterious effects of toxic systemic treatments and without the need for a transfusion.

The biotags can be administered in an effective dose to a subject with or without a contrast agent. An effective dose of a biotag with or without a contrast agent for purposes herein is determined by such considerations as are known in the art. For example, an effective amount of the biotag is that amount necessary to deliver a sufficient amount of the biotag to the cytoplasm of target cancer cells to visualize and induce target cancer cell death upon radiation. One of skill in the art can readily determine appropriate single dose sizes for systemic administration based on the size of the patient and the route of administration.

An effective dose of the biotag, with a contrast agent, can be selected according to techniques known to those skilled in the art such that a sufficient contrast enhancing effect is obtained. The targeted contrast agents can be administered by any suitable route depending on the type of procedure and anatomical orientation of the tissue being examined. Suitable administration routes include intravascular (arterial or venous) administration by catheter, intravenous injection, rectal administration, subcutaneous administration, intrathecal administration, intracisternal administration, oral administration and administration via inhalation.

Quantitative Analysis of In Vivo and In Vitro Use of Biotags

The benefit of using a targeted contrast such as that described herein is based upon the clinical and immunohistopathalogy data. For example, one cancer cell may express approximately three million EGF receptors. These numbers are equivalent to their approximate molar concentrations of 10⁻⁵ M. These values are in sharp quantitative contrast to those reflecting levels of expression of these receptors in normal cells, as ECF receptors are, in effect, not detectable on frozen sections, paraffin sections, or in cell culture (less than 10,000 receptors). This results in a signal that is 100 to 300 times higher from cancer cells than from normal cells. These antigens help to diagnose highly malignant cancers with poor prognosis and distinguish quantitatively highly malignant cancers characterized by the rapid growth, invasion, and metastasis from more benign cancers. Finally, mutations within molecules displayed on the surface of cancer cells are particularly attractive targets for potential antibody-guided contrast agents, as they are for immunotherapy. In these cases, the mutation is a specific, unique marker of the cancer.

The quantitative and qualitative differences discussed above can be determined with the aid of antibodies, their fragments, and ligands directed against the molecules present on surfaces of neoplastic cells. Such determinations have paramount importance for making a clinical diagnosis with prognostic and therapeutic consequences. Prior to the current disclosure, these differences have been assessed in vitro using diagnostic histopathology and immunohistochemistry on frozen or paraffin sections. The current disclosure describes biotags to qualitatively and quantitatively determine these differences using diagnostic immunohistochemistry in vivo via assessment by CT.

Any increase in the receptor number, or scFv (or sdFv) per receptor (no more than one IgG would label one receptor because of the steric hindrance (Malecki M et al. 2002), or number of Au atoms per nanocrystal tag will push the detection threshold into millimolar range. For broadcasting prognosis and planning therapy, it is important to determine receptor density on the cancer cells. This is established by labeling all of the domains of all the receptors and comparing with the signal received from the standard (containing the series dilutions of the known concentrations) placed next to the subject. The ratio between them allows very specific quantification of the neoplasm dynamics. For clinical purposes, this can be accomplished step-wise using individual scFv or sdFv probes against biomarker receptor domains one after another. For the integrated evaluation of the cancerous tumor, cocktails of biotags targeting various domains can be used, thus leading to multiplication of the signal to noise ratio with every biotag added to the cocktail. Dramatic increase and permanent retention of the signal recorded with CT occurs upon internalization of the biotag into the endosome, its lysosomal escape, and permanent retention within cytoplasm of cancer cells (also useful for monitoring of therapeutic effects).

In some embodiments, once the biotag has been detected as described above, the expression of the cancer biomarker in the population of cells is quantified. Quantitative analysis of each of the receptor gene expression products, their ratios, and total concentration allow physicians to broadcast rational prognosis and plan targeted therapy. Moreover, by determining the location of the receptor gene expression products on cancer cells, the biotag can serve as a targeted radio-sensitizer for delivering radiation therapy with great precision. For example, in some embodiments, detection of circulating or disseminated tumor cells in the samples of physiological fluids like blood, CSF, etc may be followed by injection iv or iCSF, etc of the same biotags for targeted delivery of such biotags having noble metal or superparamagnetic nanoparticle tags, so they can be followed by exposure to CT or MRI, respectively, to reveal cancer location. Moreover, the exposure to x-ray or magnetic radiation may be used as therapies which cause the cancer cells' deaths.

In vivo quantification of biotags. Malignant tumors may express more than with 3 million ErbB and/or Tf receptors on their surfaces being the products of upregulated gene expression. The tumor palpable during the physical examination has a volume of about 1 cc or 1 ml resulting from the growth of approximately 10 billion (or 10⁹) cells. These numbers account for 3×10¹⁵ of receptors present only on the cell surfaces in 1 ml volume of a tumor without counting receptors, which were being internalized and recycled as validated with TEM. With 100 to 3000 atoms of gold tagging each biotag targeted on one ErbB or Tf receptor, this leads to accumulation of 3×10¹⁸ gold atoms in this volume. Unlike 155 kDa IgG or 50 kDa diabodies, at least three 5 to 20 kDa biotags may label one receptor. This brings the gold atom account up to 9×10¹⁸ or about 10¹⁹. At least four different types of the receptors are targeted by our biotags within ErbB family, which may double or quadruple this account or 2-4×10¹⁹ or 0.2-0.4×10²⁰ or approximately 0.2-0.4 mM. This is well within the range of detection with SPR, X-ray, Raman, and CT, which may be determined experimentally. These calculations are for the receptors present on the surface only and labeled as such with the pulse and chase experiments, but they do not account for internalization characterized by the biotags.

Rapid internalization of the biotags upon the ERBB receptors leads to their rapid clearance and synthesis of the new receptors followed by their trafficking to the cell surfaces. These processes lead to constant import of the biotags into the cells. When two cells, one expressing 3 million cells on its cell surface and the second one expressing 30,000 receptors on its surface, are exposed to the same concentration of biotags tagged with gold, the first one will generate 100× stronger signal for imaging than the latter. With the refresh rate of about 1,000 per hour, the total account for the imported biotags into the cells reaches 0.2-0.4×10²³ or 0.2-0.4M. This catapults the concentration of the gold atoms tagging biotags to molar (M) range with a signal to noise ratio to 100/1. This calculation accounts for average recycling of the receptors, during which time, the biotags pass through the endosomal recycling pathway, and subsequently escape from these pathway to saturate cell cytoplasm with gold atoms

Presence of endosomal escape signals on the biotags results in their escape from the endosome-to-lysosome pathways, while entering the cytoplasm. They remain retained there. With almost entire clearance of the scFv from blood within one hour, the residual signal from the presence of the biotags in the circulation is minimal or absent, while the signal from the biotags tagged with nanogold retained within the cells remain unchanged. This catapults the signal to noise ratio far within the detection range of Raman, x-ray, CT, MRI and NMR.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. Further, all references cited above and in the examples below are hereby incorporated by reference in their entirety, as if fully set forth herein.

### Example 1

### Generation of scFv, sdFv, CDR and SDR Modified CDR Biomarker Binding Domains

scFvs and sdFvs against ErbB 1-4 and TfR were constructed by generating combinatorial display libraries using HEK293 cell, phage and mRNA displays.

First, B cells were isolated from cancer patients. Cancer patients' blood was drawn as small aliquots under the informed consent based upon the IRB approved protocol. To 2 ml of anticoagulant-treated blood, 2 ml of balanced salt solution were added and mixed. Unto the top of 3 ml of the Ficoll-Paque Plus in Falcon tube, 4 ml of diluted blood were layered without mixing. The samples were centrifuged at 400 g for 30-40 minutes at 18-20° C. This led to separation of the sample into four layers: 1. plasma (top), 2. lymphocytes, 3. Ficoll-Paque Plus, and 4. granulocytes, erythrocytes. After discarding the plasma, the lymphocyte layer was transferred to the new Falcon tube, to which at least 3 volumes of balanced salt solution were added and mixed. The sample was centrifuged at 400 g for 10 minutes at 18-20° C. The supernatant was removed. The lymphocytes were resuspended in 6-8 ml balanced salt solution. The cells were counted on the Beckman Coulter cell counter.

The B cells were isolated by negative selection. Non-B cells, i.e., T cells, NK cells, monocytes, dendritic cells, granulocytes, platelets, and erythroid cells depletion was performed with antibodies against CD2, CD14, CD16, CD36, CD43, and CD23 tagged with our magnetic beads. This left the sample with a pure population of untouched B cells. This was validated by labeling of B cells with CD19 and CD20. The samples were further processed or stored in liquid nitrogen.

After extracting total RNA from the isolated lymphocytes using an RNeasy Mini Kit (Qiagen), RT-PCR was performed to amplify human antibody complementary determining regions (CDRs), specificity determining residues (SDR) and framework regions (FRs). cDNA was prepared using SuperScript™ III First-Strand Synthesis System (Invitrogen). cDNA may alternatively be obtained by a Cells-To-cDNA kit from Qiagen. Approximately, 5 pg to 25 μg of RNA or mRNA was reverse transcribed into the first-strand cDNA.

The CDR and FR cDNA was then amplified by PCR. The primers were selected from those published (Barbas C F, 3rd, Burton D R, Scott J K, Silverman G J, 2001) after analysis of sequences data base (Kabat, 1991, Chothia et al. 1989, 1988). Examples of primers included, but were not limited to these outlined below:

Primers for CDR1:
H1-Forward:
(SEQ ID NO: 1)
5′-GAG GAG GAG GAG GAG GAG GCG GGG CCC AGG CGG
CCC AGG TGC AGC TGG TGC-3′;
H1-Reverse:
(SEQ ID NO: 2)
5′-GCG GAC CCA GCT CAT TTC ATA AKM AKM GAA AKM GAA
AKM AGA GGC TGC ACA GGA GAG-3′
Primers for CDR2:
H2-Forward1:
(SEQ ID NO: 3)
5′-GAA ATG AGC TGG GTC CGC CAG GCT CCA GGA CAA
SGS CTT GAG TGG-3′;
H2-Forward2:
(SEQ ID NO: 4)
5′-GAA ATG AGC TGG GTC CGC CAG GCT CCA GGG AAG
GCC CTG GAG TGG-3′;
H2-Forward3:
(SEQ ID NO: 5)
5′-GAA ATG AGC TGG GTC CGC CAG GCT CCA GGG AAG
GGN CTR GAG TGG-3′;
H2-Reverse1:
(SEQ ID NO: 6)
5′-ATT GTC TCT GGA GAT GGT GAC CCT KYC CTG RAA
CTY-3′;
H2-Reverse2:
(SEQ ID NO: 7)
5′-ATT GTC TCT GGA GAT GGT GAA TCG GCC CTT CAC
NGA-3′;
H2-Reverse3:
(SEQ ID NO: 8)
5′-ATT GTC TCT GGA GAT GGT GAC TMG ACT CTT GAG
GGA-3′;
H2-Reverse4:
(SEQ ID NO: 9)
5′-ATT GTC TCT GGA GAT GGT GAC STG GCC TTG GAA
GGA-3′;
H2-Reverse5:
(SEQ ID NO: 10)
5′-ATT GTC TCT GGA GAT GGT AAA CCG TCC TGT GAA
GCC-3′;
Primers for CDR3:
H3-Forward1:
(SEQ ID NO: 11)
5′-ACC CTG AGA GCC GAG GAC ACR GCY TTR TAT TAC
TGT-3′;
H3-Forward2:
(SEQ ID NO: 12)
5′-ACC CTG AGA GCC GAG GAC ACA GCC AYR TAT TAC
TGT-3′;
H3-Forward3:
(SEQ ID NO: 13)
5′-ACC CTG AGA GCC GAG GAC ACR GCY GTR TAT TAC
TGT-3′;
H3-Reverse:
(SEQ ID NO: 14)
5′-GTG GCC GGC CTG GCC ACT TGA GGA GAC GGT GAC
C-3′
Primers for other CDRs
CDR-H1-Forward
(SEQ ID NO: 45)
5′-CTC TGG ATT CAC CTT TAG CRR TTA TKM TAT GAG
CTG GGT CCG CCA GGC TCC AG-3′;
CDR-H2-Forward
(SEQ ID NO: 46)
5′-GGG CTG GAG TGG GTC TCA KBG ATC TMT YMT RRT
RRT RGT ART AHA TAT TAC GCT GAT TCT GTA AAA GGT
CGG TTC ACC ATC TCC AGA G-3′;
CDR-H3-9-Reverse
(SEQ ID NO: 47)
5′-CTG GCC CCA GTA GTC GAA MNN MNN MNN MNN
TYT CGC ACA GTA ATA CAC GGC-3′;
CDR-H3-14-Reverse
(SEQ ID NO: 48)
5′-CTG GCC CCA GTA GTC GAA MNN MNN MNN MNN
MNN MNN MNN AVS AYC TYT CGC ACA GTA ATA CAC
GGC-3′;
CDR-H3-20-Reverse
(SEQ ID NO: 49)
5′-CTG GCC CCA GAC GTC CAT ASC ATH AKM AKA
AKA MNN MNN MNN MNN MNN MNN MNN AMB AVB ANV TYT
CGC ACA GTA ATA CAC GGC-3′;
CDR-H3-20SS-Reverse
(SEQ ID NO: 50)
5′-CTG GCC CCA GAC GTC CAT ASC ATH AKM AKA
AKA ACA MNN MNN MNN MNN ACA MNN AMB AVB ANC TYT
CGC ACA GTA ATA CAC GGC-3′;
CDR-L1-Forward
(SEQ ID NO: 51)
5′-GAG GGT CAC CAT CTC TTG TAS TGG CTC TTC ATC
TAA TAT TGG CAR TAA TDM TGT CWM CTG GTA CCA GCA
GCT CCC AG-3′;
CDR-L2-Forward
(SEQ ID NO: 52)
5′-CCC AAA CTC CTC ATC TAT KMT RAT ART MAK CGG
CCA AGC GGG GTC CCT GAC CGA TTC-3′;
CDR-L3-Reverse
(SEQ ID NO: 53)
5′-GAG GCT GAT TAT TAC TGT GST DCT TGG GAT KMT
AGC CTG ART GST TAT GTC TTC GGC GGA GGC-3′;
Primers for FRs
FR3-Forward:
(SEQ ID NO: 15)
5′-ACC ATC TCC AGA GAC AAT TCC-3′
FR3-Reverse:
(SEQ ID NO: 16)
5′-GTC CTC GGC TCT CAG GGT G-3′
FR-H1-Forward:
(SEQ ID NO: 54)
5′-GAG GTG CAG CTG TTG GAG TCT GGG GGA GGC TTG
GTA CAG CCT GGG GGG TCC CTG-3′;
FR-H1-Reverse:
(SEQ ID NO: 55)
5′-GCT AAA GGT GAA TCC AGA GGC TGC ACA GGA GAG
TCT CAG GGA CCC CCC AGG CTG-3′;
FR-H2-Reverse:
(SEQ ID NO: 56)
5′-TGA GAC CCA CTC CAG CCC CTT CCC TGG AGC CTG
GCG GAC CCA-3′;
FR-H3-Reverse:
(SEQ ID NO: 57)
5′-GGC TGT TCA TTT GCA GAT ACA GCG TGT TCT TGG
AAT TGT CTC TGG AGA TGG TGA ACC G-3′;
FR-H3-Forward:
(SEQ ID NO: 58)
5′-GTA TCT GCA AAT GAA CAG CCT GAG AGC CGA GGA
CAC GGC CGT GTA TTA CTG TGC G-3′;
JH-15-Forward:
(SEQ ID NO: 59)
5′-T-TCG ACT ACT GGG GCC AGG GTA CAC TGG TCA CCG
TGA GCT CA-3′;
JH-6-Forward:
(SEQ ID NO: 60)
5′-ATG GAC TGC TGG GGC CAG GGT ACA CTG GTC ACC
GTG AGC TCA-3′;
FR-L1-Forward:
(SEQ ID NO: 61)
5′-CAG TCT GTG CTG ACT CAG CCA CCC TCA GCG TCT
GGG ACC CCC-3′;
FR-L1_Reverse:
(SEQ ID NO: 62)
5′-ACA AGA GAT GGT GAC CCT CTG CCC GGG GGT CCC
AGA CGC TGA G-3′;
FR-L2-Reverse:
(SEQ ID NO: 63)
5′-ATA GAT GAG GAG TTT GGG GGC CGT TCC TGG GAG
CTG CTG GTA CCA G-3′;
FR-L3-Reverse:
(SEQ ID NO: 64)
5′-GAT GGC CAG GGA GGC TGA GGT GCC AGA CTT GGA
GCC AGA GAA TCG GTC AGG GAC CCC-3′;
FR-L3-F
(SEQ ID NO: 65)
5′-TCA GCC TCC CTG GCC ATC AGT GGG CTC CGG TCC GAG
GAT GAG GCT GAT TAT TAC TGT G-3′;
JL-Forward:
(SEQ ID NO: 66)
5′-TAT GTC TTC GGC GGA GGC ACC AAG CTG ACG GTC CTA
GGC-3′;
FRH3-short-Reverse:
(SEQ ID NO: 67)
5′-CGC ACA GTA ATA CAC GGC C-3′;
JH15-short-Forward:
(SEQ ID NO: 68)
5′-TTC GAC TAC TGG GGC CAG-3′;
JH6-short-Forward:
(SEQ ID NO: 69)
5′-ATG GAC GTC TGG GGC CAG GGT ACA CTG-3′;
pC3X-Forward:
(SEQ ID NO: 70)
5′-GCA CGA CAG GTT TCC CGA C-3′;
pC3X-Reverse:
(SEQ ID NO: 71)
5′-AAC CAT CGA TAG CAG CAC CG-3′;
H1-Reverse:
(SEQ ID NO: 72)
5′-GCT AAA GGT GAA TCC AGA G-3′;
H2-Forward:
(SEQ ID NO: 73)
5′-CTG GGT CCG CCA GGC TCC AG-3′;
H2-Reverse:
(SEQ ID NO: 74)
5′-TGA GAC CCA CTC CAG CCC-3′;
H3-Forward:
(SEQ ID NO: 75)
5′-CGG TTC ACC ATC TCC AGA G-3′;
L1-Reverse:
(SEQ ID NO: 76)
5′-CAA GAG ATG GTG ACC CTC-3′;
L2-Forward:
(SEQ ID NO: 77)
5′-CTG GTA CCA GCA GCT CCC AG-3′;
L2-Reverse:
(SEQ ID NO: 78)
5′-ATA GAT GAG GAG TTT GGG-3′;
L3-Forward:
(SEQ ID NO: 79)
5′-GGG GTC CCT GAC CGA TTC-3′;
L3-Reverse:
(SEQ ID NO: 80)
5′-CAC AGT AAT AAT CAG CCT C-3′;
JL-short-Forward:
(SEQ ID NO: 243)
5′-TAT GTC TTC GGC GGA GGC-3′;

Using the cDNA and combinations of these primers, the CDRs and FRs from cDNA samples were amplified using standard PCR protocols including (1) preparing the following mixture in thin wall PCR tubes: ddH2O (23-×μl), 2× High Fidelity PCR Master (25 μl), Forward primer (25 μM) 1 μl, Reverse primer (25 μM, 1 μl), and cDNA×μl (˜1 μg); and (2) cycling on ABI 7900 or 7500 FAST: (a) 4 min at 94° C.; (b) 45 sec at 94° C.; 45 sec at 55° C.; 1 min at 72° C.×30 cycles; (c) 5 min at 72° C.

The amplicons were run on 2% agarose gel, stained with SybrGold, and imaged with Storm 840. These primers were either cloned under used for diversification after PCR introducing the following restriction sites:

(SEQ ID NO: 17)
Sfi I: 5′ . . . GGCCNNNN*NGGCC . . . 3′;
and
(SEQ ID NO: 18)
SacII: 5′ . . . CCGC*GG . . . 3′;

and then assembled into the plasmids coding: complementarity determining regions (CDR), specificity determining residues (SDR) (determined based upon modeling of docking CDR into receptors using MOE software developed by Chemical Computing Group), single chain variable fragments (scFv) or single domain variable fragments (sdFv) shown in Tables 1 and 2 above.

HEK293, phage, and mRNA displays. The PCR amplicons digested with the SfiI and SaclI (New England Biolabs, Ipswich, Mass.), gel purified, and ligated into the pDisplay (Invitrogen), which contains a PDGFR anchor. The ligation mix was used to transform E. coli TOP10 cells (Invitrogen). Each transformation produced surface display library containing ˜10̂6 clones. This was further diversified by mutations and gene shuffling. DNA was recovered with Miniprep from Qiagen.

HEK293T cells were grown in DMEM with DCS and were transfected using Lipofectamine Plus. After 72 h, they were labeled with antimyc and purified receptor protein tagged with magnetic beads or fluorochromes. This allowed isolation of positive expressors from the medium. DNA was recovered from each clone in preparation for determination of affinity constant after HEK293T expression and for sequencing.

Phagemid pComb3X cut with SfiI was used to clone CDR and FR after multiple rounds of PCR with overlap extension and to get CDRs and FRs together. The inserts were ligated into the vector with T4 ligase followed by desalting with Amicon Ultra-4. TG1 electroporation-competent cells were transfected with desalted ligations by electroporation and grown in 2YT medium. Qiagen HiSpeed Plasmid Maxi Kit was used for phagemid preparation.

mRNA display and expression was performed as previously described (Wilson, Keefe, and Szostak, 2001). Selection of internalizing clones of scFv or sdFv or ligands was performed as previously described (Poul 2009).

### Example 2

### Generation of Noble Metal-Tagged scFv Biotags

Assembly of multidomain, macromolecular clusters. SwissProt and NCBI databases were used to determine the following functional domains that target intracellular targeting functions: internalization domain, endosomal escape domain, lysosomal escape domain, metal binding domain (MBD). These domains were synthesized on the ABI oligopeptide synthesizer or generated by phage display as previously described (Newton 2009).

Internalization domain sequences include, but are not limited to:

| | |
|---|---|
| YHWYGYTPQNVI | (SEQ ID NO: 19) |
| NPVVGYIGERPQYRDL | (SEQ ID NO: 20) |
| ICRRARGDNPDDRCT | (SEQ ID NO: 21) |

Endosomal escape domain sequences include, but are not limited to:

| | |
|---|---|
| GIGAVLKVLTTGLPALISWIKRKRQQ | (SEQ ID NO: 22) |
| GRKKRRQRRRPPQ | (SEQ ID NO: 23) |
| GLFGAIAGFIENGWEGMIDGWYG | (SEQ ID NO: 24) |

Lysosomal escape domain sequences include, but are not limited to:

| | |
|---|---|
| CHK6HC; | (SEQ ID NO: 25) |
| H5WYG | (SEQ ID NO: 26) |

Metal binding domains include Au binding domains, Gd or Eu binding domains, B binding domains, Ni, Co, Fe, Fe₃O₄, and Fe₂O₃ binding and Au binding domains are also applicable for Fe/Au shelled in core/shell nanoparticles. Metal binding domains include, but are not limited to:

| | |
|---|---|
| (Gly-)ₙ-Cys | (SEQ ID NO: 27) |
| (Gly-Arg-)ₙ-Cys | (SEQ ID NO: 28) |
| (Gly-Lys-)ₙ-Cys | (SEQ ID NO: 29) |
| (Gly-Asp-Gly-Arg)ₙ-Cys | (SEQ ID NO: 30) |
| (Gly-Glu-Gly_Arg)ₙ-Cys | (SEQ ID NO: 31) |
| (Gly-Asp-Gly-Lys)ₙ-Cys | (SEQ ID NO: 32) |
| (Gly-Glu-Gly-Lys)ₙ-Cys | (SEQ ID NO: 33) |

B binding domains suitable for BNT include, but are not limited to: MAP16-B.

Gd or Eu binding domains suitable for Gd MRI and NMR and biotag guided therapy include, but are not limited to:

(SEQ ID NO: 34)
(Glu-Glu-Glu-Glu-Glu)ₙ
(SEQ ID NO: 35)
(Glu-Glu-Glu-Glu-Glu-Glu)ₙ
(SEQ ID NO: 36)
(Asp-Asp-Asp-Asp-Asp)ₙ
(SEQ ID NO: 37)
(Asp-Asp-Asp-Asp-Asp-Asp)ₙ
(SEQ ID NO: 38)
Phe-His-Cys-Pro-Tyr-Asp-Leu-Cys-His-Ile-Leu

Ni and Co binding domains include, but are not limited to:

| | |
|---|---|
| (Gly-Asp-Gly-Arg)ₙ-(His)5,6 | (SEQ ID NO: 39) |
| (Gly-Glu-Gly_Arg)ₙ-(His)5,6 | (SEQ ID NO: 40) |
| (Gly-Asp-Gly-Lys)ₙ-(His)5,6 | (SEQ ID NO: 41) |
| (Gly-Glu-Gly-Lys)ₙ-(His)5,6 | (SEQ ID NO: 42) |
| (Gly-Arg-)ₙ-(His)5,6 | (SEQ ID NO: 43) |
| (Gly-Lys-v-(His)5,6 | (SEQ ID NO: 44) |

Beckman BIOMEK FX Span-8 and 96 Channel Robotic System was loaded with each of the domains within a separate channel. In particular one of the channels contained the noble metal nanoparticles (e.g., gold) or superaparamagnetic core shell nanoparticles. Each of these domains contained a metal binding domain (MBD) at the amino or carboxyl terminus as detailed below. The sequence of the processing allowed addition of the single domain to a single particle at a time. Alternatively, microfluidic system was used with the identical aim. As a result, heterospecific mono-, di-, tri-, etc—mer scFvs, sdFvs, CDRs, SDR modified CDRs and/or internalizing ligands (e.g., truncated EGF or Tn) were assembled and tested, while firmly anchored to the nanoparticle as the core structure. Some constructs led to expression of fusion proteins, but their metal binding domain (MBD) at the carboxyl or amino terminus served as the anchors to the nanoparticles.

Targeting domains for the ligands EGF and/or HRG may be synthesized on the ABI peptide synthesizer with the sequences available from GenBank and EMBL. To suppress the ability of cancer cells to respond to reactive oxygen species, scFv and sdFv against catalase, CuZn Superoxide Dismutase, Mn Superoxide Dismutase, and GPX were used due to their application in cancer suicide gene therapy and synthesized as previously described (Malecki 2007).

Manufacturing of pure noble metal nanoparticles. Nanoparticles derived from noble metals Au, Pt, Pd and Ag were generated by laser ablation of 99.99% purity metal foils in a chamber filled with deionized water under continuous flow as described previously (Malecki 1996). Some variability in sizes was compensated by gradient ultracentrifugation, which also resulted in their condensation.

Noble metal tagged scFv biotags. Plasmid constructs were generated as described previously (Malecki et al. 2002). Briefly, biotag constructs having coding sequences comprising scFvs targeting ErbB1-4 or TfR (i.e., binding domain) extended with internalization signals (i.e., internalization domain), endosomal/lysosomal escape signals (endosomal escape domain and lysosomal escape domain), and histidines, glutamates, asparagnines, and cysteines (MBD) were selected from surface display libraries as described above. Constructs for scFvs, sdFv, CDR, or/and SDR modified CDR were electroporated into human myelomas, CHO and/or HEK 293 cells. Expression of these constructs resulted in the surface display of the products. The expressor clones were selected, plasmids purified, and the sequences amplified as described above. This was followed by cloning without surface anchoring sequences, but by secretion into the medium scFvs, sdFv, CDR, or SDR modified CDR. The extended coding sequences were then cloned into pM vectors designed with the following: CMV immediate early promoter, SV40 poly(A) termination, and neomycin-resistance. Constructs for these fragments were then electroporated into human myelomas for expression of the scFv, sdFv, CDR or SDR modified CDR. The myelomas were cultured in modified roller bottles according to standard protocols. Expression of the constructs by the myeloma resulted in the production and secretion of scFv. Alternatively, selection of biotag constructs were conducted via in vitro evolution involving phage display, yeast display, myeloma display, and/or ribosomal display. The selection method had no implication for the choice of expression, which was conducted in CHO and HEK 293 cells according to established protocols. Alternatively, cell free expression systems were used according to the standard protocols.

Chelating sites on scFvs were then covalently bound to gold nanoparticles to form gold-tagged biotags as described above. While the current example provides for the production of gold nanoparticles and gold-tagged biotags, nanoparticles using, other noble metals (e.g., Pt, Pd, Ag) were successfully manufactured according to previously developed methods well known to the technicians skilled in the art (Malecki 1996),. Purification of the gold-tagged biotags from non-bound metal particles was accomplished using affinity columns.

Determination of noble metal atoms per nanoparticle and number of nanoparticles tagging scFv. The number of atoms per nanoparticle was determined by measuring the diameter with FEEFTEM (Titan) or EFTEM (LEO912) or FESTEM (HB501) at zero loss followed by measuring MDN with EDX and/or EELS of the beam parked over the nanoparticle using the Si drifted detector or ccd chip (Noran, Zeiss or Gatan, respectively). The ratios of nanoparticles to scFv was determined by ratios between the noble metal nanoparticle and carbon counts from EDX and EELS in Zeiss 912 or Titan or VG equipped with Zeiss or Gatan software or with SPR.

### Example 3

### Generation of Superparamagnetic Metal-Tagged Single Chain Variable Fragment (scFv) Biotag

Plasmid constructs were described as previously described (Malecki et al. 2002 above). Coding sequences for variable fragment antibodies (scFvs) targeting ErbB 1-4 and TfR (and related variants or mutants), extended with internalization signals, endosomal/lysosomal escape signals, MBDs and cell surface anchor sequences were selected from the surface displayed libraries cloned into pM vectors designed with CMV immediate early promoter, Kozak sequence, SV40 poly(A) termination, and neomycin-resistance. Constructs an scFv, sdFv, CDR or SDR modified CDR were electroporated into human myelomas, CHO and/or HEK 293 cells. Expression of these constructs resulted in the surface display of the products. The expressor clones were selected, plasmids purified, and the sequences amplified as described above. This was followed by cloning without surface anchoring sequences, but by secretion into the medium an scFv, sdFv, CDR or SDR modified CDR. Chelating sites were saturated with metal ions: Gd, Eu, Ni, Co, Fe, Fe₃O₄ or with core shell, Au shelled, superparamagnetic nanoparticles. Purification from non-bound metal was performed on affinity columns. The myelomas were cultured in modified roller bottles (Sigma) Wave bioreactors or bioreactors (New Brunswick) according to standard protocols. Alternatively, cell free expression systems were used according to standard protocols.

Determination of metal atoms incorporated into chelating sites. For Example, the scFv chelating sites were saturated with Gd. Subsequently, these samples were purified on the affinity columns. Finally, they were analyzed with electron energy loss spectral imaging (EELS) and xray dispersive spectroscopy to determine total C to Gd ratio or in other words, the number of Gd atoms per scFv molecule.

Alternatively, the scFvs were altered through carboxyl terminal derivatization with Iodine (I) and their chelated sites saturated with Gd. Subsequently, these samples were purified on the gels as outlined below. They were analyzed using ratios between I and Gd using EDX and EELS.

### Example 4

### Validation of a Noble or Superparamagnetic Metal-Tagged Single Chain Variable Fragment (scFv) Biotag for Use in Detecting Cancer Cells In Vivo or In Vitro

The following materials and methods are used for the validation experiments described herein, but also apply to the experiments described in Examples 5 and 6, below.

Cell cultures. Many cell lines have been used to test the biotags described herein. Examples of such cell lines shown are shown in Table 3, and were grown in media recommended by ATCC in incubators (New Brunswick, Fisher, Napco) in saturated humidity, 37 deg C., 5% CO2. All cell lines were obtained from ATCC unless otherwise noted.

TABLE 3
Cell Lines

| | |
|---|---|
| Cell Lines that | SKBR3 from ATCC as HTB30 (overexpressed strongly) |
| Overexpress HER2 | UACC893 (20× gene amp) |
| | UACC812 (15× gene amp) |
| | CRL2338 from ATCC with designation HCC1954 (overexpressed strongly) |
| | AU565 from ATCC as CRL2351 (overexpressed strongly) |
| | MAC117 (gene amp 7×) |
| | MDA-MB453 (a bit more than MCF7, just above base ~3×) |
| | BT474 from ATCC as CRL |
| | CRL2340 from ATCC HCC2157 |
| | HCC2218 from ATCC as CRL2343 |
| | BT483 |
| Cell Lines that express a | HTB22 from ATCC with designation MCF7 (base ) |
| Basal Levels of HER2 | HBL100 |
| | MB231 |
| | HCC202 (basal or overexpressed) |
| | CRL 2320 HCC1008 from ATCC as (basal or overexpressed) metastatic |
| | NCl-H23 (basal or overexpressed) lung cancer |
| Cell Lines that are | CRL2314 from ATCC with designation HCC38 |
| negative for HER2 | CRL2315 HCC70 from ATCC |
| | CRL2321 HCC1143 from ATCC |
| | CRL2322 HCC1187 from ATCC |
| | CRL2324 HCC1395 from ATCC |
| | CRL2326 HCC1419 from ATCC |
| | CRL2327 HCC1428 from ATCC |
| | CRL2329 HCC1500 from ATCC |
| | CRL2330 HCC1569 from ATCC |
| | CRL2331 HCC1599 from ATCC |
| | CRL2336 HCC1937 from ATCC as (BRCA mut) |
| | CRL2343 HCC2218 from ATCC |
| Cell Lines that | HBE135_E6E7 from ATCC as CRL2741 (also high TGF) bronchial ducts |
| Overexpress EGFR | |
| Cell Lines that express a | CRL2918 from ATCC designation Nm2C5 EGFR pos (basal or over) |
| Basal Levels of EGFR | CRL2919 from ATCC designation Nm2C5 gfp EGFR pos (basal or over) |
| | M4A4 (basal or over) |
| | NCl-H23 (basal or over) |
| | Mutation A750del in EGFR CRL2868 adenocarcinoma |
| | Mutation A751del in EGFR CRL2869 adenocarcinoma |
| | Mutation A751del in EGFR CRL2871 adenocarcinoma |
| | HTB127 from ATCC with designation MDA-MB-330 (basal or over) |
| | HTB132 from ATCC with designation MDA-MB-468 (basal or over) |
| | HTB26 from ATCC designated MDA_MB_231 (basal or over) |
| Reference Cell Lines | EGFR A431 2-6 × 10⁶ receptors per cell |
| | HER2 BT474 6-10 × 10⁵ receptor per cell |
| | EGFR Normal breast primary culture 8% of A431 |
| | HER2 Normal breast primary culture 3% of A431 |
| Cell Line with a Mutation | Glioma |
| of EGFRvIII | |

Several of the cell lines used in the experiments described herein are further described. The cell lines TOV-112D CRL-11731 and CRL-117320V-90 were derived from primary malignant adenocarcinomas of the ovary at grade 3, stage IIIC. They were cultured in a 1:1 mixture of MCDB 105 medium and Medium 199, 85%; donor bovine serum 15% (ATCC). The cells were tumorigenic in nude mice. They formed colonies and spheroids when cultured in soft agar. The cells tested positive for HER2/neu and p53 mutation.

The cell line NIH OVCAR-3 HTB-161 was derived from the cells in ascites of a patient with malignant adenocarcinoma of the ovary. The cell line was grown in RPMI-1640 Medium (ATCC) supplemented with 0.01 mg/ml bovine insulin and donor bovine serum to a final concentration of 20%. The epithelial cells were positive for estrogen and progesterone receptor. They formed tumors in nude mice.

The cell line CRL-2340 HCC2157 was derived from the ductal carcinoma of the mammary gland tumor classified as TNM stage IIIA, grade 2, with lymph node metastasis. The cells were grown in a 1:1 mixture of Ham's F12 medium with 2.5 mM L-glutamine and Dulbecco's Modified Eagle's Medium adjusted to contain 1.2 g/L sodium bicarbonate with additional supplements (ATCC).

The cell line MCF7 HTB-22. The cells are positive for estrogen receptor and express WNT7B oncogene. The medium to culture this cell line is Eagle's Minimum Essential Medium (ATCC) with these added components: 0.01 mg/ml bovine insulin; donor bovine serum to a final concentration of 10%.

The cell line 184A1 CRL-8798 was originally established from normal mammary tissue and was transformed to benzopyrene. The line appears to be immortal, but is not malignant. The line grows in Mammary Epithelial Growth Medium (MEGM) (Clonetics) supplemented with 0.005 mg/ml transferrin and 1 ng/ml cholera toxin.

The normal, adherent fibroblast cell line Detroit 573 CCL-117 was derived from skin. It is grown in Minimum essential medium (Eagle) in Earle's BSS with non-essential amino acids (ATCC), sodium pyruvate (1 mM) and lactalbumin hydrolysate (0.1%), 90%; fetal bovine serum, 10%. The cells were grown into spheroids within a synthetic extracellular matrix.

Viability tests and doubling times. The cells were stained with Hoechst vs PI and counted on Beckman Coulter flow cytometer to determine ratios between total number of cells and dead cells at 24 hour intervals to determine doubling times and viability.

Selection of clones with high metastatic potential. For the in vitro studies described herein, cell lines described above were grown as described above. They were resuspended and spilled over the endothelial cells grown over extracellular basement membrane as described in the details previously (Malecki et al. 1989). After short incubation at 37° C., the cells cultures were rinsed with media, while removing non-adherent cancer cells. The attached cells were resuspended again and split into single clones grown in multiwell plates. These enriched clones were used for further studies because they imitated the metastatic clones of the lines derived from the primary tumor.

Patients' blood, lymph, peritoneal, pleural, and cerebrospinal fluids. Physiological fluids (blood, lymph, peritoneal, pleural and cerebrospinal fluids) were collected according to the standard clinical protocols. They were mixed with biotags as described above. They were tested with NMR, MRI, SPR, x-ray, CT, Raman, FCM, fluorescence confocal as described herein.

Isolation of receptors. Receptors for ErbB 1-4 (ErbB 1-4) and TfR (and related variants or mutants) were isolated from the ovarian, breast, testicular, brain cancer cells lines as previously described (Culouscou at al. 1993; Kraus et al. 1989; Prigent et al. 1992; Mori et al. 1987; Stern et al. 1986; Akiyama et al 1986). They were used for in vitro evolution, selection, affinity purification, and testing of the raised. The gene copy number and loci and the number of transcripts were evaluated by in situ hybridization and RT-PCR using the probes and primers listed below and according to protocols as previously described (Malecki 1996).

Immunolabeling. Cell spheroids grown in the culture were spun down at 300×g. The cells were resuspended in the donor serum or whole blood to which superparamagentic scFv were added. Upon completion of labeling, the cells were rinsed with PBS. They were studied with CT, MRI or NMR or alternatively processed by freezing in preparation for laser scanning confocal microscopy (LSCM) or EDXSI or EELS. Alternatively, cell lysates electrotransferred onto PVDF membranes were immunolabeled with scFv with or without chelated metal atoms.

Freezing and freeze-substitution of cell spheroids. The details of cryoimmobilization of cultures of cell spheroids by freezing are described previously and are only briefly presented here (Malecki 1992). Briefly, cells were injected into chambers were rapidly frozen in nitrogen slurry down to down to −196° C. The frozen samples were placed into methanol that was precooled to −90° C. in the freezer (ThermoNoran). Temperatures were maintained at −90° C., −35° C., and 0° C. for 48 hours. Infiltration with Lowicryl preceded polymerization with UV at −35° C. and ultramicrotomy. Alternatively, critical point drying was followed by fast atom beam sputter coating (IonTech).

Native electrophoresis. A 2% agarose gel was poured using a 10 mM Tris, 31 mM NaCl buffer of varying pH that did not contain any denaturing agents. The samples in their native state were loaded after being mixed with glycerol to add density without denaturing the proteins. The gel was run in the same buffer used for pouring the agarose at 60 mAmps until the desired separation was reached as determined by the presence of fluorescent markers with a molecular weight higher and lower than the scFv tested. The gel was then stained for 30 minutes in Sypro Tangerine Gel Stain (Invitrogen) diluted in the running buffer before imaging using a FluorImager (Molecular Dynamics).

SDS-PAGE. Electrophoresis was run on an 8-12% polyacrylamide gel. Several 0.75 thick combs with the 2 mm lanes were loaded with standard, cell culture lysates. The samples, after mixing with SDS and with or without DTT containing sample buffers (Sigma) were loaded into the wells. The gels were run using a Tris/Glycine/SDS/DTT running buffers. After the run, the gels were stained with colloidal silver or Sypro Tangerine for imaging using a FluorImager (Molecular Dynamics).

Electrotransfer. After electrophoresis, the samples were immediately transferred onto PVDF. The immunoblotting was performed with the Mini Trans-Blot Cell (Bio-Rad) within CAPS: 10 mM 3-[Cyclohexylamino]-1-propanesulfonic acid (CAPS), Tris/glycine transfer buffer 25 mM Tris base, 192 mM glycine, pH 8.3. Prior to the transfer, the cooling units were stored with deionized water at −20 C. Immediately after electrophoresis the gel, membrane, filter papers and fiber pads were soaked in transfer buffer for 5-10 minutes. The pre-cooled transfer units were filled with cooled transfer buffer and the electrotransfer proceeded at 350 mA.

Laser scanning confocal fluorescence microscopy and fluorometry. (LSCM) The three-dimensional stacks of the cells labeled with scFv against ErbB1-4 were imaged with the Olympus or Leica laser scanning confocal systems. Excitation wavelengths were used: 337, 488, 543, and 588 nm. Alternatively, reflected or Raman optics were used. Images were acquired with Kernel filtration and deconvolution of the data was followed by 3D or cascade display for analysis. For cytofluorometry and/or sorting the cells were labeled either with an scFv, sdFv, CDR or SDR modified CDR modified with standard fluorochromes (FITC, Cy5, Cy7, etc) or chelated Eu, Tb, etc, and detected with cytofluorometer or Sorter both from Becton&Dickinson or Beckman Coulter. The metal chelates provided not only very stable fluorescence, but also were available for validation of their distribution with spectral elemental mapping using EDXSI.

Spectral Mapping Using Energy Dispersive X-Ray Analysis Spectroscopic Imaging (EDXSI) and Electron Energy Loss Spectroscopic Imaging (EELSI). Supramolecular architecture analysis of the scFv against ErbB1-4 was performed with Field Emission Scanning Electron Microscope with Energy Dispersive X-Ray Spectral Imaging System (EDXSI)-Hitachi 3400. Complete elemental spectra were acquired for every pixel of the scans to create the elemental databases. From this, after selecting an element specific energy window, the map of this element atoms distribution was extracted and ZAF correction calculated (NIST). As scFv tagged with superparamagnetic metal particles (nanoclusters or core-shell nanoparticles) or noble metal nanoparticles were tagged or incorporated into their structures, their location was determined based upon spectral elemental maps superimposed over molecular architecture with zero loss or carbon edge tuning (Malecki 1995, Malecki et al 2001).

Purity of elemental composition and geometry of gold nanoparticles were evaluated with EDXSI using Vacuum Generators 501, Hitachi S900, and JEOL 1540 instruments under control of Gatan, Voyager software.

X-ray, atomic absorption spectroscopic, surface plasmon resonance (SPR) detection, centrifugation, and selection. One molecule of scFv tagged with one gold nanoparticle consisting of 100 atoms of gold with the diameter 1.59A and mass 197 amu each increased mass of scFv tagged up to 19,966 Da and that consisting of 1000 atoms up to 196,667Da. For 2M ErbB receptor single domains on cancer cell surface, multiplied by number of domains per one receptor, multiplied by internalized scFv tagged with nanoparticles of Au, the cell mass significantly increased, more than 1B times, in response to gravity during centrifugation at low g, compared to unlabeled non-cancerous cells. This did lead to very simple and rapid separation of cancer cells labeled with scFv tagged with Au from the aliquot of the patient's blood. Supernatant was used for hematological analysis, while pellet with cancer cells used for oncological analysis. Presence of cancer cells was detected in multiple ways: surface plasmon resonance on the pellet, electron induced x-ray spectra, transfer on a glass slide for light microscopy, dispersing into a solution for flow cytometry (direct flow cytometry was also conducted on the entire samples for comparison), Raman spectroscopy, passing into the microfluidic channels crossing the sensor's path, or injecting into cell counting chamber or running flow cytometry based upon scattering or after introducing fluorescent stains as detailed above.

CT—Computed x-ray Tomography. For evaluating relative contrast agents in CT, solutions of 1M, 0.1M, 0.01M, and 0.001M, 0.0001M sodium iodide (equivalent of commercial contrast agents), calcium chloride (equivalent of bones), gold chloride, and gold nanoparticles of various sizes in deionized water were dispensed into the wells of microarray plates. Additional rows contained blood, physiological saline, while an additional row was left empty, i.e., to contain air.

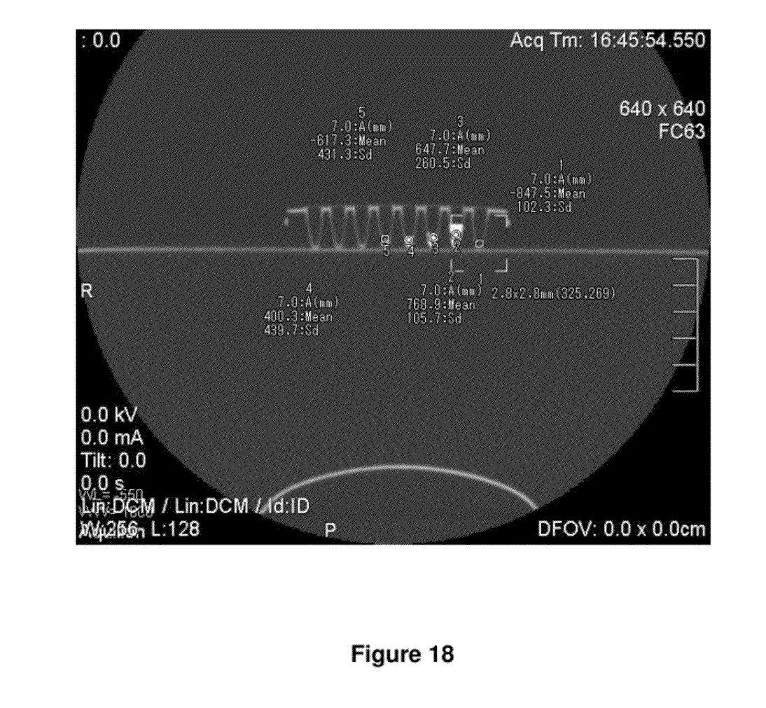

Computed tomography was pursued with Toshiba Aquilion 64-slice clinical scanner. Initial settings were as follows: voltage 120 peak kV, current 40 mA, exposure time of 0.6 s, slice setting 0.5 mm (the slices that were thereafter compressed into 2 mm display images), (modifications of these settings were indicated in the figure legends). ImageQuantTL® version 1.1.0.1 was used to evaluate relative peak pixel intensity of the samples on the computed tomography images utilizing a 0 to 255 level grayscale. The Aquilion scanner may also record phantoms for use in detecting biomarker density by measuring the signal intensity of the biotags in Haunsfield units (see, e.g., FIG. 18).

Nuclear magnetic resonance and selection. The wide-bore nuclear magnetic resonance (NMR) spectrometer operated at 9T (Brucker) with a mouse-cage resonator was used to evaluate relative relaxivity of the samples based upon T1 measurements. T1 spin lattice relaxation time calculated using inversion recovery pulse sequence was measured using inversion recovery imaging with TI=50-4000 ms in 100 ms increments. T1 was also calculated from T1-weighted fluid-attenuated inversion recovery (T1-FLAIR) sequence (Tr/Te/Flip=2210/9.6/90), as well as standard T1-weighted imaging sequences (Tr/Te/Flip=400/6/90).

For single cell detection, a small table top NMR spectrometer was used at 0.5T. After labeling with superparamagnetic scFv, the blood sample containing labeled cancer cells was injected into microfluidic channel of 20 micron in diameter, which was placed with the field. Passage of the single cell, which was labeled with superparamagnetic scFv, was determined by the spectral response and recorded.

Alternatively, a tube or plate containing an aliquot of the patient's blood supplemented with a varying number of cancer cells labeled with the superparamagnetic scFv against ErbB1-4 may be placed in the magnetic field of a magnetic source. The labeled cells were retained in the magnetic field, while the non-labeled blood cells were withdrawn (FIG. 16). After rinsing with PBS, the labeled cancer cells were retained for further studies on the counting chamber, fluorometer, and/or confocal.

Calculation of receptor number per cancer tumor volume. To determine the number of the receptors per cells, the cells were labeled with IgG, Fab, and our scFv for fluorescent, NMR, SPR, ELISA and RIA, assays, which were performed according to standard techniques.

RT-PCR for ErbB1-4 and TFR gene expression ratios. The cell cultures were homogenized and mRNA reverse transcribed to cDNA. After mixing cDNA with primers and salts, the samples were loaded onto ABI Fast 7500 of 7900 thermal cycler. The transcript numbers were compared using standard ABI software.

PCR for ErbB 1-4 gene copy numbers. The cell cultures were homogenized and mRNA reverse transcribed to cDNA. After mixing cDNA with primers and salts, the samples were loaded onto ABI Fast 7500 of 7900 thermal cycler. The transcript numbers were compared.

Fluorescent In Situ Hybridization for evaluation of the gene copy numbers. The cells in cultures were arrested in metaphase with taxol. They were fixed with methanol/acetic acid mixture and splash spread onto glass cover slips and dried. After protease and formamide treatment, they were hybridized with DNA probes tagged with either nanogold, superparamagnetic nanoparticles (e.g. Eu) or fluorochrome (FITC, Rhodamine, Cy3, Cy5). They were imaged with confocal microscopy either in fluoro- or reflected mode.

Primers and probes to ErbB 1-4 and TFR. Primers and probes used for RT-PCR and fluorescent in situ hybridization may include, but are not limited to those found in Table 4 below. In the table, “len” is the primer or oligo length, “tm” is the melting temperature of the primer or oligo, “gc %” is the percent of G or C bases in the primer or oligo, “any” is the self-complementarity of the primer or oligo, taken as a measure of its tendency to anneal to itself or form secondary structure, “3” is the 3′ self-complementarity of the primer or oligo, taken as a measure of its tendency to form a primer-dimer with itself, and “seq” is the sequence of the primer or oligo, always from right to left, 5′ to 3′. Additional primers and probes that may be used in accordance with the methods described herein can be found in Appendix A, which is hereby incorporated by reference as is fully set forth herein.

TABLE 4
Primers and probes to ErbB 1-4 and TfR.

| | OLIGO | len | tm | gc % | any | 3′ | seq |
|---|---|---|---|---|---|---|---|
| ErbB1 | LEFT PRIMER | 20 | 60.01 | 50.00 | 6.00 | 1.00 | Cagcgctaccttgtcattca (SEQ ID NO: 298) |
| | RIGHT PRIMER | 20 | 60.00 | 55.00 | 7.00 | 2.00 | Tgcactcagagagctcagga (SEQ ID NO: 298) |
| | HYB OLIGO | 20 | 60.08 | 45.00 | 8.00 | 3.00 | gaatgcatttgccaagtcct (SEQ ID NO: 299) |
| ErbB1 | LEFT PRIMER | 20 | 60.00 | 55.00 | 3.00 | 1.00 | gggctcacagcaaacttctc (SEQ ID NO: 300) |
| | RIGHT PRIMER | 20 | 60.02 | 50.00 | 7.00 | 0.00 | aagccagactcgctcatgtt (SEQ ID NO: 301) |
| | HYB OLIGO | 20 | 60.00 | 55.00 | 2.00 | 2.00 | acacacacacacacacaccg (SEQ ID NO: 302) |
| ErbB1 | LEFT PRIMER | 20 | 60.00 | 55.00 | 3.00 | 1.00 | ggctcacagcaaacttctcc (SEQ ID NO: 303) |
| | RIGHT PRIMER | 20 | 60.02 | 50.00 | 7.00 | 0.00 | aagccagactcgctcatgtt (SEQ ID NO: 301) |
| | HYB OLIGO | 20 | 60.00 | 55.00 | 2.00 | 2.00 | acacacacacacacacaccg (SEQ ID NO: 302) |
| ErbB1 | LEFT PRIMER | 20 | 59.97 | 50.00 | 4.00 | 2.00 | acttgacaggggaaacatgc (SEQ ID NO: 304) |
| | RIGHT PRIMER | 20 | 60.00 | 55.00 | 3.00 | 3.00 | caaggtctgggaaccactgt (SEQ ID NO: 305) |
| | HYB OLIGO | 20 | 60.09 | 40.00 | 4.00 | 2.00 | ttgcacaattccaaccttga (SEQ ID NO: 306) |
| ErbB1 | LEFT PRIMER | 20 | 60.00 | 55.00 | 3.00 | 1.00 | ggctcacagcaaacttctcc (SEQ ID NO: 303) |
| | RIGHT PRIMER | 20 | 59.97 | 50.00 | 4.00 | 1.00 | gcatgtttcccctgtcaagt (SEQ ID NO: 307) |
| | HYB OLIGO | 20 | 60.00 | 55.00 | 2.00 | 2.00 | acacacacacacacacaccg (SEQ ID NO: 302) |
| ErbB1 | LEFT PRIMER | 20 | 60.00 | 55.00 | 3.00 | 1.00 | gggctcacagcaaacttctc (SEQ ID NO: 300) |
| | RIGHT PRIMER | 20 | 59.97 | 50.00 | 4.00 | 1.00 | gcatgtttcccctgtcaagt (SEQ ID NO: 307) |
| | HYB OLIGO | 20 | 60.00 | 55.00 | 2.00 | 2.00 | acacacacacacacacaccg (SEQ ID NO: 302) |
| ErbB2/ | LEFT PRIMER | 20 | 59.99 | 55.00 | 2.00 | 0.00 | ccataacacccacctctgct (SEQ ID NO: 308) |
| HER2 | RIGHT PRIMER | 20 | 59.95 | 55.00 | 6.00 | 3.00 | actggctgcagttgacacac (SEQ ID NO: 309) |
| | HYB OLIGO | 20 | 60.06 | 55.00 | 4.00 | 1.00 | accaagctctgctccacact (SEQ ID NO: 310) |
| ErbB2/ | LEFT PRIMER | 20 | 59.94 | 55.00 | 8.00 | 0.00 | acacagcggtgtgagaagtg (SEQ ID NO: 311) |
| HER2 | RIGHT PRIMER | 20 | 60.09 | 65.00 | 4.00 | 0.00 | aggccaggggtagagagtag (SEQ ID NO: 312) |
| | HYB OLIGO | 20 | 59.65 | 55.00 | 3.00 | 3.00 | tcagaccctcttgggaccta (SEQ ID NO: 313) |
| ErbB2/ | LEFT PRIMER | 20 | 60.16 | 55.00 | 3.00 | 3.00 | gcctccacttcaaccacagt (SEQ ID NO: 314) |
| HER2 | RIGHT PRIMER | 20 | 59.99 | 55.00 | 4.00 | 2.00 | cccacgtccgtagaaaggta (SEQ ID NO: 315) |
| | HYB OLIGO | 20 | 60.31 | 55.00 | 5.00 | 2.00 | tgtgactgcctgtccctaca (SEQ ID NO: 316) |
| ErbB2/ | LEFT PRIMER | 20 | 59.84 | 55.00 | 4.00 | 0.00 | cccagctctttgaggacaac (SEQ ID NO: 317) |
| HER2 | RIGHT PRIMER | 20 | 59.91 | 50.00 | 8.00 | 0.00 | agccagctggttgttcttgt (SEQ ID NO: 318) |
| | HYB OLIGO | 20 | 59.89 | 55.00 | 10.00 | 3.00 | agcttcgaagcctcacagag (SEQ ID NO: 319) |
| ErbB2/ | LEFT PRIMER | 20 | 59.91 | 55.00 | 4.00 | 3.00 | tggggagagagttctgagga (SEQ ID NO: 320) |
| HER2 | RIGHT PRIMER | 20 | 60.16 | 50.00 | 7.00 | 1.00 | acagatgccactgtggttga (SEQ ID NO: 321) |
| | HYB OLIGO | 20 | 60.16 | 57.89 | 8.00 | 8.00 | gactgctgccatgagcagt (SEQ ID NO: 322) |
| ErbB2/ | LEFT PRIMER | 20 | 59.84 | 55.00 | 4.00 | 0.00 | cccagctctttgaggacaac (SEQ ID NO: 317) |
| HER2 | RIGHT PRIMER | 20 | 59.87 | 55.00 | 4.00 | 0.00 | ggatcaagacccctcctttc (SEQ ID NO: 323) |
| | HYB OLIGO | 20 | 59.89 | 55.00 | 10.00 | 3.00 | agcttcgaagcctcacagag (SEQ ID NO: 319) |
| ErbB2/ | LEFT PRIMER | 20 | 59.99 | 55.00 | 2.00 | 0.00 | ccataacacccacctctgct (SEQ ID NO: 308) |
| HER2 | RIGHT PRIMER | 20 | 59.95 | 55.00 | 6.00 | 3.00 | actggctgcagttgacacac (SEQ ID NO: 309) |
| | HYB OLIGO | 20 | 60.06 | 55.00 | 4.00 | 1.00 | accaagctctgctccacact (SEQ ID NO: 310) |
| ErbB2/ | LEFT PRIMER | 20 | 59.93 | 50.00 | 5.00 | 3.00 | ccatctgcaccattgatgtc (SEQ ID NO: 324) |
| HER2 | RIGHT PRIMER | 20 | 60.02 | 60.00 | 3.00 | 1.00 | gagcggtagaaggtgctgtc (SEQ ID NO: 325) |
| | HYB OLIGO | 20 | 59.97 | 50.00 | 4.00 | 4.00 | cgggagttggtgtctgaatt (SEQ ID NO: 326) |
| ErbB2/ | LEFT PRIMER | 20 | 60.05 | 55.00 | 2.00 | 0.00 | ccctcatccaccataacacc (SEQ ID NO: 327) |
| HER2 | RIGHT PRIMER | 20 | 59.95 | 55.00 | 6.00 | 3.00 | actggctgcagttgacacac (SEQ ID NO: 309) |
| | HYB OLIGO | 20 | 60.06 | 55.00 | 4.00 | 1.00 | accaagctctgctccacact (SEQ ID NO: 310) |
| ErbB2/ | LEFT PRIMER | 20 | 60.05 | 50.00 | 3.00 | 2.00 | cgcttttggcacagtctaca (SEQ ID NO: 328) |
| HER2 | RIGHT PRIMER | 20 | 60.07 | 55.00 | 5.00 | 3.00 | tcccggacatggtctaagag (SEQ ID NO: 329) |
| | HYB OLIGO | 20 | 59.93 | 45.00 | 6.00 | 2.00 | aattccagtggccatcaaag (SEQ ID NO: 330) |
| ErbB2/ | LEFT PRIMER | 20 | 59.93 | 45.00 | 6.00 | 2.00 | aattccagtggccatcaaag (SEQ ID NO: 330) |
| HER2 | RIGHT PRIMER | 20 | 60.07 | 55.00 | 5.00 | 3.00 | tcccggacatggtctaagag (SEQ ID NO: 329) |
| | HYB OLIGO | 20 | 60.14 | 55.00 | 5.00 | 2.00 | ggtgacacagcttatgccct (SEQ ID NO: 331) |
| ErbB2/ | LEFT PRIMER | 20 | 59.93 | 45.00 | 6.00 | 2.00 | aattccagtggccatcaaag (SEQ ID NO: 330) |
| HER2 | RIGHT PRIMER | 20 | 59.93 | 50.00 | 5.00 | 3.00 | tttcccggacatggtctaag (SEQ ID NO: 332) |
| | HYB OLIGO | 20 | 60.14 | 55.00 | 5.00 | 2.00 | ggtgacacagcttatgccct (SEQ ID NO: 331) |
| ErbB3 | LEFT PRIMER | 20 | 59.95 | 60.00 | 3.00 | 3.00 | gagcccagaggagaagact (SEQ ID NO: 333) |
| | RIGHT PRIMER | 20 | 59.99 | 55.00 | 6.00 | 0.00 | tctgatgcgacagacactcc (SEQ ID NO: 334) |
| | HYB OLIGO | 20 | 59.83 | 60.00 | 3.00 | 0.00 | gagtctgagtgttcggaggg (SEQ ID NO: 335) |
| ErbB3 | LEFT PRIMER | 20 | 59.93 | 50.00 | 4.00 | 2.00 | aattgactggagggacatcg (SEQ ID NO: 336) |
| | RIGHT PRIMER | 20 | 60.12 | 55.00 | 3.00 | 3.00 | ggagcacagatggtcttggt (SEQ ID NO: 337) |
| | HYB OLIGO | 20 | 59.87 | 50.00 | 4.00 | 2.00 | aggacaatggcagaagctgt (SEQ ID NO: 338) |
| ErbB3 | LEFT PRIMER | 20 | 59.93 | 50.00 | 4.00 | 2.00 | aattgactggagggacatcg (SEQ ID NO: 336) |
| | RIGHT PRIMER | 20 | 60.26 | 55.00 | 3.00 | 1.00 | aggagcacagatggtcttgg (SEQ ID NO: 339) |
| | HYB OLIGO | 20 | 59.87 | 50.00 | 4.00 | 2.00 | aggacaatggcagaagctgt (SEQ ID NO: 338) |
| ErbB3 | LEFT PRIMER | 20 | 59.87 | 50.00 | 4.00 | 2.00 | aggacaatggcagaagctgt (SEQ ID NO: 338) |
| | RIGHT PRIMER | 20 | 60.32 | 60.00 | 4.00 | 1.00 | cgaggtacacaggctccact (SEQ ID NO: 340) |
| | HYB OLIGO | 20 | 60.12 | 55.00 | 3.00 | 2.00 | accaagaccatctgtgctcc (SEQ ID NO: 341) |
| ErbB3 | LEFT PRIMER | 20 | 59.68 | 50.00 | 8.00 | 2.00 | ggaagtttgccatcttcgtc (SEQ ID NO: 342) |
| | RIGHT PRIMER | 20 | 59.87 | 50.00 | 4.00 | 0.00 | acagcttctgccattgtcct (SEQ ID NO: 343) |
| | HYB OLIGO | 20 | 59.93 | 50.00 | 4.00 | 2.00 | aattgactggagggacatcg (SEQ ID NO: 336) |
| ErbB3 | LEFT PRIMER | 20 | 60.34 | 60.00 | 6.00 | 2.00 | gagggacccaggtctacgat (SEQ ID NO: 344) |
| | RIGHT PRIMER | 20 | 59.87 | 50.00 | 4.00 | 0.00 | acagcttctgccattgtcct (SEQ ID NO: 343) |
| | HYB OLIGO | 20 | 59.93 | 50.00 | 4.00 | 2.00 | aattgactggagggacatcg (SEQ ID NO: 336) |
| ErbB4 | LEFT PRIMER | 20 | 60.04 | 45.00 | 3.00 | 0.00 | tttcgggagtttgagaatgg (SEQ ID NO: 345) |
| | RIGHT PRIMER | 20 | 59.97 | 50.00 | 7.00 | 2.00 | gaaactgtttgccccctgta (SEQ ID NO: 346) |
| | HYB OLIGO | 20 | 60.04 | 50.00 | 4.00 | 4.00 | aagatggaagatggcctcct (SEQ ID NO: 347) |
| ErbB4 | LEFT PRIMER | 20 | 59.91 | 45.00 | 5.00 | 2.00 | ggtgaatttcgggagtttga (SEQ ID NO: 348) |
| | RIGHT PRIMER | 20 | 59.97 | 50.00 | 7.00 | 2.00 | gaaactgtttgccccctgta (SEQ ID NO: 346) |
| | HYB OLIGO | 20 | 60.04 | 50.00 | 4.00 | 4.00 | aagatggaagatggcctcct (SEQ ID NO: 347) |
| ErbB4 | LEFT PRIMER | 20 | 59.97 | 45.00 | 5.00 | 2.00 | ggtgcttttggaacggttta (SEQ ID NO: 349) |
| | RIGHT PRIMER | 20 | 59.84 | 55.00 | 4.00 | 0.00 | aaccggactaggtgtggatg (SEQ ID NO: 350) |
| | HYB OLIGO | 20 | 59.69 | 45.00 | 4.00 | 3.00 | caaggcaaatgtggagttca (SEQ ID NO: 351) |
| ErbB4 | LEFT PRIMER | 20 | 59.97 | 45.00 | 5.00 | 2.00 | ggtgcttttggaacggttta (SEQ ID NO: 349) |
| | RIGHT PRIMER | 20 | 59.84 | 55.00 | 4.00 | 2.00 | caaccggactaggtgtggat (SEQ ID NO: 352) |
| | HYB OLIGO | 20 | 59.69 | 45.00 | 4.00 | 3.00 | caaggcaaatgtggagttca (SEQ ID NO: 351) |
| ErbB4 | LEFT PRIMER | 20 | 60.15 | 55.00 | 7.00 | 2.00 | ccagaccaatgtctgtcgtg (SEQ ID NO: 353) |
| | RIGHT PRIMER | 20 | 60.04 | 50.00 | 4.00 | 0.00 | aggaggccatcttccatctt (SEQ ID NO: 354) |
| | HYB OLIGO | 20 | 60.04 | 45.00 | 3.00 | 0.00 | tttcgggagtttgagaatgg (SEQ ID NO: 345) |

Screening for mutations. Genomic DNA was isolated from cells in cultures and digested. Primers selected to flank selected regions of ErbB1-4 and TfR coding sequences were amplified and sequenced.

Validation of Gold Nanoparticle-Tagged Anti-ErbB and Anti-TFR scFv Biotags (Au*Biotag)

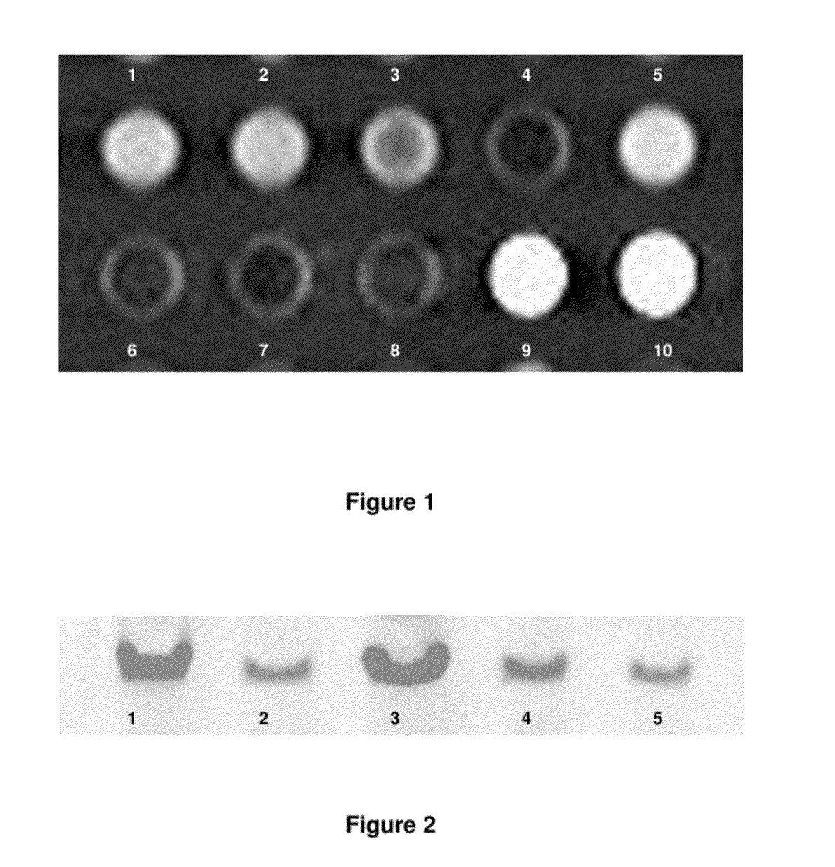

Several cancer cell lines were grown in extracellular matrix to validate the detection of cancer cells in CT with biotags tagged with gold nanoparticles. Each well contained a different cell line (AU565 (1), UACC812 (2), MDA-MB453 (3), basal level control (4), UACC893 (5), normal breast culture cells (6), connective and epithelial tissue normal control cells (7-8), DKBR3 (9), and CRL2338 (10); FIG. 1). They were labeled with the anti ErbB scFv tagged with gold clusters (Au*biotags). Immersed in serum, they were imaged with CT to determine the level of gene expression product for each cell line. Results are shown in FIG. 1

Cells strongly over-expressing ErbB 1-4, (i.e., having a high number of ErbB 1-4 gene expression products) that are labeled with an anti-ErbB Ag*biotag, and appear as bright spots in the CT (FIG. 1). Brighter spots are indicative of a higher the number of ErbB 1-4 gene expression products on the cells, which in turn means a brighter spot is indicative of more malignant cells. Thus, brightness is determinative of cell malignancy. This is a much more accurate determination of malignancy than the radionuclide, 18FDG, used in PET, because 18FDG is only indicative of increased metabolism, not malignancy. Computed tomography was pursued with Toshiba Aquilion 64-slice clinical scanner. Initial settings were as follows: voltage 120 peak kV, current 40 mA, exposure time of 0.6 s, slice setting 0.5 mm (the slices that were thereafter compressed into 2 mm display images). ImageQuantTL® version 1.1.0.1 was used to evaluate relative peak pixel intensity of the samples on the computed tomography images utilizing a 0 to 255 level grayscale.

Additionally, the expression level of the EGF receptor, HER2, in several cancer cells (MDA453, SKBR3, MCF7) was determined by electroblotting after being labeled with the Au*biotags (FIG. 2). Cell lines were grown in ECM matrix. After lysis and electrophoresis in 2% agarose, they were transferred onto the PVDF membranes. They were labeled with biotags for HER2 tagged with Au nanoparticles. The intensity of the bands reflects levels of gene expression products in these cells.

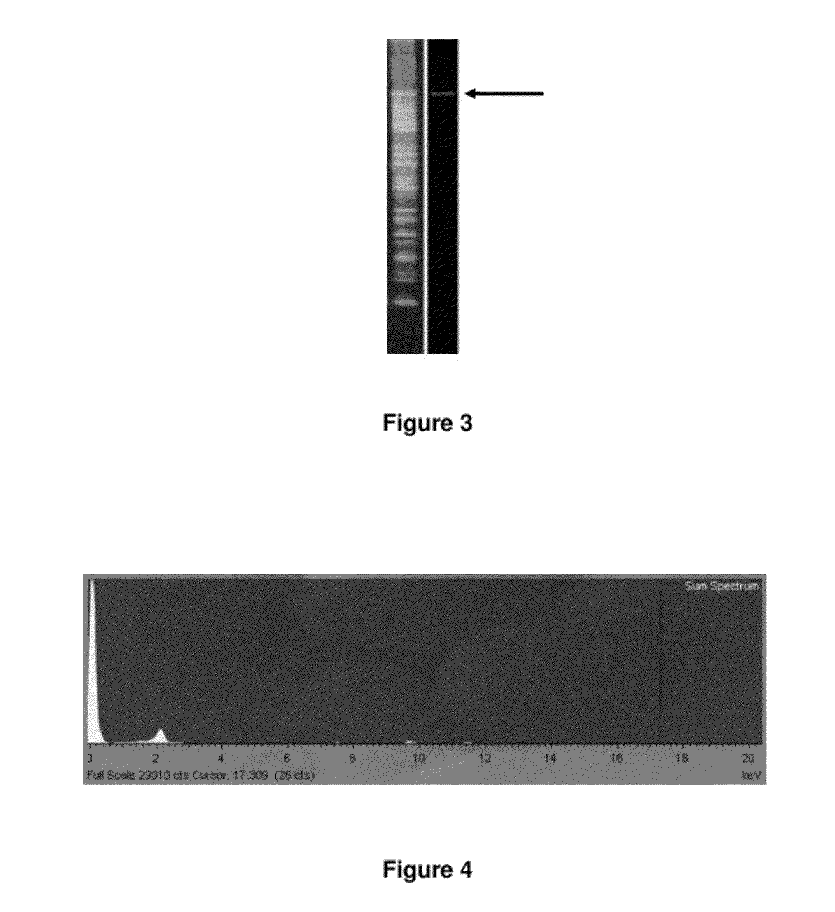

The Au*biotags exclusively target the receptors in the ErbB family (HER2 shown). Exquisite specificity is a characteristic for the biotags described herein. For example, specificity toward HER2 by the Au*biotag is illustrated in FIG. 3. After lysis and electrophoresis, the SKBR3 cancer cells grown in culture were stained with silver stain showing all the proteins contributing to the cell structure (left panel). After electroblotting onto PVDF membranes, the proteins were labeled with antiHER2 Au*biotags. The unique specificity of anti ErbB is demonstrated by a single band (right panel, arrow), indicating that only one domain within one protein receptor was labeled. The image was acquired using a Storm 840, Molecular Dynamics imaging system.

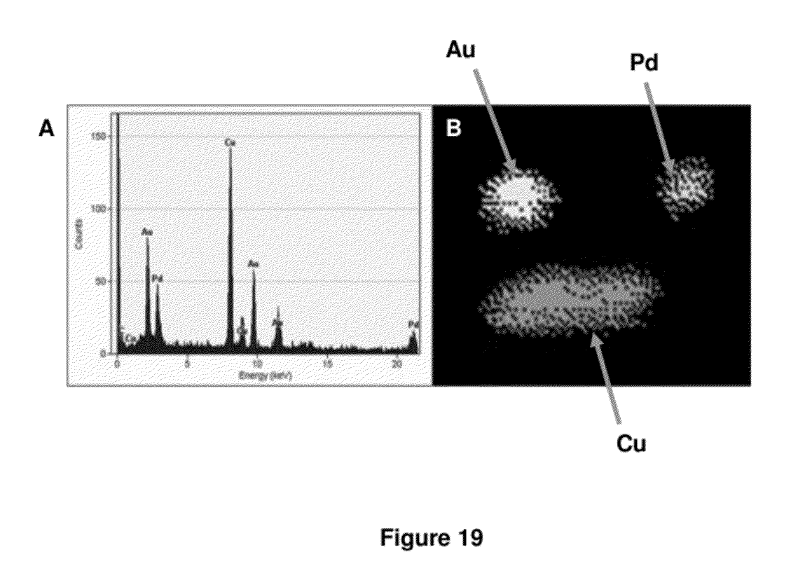

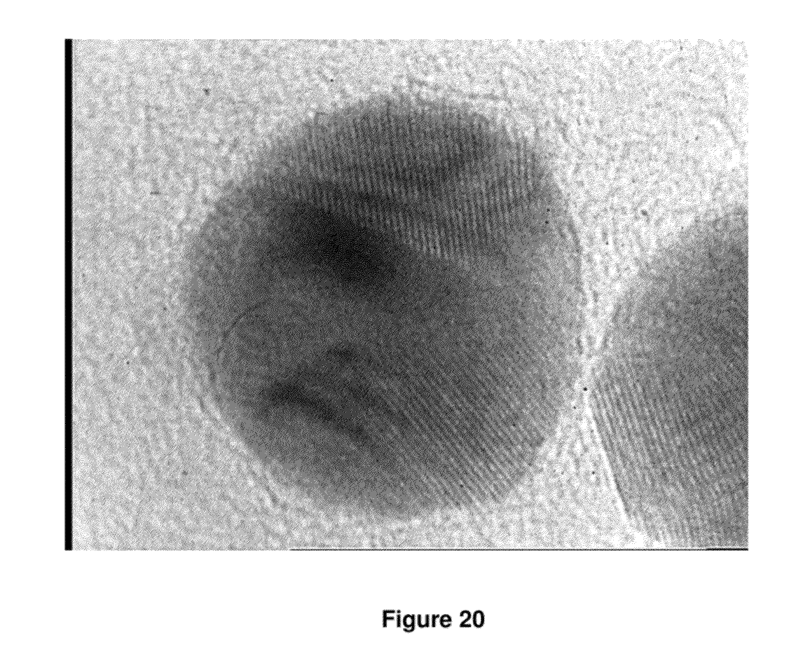

The composition of the biotags was validated by electron microscopy. biotags harboring nanoparticles of different metals were mixed together and sprayed onto a carrier, frozen and freeze-dried. An example image of Biotag harboring Au crystals is shown in FIG. 20. Stacks of data were then acquired at different energies by a FESTEM HB501 equipped with a Noran EDX system. After acquisition or on the fly, the data were analyzed using Noran software to select and display the spectra specific for particular elements. As shown in FIG. 19A, the integrated spectrum shows energy peaks for Au, Pd, Cu (FIG. 19A) and the composition of the individual biotags was gated for its specific element (FIG. 19B).

Electron microscopy was also used to show that the Au*biotags are selectively and permanently tagged with gold nanoparticles. FIG. 4 shows the elemental composition of the Au*biotags. Biotags tagged with Au nanocrystals were placed in chambers, then rapidly frozen and freeze-dried. A spectrum was generated using a INCA x-sight ISO 9001. The spectrum was taken at 21 kV, 71 point spot size, and a 20 mm working distance. The image and spectra were generated using INCA-Analyzer software. EDX validated the clean elemental composition of the anti ErbB scFv tagged with Au. The peak at ˜300 eV represents carbon (from scFv), while the peaks at 2.1 keV, 9.7 keV, and 11.5 keV represent gold (from nanocrystal tag) (FIG. 4).

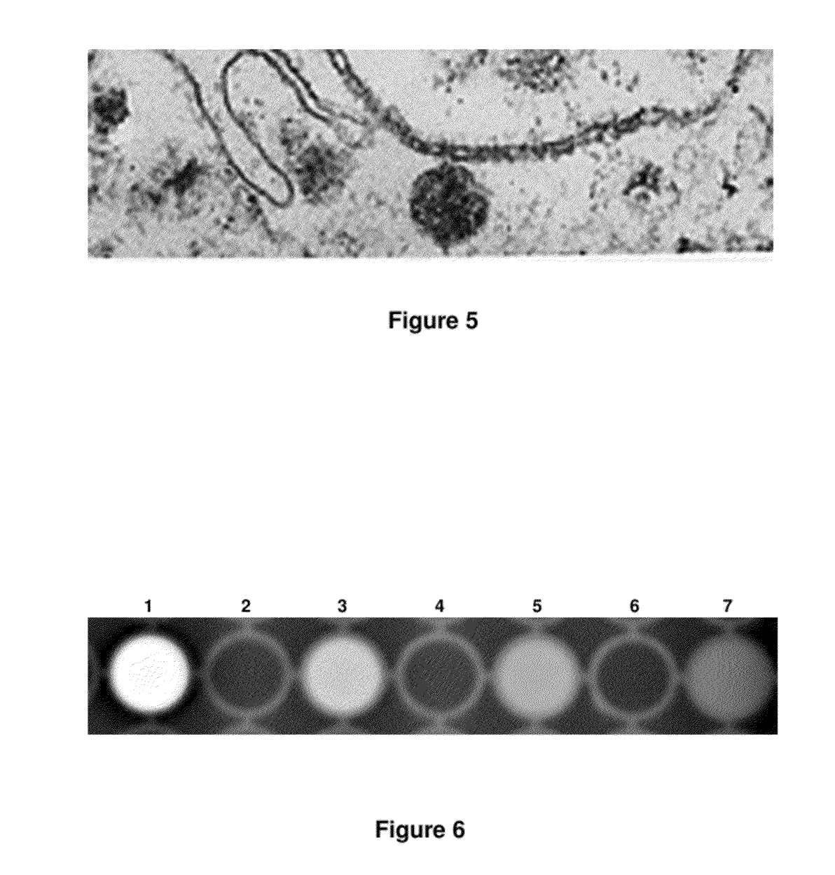

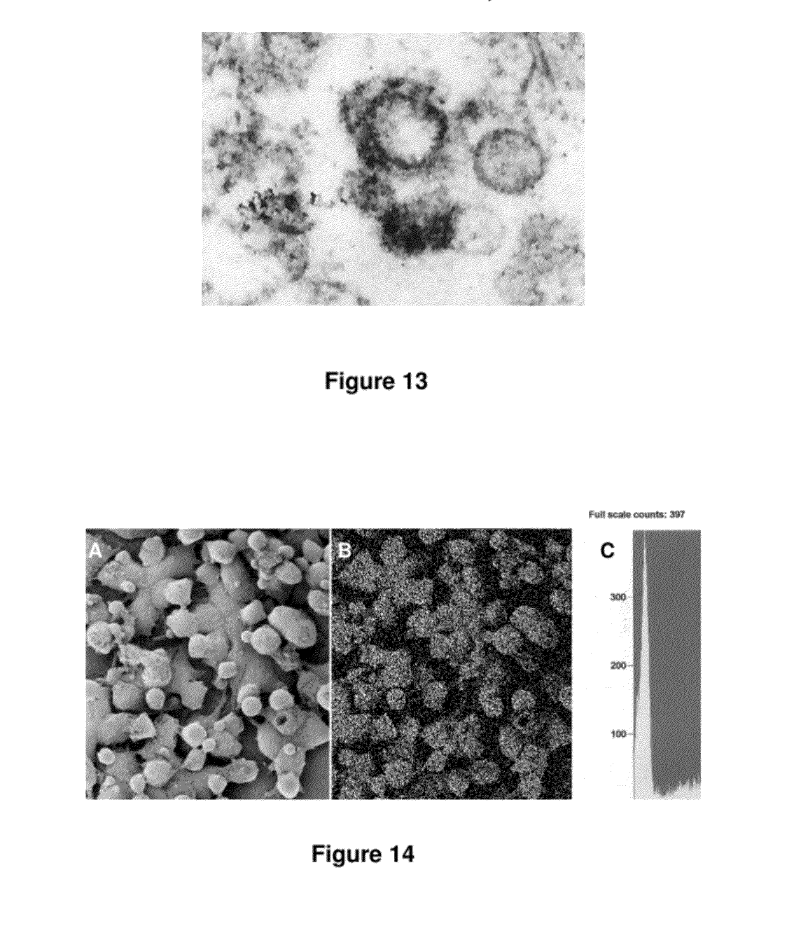

Further, the mechanisms involved were determined to be related to internalization of the probe by the cells after binding the receptor. Au*biotags undergo rapid internalization by SKBR3 cells. Cancer cells were grown as monolayers on ECM. A pulse-chase experiment was then conducted. The cells were then labeled with an antiHER2 Au*biotag for 3 min followed by thorough rinsing. The cells were then rapidly frozen, freeze-substituted, embedded, and processed for ultrastructural analysis. They were imaged with the laser scanning confocal microscope with the image acquisition in the reflection mode (FIGS. 5 and 13). The endosomes containing Au*biotags are reflected by the laser of the confocal microscope, creating little “mirrors” that give a very strong signal.

As it is clear from FIGS. 5 and 13, the scFv tagged with gold nanoparticles (Au*biotags) were internalized very efficiently. These images illustrate that the biotags are internalized very fast. Moreover, they escaped from the endosome early on, due to the endosomal escape domain. After being released from endosomes they are retained in the cytoplasm without being recycled to the cell surface and exterior, which contributes to the substantial increase of the Au content inside the cells while having no harm on the cell metabolism as demonstrated by no change in doubling time and thymidine incorporation. Additionally, the cells, after labeling with scFv tagged with Au were washed with PBS and retained in cultures for 24 hours. The media was then collected, and the EDX spectra of the media was examined. No gold was released from the cells into the media, further demonstrating that binding and internalizing the biotag resulted in permanently tagging the cells. Three elements are likely important for the success of the biotags described herein: high specificity of scFv retained after tagging with gold, internalization of scFv, and escape from endocytotic/lysosomal pathways. Together, these elements result in permanent tagging of the cancer cells. Permanent tagging by the biotags establishes an effective tool for use in various detection, diagnosis, therapy and prognosis practices in clinical and experimental medicine.

Various levels of gene expression can be detected in an x-ray, which is similar to screening with a standard mammography exam. Cultured SKBR3 cells were labeled with biotags tagged with gold nanoparticles and the content of gold was measured to determine 1.1 M concentration. Thereafter, the sample was diluted 10× and so were subsequent dilutions. The radiogram shows that concentrations as little as 1.1 mM can be still detected (FIG. 6). This experiment shows that the signal can be enhanced 1000× during mammography, exceeding the sensitivity of routine mammography with x-rays, CT, MRI, and approaching that of PET and SPECT, but without risks of administrations of radioactive substances to the patient's body and without the need of dedicated facilities to perform such examinations (as PET or MRI), and without the need of monitoring patient's urine and feces being radioactive.

Further, various sizes of tumors, including tumors smaller than can be detected by mammography, can be detected by x-ray diagnostic methods such as CT using the biotags described herein. An exemplar CT phantom as illustrated in FIG. 18 mimics a CT phantom used to detect different sizes of cancer tumors. Cultured SKBR3 cells were labeled with antiHER2 biotags. After rinsing and counting, they were injected into PCR plates. The cells were plated at different volumes (FIG. 18, left to right: 25 μl, 50 μl, 100 μl and 200 μl). The phantom was placed within the Aquilion clinical CT operated at 120 kV. Stacks of 2 mm slices were acquired. Even the smallest volume, 25 microliters, which corresponds to a radius of 1.8 mm, may be detected by CT when the cells are labeled with a biotag targeting a cancer biomarker. Currently routine mammography only detects tumors reaching one inch in diameter or 25 mm (˜7238 mm³ or 7238 microliters).

Validation of Superparamagnetic Metal-Tagged Anti-ErbB and antiTfR scFv Biotags

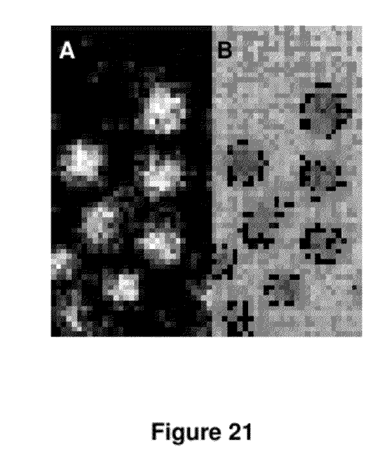

The composition of the superparamagnetic biotags was validated by electron microscopy. For example, biotags harboring core-shell ((Fe³)₄/Fe²O³—Au) crystal was frozen and freeze-dried onto a carrier film supported by a 1000 mesh grid. The Biotag was imaged by the EFTEM Zeiss at zero loss (FIG. 21B) and with the energy spectrum filtered for Fe (FIG. 21A) and acquired on Fuji film. The image shows Fe cores, which validates the composition of Biotags harboring superparamagnetic nanoparticles.

The cell lines in this study, TOV-112D CRL-11731, OV-90 CRL-11732, CRL-2340 HCC2157, NIH OVCAR-3, HTB-161, MCF7 HTB-22, 184A1 CRL-8798, Detroit 573 CCL-117 cells and cell spheroids were cultured and labeled with anti-HER2/neu superparamagnetic scFv antibodies. Cultured cells were labeled with scFv chelating Gd or Eu atoms (Gd*biotags; Eu*biotags) and were rapidly frozen. Frozen cells were freeze-substituted with no metal incorporation, infiltrated, and embedded. The distribution of scFv harboring metal atoms in ultrathin sections or cell whole mounts was examined with elemental mapping systems (FIG. 14A). The scFv chelating Gd atoms were anchored to the cell surface receptors as shown in FIG. 14B. Thereafter, they were visualized by mapping Gd. This is due to the acquisition of the full spectrum for every pixel of the scan to create the elemental data base. Thereafter, an energy window selected for Gd allowed for extracting element distribution within the entire image-element distribution map, or spectra (FIG. 14C). This elemental map based antibody distribution was projected onto the cell surface ultrastructure to determine localization of superparamagnetic scFv at the molecular level.

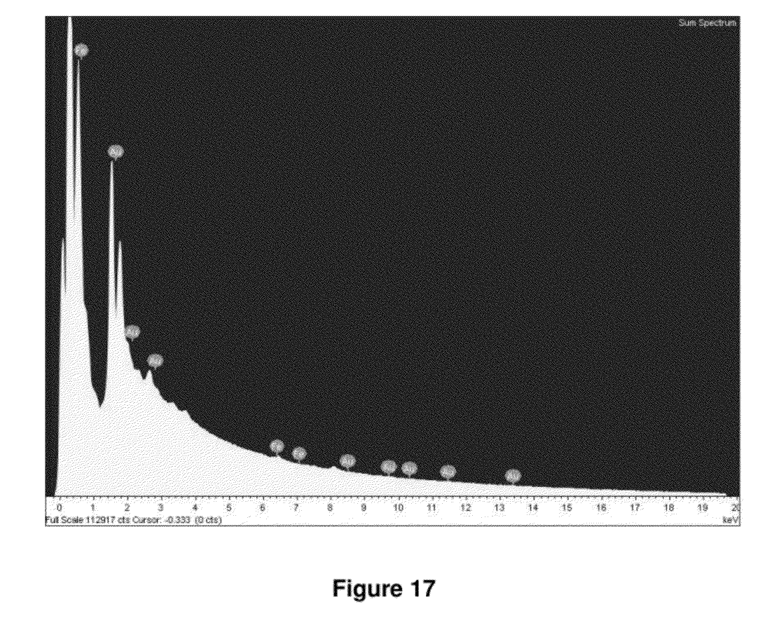

In another experiment, ovarian cancer cells were labeled with antiHER2*Fe₃O₄/Au (core-shell) superparamagnetic scFv. Energy dispersive x-ray spectrum collected from the present in the blood cancer cells, which were labeled with antiHER2 scFv tagged with superparamagnetic core-shell iron oxide-gold nanoparticles (FeAu*biotag) and isolated with a magnet (FIG. 16), while all the blood leftovers were washed away with PBS. Labeling of cells with scFv tagged with superparamagnetic nanoparticles makes them susceptible to magnetic field. Therefore, all the elements constituting blood or lymph are separated very effectively. The shell of gold protects the cells against any toxic effects. The intense peaks of Fe and Au in the spectrum indicate presence of the superparamagnetic scFv internalized and escaped into the cytoplasm, while creating a permanent magnetically detected reporter for these cancer cells, wherever and whenever they go (FIG. 17).

Isolated cells can be grown in culture and be tested for the most effective therapy. This provides the ability of the biotags to be used in the context of individualized, personal, clinical medicine. Further, tagged cells are may be isolated for genomic and proteomic analysis, thus establishing a platform for designing pharmacogenomic therapy.

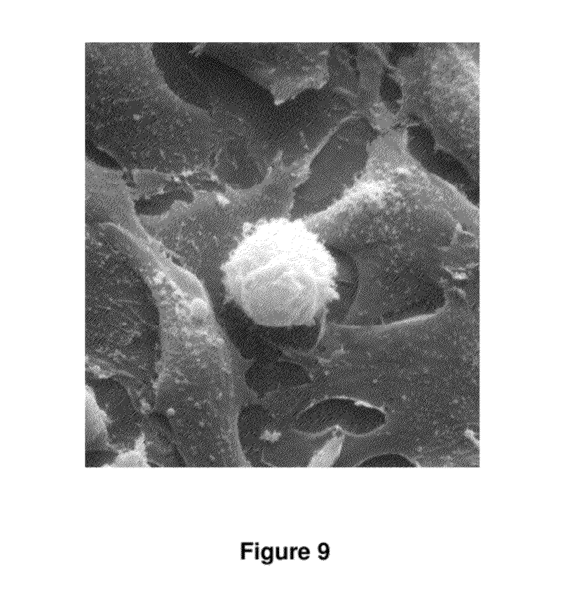

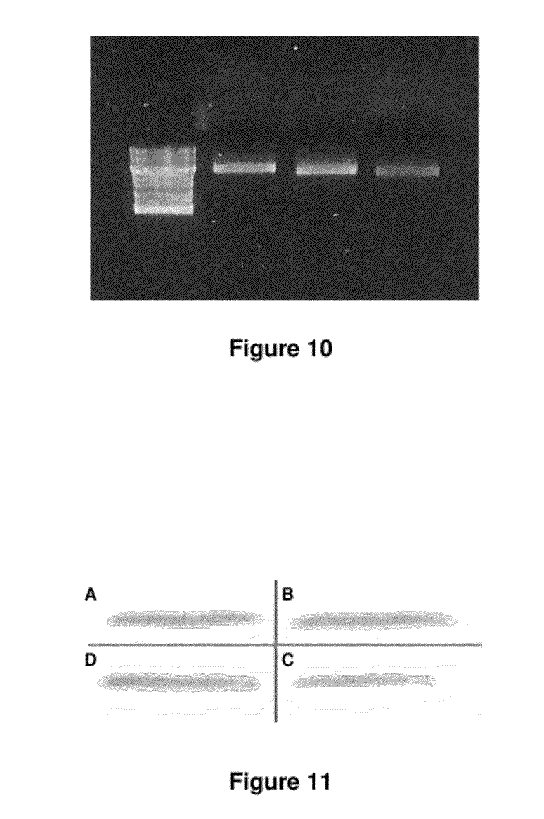

The studied cells have a very high potential to form metastases as shown in FIG. 9. The image represents confirmation in vitro of the data collected and provided by ATCC from the experiments in vivo, concerning metastatic potential of these cell lines. The cell lines served two purposes: the receptors for EGF 1-4 were isolated to load the pans in the in vitro evolution pursuits towards generating scFv. Moreover, they were also used as a test for specificity of the generated scFv as shown in FIG. 11. The coding sequences for scFv which were directed against non-overlapping domains of ERBB are shown in FIG. 10. These scFv were tagged with ultra pure gold nanoparticles, and retained their specificity of targeting as demonstrated in FIG. 11.

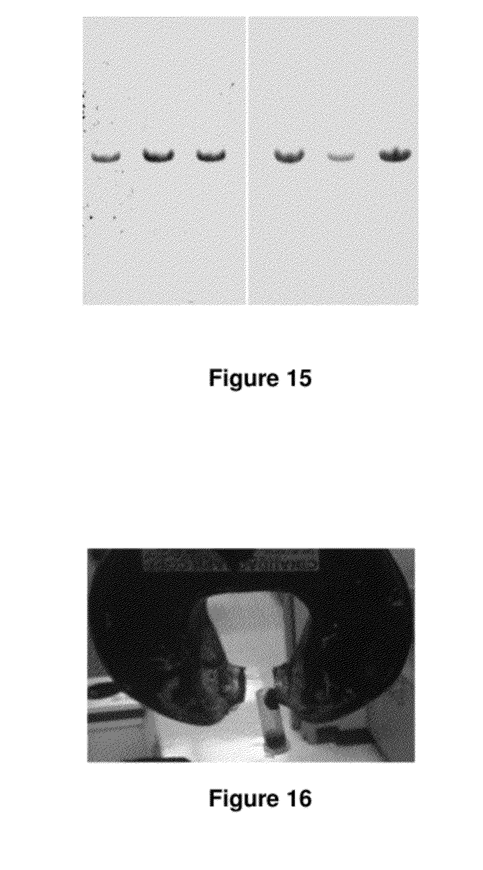

High specificity of superparamagnetic scFv was also confirmed on Western blots from cell lysates. Exquisite single bands were clear indications of high specificity of the engineered scFv (FIG. 15). All the combinations resulted in the same labeling patterns. Most importantly, the blots demonstrated that no other proteins in the entire cell lysate were labeled with scFvs. The scFv retained specificity towards targeted ErbB receptors, even after Gd coordination. Moreover, the background was entirely label free. The ultimate test for attaining the project objective was the effect which superparamagnetic antibodies anchored to the receptors on cell surfaces might have on local relaxivity.

Table 5 shows data from representative experiments. Refined measurements were conducted on wide-bore Bruker (Table 5). Importantly, we observed significant increase in water relaxivity. That resulted in the change in relaxivity was proportional to the number of Gd chelated by MBD into the scFv. The relaxivity of water protons was about 200 mM⁻¹ s⁻¹ at 9.4 T. This study created the basis for a simple, fast detection of cancer cells in physiological fluids, (e.g., circulating tumor cells (CTC) in blood or disseminating tumor cells (DTC) in CSF) The CTC or DTC may be detected with NMR this way based upon reading the changed relaxivites of the samples in vitro or detected by portable magnetic resonance devices. This indicates that the high relaxivities result in MRI contrast changes at antibody concentrations as little as 0.1 uM, which is sufficient for imaging of receptors in vivo. It was demonstrated that the scFv with Gd are capable of labeling brain cancer glioma cells in vitro. In cell culture studies, a significant contrast-to-noise ratio (CNR) enhancement has been observed as a result of using superparamagnetic scFv. Therefore, these scFv-based receptor targeting contrast agents created a clinically relevant change in relaxivity detectable in NMR and/or MRI (Table 5).

TABLE 5
Differences in T1 relaxation times, between unlabeled physiological fluids
and tissues versus GE paramagnetic antibody labeled cells.

| Fluid/Tissue | ΔT1 time** (s) | |
|---|---|---|
| Water | 3.210 +/− 0.031 | s |
| Serum | 2.273 +/− 0.024 | s |
| Detroit fibroblasts culture | 1.598 +/− 0.015 | s |
| Ovarian cancer TOV-112D CRL-11731 | 1.303 +/− 0.011 | s |
| Ovarian cancer TOV-112D CRL-11731 + | 393.626 +/− 0.028 | ms |
| anti HER2/neu scFv_{Gd} | | |
| Breast cancer CRL-2340 HCC2157 | 1.219 +/− 0.013 | s |
| Breast cancer CRL-2340 HCC2157 + | 428.327 +/− 0.039 | ms |
| anti HER2/neu scFv_{Gd} | | |

**Measurements of T1 relaxation times change induced by superparamagnetic scFv in [s] by inversion recovery with 400 MHz at 9.4 T on 28 mm wide-bore Bruker.

To summarize, significant differences were noticed in the signal strength generated between unlabeled ECM, fibroblasts, ovarian and breast cancer cells after labeling with superparamagnetic biotags. Moreover, the signal strength generated in 0.5T NMR was sufficiently strong to detect passage of a single cancer cell through the microfluidic channel, micropipes or blood vessels.

Practical utility of the embodiments described herein is associated with the features of the biotag, in that loading cancer cells with permanently internalized and endosome escaped superparamagnetic scFv ensures that no false negative result would be obtained for the patient suffering from presence of cancer cells circulating in the blood, lymph, peritoneal fluid, cerebrospinal fluid, or any other physiological or pathological fluids.

### Example 5

### Diagnosis and Eradication of Tumors Using Superparamagnetic, Fluorescent, or Noble Metal Tagged Biotags In Vivo

Specific signal to background noise ratio is the main factor used to discriminate the structure labeled with the element tagged antibody guided contrast agent from the unlabeled structures surrounding it. The biotags described herein generate a label-free background (i.e., no non-specific labeling) because of their ability to be internalized and permanently label cancer cells.

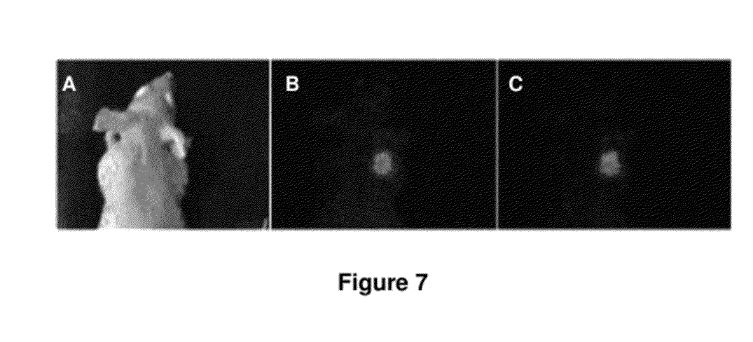

In Vivo Molecular Imaging in mice and rats. Nude mice were injected on the right shoulder with cancer cells that overexpress ErbB 1-4 and/or TfR and the tumors were allowed to progress. Alternatively, the nude mice may be implanted with cancer xenografts that are positive for ErbB2. Tumors were allowed to progress. FIG. 7A (left) shows a nude mouse imaged in diffused light (left panel). A single bolus of a cocktail containing antiErbB1-4 biotags tagged with Au nanoparticles was injected into the nude mouse tail vein. After the injection of the Au*biotags, the mouse was imaged by fluorescence Raman, wherein the tumor was brightly detected with negligible background (FIG. 7B, center panel). This result illustrates that the biotags tagged with gold specifically target cancer cells overexpressing ErbB2/HER2 in vivo, and can accurately detect and diagnose cancer by the presence of a malignant tumor in a nude mouse.

After detection of the tumor, the mouse was injected with transgenes that block antioxidant enzymes (anti-ROS enzyme blockers) antiCatalase, antiSOD, and antiGPX. The mouse was then subjected to x-ray irradiation followed by a single bolus injection of a biotag targeting phosphatidylserine, an apoptosis marker. FIG. 7C (right panel) is an x-ray that shows rapid induction of apoptosis as illustrated by the phosphatidylserine biotag in cancer cells after administration of the biotag therapy. Different wavelengths of emission (color) or different energy of radionuclide were used to distinguish between the diagnostic biotags and those tracking the apoptosis and necrosis.

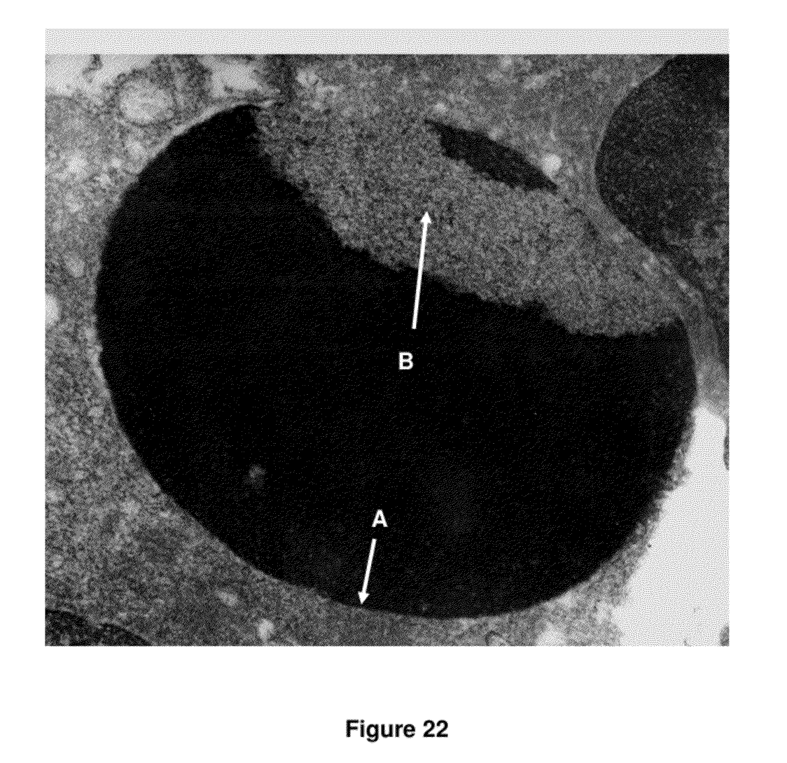

A treated tumor saturated with biotags, further sensitized with scFv anti-ROS enzyme blockers (antiCatalase, antiSOD and antiGPX) and exposed to radiation was sampled with a fine needle biopsy. The biopsy was rapidly and high-pressure frozen and freeze-substituted. Ultrathin sections were prepared, and imaged using FESTEM and 100 kV as previously described (Malecki 1992). Complete collapse of chromatin against the nuclear membrane, a hallmark of apoptosis, is shown in FIG. 22. The cytoplasm is also show filled with biotags. This image establishes that the biotags described herein are selective radiosensitizers and induce apoptosis and/or necrosis in cancer cells treated with x-ray radiation after targeted delivery of the biotags.

Computed tomography was pursued with a Toshiba Aquilion 64-slice clinical scanner. Initial settings were as follows: voltage 120 peak kV, current 40 mA, exposure time of 0.6 s, slice setting 0.5 mm (the slices that were thereafter compressed into 2 mm display images), (modifications of these settings were indicated in the figure legends). ImageQuantTL® version 1.1.0.1 was used to evaluate relative peak pixel intensity of the samples on the computed tomography images utilizing a 0 to 255 level grayscale.

Although this example is directed to an experiment using a gold nanoparticle and x-ray based imaging techniques, the experiment may alternatively be carried out using biotags tagged with superparamagnetic nanoparticles, magnetic resonance techniques and subjecting the subject to electromagnetic radiation.

Effective and lethal dose determinations. Having approved IACUC protocols, the mice and rats were injected via tail veins with increasing concentrations of biotags tagged with Au nanoparticles in single or multiple bolus of up to 3M molarity. There were no effects on their behavior or life span.

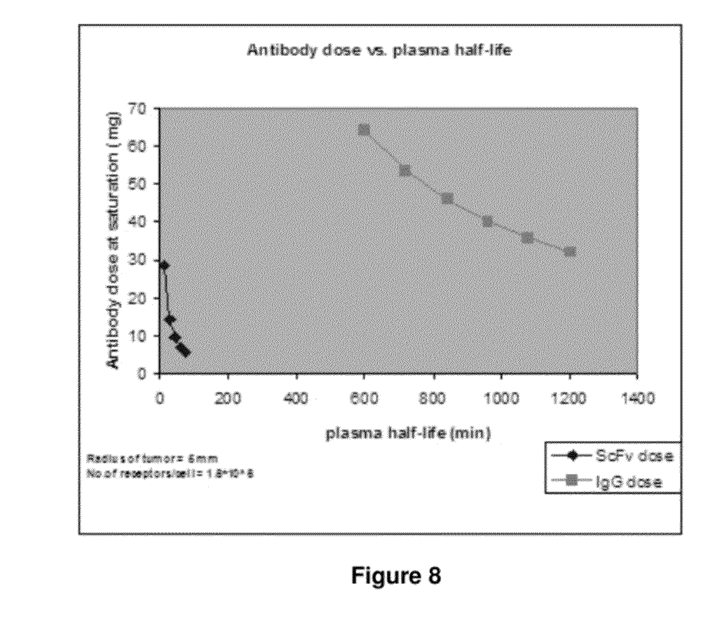

Clearance rates. The scFv Au*biotag rate of clearance in the blood as compared to larger antibody molecules was tested. Rapid clearing of the scFv Au*biotags results in a clear background for imaging, which cannot be accomplished with Fab or IgG. FIG. 8 shows the clearance rates of non-internalizing scFv and IgG from plasma. A faster clearance is associated with a more rapid clearance of the background and improves signal to noise ratio. When the Au*biotags are internalized, the specific signal was retained in the cancer cells indefinitely against entirely clear background. The experiment was done on MDA431 cells using a scFv based probe versus IgG without internalization in vivo in a 250 g rat.

### Example 6

### In Vitro Detection of Metastatic Cancer Cells

Specific signal to background noise ratio is the main factor to discriminate the structure labeled with the element tagged antibody guided contrast agent from the unlabeled structures surrounding it. Therefore, the biotags were engineered in such a way that they would generate label-free background, i.e., no non-specific labeling. As described earlier and applied here, it has been accomplished by selecting clones using short receptor domain sequence libraries, purification prior to and after derivatization, evaluation of antibody affinity on native electrophoresis and blue blots, and validation of the data with EDXSI. This complex approach resulted in very specific localization of superparamagnetic scFv on and within metastasizing cancer cells.

The studied cells have a very high potential to form metastases as shown in FIG. 9. The image represents confirmation in vitro of the data collected and provided by ATCC from the experiments in vivo, concerning metastatic potential of these cell lines. The cell lines served two purposes: the receptors for EGF 1-4 were isolated to load the pans in the in vitro evolution pursuits towards generating scFv. Moreover, they were also used as test for specificity of the generated scFv as shown in FIG. 11. The coding sequences for scFv which were directed against non-overlapping domains of ErbB are shown in FIG. 10. These scFv were tagged with ultra pure gold nanoparticles, and retained their specificity of targeting as demonstrated in FIG. 11.

Detection of metastatic cells by testing of labeling specificity and efficiency of cancer cells suspended in human blood is described herein. Blood samples were drawn from volunteers under IRB protocol and incubated with several of the cancer cell lines used above (AU565, UACC812, MDA-MB453, UACC893 (20× gene amp), CRL2338, MDA453, MCF7 normal breast culture cells and connective and epithelial tissue normal control cells) known to form metastases. Other cell lines from brain, breast, testicular cancers were also used. These cells have a very high potential to form metastases as shown in FIG. 9. The image represents confirmation in vitro of the data collected and provided by ATCC from the experiments in vivo, concerning metastatic potential of these cell lines.

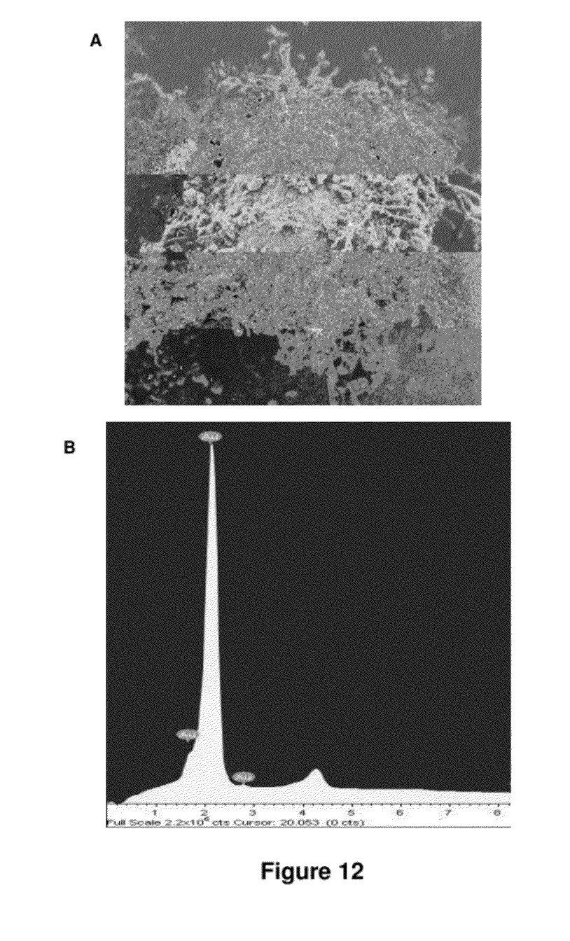

Thereafter, the scFv tagged with gold nanoparticles was added to the cancer cells incubated with blood followed by incubation at 37° C. for 1 hour. The blood aliquots were then spun down. In the retained pellets, cancer cells were present as shown in FIG. 12A. Energy dispersive x-ray analysis showed presence of gold in these cells as shown in the spectrum in FIG. 12B. This experiment demonstrated that it is possible to detect the presence of several different types of cancer cells in blood in vitro. The presence of cancer cells in the blood sample indicates dissemination of cancer cells from a primary tumor that may lead to metastasis. Similarly, the ovarian, testicular, and brain cancer cells from the patients' physiological fluids were labeled with an scFv, sdFv, CDR or SDR modified CDR tagged with superparamagnetic, noble, and fluorescent (some superparamagnetic nanoparticles show non-fading fluorescence). Their presence was detected with the SPR, X-ray, NMR, and fluorometry as described above. They create the basis for the instant detection of cancer cells and diagnosis of their malignancy. Therefore, the technologies described in the examples above create great potential utility for clinical and laboratory oncology. The examples are directly applicable to detecting disseminating, circulating, metastatic cancer cells from blood, lymph, SCF or IPF samples taken from cancer patients. Further, hematologic neoplasm cancer cells may also be detected because they are associated with blood, lymph, and marrow. The metastatic cancer cells may also be used in further experiments that may be used to develop personalized medical profiles of the cancer patients. Such profiles are based upon personalized, pharmacogenomic therapy approaches, which involve crafting therapy according to the targeted delivery and genetic profiles of the patient.

### Example 7

### Depletion and Eradication of Metastasizing Cancer Cells from Blood and Lymph Ex Vivo Using Superparamagnetic Biotags

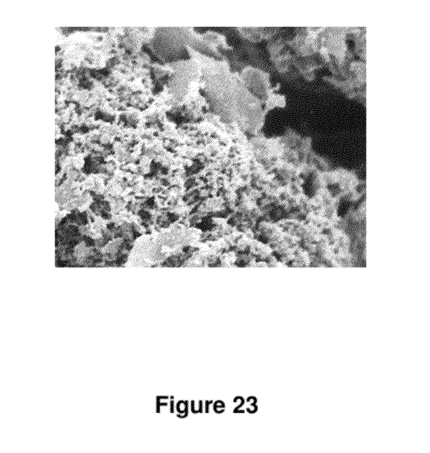

Cancer cells that were combined with healthy donor blood were circulated by a peristaltic pump through heparinized polypropylene tubes that were connected to a container. The container was placed within the magnetic field of an electromagnetic radiation source and while the rest of the set-up was protected by Faraday cage. The cancer cells were retained in the container by the magnetic field, thereby depleting the blood of cancer cells. In addition and/or alternatively, antioxidative enzyme blockers (antiCAT, antiSOD, antiGPX) targeting cancer cells were added to the circulating blood with the superparamagnetic core-noble metal shell (Fe₃O₄/Fe₂O₃—Au)*Biotags were added to the circulation. As the cancer cells internalized the biotags, they became more sensitive to the effects of the AC electromagnetic radiation. This resulted in the induction of apoptosis in the cancer cells as demonstrated by the formation of the cell surface membrane blebs, which are a sign of cellular apoptosis (FIG. 23).

### Example 8

### Depletion and Eradication of Metastasizing Cancer Cells from Blood and Lymph Ex Vivo Using Noble Metal Harboring Biotags

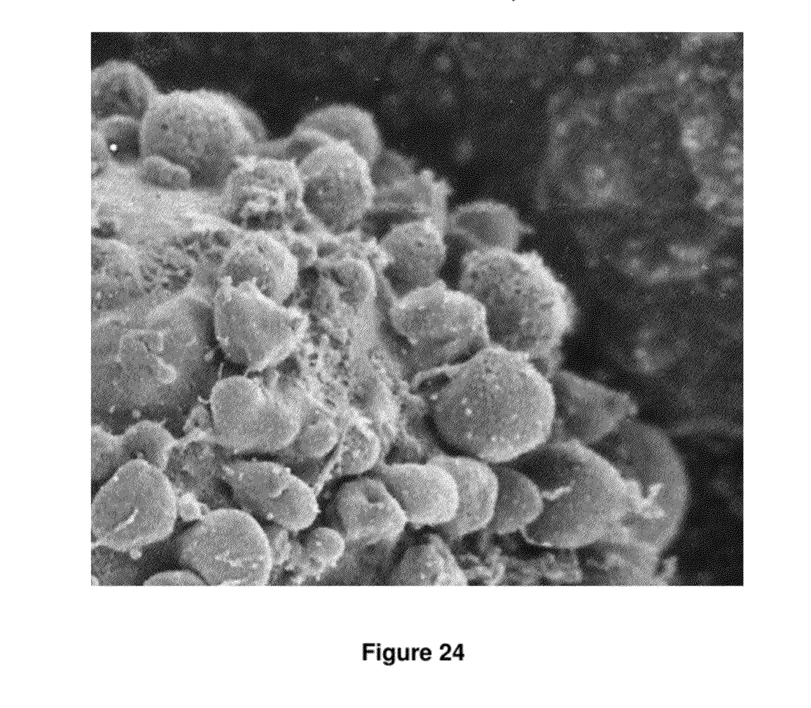

Cancer cells that were combined with healthy donor blood were circulated by a peristaltic pump through heparinized polypropylene tubes that were connected to a container. The container was placed within the projectiles of the X-ray radiation source, while the rest of the set-up was protected by lead (Pb) shielding, exposing only the portion of the blood flowing through the exposed area to the X-ray radiation. Antioxidative enzyme blockers (antiCAT, antiSOD, antiGPX) that target cancer cells and Au*biotags were then added to the circulation. As the cancer cells internalized the biotags, they became more sensitive to the X-ray radiation, inducing DNA strand breaks. This resulted in the induction of apoptosis in the cancer cells as demonstrated by the formation of the cell surface membrane blebs, which are a sign of cellular apoptosis (FIG. 24).

### Example 9

### Instant Diagnosis of EGFRvIII Positive Brain Cancers Based Upon NMR of Cells from Cerebrospinal Fluid Labeled with Superparamagnetic, Genetically Engineered, Single Chain Variable Fragment (s*scFv) Antibodies

An instant and sensitive test for clinical laboratories was developed herein, which would allow clinicians to instantly diagnose patients suitable for immunotherapies, while avoiding the trauma of the invasive diagnostic procedures to the patients that are not suitable for such treatment. Superparamagnetic, genetically engineered, single chain variable fragment antibodies targeting EGFRvIII (s*scFv) were designed using technology developed previously (Malecki et al. 2001). The superparamagnetic s*scFv consist of heterospecific and multifunctional domains as described above. Therefore, they retain high specificity towards the targets, while rendering superparamagnetic coercivity, thus strongly enhancing relaxivity.

Materials and Methods

Cerebrospinal fluid (CSF). The cerebrospinal fluid (CSF) was elicited according to the standard neurological procedures. A cohort of 50 patients was studied, who were organized in three groups: (1) 11 patients were diagnosed with brain cancers (BC): Glioblastoma multiforme (GB), Anaplastic astrocytoma (AO), or Anaplastic oligendroglioma (AO), which were positive for epidermal growth factor receptor variant III mutation gene (BC EGFRvIII+); (2) 14 patients with brain cancers, which were EGFR negative (BC EGFRvIII−); (3) 23 of patients diagnosed with other neurological disease (OND), which were all EGFR negative (OND EGFRvIII−). The elicited volumes of CSF varied, but the final pressure never reached below 60 mm H₂O and never less than 50% of the opening pressure. The samples were immediately labeled and either processed directly or rapidly frozen and stored in liquid nitrogen.

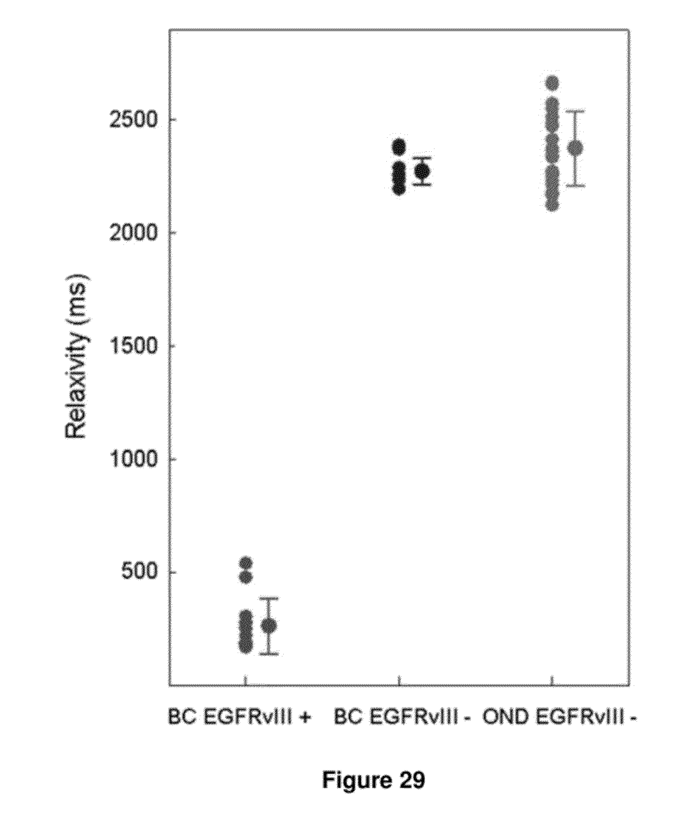

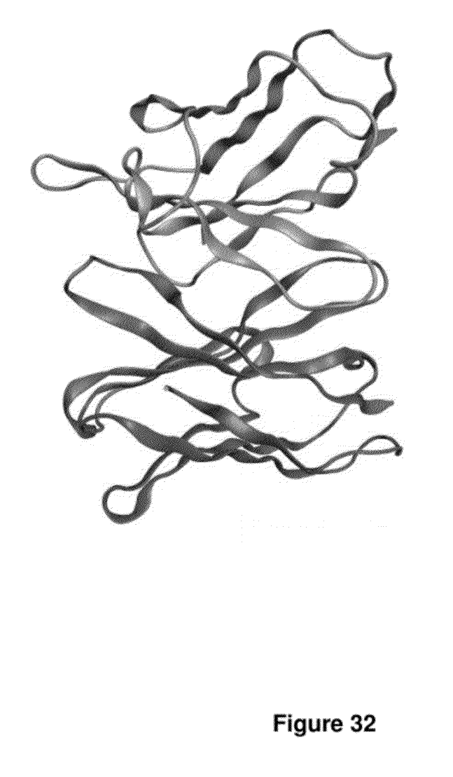

Superparamagnetic, genetically engineered scFv. The details of the methods used for genetically engineering the superparamagnetic scFv used herein were previously described (Malecki et al. 2001). Briefly, the pooled white blood cells from the patients suffering from cancers were used to create the libraries of complementarity determining regions (CDR) and framework regions (FWR). They were cloned and expressed in human myelomas. Selection of clones showing specificity toward EGFRvIII (e.g., SEQ ID NO: 207-224; SEQ ID NO:286-291) was pursued on pans anchoring the recombinant, extracellular domains of these antigens and validated on the EGFRvIII positive single cell arrays (FIG. 32). The DNA constructs were further engineered to contain coding sequences for metal binding domains, e.g., Au, Pt, Eu, Gd, or Tb chelating domains as described earlier (Malecki et al. 2001). The heterospecific scFv coding constructs were expressed in human myelomas. The superparamagnetic nanoparticles, core-shell or organometallic cluster types (Fe₃O₄—Au, Gd, Eu, Tb, etc), were prepared by laser ablation. They were chelated by the metal binding domains of scFv by facilitated, covalent binding to render them superparamagnetic. These clusters were tested on the single cell arrays, on immunoblots, as well as with energy dispersive x-ray spectroscopy and energy filtering transmission electron microscopy as in the very details described elsewhere (Malecki et al. 2001). FIGS. 28 and 29 are related to a biotag having a Eu reporter tag and an scFv biomarker binding domain that may have one or more or a combination of the amino acid sequences SEQ ID NO:250 and 289.

Testing specificity of labeling with antiEGFRvIII superparamagnetic scFv on immunoblots. The cells from CSF were disintegrated by sample buffer and electrophoresed in 2% agarose gel within 10 mM Tris, 31 mM NaCl buffer. Immediately afterwards, the cell lysates separated by electrophoresis were electro-transferred onto the PVDF membranes within CAPS buffer (10 mM 3-[Cyclohexylamino]-1-propanesulfonic acid (CAPS), Tris/glycine transfer buffer 25 mM Tris base, 192 mM glycine, pH 8.3) using an electrotransfer unit (Amersham). Thereafter, the membranes carrying transferred proteins were soaked within the human serum containing s*scFv_{EGFRvIII}. The bands could be watched being labeled. Thereafter, visibility of the bands was further strengthened by gold enhancement. The images of developed blots were acquired with Fluoroimager (Molecular Dynamics) or Storm 840 (Amersham).

Confirmation of the scFv integrity with Energy Dispersive X-ray Elemental Spectroscopy. After completion of blotting, the PVDF membranes carrying the labeled bands were freeze-dried within the oil-free vacuum system. After reaching 10×10̂8 Pa, they were quickly transferred within the nitrogen holder into the column of the Field Emission Scanning Electron Microscope (Zeiss 1540 or JEOL 6000 or Hitachi 3400) equipped with Energy Dispersive X-ray Spectroscope. Complete elemental spectra were acquired for every pixel of the scans to create the elemental databases. From them, after selecting an element specific energy window, the map of this element atoms' distribution was calculated with ZAF correction (NIST). As the antiEGFRvIII scFv were tagged with superparamagnetic metals, then exogenous elements within them were incorporated into their structure. Tangerine, the most sensitive protein stain was used to determine distribution of proteins (Molecular Probes). Thereafter, the integrity of scFv organometallic clusters was determined in EDX by co-localization of the peaks. Furthermore, the location of the scFv was determined based upon the elemental maps. The spectral maps were acquired at 3 kV operating voltage to acquire the first energy peaks and displayed as elemental maps with the details described (Malecki et al. 2001).

Measuring relaxivities of the cells from CSF labeled with superparamagnetic scFv antibodies within NMR. The s*scFv were mixed with the CSF sample, gently vortexed, and spun down into a pellet at low g. The pellets were re-suspended within a buffer having a composition similar to CSF, i.e., supplemented with protein 30 mg/dL and glucose 60 mg/dL. The samples were dispensed into the magnetism-free NMR tubes and inserted into the NMR spectrometer (Bruker) or the Magnetic Resonance Imaging Scanner operated in the non-imaging, NMR mode (GE, Philips). For data acquisition, inversion-recovery and spin-echo pulse sequences were applied and relaxation times (T1) calculated as in the details described (Ibrahim et al. 1998; Melhem et al. 1999).

Results

The engineered, superparamagnetic, single chain variable fragment antibodies (s*scFv_{EGFRvIII}) specifically targeted epidermal growth factor receptor variant III (EGFRvIII) mutated gene expression products. To show this, the U87 human glioblastoma line was cultured to over-express the gene for the wild type epidermal growth factor receptor (EGFR). For comparison, the U87_{EGFRvIII} line was also cultured, which was transgenically expressing epidermal growth factor mutant III gene. Immunoblots from both lines labeled with s*scFv_{EGFRvIII} are illustrated in FIG. 28, lanes a-b. The lane “a” in this figure corresponds to U87 expressing EGFRwt. It shows no signs of labeling. The lane “b” contains one single band at 145 kDa, which is specific for the transgenically expressed EGFRvIII in the human glioblastoma U87_{EGFRvIII}. These results show that the superparamagnetic scFv is specific for EGFRvIII.

Next, to verify whether the s*scFv_{EGFRvIII} that was responsible for revealing bands of the mutated receptors in FIG. 28 were associated with chelating superparamagnetic ions of Eu or Gd or Fe. For that purpose, energy dispersive x-ray spectral imaging (EDXSI) was used. The distribution of these metals determined had the same specific energy peak as that of the scFv (not shown), illustrating that scFv chelating domains were efficiently coordinating superparamagnetic nanoparticles and ions.

Next, the relaxivities of U87 and U87_{EGFRvIII} were determined based on the effects of labeling the cells with s*scFv_{EGFRvIII}. Cells from both lines with our superparamagnetic s*scFv_{EGFRvIII} were used while maintaining them in the CSF buffer. The relaxation times (T1) were measured in NMR. T1 for the U87 were 2200-2500 ms, which were similar to the published values of CSF buffer alone. T1 for samples containing U87_{EGFRvIII} labeled with s*scFv_{EGFRvIII} were in the range of 200-400 ms. This was a statistically significant difference. This high difference allowed us very reliable to identify EGFRvIII expressing cultures from non-expressing, based upon relaxation times measured in NMR. Having these basic tests completed the cells from the CSF samples of the patients were analyzed.

Cerebrospinal fluid (CSF) samples from patients suffering focal neurological symptoms, were analyzed in clinical chemistry laboratories. As shown in FIG. 29, for the purpose of the data analysis, the results were later classified into three groups: patients diagnosed with the brain cancer expressing mutated gene-EGFRvIII positive (EGFRvIII+); patients diagnosed with the brain cancer not expressing or not having detected mutated gene-EGFRvIII negative (EGFRvIII−); patients with other neurological diseases, but not neoplasms (OND), e.g., Brain Strokes or Multiple Sclerosis (MS).

Small aliquots of were taken from the main batch from each patient based upon the approval Institutional Review Board and the signed Informed Consent form. The cells from the first aliquot were immediately labeled with s*scFv_{EGFRvIII} for measuring relaxation times with nuclear magnetic resonance (NMR). The cells from the second aliquot were lysed for electrophoresis and immunoblotting.

The relaxation times of the cells labeled cells with s*scFv_{EGFRvIII} and measured in NMR are compiled in FIG. 29. Three repeats for each sample assured accuracy of the measurements and calculation of standard deviation. These measurements revealed striking differences between the EGFRvIII positive and negative cancer cells. On average the relaxation times of the cells within CSF buffer were in the ranges of 2439-2728 ms. These values were similar to measured for U87 cells expressing EGFRwt, but not EGFRvIII. They were also very similar to the values of relaxation times published in the literature.

In parallel, the cells from similar aliquots of cells from CSF were promptly homogenized, electrophoresed, and transferred to follow by immunoblotting with s*scFv_{EGFRvIII}. The representative blots are illustrated in FIG. 28, lanes c-e. The strong band of the protein with mw 145 kDa in the lane “d” identifies the brain cancer cells strongly expressing EGFRvIII. Importantly, except that one strong band, there are no signs of any labeling along the entire lane. This is indicative of the very specific and exclusive labeling of EGFRvIII with our s*scFv_{EGFRvIII}. To the contrary there is no label on the lane “c”, in FIG. 28. It illustrates the immunoblot of the brain cancer cells, which apparently do not express EGFRvIII, thus were designated as the EGFRvIII negative. Similarly, there is no band of EGFRvIII in the lane “e” in FIG. 28. This immunoblot comes from the lysates of the CSF cells, which were obtained from the patients clinically diagnosed with other neurological diseases (OND) e.g., Brain Strokes (BS), or Multiple Sclerosis (MS). They were also designated as the EGFRvIII negative. In both immunoblots of EGFRvIII negative cells, there are no molecules labeled anywhere in that background. It is of critical significance, from the stand point of diagnostic applications, that these s*scFv were not cross-reacting with any other domains of other molecules. They were capable to uniquely identify the EGFRvIII positive cells. The results of all immunoblots for the patients were compiled and a clinical diagnosis was determined for each patient. 16 patients out of 50 were diagnosed clinically with the brain cancers. They were identified clinically as Glioblastoma multiforme (GB), Anaplastic astrocytoma (AA), and Anaplastic oligodenroglioma (AO). However, in 9 cases the brain cancer cells expressed detectable levels of EGFRvIII mutant gene expression products. This corresponds to the percentages reported in other studies. The remaining brain cancers were EGFRvIII negative. The immunoblots of cells from the patients with the clinical diagnoses of other neurological diseases, Brain Strokes and Multiple Sclerosis among them, were all EGFRvIII negative. They also served as the clinically relevant control in our study. Therefore, the s*scFv_{EGFRvIII}, used herein was able to identify, on immunoblots of the cells from CSF, the cells expressing the mutated variant of the EGFRvIII gene expression products.

In addition, measurements of relaxation times in NMR were performed on the cells from CSF, which were labeled with the superparamagnetic scFv targeting EGFRvIII (s*scFv_{EGFRvIII}). The measurements are compiled in FIG. 29. Even prior to the results of immunoblots and completion of the clinical diagnoses, it was observed that after labeling with s*scFv_{EGFRvIII}, samples from some of the patients caused the dramatic shortening of relaxation times. These relaxation times varied greatly from 173 ms to 487 ms (FIG. 29, BT EGFRvIII+). These samples were later identified as coming from the patients, who were later diagnosed with the EGFRvIII positive brain cancers including GB and AA. The readings in the other group were in a sharp contrast to those values, as their readings were similar to those of the CSF buffer alone and ranged from 2199-2389 ms (FIG. 29, BT EGFRvIII−). These samples were later identified as coming from the patients who were clinically diagnosed with the EGFRvIII negative brain cancers. Similarly, the long relaxation times ranging from 2200-2500 ms, were recorded on the samples, which were later identified as obtained from the patients diagnosed with other neurological diseases (FIG. 29, OND). These significant shortenings of relaxation times (T1) were recorded on the brain cancer cells labeled with s*scFv_{EGFRvIII}, which were identified clinically and on immunoblots as EGFRvIII+, when in comparison to the other brain cancer cells elicited from the patients, who were clinically and immunologically diagnosed as EGFRvIII negative. By comparison, there were almost no differences in the relaxation times between EGFRvIII negative cancers and OND. Therefore, presence of the EGFRvIII positive cells in CSF may be detected with NMR. In cases of pleocytosis of CSF, they could be easily distinguished from inflammatory cells. This analysis may form a stand alone diagnosis or may be a complement to existing diagnostic tests for detection of EGFRvIII positive tumors. Statistically significant differences between the relaxation times recorded for the EGFRvIII positive cells and the EGFRvIII negative were apparent (p, 0.001). Therefore, these changes in relaxation times reflecting presence or absence of EGFRvIII gene expression products provide the clinically relevant information concerned with the brain cancer cells from the cerebrospinal fluids of the patients.

To summarize, a minimally invasive and reliable test for identifying presence of EGFRvIII mutated gene expression products in the cells elicited from the cerebrospinal fluids of the patients was developed. This should help with instant diagnoses of the patients suffering from these most aggressive brain cancers and with qualifying them for EGFRvIII targeted therapies.

Success of this work can be attributed to the high specificity of the genetically engineered s*scFv. Their high specificity resulted in heavy and specific labeling of the mutated receptors. This was also associated with the supreme sensitivity through gold enhancement resulting in minimizing false negatives. It also secured the complete absence of non-specific labeling of cells without mutations, thus eliminated a possibility of false positives. In translation into NMR reading, the signal to noise ratio was remarkably high. The high affinity of these antibodies was shifting the dynamic on/off balance; thus enhancing conditions for T1 acquisition. Further, the small size of these scFv helped in overcoming steric hindrance forces and packing onto the receptors. That increase in packing or labeling density was also seen on the images from scanners. The labeling density was much higher with scFv, than it was with Fab or IgG. In this study, it translated into the significant concentration of superparamagnetic organometallic clusters or nanoparticles tagging scFv on surfaces of the cells.

This work opens also new avenues for in vivo studies involving the s*scFv antibody guided contrast. The labeling of cells with the superparamagnetic clusters resulted in significant changes of the relaxivity reflected in shortening of T1 and strengthening of the generated signal. If injected into the patients, this effect would be perceived as the bright spots on the screen of MRI scanners. Specific signal to background noise ratio and appropriate pulse sequence eliciting maximum resonance of the targeted molecules are the main factor to discriminate, the structure labeled with the element tagged recombinant antibody guided contrast agent from the unlabeled structures surrounding it. The high specificity demonstrated on the immunoblots would translate into the very specific, high signal to noise ratio (SNR) in the clinical MRI scanners. Image guided therapy, targeted therapeutics, or magnetic hyperthermia therapy could follow.

### Example 10

### Screening for and Instant Diagnosis of EGFRvIII Positive Ovarian Cancers Based Upon NMR of Cells from Peritoneal Effusions Labeled with Genetically Engineered, Superparamagnetic scFv Antibodies

A specific, sensitive, simple, minimally invasive clinical laboratory test is provided herein, which establishes a diagnostic screening test for women with high susceptibility to developing ovarian cancer, while minimizing trauma to the patients. Superparamagnetic, genetically engineered, single chain variable fragment antibodies targeting EGFRvIII (s*scFv) were designed using technology developed previously (Malecki et al. 2001). The superparamagnetic s*scFv consist of heterospecific and multifunctional domains as described above. Therefore, they retain high specificity towards the targets, while rendering superparamagnetic coercivity, thus strongly enhancing relaxivity. The test described below screens patients suspected of developing ovarian cancers by analyzing their peritoneal washings in NMR, which opens the routes for immediate refinement of diagnoses with MRI and for scFv guided therapies.

Materials and Methods

Peritoneal fluid (PF). Paracentesis of the peritoneal fluid (PF) was performed according to the standard surgical procedures. The PF samples were obtained with the IRB approval and with the patients' Informed Consent Forms signed. A cohort of 50 patients was studied, who were organized in three groups: (1) 21 patients were diagnosed with various stages of ovarian cancers (OC), which were positive for epidermal growth factor receptor variant III mutation gene (OC EGFRvIII+); (2) 14 patients with ovarian cancers, which were EGFR negative (OC EGFRvIII−); (3) 15 of patients diagnosed with other disease within abdominal cavity (OD), which were all EGFR negative (OD EGFRvIII−). The samples were immediately labeled with the superparamagnetic single chain variable fragment antibodies targeting EGFRvIII (s*scFv_{EGFRvIII}) or rapidly frozen and stored in liquid nitrogen.

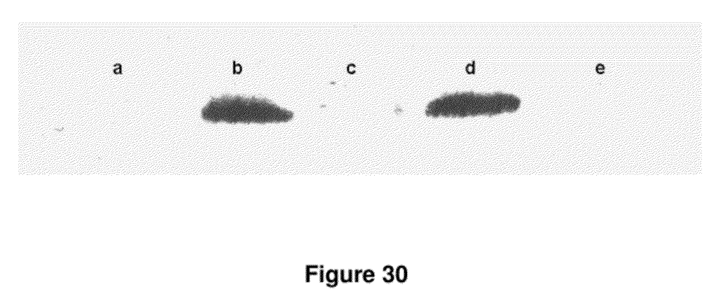

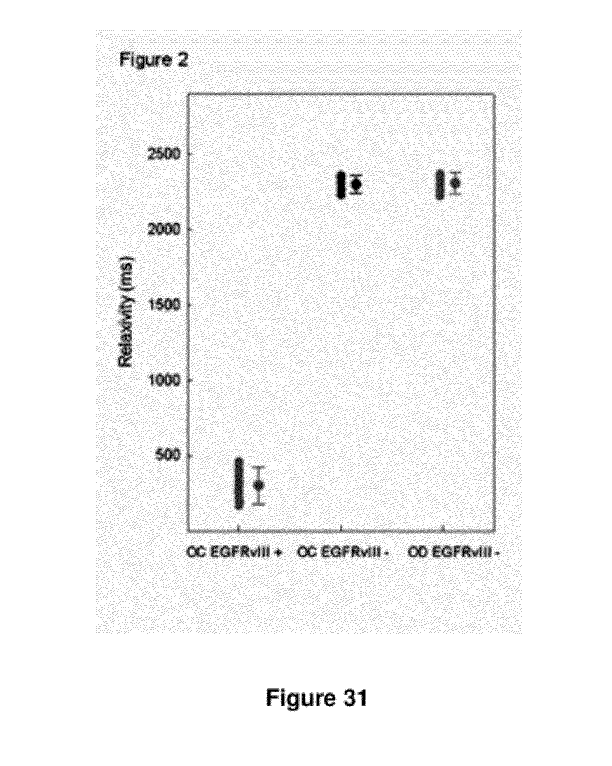

Superparamagnetic, genetically engineered scFv. Pooled white blood cells (WBC) from the patients suffering from cancers were used to create the libraries of complementarity determining regions (CDR) and framework regions (FWR). They were cloned and expressed in human myelomas. Selection of clones showing specificity toward EGFRvIII and EGFR wt was pursued on pans anchoring the single cell arrays (e.g., SEQ ID NO: 207-224; SEQ ID NO:286-291). Thereafter, DNA constructs were engineered to include coding sequences for metal binding domains (Malecki et al. 2001). The heterospecific scFv coding constructs were expressed in human myelomas. The superparamagnetic nanoparticles, core-shell or organometallic cluster types (Fe₃O₄—Au, Gd, Eu, Tb, etc), were prepared by laser ablation. They were chelated by the metal binding domains of scFv by facilitated, covalent binding to render them superparamagnetic, thus to become superparamagnetic biotags of EGFRvIII positive (often called oncotags) or EGFRwt positive cells EGFR (s*scFv_{EGFRvIII} and s*scFv_{EGFRwt} respectively). These clusters were tested on the single cell arrays, immunoblots, qPCR, EDX and ESI as described (Malecki et al. 2001). FIGS. 30 and 31 were produced with a biotag having a Eu reporter tag and an scFv biomarker binding domain having the amino acid sequences SEQ ID NO:250 and 289.

Primary Cultures of Cancers of the Ovaries. During the surgical biopsy and after initial evaluation by surgical pathologist on site, small pieces of tissue were collected into the Dulbecco Modified Essential Medium within cell culture flasks. The outgrowing ovarian cancer cultured cells (OCC) were maintained within the cell culture incubators at 37 deg. C., saturated humidity, and mixtures of CO2/02 gases (New Brunswick). The cells expressed approximately 0.5-3 million EGFRwt per cell as tested with immunoblots and mRNA (OCC_{EGFRwt}). They were transduced with the EGFRvIII gene under CMV promoter to express EGFRvIII transgene expression products (EGFRvIIItg). Some of the cells expressed de novo EGFRvIII (OCC_{EGFRvIII}), when acquired from the patients diagnosed with EGFRvIII+ ovarian cancers.

Testing specificity of labeling with antiEGFRvIII superparamagnetic scFv on immunoblots. The cells from PF were either frozen in liquid nitrogen, or disintegrated within the sample buffers for protein analysis or for total mRNA extraction. The proteins within the sample buffer were electrophoresed and immediately afterwards electro-transferred onto the PVDF membranes using an electrotransfer unit (Amersham). The membranes carrying transferred proteins were soaked within the human serum containing s*scFv_{EGFRvIII}. Thereafter, visibility of the bands was further strengthened by gold enhancement. The images of developed blots were acquired with Fluoroimager (Molecular Dynamics) or Storm 840 (Amersham).

Confirmation of the scFv integrity with Energy Dispersive X-ray Elemental Spectroscopy. The PVDF membranes carrying the labeled bands were freeze-dried within the oil-free vacuum system. After reaching 10×10⁸ Pa, they were quickly transferred within the nitrogen holder into the column of the Field Emission Scanning Electron Microscope (Zeiss 1540 or JEOL 6000 or Hitachi 3400) equipped with Energy Dispersive X-ray (EDX) Spectroscope. Complete elemental spectra were acquired for every pixel of the scans to create the elemental databases. As the antiEGFRvIII and antiEGFRwt scFv were tagged with superparamagnetic metals, then exogenous elements within them were incorporated into their structure. Ruthenium-based ultra-sensitive stain for all proteins was used to determine distribution of all proteins (a gift from Prof. J. Lakowicz). Integrity of scFv organometallic clusters was determined by co-localization of the energy peaks (Malecki et al. 2001).

Measuring relaxivities of the cells from PF labeled with superparamagnetic scFv antibodies within NMR. The s*scFv were mixed with PF, gently vortexed, and spun down into a pellet at low g. The pellets were re-suspended within a PF buffer, i.e., supplemented with proteins and glucose. The samples were dispensed into the magnetism-free NMR tubes and inserted into the NMR spectrometer (Bruker) or the Magnetic Resonance Imaging Scanner operated in the non-imaging, NMR mode (GE, Philips). For data acquisition, inversion-recovery and spin-echo pulse sequences were applied and relaxation times (T1) calculated as described (Ibrahim et al. 1998; Melhem et al. 1999).

Results

The engineered, superparamagnetic, single chain variable fragment antibodies (s*scFv_{EGFRvIII}) specifically targeted epidermal growth factor receptor variant III (EGFRvIII) mutated gene expression products. To show this, an ovarian carcinoma culture was established, which was tested as being positive for the wild type epidermal growth factor receptor (EGFRwt) based upon testing of transcription with RT qPCR of the total mRNA and of translation on immunoblots on the cell lysates, but was negative for the mutation variant III (EGFRvIII). Immunoblots from both lines labeled with s*scFv_{EGFRvIII} are illustrated in FIG. 30, lanes a-b. The lane, which corresponds to cultured cells expressing EGFRwt, but not EGFRvIII shows no signs of labeling (FIG. 30, lane a). The single band at 145 kDa, which is specific for the transgenically expressed truncated version of the receptor, is present on the lane for EGFRvIII positive cells (FIG. 30, lane b), illustrating that the superparamagnetic s*scFv_{EGFRvIII} is indeed very specific for EGFRvIII.

Next, it was verified that the s*scFv_{EGFRvIII} that was responsible for revealing bands of the mutated receptors in FIG. 30, were associated with chelating superparamagnetic ions of Eu, Tb, Gd, or Fe, while retaining specificity towards binding exclusively EGFRvIII. For that purpose, energy dispersive x-ray spectral imaging (EDXSI) was used. The distribution of these metals determined had the same specific energy peak was identical to that of scFv (not shown), illustrating that scFv chelating domains are efficiently coordinating superparamagnetic nanoparticles and ions.

Next, the relaxivities of the cells with s*scFv_{EGFRvIII} were determined with the nuclear magnetic resonance (NMR). For that purpose, cells from both lines were labeled with the superparamagnetic s*scFv_{EGFRvIII}, while maintaining them in the PF buffer. The relaxation times (T1) for the OCC_{ERGFRwt} were 2200-2500 ms, which was similar to the published values of the physiological buffer alone. T1 for samples containing OCC_{EGFRvIII} labeled with s*scFv_{EGFRvIII} were in the range of 180-480 ms. These differences were statistically significant. The differences that high allowed for reliable identification of EGFRvIII expressing cultures from non-expressors, based upon relaxation times measured in NMR. Having these three basic tests completed, the cells from the peritoneal fluid samples of the patients were analyzed.

Patients suspected of having ovarian cancer based on a peritoneal effusion detected during physical examination, were referred to collection of cells for cytopathology. Based upon peritoneal washings' cytopathology and tissue immunohistopathology, as shown in FIGS. 30 and 31, for the purpose of the data analysis, the results were later classified into three groups: patients diagnosed with the ovarian cancer (stages I-IV) expressing mutated gene—EGFRvIII positive (EGFRvIII+); patients diagnosed with the ovarian cancer not expressing or not having detected mutated gene expression product—EGFRvIII negative (EGFRvIII−); patients with other abdominal diseases, but not neoplasms (OD). Small aliquots of PF were taken from the main batch from each patient based upon the approval Institutional Review Board and the signed Informed Consent form. The cells from the first aliquot were immediately labeled with s*scFv_{EGFRvIII} for measuring relaxation times with nuclear magnetic resonance (NMR). The cells from the second aliquot were lysed for electrophoresis and immunoblotting.

The cells from PF were promptly homogenized, electrophoresed, and transferred to follow by immunoblotting with s*scFv_{EGFRvIII}. The representative blots are illustrated in FIG. 30, lanes c-e. The strong band of the protein with mw 145 kDa (FIG. 30, lane d) identifies the ovarian cancer cells strongly expressing EGFRvIII. Importantly, except that one strong band, there are no signs of any labeling along the entire lane. This is indicative of the very specific and exclusive labeling of EGFRvIII with the s*scFv_{EGFRvIII}. To the contrary there is no label on the other lane (FIG. 30, lane c). It illustrates the immunoblot of the ovarian cancer cells, which apparently do not express EGFRvIII, thus were designated as the EGFRvIII negative. Similarly, there is no band of EGFRvIII in the next lane (FIG. 30, lane e). This immunoblot comes from the lysates of the cells, which were obtained from the patients clinically diagnosed with other diseases (OD) of non-neoplasm origin. They were also designated as the EGFRvIII negative. In both immunoblots of EGFRvIII negative cells, there are no molecules labeled anywhere in that background. It is of critical significance, from the stand point of diagnostic applications, that these s*scFv were not cross-reacting with any other domains of other molecules. They were capable to uniquely identify the EGFRvIII positive cells. The results of all immunoblots for the patients were compiled and a clinical diagnosis was made for each patient. 35 patients out of 50 were diagnosed clinically with the ovarian cancers. In 21 cases, the studied ovarian cancer cells expressed detectable levels of EGFRvIII mutant gene expression products. This corresponds to the percentages reported in other studies. The remaining ovarian cancers were EGFRvIII negative. The immunoblots of cells from the patients with the clinical diagnoses of other diseases were all EGFRvIII negative. They also served as the clinically relevant control in our study. Therefore, the s*scFv_{EGFRvIII} used herein were able to identify, on immunoblots of the cells from PF, the cells expressing the mutated variant of the EGFRvIII gene expression products.

In addition, measurements of relaxation times in NMR were performed on the cells from PF, which were labeled with the superparamagnetic scFv targeting EGFRvIII (s*scFv_{EGFRvIII}). The measurements are compiled in FIG. 31 after calculation of standard deviations from three runs and plotting as a graph (FIG. 31). (Sigma software). Even prior to the results of immunoblots and completion of the clinical diagnoses, we observed that after labeling with s*scFv_{EGFRvIII}, samples from some of the patients caused the dramatic shortening of relaxation times. These relaxation times varied greatly from 173 ms to 487 ms (FIG. 31, OC EGFRvIII+). These samples were later identified as coming from the patients, who were diagnosed with the EGFRvIII positive ovarian cancer. The readings in the other group were in a sharp contrast to those values, as their readings were similar to those of the PF buffer alone and ranged from 2199-2389 ms (FIG. 31, OC EGFRvIII−). These samples were later identified as coming from the patients, who were clinically diagnosed with the EGFRvIII negative ovarian cancer. Similarly, the long relaxation times ranging from 2200-2500 ms, were recorded on the samples, which were later identified as obtained from the patients diagnosed with other diseases (FIG. 31, OD EGFRvIII−). These samples were later identified as coming from the patients, who were clinically diagnosed with the EGFRvIII negative ovarian cancer. Similarly, the long relaxation times ranging from 2193-2397 ms, were recorded on the samples, which were later identified as obtained from the patients diagnosed with other diseases (FIG. 31, OD EGFRvIII−). These significant shortenings of relaxation times (T1) were recorded on the ovarian cancer cells labeled with s*scFv_{EGFRvIII}, which were identified clinically and on immunoblots as EGFRvIII+, when in comparison to the other ovarian cancer cells elicited from the patients, who were clinically and immunologically diagnosed as EGFRvIII negative. By comparisons, there were almost no differences in the relaxation times between EGFRvIII negative cancers and OD. Therefore, presence of the EGFRvIII positive cells in PF could be easily discovered with NMR. In cases of pleocytosis of PF, they could be easily distinguished from inflammatory cells. This is a great complement to the existing diagnostic tests for detection of EGFRvIII positive tumors. Statistically significant differences between the relaxation times recorded for the EGFRvIII positive cells and the EGFRvIII negative were apparent (p, 0.001). Therefore, these changes in relaxation times reflecting presence or absence of EGFRvIII gene expression products provide the clinically relevant information concerned with the ovarian cancer cells from the patients.

To summarize, a minimally invasive and reliable test for identifying presence of EGFRvIII mutated gene expression products in the cells elicited from the cerebrospinal fluids of the patients was developed. This should help with instant diagnoses of the patients suffering from this most aggressive ovarian cancer and with qualifying them for EGFRvIII targeted therapies.

Success of this work can be attributed to the high specificity, affinity, and small size of the engineered scFv. Their high specificity resulted not only in heavy labeling of the receptors, but also in reduced non-specific labeling of other cells. Therefore, the signal to noise ratio was remarkably high. The high affinity of these antibodies was shifting the dynamic on/off balance; thus enhancing conditions for T1 acquisition. Finally, the small size of these scFv helped in overcoming steric hindrance forces and packing onto the receptors. That increase in packing or labeling density was also seen on the images from Phosphorimager and EDXSI. The labeling density was much higher with scFv, than it was with Fab or IgG. In this study, it translated into the significant concentration of superparamagnetic nanoparticles tagging scFv on surfaces of the cells, which resulted in significant enhancement of relaxivity.

Contrary to all of the other methods of antibody derivatization for imaging, diagnosis, and therapy, which involve incorporation of reporting agents, which are changing properties of these antibodies, in this work the highly specific domains are specific integral parts of superparamagnetic scFv, but completely separate from antigen binding domains. Therefore, they retain their bio-kinetic properties and binding properties after tagging superparamagnetic clusters or nanoparticles. Further, affinity purification on single cell arrays, which follows derivatization, secures elimination of all molecules, which might have altered their properties.

A significant feature of test described above relies upon the fact that our s*scFv target extracellular domains of the cell surface receptors. Therefore, it effectively complements clinical tests based upon immunohistopathology, cytopathology, and analysis of proteomes and genomes of the cells, Therefore, s*scFv can be potentially used not only for instant ex vivo diagnostic endeavors, but also for enrichment of cytopathology samples from peritoneal effusion through electromagnetically activated cell sorting (EACS) and fluorescently activated cell sorting (FACS) followed by their proteomic and genomic analysis and designing personalized therapies. Moreover, s*scFv are excellent candidates for molecular imaging as the EGFRvIII or EGFRwt targeting contrast agents within MRI clinical scanners. Thereafter, they are also good candidates for pursuit targeted therapy through magnetic field induced targeted hyperthermia.

### Example 10

### Isolation of Circulating Tumor Cells (CTC) Based Upon Levels of Gene Expression Products (“Liquid Biopsy”)

Emerging qualitative and quantitative differences in gene expressions between cancer and healthy cells serve as the bases for biomarkers based diagnostics and targeted therapy. Herein a “liquid biopsy” is provided for isolating circulating tumor cells (CTC) from a physiological fluid sample from a subject (e.g., blood, lymph, CSF) based upon differences in the number of molecules or biomarkers—gene expression products—expressed by the cancer cells.

Isolation of CTC Through the Positive Selection Based Upon Overexpression of TfR, ER, ERBB1-4, PSMA, RON by Cancer Cells.

Single chain variable fragment (scFv), sdFv, CDR, and/or complementary domain oligopeptides (CDO) were genetically engineered from the libraries generated from the B cells of immunized patients.

Scfv, sdFv, CDR, and/or CDO were targeting: Transferrin receptor (TfR), ERBB1-4, TfR, ER, ERBB1-4, PSMA, RON.

Scfv, SdFv, CDR, and/or CDO were modified to contain: (a) a specific binding domain capable of direct, domain specific binding of nanoparticles, radionuclides beta, radionuclides gamma, fluorochromes or (b) antiBiotin single chain variable fragment (as described by Malecki et al. PNAS 2002).

Monodisperse reporters consisting of: nanoparticles consisting of atoms of noble elements (e.g., Au, Ag, Pt, Pd, etc) after being manufactured by previously described technologies of laser ablation and possessing identical masses with very uniformly mono-disperse diameters;
(a) core/shell superparamagnetic/noble elements (e.g., Fe, Ni, Gd, Eu, etc) and possessing identical masses with very uniformly mono-disperse magnetism;
(b) fluorochrome nanoparticles (e.g., Eu, etc) and possessing identical or nearly identical masses with very uniformly mono-disperse fluorescence;
(c) gamma (e.g., I¹²⁵, etc) and possessing identical or nearly masses with very uniformly mono-disperse radiation;
(d) beta (e.g., Cu64, etc) and possessing identical masses or nearly identical with very uniformly mono-disperse radiation.
(e) biotags were manufactured by linking scfv, sdFv, CDR, and/or CDO with reporters. Therefore, biotags could be massive, superparamagnetic, fluorescent, radioactive. Nevertheless, each of biotag has uniform, mono-disperse reporter, so that after labeling the labeled cancer cells were carrying the number of biotags strictly proportional to the number of the receptors and that was proportional to the number of the reporters recorded by the reading devices: (a) nanoparticles counter, edx, or surface plasmon resonance; (b) NMR or edx; (c) spectrophotometer of plate reader; (d) gamma camera or scintillation counter; (e) scintillation counter.

Blood was drained from the cancer patients per IRB and ICF. The blood was run through the Ficoll or antiABRh columns/beads to eliminate RBC. The buffy coat was mixed with either massive, superparamagnetic, fluorescent, radioactive various temps e.g., 4 or 37 deg C. for variable times e.g., 15 min.
A. The density gradient was laid into the centrifuge tubes. The massive tags labeled buffy coat was laid over the top of the gradient. The samples were placed into the centrifuge. The centrifugation was set for variable time e.g., 30 min at variable g, e.g., 10-100 k×g. Every layer of the density gradient contained cancer cells or healthy cells with the same number of receptors. The intensity was read on the spr.
B. The density gradient was laid into the centrifuge tubes. The superparamagnetic tags labeled buffy coat was laid over the top of the gradient. The samples were placed into the centrifuge or gradient magnetic field. Every layer of the density gradient contained cancer cells or healthy cells with the same number of receptors. The intensity was read on the NMR.
C. The density gradient was laid into the centrifuge tubes. The fluorescent biotags labeled buffy coat was laid over the top of the gradient. After the spin or magnet, the every layer of the density gradient contained cancer cells or healthy cells with the same number of receptors. The intensity was read on the spectrophotometer.
D. The density gradient was laid into the centrifuge tubes. The gamma biotags labeled buffy coat was laid over the top of the gradient. The vials were placed into the reporter amount recording device: gamma scintillation counter. Every layer of the density gradient contained cancer cells or healthy cells with the same number of receptors.
E. The density gradient was laid into the centrifuge tubes. The superparamagnetic tags labeled buffy coat was laid over the top of the gradient. The vials were placed into the reporter amount recording device: beta scintillation counter. Every layer of the density gradient contained cancer cells or healthy cells with the same number of receptors.

The cells with identical or approximately the same number of gene expression products were sucked out of the tubes one layer at a time. The number of cells counted with cell counter. The individual cells were separated on microarray, FACS, cloning plate or other suitable method known in the art. They were ready for assessing the number of receptors per cell, qPCR, CGH, IF, microarray, etc.

Isolation of CTC through the negative selection based upon expression of CD45, CD19, CD20 by healthy cells.

Scfv, SdFv, CDR, and/or CDO were targeting CD45, CD19, CD20: Reporters were as described as above.

After proceeding as described above for A or B, the unlabeled (except B cell cancers) cancer cells were collected in the top layer of the gradient. They were sucked out of this layer. All the other cells were spun down to the denser layers.

The recovered CTC were further studied as described in the examples above.

### REFERENCES

The references listed below and all referenced cited above are hereby incorporated in their entirety by reference as if fully set forth herein.
[1] M. H. Roh, D. Kindelberger, and C. P. Crum, “Serous tubal intraepithelial carcinoma and the dominant ovarian mass: clues to serous tumor origin?” American Journal of Surgica Pathology, vol. 33, no. 3, pp. 376-383, 2009.
[2] K. M. Feeley and M. Wells, “Precursor lesions of ovarian epithelial malignancy,” Histopathology, vol. 38, no. 2, pp. 87-95, 2001.
[3] I. Dimova, B. Zaharieva, S. Raitcheva, R. Dimitrov, N. Doganov, and D. Toncheva, “Tissue microarray analysis of EGFR and erbB2 copy number changes in ovarian tumors,” International Journal of Gynecological Cancer, vol. 16, no. 1, pp. 145-151, 2006.
[4] C.-K. Lin, T.-K. Chao, C.-P. Yu, M.-H. Yu, and J.-S. Jin, “The expression of six biomarkers in the four most common ovarian cancers: correlation with clinicopathological parameters,” APMIS, vol. 117, no. 3, pp. 162-175, 2009.
[5] H. Brustmann, “Epidermal growth factor receptor expression in serous ovarian carcinoma: an immunohistochemical study with galectin-3 and cyclin D1 and outcome,” International Journal of Gynecological Pathology, vol. 27, no. 3, pp. 380-389, 2008.
[6] H. Lassus, H. Sihto, A. Leminen, et al., “Gene amplification, mutation, and protein expression of EGFR and mutations of ERBB2 in serous ovarian carcinoma,” Journal of Molecular Medicine, vol. 84, no. 8, pp. 671-681, 2006.
[7] N. J. Maihle, A. T. Baron, B. A. Barrette, et al., “EGF/ErbB receptor family in ovarian cancer,” Cancer Treatment and Research, vol. 107, pp. 247-258, 2002.
[8] Y. Yarden and A. Ullrich, “Growth factor receptor tyrosine kinases,” Annual Review of Biochemistry, vol. 57, pp. 443-478, 1988.
[9] J. Boonstra, P. Rijken, B. Humbel, F. Cremers, A. Verkleij, and P. Van Bergen en Henegouwen, “The epidermal growth factor,” Cell Biology International, vol. 19, no. 5, pp. 413-430, 1995.
[10] N. Prenzel, O. M. Fischer, S. Streit, S. Hart, and A. Ullrich, “The epidermal growth factor receptor family as a central element for cellular signal transduction and diversification,” Endocrine-Related Cancer, vol. 8, no. 1, pp. 11-31, 2001.
[11] Y. Yarden, “The EGFR family and its ligands in human cancer: signalling mechanisms and therapeutic opportunities,” European Journal of Cancer, vol. 37, supplement 4, pp. S3-S8, 2001.
[12] J. M. Lafky, J. A. Wilken, A. T. Baron, and N. J. Maihle, “Clinical implications of the ErbB/epidermal growth factor (EGF) receptor family and its ligands in ovarian cancer,” Biochimica et Biophysica Acta, vol. 1785, no. 2, pp. 232-265, 2008.
[13] A. Zaczek, B. Brandt, and K. P. Bielawski, “The diverse signaling network of EGFR, HER2, HER3 and HER4 tyrosine kinase receptors and the consequences for therapeutic approaches,” Histology and Histopathology, vol. 20, no. 3, pp. 1005-1015, 2005.
[14] E. Tzahar, H. Waterman, X. Chen, et al., “A hierarchical network of interreceptor interactions determines signal transduction by Neu differentiation factor/neuregulin and epidermal growth factor,” Molecular & Cellular Biology, vol. 16, no. 10, pp. 5276-5287, 1996.
[15] P. M. Guy, J. V. Platko, L. C. Cantley, R. A. Cerione, and K. L. Carraway III, “Insect cell-expressed p180 (erbB3) possesses an impaired tyrosine kinase activity,” Proceedings of the National Academy of Sciences of the United States of America, vol. 91, no. 17, pp. 8132-8136, 1994.
[16] G. D. Plowman, G. S. Whitney, M. G. Neubauer, et al., “Molecular cloning and expression of an additional epidermal growth factor receptor-related gene,” Proceedings of the National Academy of Sciences of the United States of America, vol. 87, no. 13, pp. 4905-4909, 1990.
[17] S. L. Sierke, K. Cheng, H.-H. Kim, and J. G. Koland, “Biochemical characterization of the protein tyrosine kinase homology domain of the ErbB3 (HER3) receptor protein,” Biochemical Journal, vol. 322, no. 3, pp. 757-763, 1997.
[18] A. Citri, K. B. Skaria, and Y. Yarden, “The deaf and the dumb: the biology of ErbB-2 and ErbB-3,” Experimental Cell Research, vol. 284, no. 1, pp. 54-65, 2003.
[19] S. Morandell, T. Stasyk, S. Skvortsov, S. Ascher, and L. A. Huber, “Quantitative proteomics and phosphoproteomics reveal novel insights into complexity and dynamics of the EGFR signaling network,” Proteomics, vol. 8, no. 21, pp. 4383-4401, 2008.
[20] T. E. Adams, N. M. McKern, and C. W. Ward, “Signalling by the type 1 insulin-like growth factor receptor: interplay with the epidermal growth factor receptor,” Growth Factors, vol. 22, no. 2, pp. 89-95, 2004.
[21] H. E. Jones, J. M. W. Gee, I. R. Hutcheson, J. M. Knowlden, D. Barrow, and R. I. Nicholson, “Growth factor receptor interplay and resistance in cancer,” Endocrine-Related Cancer, vol. 13, supplement 1, pp. S45-S51, 2006.
[22] L. Qiu, C. Zhou, Y. Sun, et al., “Crosstalk between EGFR and TrkB enhances ovarian cancer cell migration and proliferation,” International Journal of Oncology, vol. 29, no. 4, pp. 1003-1011, 2006.
[23] A. Gschwind, E. Zwick, N. Prenzel, M. Leserer, and A. Ullrich, “Cell communication networks: epidermal growth factor receptor transactivation as the paradigm for interreceptor signal transmission,” Oncogene, vol. 20, no. 13, pp. 1594-1600, 2001.
[24] D. Shida, J. Kitayama, K. Mori, T. Watanabe, and H. Nagawa, “Transactivation of epidermal growth factor receptor is involved in leptin-induced activation of Janus-activated kinase 2 and extracellular signal-regulated kinase 1/2 in human gastric cancer cells,” Cancer Research, vol. 65, no. 20, pp. 9159-9163, 2005.
[25] C. D. Andl and A. K. Rustgi, “No one-way street: crosstalk between E-cadherin and receptor tyrosine kinase (RTK) signaling: a mechanism to regulate RTK activity,” Cancer Biology and Therapy, vol. 4, no. 1, pp. 28-31, 2005.
[26] S. Cabodi, L. Moro, E. Bergatto, et al., “Integrin regulation of epidermal growth factor (EGF) receptor and of EGFdependent responses,” Biochemical Society Transactions, vol. 32, no. 3, pp. 438-442, 2004.
[27] J. Riedemann, M. Takiguchi, M. Sohail, and V. M. Macaulay, “The EGF receptor interacts with the type 1 IGF receptor and regulates its stability,” Biochemical and Biophysical Research Communications, vol. 355, no. 3, pp. 707-714, 2007.
[28] E.-M. Hur, Y.-S. Park, B. D. Lee, et al., “Sensitization of epidermal growth factor-induced signaling by bradykinin is mediated by c-Src: implications for a role of lipid microdomains,” The Journal of Biological Chemistry, vol. 279, no. 7, pp. 5852-5860, 2004.
[29] Q. Zhang, S. M. Thomas, V. W. Y. Lui, et al., “Phosphorylation of TNF-α converting enzyme by gastrin-releasing peptide induces amphiregulin release and EGF receptor activation,” Proceedings of the National Academy of Sciences of the United States of America, vol. 103, no. 18, pp. 6901-6906, 2006.
[30] S. Miyamoto, H. Yagi, F. Yotsumoto, T. Kawarabayashi, and E. Mekada, “Heparin-binding epidermal growth factorlike growth factor as a novel targeting molecule for cancer therapy,” Cancer Science, vol. 97, no. 5, pp. 341-347, 2006.
[31] S. Yu, M. M. Murph, Y. Lu, et al., “Lysophosphatidic acid receptors determine tumorigenicity and aggressiveness of ovarian cancer cells,” Journal of the National Cancer Institute, vol. 100, no. 22, pp. 1630-1642, 2008.
[32] G. B. Mills and W. H. Moolenaar, “The emerging role of lysophosphatidic acid in cancer,” Nature Reviews Cancer, vol. 3, no. 8, pp. 582-591, 2003.
[33] X. Fang, M. Schummer, M. Mao, et al., “Lysophosphatidic acid is a bioactive mediator in ovarian cancer,” Biochimica et Biophysica Acta, vol. 1582, no. 1-3, pp. 257-264, 2002.
[34] X. Yu, L. Liu, B. Cai, Y. He, and X. Wan, “Suppression of anoikis by the neurotrophic receptor TrkB in human ovarian cancer,” Cancer Science, vol. 29, no. 3, pp. 543-552, 2008.
[35] J. Vermeij, E. Teugels, C. Bourgain, et al., “Genomic activation of the EGFR and HER2-neu genes in a significant proportion of invasive epithelial ovarian cancers,” BMC Cancer, vol. 8, article 3, 2008.
[36] S. Stadlmann, U. Gueth, U. Reiser, et al., “Epithelial growth factor receptor status in primary and recurrent ovarian cancer,” Modern Pathology, vol. 19, no. 4, pp. 607-610, 2006.
[37] R. J. Schilder, M. W. Sill, X. Chen, et al., “Phase II study of gefitinib in patients with relapsed or persistent ovarian or primary peritoneal carcinoma and evaluation of epidermal growth factor receptormutations and immunohistochemical expression: a Gynecologic Oncology Group Study,” Clinical Cancer Research, vol. 11, no. 15, pp. 5539-5548, 2005.
[38] D. K. Moscatello, M. Holgado-Madruga, A. K. Godwin, et al., “Frequent expression of a mutant epidermal growth factor receptor in multiple human tumors,” Cancer Research, vol. 55, no. 23, pp. 5536-5539, 1995.
[39] K. D. Steffensen, M. Waldstrøm, D. Olsen, et al., “Mutant epidermal growth factor receptor in benign, borderline, and malignant ovarian tumors,” Clinical Cancer Research, vol. 14, no. 11, pp. 3278-3282, 2008.
[40] C.-H. Lee, D. G. Huntsman, M. C. U. Cheang, et al., “Assessment of Her-1, Her-2, and Her-3 expression and Her-2 amplification in advanced stage ovarian carcinoma,” International Journal of Gynecological Pathology, vol. 24, no. 2, pp. 147-152, 2005.
[41] J. S, Nielsen, E. Jakobsen, B. Hølund, K. Bertelsen, and A. Jakobsen, “Prognostic significance of p53, Her-2, and EGFR overexpression in borderline and epithelial ovarian cancer,” International Journal of Gynecological Cancer, vol. 14, no. 6, pp. 1086-1096, 2004.
[42] A.-R. Hanauske, C. L. Arteaga, G. M. Clark, et al., “Determination of transforming growth factor activity in effusions from cancer patients,” Cancer, vol. 61, no. 9, pp. 1832-1837, 1988.
[43] K. Morishige, H. Kurachi, K. Amemiya, et al., “Evidence for the involvement of transforming growth factor α and epidermal growth factor receptor autocrine growth mechanism in primary human ovarian cancers in vitro,” Cancer Research, vol. 51, no. 19, pp. 5322-5328, 1991.
[44] H. Kurachi, H. Adachi, K.-I. Morishige, et al., “Transforming growth factor-α promotes tumor markers secretion from human ovarian cancers in vitro,” Cancer, vol. 78, no. 5, pp. 1049-1054, 1996.
[45] M. Ueda, H. Fujii, K. Yoshizawa, et al., “Effects of sex steroids and growth factors on invasive activity and 5_-deoxy-5-fluorouridine sensitivity in ovarian adenocarcinoma OMC-3 cells,” Japanese Journal of Cancer Research, vol. 89, no. 12, pp. 1334-1342, 1998.
[46] E. Henic, M. Sixt, S. Hansson, G. Høyer-Hansen, and B. Cassl'en, “EGF-stimulated migration in ovarian cancer cells is associated with decreased internalization, increased surface expression, and increased shedding of the urokinase plasminogen activator receptor,” Gynecologic Oncology, vol. 101, no. 1, pp. 28-39, 2006.
[47] L.-Z. Liu, X.-W. Hu, C. Xia, et al., “Reactive oxygen species regulate epidermal growth factor-induced vascular endothelial growth factor and hypoxia-inducible factor-1α expression through activation of AKT and P70S6K1 in human ovarian cancer cells,” Free Radical Biology and Medicine, vol. 41, no. 10, pp. 1521-1533, 2006.
[48] M. Campiglio, S. Ali, P. G. Knyazev, and A. Ullrich, “Characteristics of EGFR family-mediated HRG signals in human ovarian cancer,” Journal of Cellular Biochemistry, vol. 73, no. 4, pp. 522-532, 1999.
[49] D. Ye, J. Mendelsohn, and Z. Fan, “Augmentation of a humanized anti-HER2 mAb 4D5 induced growth inhibition by a human-mouse chimeric anti-EGF receptor mAb C225,” Oncogene, vol. 18, no. 3, pp. 731-738, 1999.
[50] F. Vacca, A. Bagnato, K. J. Cart, and R. Tecce, “Transactivation of the epidermal growth factor receptor in endothelin-1-induced mitogenic signaling in human ovarian carcinoma cells,” Cancer Research, vol. 60, no. 18, pp. 5310-5317, 2000.
[51] A. Bagnato, R. Tecce, C. Moretti, V. Di Castro, D. Spergel, and K. J. Catt, “Autocrine actions of endothelin-1 as a growth factor in human ovarian carcinoma cells,” Clinical Cancer Research, vol. 1, no. 9, pp. 1059-1066, 1995.
[52] S. Moraitis, S. P. Langdon, and W. R. Miller, “Endothelin expression and responsiveness in human ovarian carcinoma cell lines,” European Journal of Cancer Part A, vol. 33, no. 4, pp. 661-668, 1997.
[53] M. Shichiri, Y. Hirata, T. Nakajima, et al., “Endothelin-1 is an autocrine/paracrine growth factor for human cancer cell lines,” The Journal of Clinical Investigation, vol. 87, no. 5, pp. 1867-1871, 1991.
[54] M. Colomiere, A. C. Ward, C. Riley, et al., “Cross talk of signals between EGFR and IL-6R through JAK2/STAT3 mediate epithelial-mesenchymal transition in ovarian carcinomas,” British Journal of Cancer, vol. 100, no. 1, pp. 134-144, 2009.
[55] L. Rosan'o, R. Cianfrocca, S. Masi, et al., “β-arrestin links endothelin A receptor to β-catenin signaling to induce ovarian cancer cell invasion and metastasis,” Proceedings of the National Academy of Sciences of the United States of America, vol. 106, no. 8, pp. 2806-2811, 2009.
[56] J. J. Kang, Y. P. Soon, H. S. Ji, et al., “Lysophosphatidic acid receptor 2 and Gi/Src pathway mediate cell motility through cyclooxygenase 2 expression in CAOV-3 ovarian cancer cells,” Experimental and Molecular Medicine, vol. 40, no. 6, pp. 607-616, 2008.
[57] T. C. Hamilton, R. C. Young, and K. G. Louie, “Characterization of a xenograft model of human ovarian carcinoma which produces ascites and intraabdominal carcinomatosis in mice,” Cancer Research, vol. 44, no. 11, pp. 5286-5290, 1984.
[58] K. Carson, T. J. Shaw, K. V. Clark, D.-S. Yao, and B. C. Vanderhyden, “Models of ovarian cancer—are we there yet?” Molecular and Cellular Endocrinology, vol. 239, no. 1-2, pp. 15-26, 2005.
[59] W. Shan and J. Liu, “Epithelial ovarian cancer: focus on genetics and animal models,” Cell Cycle, vol. 8, no. 5, pp. 731-735, 2009.
[60] K. D. S. Stakleff and V. E. Von Gruenigen, “Rodent models for ovarian cancer research,” International Journal of Gynecological Cancer, vol. 13, no. 4, pp. 405-412, 2003.
[61] B. C. Vanderhyden, T. J. Shaw, and J.-F. Ethier, “Animal models of ovarian cancer,” Reproductive Biology and Endocrinology, vol. 1, article 67, 2003.
[62] W. U. Gardner, “Tumorigenesis in transplanted irradiated and nonirradiated ovaries,” Journal of the National Cancer Institute, vol. 26, pp. 829-853, 1961.
[63] T. Krarup, “Oocyte destruction and ovarian tumorigenesis after direct application of a chemical carcinogen (9:0-dimethyl-1:2-benzanthrene) to the mouse ovary,” International Journal of Cancer, vol. 4, no. 1, pp. 61-75, 1969.
[64] K. F. Roby, C. C. Taylor, J. P. Sweetwood, et al., “Development of a syngeneic mouse model for events related to ovarian cancer,” Carcinogenesis, vol. 21, no. 4, pp. 585-591, 2000.
[65] E. C. Holland, W. P. Hively, R. A. DePinho, and H. E. Varmus, “A constitutively active epidermal growth factor receptor cooperates with disruption of G1 cell-cycle arrest pathways to induce glioma-like lesions in mice,” Genes & Development, vol. 12, no. 23, pp. 3675-3685, 1998.
[66] K. Politi, M. F. Zakowski, P.-D. Fan, E. A. Schonfeld, W. Pao, and H. E. Varmus, “Lung adenocarcinomas induced in mice by mutant EGF receptors found in human lung cancers respond to a tyrosine kinase inhibitor or to down-regulation of the receptors,” Genes & Development, vol. 20, no. 11, pp. 1496-1510, 2006.
[67] D. M. Dinulescu, T. A. Ince, B. J. Quade, S. A. Shafer, D. Crowley, and T. Jacks, “Role of K-ras and Pten in the development of mouse models of endometriosis and endometrioid ovarian cancer,” Nature Medicine, vol. 11, no. 1, pp. 63-70, 2005.
[68] M. J. Palayekar and T. J. Herzog, “The emerging role of epidermal growth factor receptor inhibitors in ovarian cancer,” International Journal of Gynecological Cancer, vol. 18, no. 5, pp. 879-890, 2008.
[69] C. L. Arteaga, “Overview of epidermal growth factor receptor biology and its role as a therapeutic target in human neoplasia,” Seminars in Oncology, vol. 29, no. 5, supplement 14, pp. 3-9, 2002.
[70] C. F. Nicodemus and J. S. Berek, “Monoclonal antibody therapy of ovarian cancer,” Expert Review of Anticancer Therapy, vol. 5, no. 1, pp. 87-96, 2005.
[71] T. G. Johns, T. E. Adams, J. R. Cochran, et al., “Identification of the epitope for the epidermal growth factor receptorspecific monoclonal antibody 806 reveals that it preferentially recognizes an untethered form of the receptor,” The Journal of Biological Chemistry, vol. 279, no. 29, pp. 30375-30384, 2004.
[72] S. L1, K. R. Schmitz, P. D. Jeffrey, J. J. W. Wiltzius, P. Kussie, and K. M. Ferguson, “Structural basis for inhibition of the epidermal growth factor receptor by cetuximab,” Cancer Cell, vol. 7, no. 4, pp. 301-311, 2005.
[73] U. Murthy, A. Basu, U. Rodeck, M. Herlyn, A. H. Ross, and M. Das, “Binding of an antagonistic monoclonal antibody to an intact and fragmented EGF-receptor polypeptide,” Archives of Biochemistry and Biophysics, vol. 252, no. 2, pp. 549-560, 1987.
[74] Z. Fan, Y. Lu, X. Wu, and J. Mendelsohn, “Antibody-induced epidermal growth factor receptor dimerization mediates inhibition of autocrine proliferation of A431 squamous carcinoma cells,” The Journal of Biological Chemistry, vol. 269, no. 44, pp. 27595-27602, 1994.
[75] X.-D. Yang, X.-C. Jia, J. R. F. Corvalan, P. Wang, and C. G. Davis, “Development of ABX-EGF, a fully human anti-EGF receptor monoclonal antibody, for cancer therapy,” Critical Reviews in Oncology/Hematology, vol. 38, no. 1, pp. 17-23, 2001.
[76] H. Sunada, B. E. Magun, J. Mendelsohn, and C. L. MacLeod, “Monoclonal antibody against epidermal growth factor receptor is internalized without stimulating receptor phosphorylation,” Proceedings of the National Academy of Sciences of the United States of America, vol. 83, no. 11, pp. 3825-3829, 1986.
[77] V. Gr\ddot{}unwald and M. Hidalgo, “Developing inhibitors of the epidermal growth factor receptor for cancer treatment,” Journal of the National Cancer Institute, vol. 95, no. 12, pp. 851-867, 2003.
[78] R. Mandic, C. J. Rodgarkia-Dara, L. Zhu, et al., “Treatment of HNSCC cell lines with the EGFR-specific inhibitor cetuximab (Erbitux_) results in paradox phosphorylation of tyrosine 1173 in the receptor,” FEBS Letters, vol. 580, no. 20, pp. 4793-4800, 2006.
[79] Y. Lu, X. L1, K. Liang, et al., “Epidermal growth factor receptor (EGFR) ubiquitination as a mechanism of acquired resistance escaping treatment by the anti-EGFR monoclonal antibody cetuximab,” Cancer Research, vol. 67, no. 17, pp. 8240-8247, 2007.
[80] M. V. Karamouzis, J. R. Grandis, and A. Argiris, “Therapies directed against epidermal growth factor receptor in aerodigestive carcinomas,” Journal of the American Medical Association, vol. 298, no. 1, pp. 70-82, 2007.
[81] S.-M. Huang, J. M. Bock, and P. M. Harari, “Epidermal growth factor receptor blockade with C225 modulates proliferation, apoptosis, and radiosensitivity in squamous cell carcinomas of the head and neck,” Cancer Research, vol. 59, no. 8, pp. 1935-1940, 1999.
[82] Y. Kawaguchi, K. Kono, K. Mimura, H. Sugai, H. Akaike, and H. Fujii, “Cetuximab induce antibody-dependent cellular cytotoxicity against EGFR-expressing esophageal squamous cell carcinoma,” International Journal of Cancer, vol. 120, no. 4, pp. 781-787, 2007.
[83] E. Friedl\ddot{}andera, M. Barok, J. Sz\ddot{}oll″osia, and G. Vereb, “ErbBdirected immunotherapy: antibodies in current practice and promising new agents,” Immunology Letters, vol. 116, no. 2, pp. 126-140, 2008.
[84] F. Rivera, M. E. Vega-Villegas, M. F. Lopez-Brea, and R. Marquez, “Current situation of panitumumab, matuzumab, nimotuzumab and zalutumumab,” Acta Oncologica, vol. 47, no. 1, pp. 9-19, 2008.
[85] J. Mendelsohn, “Epidermal growth factor receptor inhibition by a monoclonal antibody as anticancer therapy,” Clinical Cancer Research, vol. 3, no. 12, pp. 2703-2707, 1997.
[86] C. J. Bruns, M. T. Harbison, D. W. Davis, et al., “Epidermal growth factor receptor blockade with C225 plus gemcitabine results in regression of human pancreatic carcinoma growing orthotopically in nude mice by antiangiogenic mechanisms,” Clinical Cancer Research, vol. 6, no. 5, pp. 1936-1948, 2000.
[87] P. Perrotte, T. Matsumoto, K. Inoue, et al., “Anti-epidermal growth factor receptor antibody C225 inhibits angiogenesis in human transitional cell carcinoma growing orthotopically in nude mice,” Clinical Cancer Research, vol. 5, no. 2, pp. 257-265, 1999.
[88] N. I. Goldstein, M. Prewett, K. Zuklys, P. Rockwell, and J. Mendelsohn, “Biological efficacy of a chimeric antibody to the epidermal growth factor receptor in a human tumor xenograft model,” Clinical Cancer Research, vol. 1, no. 11, pp. 1311-1318, 1995.
[89] M. Prewett, M. Rothman, H. Waksal, M. Feldman, N. H. Bander, and D. J. Hicklin, “Mouse-human chimeric antiepidermal growth factor receptor antibody C225 inhibits the growth of human renal cell carcinoma xenografts in nude mice,” Clinical Cancer Research, vol. 4, no. 12, pp. 2957-2966, 1998.
[90] J. P. Overholser, M. C. Prewett, A. T. Hooper, H. W. Waksal, and D. J. Hicklin, “Epidermal growth factor receptor blockade by antibody IMC-C225 inhibits growth of a human pancreatic carcinoma xenograft in nude mice,” Cancer, vol. 89, no. 1, pp. 74-82, 2000.
[91] D. Raben, B. Helfrich, D. C. Chan, et al., “The effects of cetuximab alone and in combination with radiation and/or chemotherapy in lung cancer,” Clinical Cancer Research, vol. 11, no. 2, pp. 795-805, 2005.
[92] S. Kim, C. N. Prichard, M. N. Younes, et al., “Cetuximab and irinotecan interact synergistically to inhibit the growth of orthotopic anaplastic thyroid carcinoma xenografts in nude mice,” Clinical Cancer Research, vol. 12, no. 2, pp. 600-607, 2006.
[93] J. L. Eller, S. L. Longo, D. J. Hicklin, et al., “Activity of antiepidermal growth factor receptormonoclonal antibody C225 against glioblastoma multiforme,” Neurosurgery, vol. 51, no. 4, pp. 1005-1014, 2002.
[94] A. D. Jensen, M. W. M\ddot{}unter, H. Bischoff, et al., “Treatment of non-small cell lung cancer with intensity-modulated radiation therapy in combination with cetuximab: the NEAR protocol (NCT00115518),” BMC Cancer, vol. 6, article 122, 2006.
[95] C. Delbaldo, J.-Y. Pierga, V. Dieras, et al., “Pharmacokinetic profile of cetuximab (Erbitux™ alone and in combination with irinotecan in patients with advanced EGFR-positive adenocarcinoma,” European Journal of Cancer, vol. 41, no. 12, pp. 1739-1745, 2005.
[96] R. J. Schilder, H. B. Pathak, A. E. Lokshin, et al., “Phase II trial of single agent cetuximab in patients with persistent or recurrent epithelial ovarian or primary peritoneal carcinoma with the potential for dose escalation to rash,” Gynecologic Oncology, vol. 113, no. 1, pp. 21-27, 2009.
[97] A. A. Secord, J. A. Blessing, D. K. Armstrong, et al., “Phase II trial of cetuximab and carboplatin in relapsed platinumsensitive ovarian cancer and evaluation of epidermal growth factor receptor expression: a Gynecologic Oncology Group study,” Gynecologic Oncology, vol. 108, pp. 493-499, 2008.
[98] J. Konner, R. J. Schilder, F. A. DeRosa, et al., “A phase II study of cetuximab/paclitaxel/carboplatin for the initial treatment of advanced-stage ovarian, primary peritoneal, or fallopian tube cancer,” Gynecologic Oncology, vol. 110, no. 2, pp. 140-145, 2008.
[99] M. V. Seiden, H. A. Burris, U. Matulonis, et al., “A phase II trial of EMD72000 (matuzumab), a humanized anti-EGFR monoclonal antibody, in patients with platinum-resistant ovarian and primary peritoneal malignancies,” Gynecologic Oncology, vol. 104, no. 3, pp. 727-731, 2007.
[100] K. T. Flaherty and M. S. Brose, “Her-2 targeted therapy: beyond breast cancer and trastuzumab,” Current Oncology Reports, vol. 8, no. 2, pp. 90-95, 2006.
[101] M. A. Bookman, K. M. Darcy, D. Clarke-Pearson, R. A. Boothby, and I. R. Horowitz, “Evaluation of monoclonal humanized anti-HER2 antibody, trastuzumab, in patients with recurrent or refractory ovarian or primary peritoneal carcinoma with overexpression of HER2: a phase II trial of the Gynecologic Oncology Group,” Journal of Clinical Oncology, vol. 21, no. 2, pp. 283-290, 2003.
[102] S. R. Young, W.-H. Liu, J.-A. Brock, and S. T. Smith, “ERBB2 and chromosome 17 centromere studies of ovarian cancer by fluorescence in situ hybridization,” Genes Chromosomes and Cancer, vol. 16, no. 2, pp. 130-137, 1996.
[103] D. Glenn, F. Ueland, A. Bicher, et al., “A randomized phase II trial with gemcitabine with or without pertuzumab (rhuMAb 2C4) in platinum-resistant ovarian cancer (OC): preliminary safety data,” Journal of Clinical Oncology, vol. 24, p. 13001, 2006.
[104] Y. Wang, J. Hailey, D. Williams, et al., “Inhibition of insulinlike growth factor-I receptor (IGF-IR) signaling and tumor cell growth by a fully human neutralizing anti-IGF-IR antibody,” Molecular Cancer Therapeutics, vol. 4, no. 8, pp. 1214-1221, 2005.
[105] E. K. Maloney, J. L. McLaughlin, N. E. Dagdigian, et al., “An anti-insulin-like growth factor I receptor antibody that is a potent inhibitor of cancer cell proliferation,” Cancer Research, vol. 63, no. 16, pp. 5073-5083, 2003.
[106] J. Singh, E. M. Dobrusin, D. W. Fry, T. Haske, A. Whitty, and D. J. McNamara, “Structure-based design of a potent, selective, and irreversible inhibitor of the catalytic domain of the erbB receptor subfamily of protein tyrosine kinases,” Journal of Medicinal Chemistry, vol. 40, no. 7, pp. 1130-1135, 1997.
[107] J. D. Moyer, E. G. Barbacci, K. K. Iwata, et al., “Induction of apoptosis and cell cycle arrest by CP-358,774, an inhibitor of epidermal growth factor receptor tyrosine kinase,” Cancer Research, vol. 57, no. 21, pp. 4838-4848, 1997.
[108] E. K. Rowinsky, “The erbB family: targets for therapeutic development against cancer and therapeutic strategies using monoclonal antibodies and tyrosine kinase inhibitors,” Annual Review of Medicine, vol. 55, pp. 433-457, 2004.
[109] C. L. Arteaga, T. T. Ramsey, L. K. Shawver, and C. A. Guyer, “Unliganded epidermal growth factor receptor dimerization induced by direct interaction of quinazolines with the ATP binding site,” The Journal of Biological Chemistry, vol. 272, no. 37, pp. 23247-23254, 1997.
[110] J. Albanell and P. Gasc'on, “Small molecules with EGFR-TK inhibitor activity,” Current Drug Targets, vol. 6, no. 3, pp. 259-274, 2005.
[111] J. M. Sewell, K. G. Macleod, A. Ritchie, J. F. Smyth, and S. P. Langdon, “Targeting the EGF receptor in ovarian cancer with the tyrosine kinase inhibitor ZD 1839 (‘Iressa’),” British Journal of Cancer, vol. 86, no. 3, pp. 456-462, 2002.
[112] R. J. Schilder, M. W. Sill, X. Chen, et al., “Phase II study of gefitinib in patients with relapsed or persistent ovarian or primary peritoneal carcinoma and evaluation of epidermal growth factor receptormutations and immunohistochemical expression: a Gynecologic Oncology Group Study,” Clinical Cancer Research, vol. 11, no. 15, pp. 5539-5548, 2005.
[113] E. M. Posadas, M. S. Liel, V. Kwitkowski, et al., “A phase II and pharmacodynamic study of gefitinib in patients with refractory or recurrent epithelial ovarian cancer,” Cancer, vol. 109, no. 7, pp. 1323-1330, 2007.
[114] D. B. Costa, S. Kobayashi, D. G. Tenen, and M. S. Huberman, “Pooled analysis of the prospective trials of gefitinib monotherapy for EGFR-mutant non-small cell lung cancers,” Lung Cancer, vol. 58, no. 1, pp. 95-103, 2007.
[115] U. Wagner, A. du Bois, J. Pfisterer, et al., “Gefitinib in combination with tamoxifen in patients with ovarian cancer refractory or resistant to platinum-taxane based therapy—a phase II trial of the AGO Ovarian Cancer Study Group (AGO-OVAR 2.6),” Gynecologic Oncology, vol. 105, no. 1, pp. 132-137, 2007.
[116] A. N. Gordon, N. Finkler, R. P. Edwards, et al., “Efficacy and safety of erlotinib HCl, an epidermal growth factor receptor (HER1/EGFR) tyrosine kinase inhibitor, in patients with advanced ovarian carcinoma: results from a phase II multicenter study,” International Journal of Gynecological Cancer, vol. 15, no. 5, pp. 785-792, 2005.
[117] H. S. Nimeiri, A. M. Oza, R. J. Morgan, et al., “Efficacy and safety of bevacizumab plus erlotinib for patients with recurrent ovarian, primary peritoneal, and fallopian tube cancer: a trial of the Chicago, PMH, and California Phase II Consortia,” Gynecologic Oncology, vol. 110, no. 1, pp. 49-55, 2008.
[118] P. A. Vasey, M. Gore, R. Wilson, et al., “A phase Ib trial of docetaxel, carboplatin and erlotinib in ovarian, fallopian tube and primary peritoneal cancers,” British Journal of Cancer, vol. 98, no. 11, pp. 1774-1780, 2008.
[119] M. W. Saif, A. Elfiky, and R. R. Salem, “Gastrointestinal perforation due to bevacizumab in colorectal cancer,” Annals of Surgical Oncology, vol. 14, no. 6, pp. 1860-1869, 2007.
[120] P. A. Vasey, G. C. Jayson, A. Gordon, et al., “Phase III randomized trial of docetaxel-carboplatin versus paclitaxelcarboplatin as first-line chemotherapy for ovarian carcinoma,” Journal of the National Cancer Institute, vol. 96, no. 22, pp. 1682-1691, 2004.
[121] W. Xia, R. J. Mullin, B. R. Keith, et al., “Anti-tumor activity of GW572016: a dual tyrosine kinase inhibitor blocks EGF activation of EGFR/erbB2 and downstream Erk1/2 and AKT pathways,” Oncogene, vol. 21, no. 41, pp. 6255-6263, 2002.
[122] K. J. Kimball, T. M. Numnum, T. O. Kirby, et al., “A phase I study of lapatinib in combination with carboplatin in women with platinum sensitive recurrent ovarian carcinoma,” Gynecologic Oncology, vol. 111, no. 1, pp. 95-101, 2008.
[123] S. Campos, O. Hamid, M. V. Seiden, et al., “Multicenter, randomized phase II trial of oral CI-1033 for previously treated advanced ovarian cancer,” Journal of Clinical Oncology, vol. 23, no. 24, pp. 5597-5604, 2005.
[124] D. Li, L. Ambrogio, T. Shimamura, et al., “BIBW2992, an irreversible EGFR/HER2 inhibitor highly effective in preclinical lung cancer models,” Oncogene, vol. 27, no. 34, pp. 4702-4711, 2008.
[125] P. Traxler, P. R. Allegrini, R. Brandt, et al., “AEE788: a dual family epidermal growth factor receptor/ErbB2 and vascular endothelial growth factor receptor tyrosine kinase inhibitor with antitumor and antiangiogenic activity,” Cancer Research, vol. 64, no. 14, pp. 4931-4941, 2004.
[126] S.-F. Huang, H.-P. Liu, L.-H. Li, et al., “High frequency of epidermal growth factor receptor mutations with complex patterns in non-small cell lung cancers related to gefitinib responsiveness in Taiwan,” Clinical Cancer Research, vol. 10, no. 24, pp. 8195-8203, 2004.
[127] T. J. Lynch, D. W. Bell, R. Sordella, et al., “Activating mutations in the epidermal growth factor receptor underlying responsiveness of non-small-cell lung cancer to gefitinib,” The New England Journal of Medicine, vol. 350, no. 21, pp. 2129-2139, 2004.
[128] J. G. Paez, P. A. J\ddot{}anne, J. C. Lee, et al., “EGFR mutations in lung, cancer: correlation with clinical response to gefitinib therapy,” Science, vol. 304, no. 5676, pp. 1497-1500, 2004.
[129] W. Pao, V. Miller, M. Zakowski, et al., “EGF receptor gene mutations are common in lung cancers from “never smokers” and are associated with sensitivity of tumors to gefitinib and erlotinib,” Proceedings of the National Academy of Sciences of the United States of America, vol. 101, no. 36, pp. 13306-13311, 2004.
[130] L. Lacroix, P. Pautier, P. Duvillard, et al., “Response of ovarian carcinomas to gefitinib-carboplatin-paclitaxel combination is not associated with EGFR kinase domain somatic mutations,” International Journal of Cancer, vol. 118, no. 4, pp. 1068-1069, 2006.
[131] S. Ramalingam, J. Forster, C. Naret, et al., “Dual inhibition of the epidermal growth factor receptor with cetuximab, an IgG1 monoclonal antibody, and gefitinib, a tyrosine kinase inhibitor, in patients with refractory non-small cell lung cancer (NSCLC): a phase I study,” Journal of Thoracic Oncology, vol. 3, no. 3, pp. 258-264, 2008.
[132] L. Toschi and F. Cappuzzo, “Understanding the new genetics of responsiveness to epidermal growth factor receptor tyrosine kinase inhibitors,” The Oncologist, vol. 12, no. 2, pp. 211-220, 2007.
[133] F. Morgillo and H.-Y. Lee, “Resistance to epidermal growth factor receptor-targeted therapy,” Drug Resistance Updates, vol. 8, no. 5, pp. 298-310, 2005.
[134] P. Duesberg, R. L1, R. Sachs, A. Fabarius, M. B. Upender, and R. Hehlmann, “Cancer drug resistance: the central role of the karyotype,” Drug Resistance Updates, vol. 10, no. 1-2, pp. 51-58, 2007.
[135] J. A. Engelman and J. Settleman, “Acquired resistance to tyrosine kinase inhibitors during cancer therapy,” Current Opinion in Genetics and Development, vol. 18, no. 1, pp. 73-79, 2008.
[136] G. Tortora, R. Bianco, G. Daniele, et al., “Overcoming resistance to molecularly targeted anticancer therapies: rational drug combinations based on EGFR and MAPK inhibition for solid tumours and haematologic malignancies,” Drug Resistance Updates, vol. 10, no. 3, pp. 81-100, 2007.
[137] I. Martinez-Lacaci, P. Garcia Morales, J. L. Soto, and M. Saceda, “Tumour cells resistance in cancer therapy,” Clinical and Translational Oncology, vol. 9, no. 1, pp. 13-20, 2007.
[138] R. Bianco, V. Damiano, T. Gelardi, G. Daniele, F. Ciardiello, and G. Tortora, “Rational combination of targeted therapies as a strategy to overcome the mechanisms of resistance to inhibitors of EGFR signaling,” Current Pharmaceutical Design, vol. 13, no. 33, pp. 3358-3367, 2007.
[139] F. Morgillo, M. A. Bareschino, R. Bianco, G. Tortora, and F. Ciardiello, “Primary and acquired resistance to anti-EGFR targeted drugs in cancer therapy,” Differentiation, vol. 75, no. 9, pp. 788-799, 2007.
[140] Z. Weihua, R. Tsan, W.-C. Huang, et al., “Survival of cancer cells is maintained by EGFR independent of its kinase activity,” Cancer Cell, vol. 13, no. 5, pp. 385-393, 2008.
[141] R. A. Gatenby and R. J. Gillies, “Why do cancers have high aerobic glycolysis?” Nature Reviews Cancer, vol. 4, no. 11, pp. 891-899, 2004.
[142] K. Krysan, J. M. Lee, M. Dohadwala, et al., “Inflammation, epithelial to mesenchymal transition, and epidermal growth factor receptor tyrosine kinase inhibitor resistance,” Journal of Thoracic Oncology, vol. 3, no. 2, pp. 107-110, 2008.
[143] M. P. Morelli, T. Cascone, T. Troiani, et al., “Sequencedependent antiproliferative effects of cytotoxic drugs and epidermal growth factor receptor inhibitors,” Annals of Oncology, vol. 16, supplement 4, pp. iv61-iv68, 2005.
[144] B. J. B. Simpson, J. Weatherill, E. P. Miller, A. M. Lessells, S. P. Langdon, and W. R. Miller, “c-erbB-3 protein expression in ovarian tumours,” British Journal of Cancer, vol. 71, no. 4, pp. 758-762, 1995.
[145] B. Tanner, D. Hasenclever, K. Stern, et al., “ErbB-3 predicts survival in ovarian cancer,” Journal of Clinical Oncology, vol. 24, no. 26, pp. 4317-4323, 2006.
[146] A. C. Hsieh and M. M. Moasser, “Targeting HER proteins in cancer therapy and the role of the non-target HER3,” British Journal of Cancer, vol. 97, no. 4, pp. 453-457, 2007.
[147] J. G. Christensen, R. E. Schreck, E. Chan, et al., “High levels of HER-2 expression alter the ability of epidermal growth factor receptor (EGFR) family tyrosine kinase inhibitors to inhibit EGFR phosphorylation in vivo,” Clinical Cancer Research, vol. 7, no. 12, pp. 4230-4238, 2001. 18 Journal of Oncology
[148] N. V. Sergina, M. Rausch, D. Wang, et al., “Escape from HER-family tyrosine kinase inhibitor therapy by the kinase inactive HER3,” Nature, vol. 445, no. 7126, pp. 437-441, 2007.
[149] Q.-B. She, D. Solit, A. Basso, and M. M. Moasser, “Resistance to gefitinib in PTEN-Null HER-overexpressing tumor cells can be overcome through restoration of PTEN function or pharmacologic modulation of constitutive phosphatidylinositol 3_-kinase/Akt pathway signaling,” Clinical Cancer Research, vol. 9, no. 12, pp. 4340-4346, 2003.
[150] R. Bianco, I. Shin, C. A. Ritter, et al., “Loss of PTEN/MMAC1/TEP in EGF receptor-expressing tumor cells counteracts the antitumor action of EGFR tyrosine kinase inhibitors,” Oncogene, vol. 22, no. 18, pp. 2812-2822, 2003.
[151] E. Massarelli, M. Varella-Garcia, X. Tang, et al., “KRASmutation is an important predictor of resistance to therapy with epidermal growth factor receptor tyrosine kinase inhibitors in non-small cell lung cancer,” Clinical Cancer Research, vol. 13, no. 10, pp. 2890-2896, 2007.
[152] A. Lievre, J.-B. Bachet, D. Le Corre, et al., “KRAS mutation status is predictive of response to cetuximab therapy in colorectal cancer,” Cancer Research, vol. 66, no. 8, pp. 3992-3995, 2006.
[153] V. A. Miller, G. J. Riely, M. F. Zakowski, et al., “Molecular characteristics of bronchioloalveolar carcinoma and adenocarcinoma, bronchioloalveolar carcinoma subtype, predict response to erlotinib,” Journal of Clinical Oncology, vol. 26, no. 9, pp. 1472-1478, 2008.
[154] V. Auner, G. Kriegsh\ddot{}auser, D. Tong, et al., “KRAS mutation analysis in ovarian samples using a high sensitivity biochip assay,” BMC Cancer, vol. 9, article 111, 2009.
[155] M. Liu, M. S. Bryant, J. Chen, et al., “Antitumor activity of SCH 66336, an orally bioavailable tricyclic inhibitor of farnesyl protein transferase, in human tumor xenograft models and wap-ras transgenic mice,” Cancer Research, vol. 58, no. 21, pp. 4947-4956, 1998.
[156] C. Desbois-Mouthon, W. Cacheux, M.-J. Blivet-Van Eggelpoel, et al., “Impact of IGF-1R/EGFR cross-talks on hepatoma cell sensitivity to gefitinib,” International Journal of Cancer, vol. 119, no. 11, pp. 2557-2566, 2006.
[157] R. Nahta, L. X. H. Yuan, Y. Du, and F. J. Esteva, “Lapatinib induces apoptosis in trastuzumab-resistant breast cancer cells: effects on insulin-like growth factor I signaling,” Molecular Cancer Therapeutics, vol. 6, no. 2, pp. 667-674, 2007.
[158] S. M. Thomas, N. E. Bhola, Q. Zhang, et al., “Cross-talk between G protein-coupled receptor and epidermal growth factor receptor signaling pathways contributes to growth and invasion of head and neck squamous cell carcinoma,” Cancer Research, vol. 66, no. 24, pp. 11831-11839, 2006.
[159] Q. Zhang, N. E. Bhola, V. W. Y. Lui, et al., “Antitumor mechanisms of combined gastrin-releasing peptide receptor and epidermal growth factor receptor targeting in head and neck cancer,”Molecular Cancer Therapeutics, vol. 6, no. 4, pp. 1414-1424, 2007.
[160] J. A. Engelman, K. Zejnullahu, T. Mitsudomi, et al., “MET amplification leads to gefitinib resistance in lung cancer by activating ERBB3 signaling,” Science, vol. 316, no. 5827, pp. 1039-1043, 2007.
[161] S. N. Holden, S. G. Eckhardt, R. Basser, et al., “Clinical evaluation of ZD6474, an orally active inhibitor of VEGF and EGF receptor signaling, in patients with solid, malignant tumors,” Annals of Oncology, vol. 16, no. 8, pp. 1391-1397, 2005.
[162] S. Skvortsov, B. Sarg, J. Loeffler-Ragg, et al., “Different proteome pattern of epidermal growth factor receptorpositive colorectal cancer cell lines that are responsive and nonresponsive to C225 antibody treatment,” Molecular Cancer Therapeutics, vol. 3, no. 12, pp. 1551-1558, 2004.
[163] S. Nelander, W. Wang, B. Nilsson, et al., “Models from experiments: combinatorial drug perturbations of cancer cells,” Molecular Systems Biology, vol. 4, article 216, 2008.
[164] W. Kassouf, C. P. N. Dinney, G. Brown, et al., “Uncoupling between epidermal growth factor receptor and downstream signals defines resistance to the antiproliferative effect of gefitinib in bladder cancer cells,” Cancer Research, vol. 65, no. 22, pp. 10524-10535, 2005.
[165] K. Wang, L. Gan, E. Jeffery, et al., “Monitoring gene expression profile changes in ovarian carcinomas using cDNA microarray,” Gene, vol. 229, no. 1-2, pp. 101-108, 1999.
[166] M. E. Schaner, D. T. Ross, G. Ciaravino, et al., “Gene expression patterns in ovarian carcinomas,”Molecular Biology of the Cell, vol. 14, no. 11, pp. 4376-4386, 2003.
[167] Z. E. Selvanayagam, T. H. Cheung, N. Wei, et al., “Prediction of chemotherapeutic response in ovarian cancer with DNA microarray expression profiling,” Cancer Genetics and Cytogenetics, vol. 154, no. 1, pp. 63-66, 2004.
[168] C. M. Coticchia, J. Yang, and M. A. Moses, “Ovarian cancer biomarkers: current options and future promise,” Journal of the National Comprehensive Cancer Network, vol. 6, no. 8, pp. 795-802, 2008.
[169] R. I. Olivier, M. van Beurden, and L. J. van't Veer, “The role of gene expression profiling in the clinical management of ovarian cancer,” European Journal of Cancer, vol. 42, no. 17, pp. 2930-2938, 2006.
[170] U. McDermott, S. V. Sharma, and J. Settleman, “Highthroughput lung cancer cell line screening for genotypecorrelated sensitivity to an EGFR kinase inhibitor,” Methods in Enzymology, vol. 438, pp. 331-341, 2008.
[171] D. Calzolari, S. Bruschi, L. Coquin, et al., “Search algorithms as a framework for the optimization of drug combinations,” PLoS Computational Biology, vol. 4, no. 12, Article ID e1000249, 2008.
[172] J. Tapper, E. Kettunen, W. El-Rifai, M. Sepp\ddot{}al\ddot{}a, L. C. Andersson, and S. Knuutila, “Changes in gene expression during progression of ovarian carcinoma,” Cancer Genetics and Cytogenetics, vol. 128, no. 1, pp. 1-6, 2001.
[173] P. Schraml, G. Schwerdtfeger, F. Burkhalter, et al., “Combined array comparative genomic hybridization and tissue microarray analysis suggest PAK1 at 11q13.5-q14 as a critical oncogene target in ovarian carcinoma,” American Journal of Pathology, vol. 163, no. 3, pp. 985-992, 2003.
[174] K. D. Steffensen, M. Waldstrøm, R. F. Andersen, et al., “Protein levels and gene expressions of the epidermal growth factor receptors, HER1, HER2, HER3 and HER4 in benign and malignant ovarian tumors,” International Journal of Oncology, vol. 33, no. 1, pp. 195-204, 2008.
[175] O\ddot{}. Alper, E. S. Bergmann-Leitner, T. A. Bennett, N. F. Hacker, K. Stromberg, and W. G. Stetler-Stevenson, “Epidermal growth factor receptor signalling and the invasive phenotype of ovarian carcinoma cells,” Journal of the National Cancer Institute, vol. 93, no. 18, pp. 1375-1384, 2001.
[176] Z. Guo, S. Cai, R. Fang, et al., “The synergistic effects of CXCR4 and EGFR on promoting EGF-mediated metastasis in ovarian cancer cells,” Colloids and Surfaces B, vol. 60, no. 1, pp. 1-6, 2007.
[177] K. R. Kalli, S. V. Bradley, S. Fuchshuber, and C. A. Conover, “Estrogen receptor-positive human epithelial ovarian carcinoma cells respond to the antitumor drug suramin with increased proliferation: possible insight into ER and epidermal growth factor signaling interactions in ovarian cancer,” Gynecologic Oncology, vol. 94, no. 3, pp. 705-712, 2004.
[178] J. Morrison, S. S. Briggs, N. Green, et al., “Virotherapy of ovarian cancer with polymer-cloaked adenovirus retargeted to the epidermal growth factor receptor,” Molecular Therapy, vol. 16, no. 2, pp. 244-251, 2008.
[179] P. A. van Dam, I. B. Vergote, D. G. Lowe, et al., “Expression of c-erbB-2, c-myc, and c-ras oncoproteins, insulin-like growth factor receptor I, and epidermal growth factor receptor in ovarian carcinoma,” Journal of Clinical Pathology, vol. 47, no. 10, pp. 914-919, 1994.
[180] K. D. Cowden Dahl, J. Symowicz, Y. Ning, et al., “Matrix metalloproteinase 9 is a mediator of epidermal growth factor dependent E-cadherin loss in ovarian carcinoma cells,” Cancer Research, vol. 68, no. 12, pp. 4606-4613, 2008.
[181] C. Cao, S. Lu, A. Sowa, et al., “Priming with EGFR tyrosine kinase inhibitor and EGF sensitizes ovarian cancer cells to respond to chemotherapeutical drugs,” Cancer Letters, vol. 266, no. 2, pp. 249-262, 2008.
[182] N. G. Cloven, A. Kyshtoobayeva, R. A. Burger, I.-R. Yu, and J. P. Fruehauf, “In vitro chemoresistance and biomarker profiles are unique for histologic subtypes of epithelial ovarian cancer,” Gynecologic Oncology, vol. 92, no. 1, pp. 160-166, 2004.
[183] C. Schindlbeck, P. Hantschmann, M. Zerzer, et al., “Prognostic impact of KI67, p53, human epithelial growth factor receptor 2, topoisomerase IIα, epidermal growth factor receptor, and nm23 expression of ovarian carcinomas and disseminated tumor cells in the bone marrow,” International Journal of Gynecological Cancer, vol. 17, no. 5, pp. 1047-1055, 2007.
[184] I. Skirnisdottir, T. Seidal, and B. Sorbe, “A new prognostic model comprising p53, EGFR, and tumor grade in early stage epithelial ovarian carcinoma and avoiding the problem of inaccurate surgical staging,” International Journal of Gynecological Cancer, vol. 14, no. 2, pp. 259-270, 2004.
[185] Z. Suo, K. Karbove, C. G. Trope, K. Metodiev, and J. M. Nesland, “Papillary serous carcinoma of the ovary: an ultrastructural and immunohistochemical study,” Ultrastructural Pathology, vol. 28, no. 3, pp. 141-147, 2004.
[186] O\ddot{}. Alper, M. L. De Santis, K. Stromberg, N. F. Hacker, Y. S. Cho-Chung, and D. S. Salomon, “Anti-sense suppression of epidermal growth factor receptor expression alters cellular proliferation, cell-adhesion and tumorigenicity in ovarian cancer cells,” International Journal of Cancer, vol. 88, no. 4, pp. 566-574, 2000.
[187] P. de Graeff, A. P. G. Crijns, K. A. TenHoor, et al., “The ErbB signalling pathway: protein expression and prognostic value in epithelial ovarian cancer,” British Journal of Cancer, vol. 99, no. 2, pp. 341-349, 2008.
[188] C. Facco, S. La Rosa, A. Dionigi, S. Uccella, C. Riva, and C. Capella, “High expression of growth factors and growth factor receptors in ovarian metastases from ileal carcinoids: an immunohistochemical study of 2 cases,” Archives of Pathology and Laboratory Medicine, vol. 122, no. 9, pp. 828-832, 1998.
[189] G. Ferrandina, F. O. Ranelletti, L. Lauriola, et al., “Cyclooxygenase-2 (COX-2), epidermal growth factor receptor (EGFR), and Her-2/neu expression in ovarian cancer,” Gynecologic Oncology, vol. 85, no. 2, pp. 305-310, 2002.
[190] B. A. Goff, J. A. Ries, L. P. Els, M. D. Coltrera, and A. M. Gown, “Immunophenotype of ovarian cancer as predictor of clinical outcome: evaluation at primary surgery and second look procedure,” Gynecologic Oncology, vol. 70, no. 3, pp. 378-385, 1998.
[191] A. Harlozinska, J. K. Bar, E. Sobanska, and M. Goluda, “Epidermal growth factor receptor and c-erbB-2 oncoproteins in tissue and tumor effusion cells of histopathologically different ovarian neoplasms,” Tumor Biology, vol. 19, no. 5, pp. 364-373, 1998.
[192] Y. Kuwashima, T. Uehara, K. Kishi, K. Shiromizu, M. Matsuzawa, and S. Takayama, “Immunohistochemical characterization of undifferentiated carcinomas of the ovary,” Journal of Cancer Research and Clinical Oncology, vol. 120, no. 11, pp. 672-677, 1994.
[193] M. Mandai, I. Konishi, M. Koshiyama, et al., “Expression of metastasis-related nm23-H1 and nm23-H2 genes in ovarian carcinomas: correlation with clinicopathology, EGFR, cerbB-2, and c-erbB-3 genes, and sex steroid receptor expression,” Cancer Research, vol. 54, no. 7, pp. 1825-1830, 1994.
[194] I. Skirnisdottir, B. Sorbe, and T. Seidal, “The growth factor receptors HER-2/neu and EGFR, their relationship, and their effects on the prognosis in early stage (FIGO I-II) epithelial ovarian carcinoma,” International Journal of Gynecological Cancer, vol. 11, no. 2, pp. 119-129, 2001.
[195] C. van Haaften-Day, P. Russell, C. M. Boyer, et al., “Expression of cell regulatory proteins in ovarian borderline tumors,” Cancer, vol. 77, no. 10, pp. 2092-2098, 1996.
[196] M. Aponte, W. Jiang, M. Lakkis, et al., “Activation of plateletactivating factor receptor and pleiotropic effects on tyrosine phospho-EGFR/Src/FAK/paxillin in ovarian cancer,” Cancer Research, vol. 68, no. 14, pp. 5839-5848, 2008.
[197] B. Nolen, A. Marrangoni, L. Velikokhatnaya, et al., “A serum based analysis of ovarian epithelial tumorigenesis,” Gynecologic Oncology, vol. 112, no. 1, pp. 47-54, 2009.
[198] J. K. Chan, H. Pham, X. J. You, et al., “Suppression of ovarian cancer cell tumorigenicity and evasion of cisplatin resistance using a truncated epidermal growth factor receptor in a rat model,” Cancer Research, vol. 65, no. 8, pp. 3243-3248, 2005.
[199] G. Ferrandina, G. Scambia, P. Benedetti Panici, et al., “Effects of dexamethasone on the growth and epidermal growth factor receptor expression of the OVCA 433 ovarian cancer cells,” Molecular and Cellular Endocrinology, vol. 83, no. 2-3, pp. 183-193, 1992.
[200] S. L. Bull Phelps, J. O, Schorge, M. J. Peyton, et al., “Implications of EGFR inhibition in ovarian cancer cell proliferation,” Gynecologic Oncology, vol. 109, no. 3, pp. 411-417, 2008.
[201] T. Servidei, A. Riccardi, S. Mozzetti, C. Ferlini, and R. Riccardi, “Chemoresistant tumor cell lines display altered epidermal growth factor receptor and HER3 signaling and enhanced sensitivity to gefitinib,” International Journal of Cancer, vol. 123, no. 12, pp. 2939-2949, 2008.
[202] B. Davidson, V. Espina, S. M. Steinberg, et al., “Proteomic analysis of malignant ovarian cancer effusions as a tool for biologic and prognostic profiling,” Clinical Cancer Research, vol. 12, no. 3, pp. 791-799, 2006.
[203] E. M. Posadas, V. Kwitkowski, H. L. Kotz, et al., “A prospective analysis of imatinib-induced c-KIT modulation in ovarian cancer: a phase II clinical study with proteomic profiling,” Cancer, vol. 110, no. 2, pp. 309-317, 2007.
[204] J.-H. Choi, K.-C. Choi, N. Auersperg, and P. C. K. Leung, “Gonadotropins upregulate the epidermal growth factor receptor through activation of mitogen-activated protein kinases and phosphatidyl-inositol-3-kinase in human ovarian surface epithelial cells,” Endocrine-Related Cancer, vol. 12, no. 2, pp. 407-421, 2005.
[205] C. Ji, C. Cao, S. Lu, et al., “Curcumin attenuates EGF-induced AQP3 up-regulation and cell migration in human ovarian cancer cells,” Cancer Chemotherapy and Pharmacology, vol. 62, no. 5, pp. 857-865, 2008.
[206] A. J. Li, D. R. Scoles, K. U. M. Armstrong, and B. Y. Karlan, “Androgen receptor cytosine-adenine-guanine repeat polymorphisms modulate EGFR signaling in epithelial ovarian carcinomas,” Gynecologic Oncology, vol. 109, no. 2, pp. 220-225, 2008.
[207] C. Porcile, A. Bajetto, F. Barbieri, et al., “Stromal cell-derived factor-1α (SDF-1α/CXCL12) stimulates ovarian cancer cell growth through the EGF receptor transactivation,” Experimental Cell Research, vol. 308, no. 2, pp. 241-253, 2005.
[208] K. Selvendiran, A. Bratasz, L. Tong, L. J. Ignarro, and P. Kuppusamy, “NCX-4016, a nitro-derivative of aspirin, inhibits EGFR and STAT3 signaling and modulates Bcl-2 proteins in cisplatin-resistant human ovarian cancer cells and xenografts,” Cell Cycle, vol. 7, no. 1, pp. 81-88, 2008.
[209] C. Zhou, L. Qiu, Y. Sun, et al., “Inhibition of EGFR/PI3K/AKT cell survival pathway promotes TSA's effect on cell death and migration in human ovarian cancer cells,” International Journal of Oncology, vol. 29, no. 1, pp. 269-278, 2006.
[210] S. D. Pack, O\ddot{}. Alper, K. Stromberg, et al., “Simultaneous suppression of epidermal growth factor receptor and c-erbB-2 reverses aneuploidy and malignant phenotype of a human ovarian carcinoma cell line,” Cancer Research, vol. 64, no. 3, pp. 789-794, 2004.
[211] X. Zhang, M.-T. Ling, H. Feng, Y. C. Wong, S. W. Tsao, and X. Wang, “Id-1 stimulates cell proliferation through activation of EGFR in ovarian cancer cells,” British Journal of Cancer, vol. 91, no. 12, pp. 2042-2047, 2004.
[212] J. V. Ilekis, J. P. Connor, G. S. Prins, K. Ferrer, C. Niederberger, and B. Scoccia, “Expression of epidermal growth factor and androgen receptors in ovarian cancer,” Gynecologic Oncology, vol. 66, no. 2, pp. 250-254, 1997.
[213] M. G. del Carmen, I. Rizvi, Y. Chang, et al., “Synergism of epidermal growth factor receptor-targeted immunotherapy with photodynamic treatment of ovarian cancer in vivo,” Journal of the National Cancer Institute, vol. 97, no. 20, pp. 1516-1524, 2005.
[214] A. A. Kamat, T. J. Kim, C. N. Landen Jr., et al., “Metronomic chemotherapy enhances the efficacy of antivascular therapy in ovarian cancer,” Cancer Research, vol. 67, no. 1, pp. 281-288, 2007.
[215] S. Miyamoto, M. Hirata, A. Yamazaki, et al., “Heparin binding EGF-like growth factor is a promising target for ovarian cancer therapy,” Cancer Research, vol. 64, no. 16, pp. 5720-5727, 2004.
[216] G. W. Rewcastle, D. K. Murray, W. L. Elliott, et al., “Tyrosine kinase inhibitors. 14. Structure-activity relationships for methyl-amino-substituted derivatives of 4-[3-bromophenyl)amino]-6-(methylaminø)-pyrido[3,4-d]pyrimidine (PD 158780), a potent and specific inhibitor of the tyrosine kinase activity of receptors for the EGF family of growth factors,” Journal of Medicinal Chemistry, vol. 41, no. 5, pp. 742-751, 1998.
[217] L. Rosano, V. Di Castro, F. Spinella, et al., “Combined targeting of endothelin a receptor and epidermal growth factor receptor in ovarian cancer shows enhanced antitumor activity,” Cancer Research, vol. 67, no. 13, pp. 6351-6359, 2007.
[218] P. W. Vincent, A. J. Bridges, D. J. Dykes, et al., “Anticancer efficacy of the irreversible EGFr tyrosine kinase inhibitor PD 0169414 against human tumor xenografts,” Cancer Chemotherapy and Pharmacology, vol. 45, no. 3, pp. 231-238, 2000.
[219] S. R. Wedge, D. J. Ogilvie, M. Dukes, et al., “ZD6474 inhibits vascular endothelial growth factor signaling, angiogenesis, and tumor growth following oral administration,” Cancer Research, vol. 62, no. 16, pp. 4645-4655, 2002
[220] Mendelsohn J and Baselga J, The EGF receptor family as targets for cancer therapy. Ongogene (2000) 19:6550-6565.
[221] Wilson D S, Keefe A D, Szostak J W. The use of mRNA display to select high-affinity protein-binding peptides. Proc Natl Acad Sci USA. 2001 Mar. 27; 98(7):3750-5.
[222] Jemal A, Siegel R, Ward E, Hao Y, Xu J, Thun M J. Cancer statistics, 2009. CA Cancer J. Clin. 2009 July-August; 59(4):225-49.
[223] Jemal A, Center M M, Ward E. The convergence of lung cancer rates between blacks and whites under the age of 40, United States. Cancer Epidemiol Biomarkers Prey. 2009 December; 18(12):3349-52.
[224] Jemal A, Center M M, Ward E, Thun M J. Cancer occurrence. Methods Mol. Biol. 2009; 471:3-29.
[225] Slamon D J, Clark G M, Wong S G, Levin W J, Ullrich A, McGuire W L. Human breast cancer: correlation of relapse and survival with amplification of the HER-2/neu oncogene. Science. 1987 Jan. 9; 235(4785):177-82.
[226] Holbro T, Beerli R R, Maurer F, Koziczak M, Barbas C F 3rd, Hynes N E. The ErbB2/ErbB3 heterodimer functions as an oncogenic unit: ErbB2 requires ErbB3 to drive breast tumor cell proliferation. Proc Natl Acad Sci USA. 2003 Jul. 22; 100(15):8933-8.
[227] Holbro T, Civenni G, Hynes N E. The ErbB receptors and their role in cancer progression. Exp Cell Res. 2003 Mar. 10; 284(1):99-110.
[228] Normanno N, Bianco C, De Luca A, Maiello M R, Salomon D S. Target-based agents against ErbB receptors and their ligands: a novel approach to cancer treatment. Endocr Relat Cancer. 2003 March; 10(1):1-21.
[229] Newton J R, Deutscher S L. In vivo bacteriophage display for the discovery of novel peptide-based tumor-targeting agents. Methods Mol. Biol. 2009; 504:275-90.
[230] Poul M A. Selection of antibodies able to rapidly enter mammalian cells. Methods Mol. Biol. 2009; 562:155-63.
[231] Brun E, Sanche L, Sicard-Roselli C. Parameters governing gold nanoparticle X-ray radiosensitization of DNA in solution. Colloids and Surfaces B: Biointerfaces. 2009; 72:128-134.
[232] Malecki M, Hsu A, Truong L, Sanchez S. Molecular immunolabeling with recombinant single-chain variable fragment (scFv) antibodies designed with metal-binding domains. Proc Natl Acad Sci USA. 2002 Jan. 8; 99(1):213-8.
[233] Malecki M, Trembacz H, Szaniawska B, Przybyszewska M, Janik P. Vascular endothelial growth factor and soluble FLT-1 receptor interactions and biological implications. Oncol Rep. 2005 December; 14(6):1565-9.
[234] Malecki M, Przybyszewska M, Janik P. In vitro and in vivo study of the expression vector encoding vascular endothelial growth factor. Arch Immunol Ther Exp (Warsz). 2001; 49(3):243-6.
[235] Malecki M. Preparation of plasmid DNA in transfection complexes for fluorescence and electron spectroscopic imaging. Scanning Microsc Suppl. 1996; 10:1-16.
[236] Malecki M, Greaser M L. Scanning electron microscopy of muscle myofibrils after high pressure freezing and freeze-substitution-staining. Scanning Microsc. 1993 March; 7(1):115-27; discussion 127-8.
[237] Malecki M. High voltage electron microscopy and low voltage scanning electron microscopy of human neoplastic cell culture. Scanning Microsc Suppl. 1991; 5(4): S53-73.
[238] Mourad P D, Farrell L, Stamps L D, Chicoine M R, Silbergeld D L. Why are systemic glioblastoma metastases rare? Systemic and cerebral growth of mouse glioblastoma. Surg Neurol. 2005 June; 63(6):511-9; discussion 519.
[239] Weinberg R A. Cancer Cell. 2008 Oct. 7; 14(4):283-4. Leaving home early: reexamination of the canonical models of tumor progression.
[240] Podsypanina K, Du Y C, Jechlinger M, Beverly L J, Hambardzumyan D, Varmus H. Seeding and propagation of untransformed mouse mammary cells in the lung. Science. 2008 Sep. 26; 321(5897):1841-4.
[241] Paterlini P. Clinical implications of spontaneous and iatrogenic dissemination of tumor cells in patients with primary liver cancer]. C R Seances Soc Biol Fil. 1998; 192(2):283-8.
[242] Louha M, Poussin K, Ganne N, Zylberberg H, Nalpas B, Nicolet J, Capron F, Soubrane O, Vons C, Pol S, Beaugrand M, Berthelot P, Franco D, Trinchet J C, Bréchot C, Paterlini P. Spontaneous and iatrogenic spreading of liver-derived cells into peripheral blood of patients with primary liver cancer. Hepatology. 1997 October; 26(4):998-1005.
[243] Thomas J. Lynch, M.D., Daphne W. Bell, Ph. D., Raffaella Sordella, Ph. D., Sarada Gurubhagavatula, M.D., Ross A. Okimoto, B.S., Brian W. Brannigan, B.A., Patricia L. Harris, M.S., Sara M. Haserlat, B.A., Jeffrey G. Supko, Ph. D., Frank G. Haluska, M.D., Ph. D., David N. Louis, M.D., David C. Christiani, M.D., Jeff Settleman, Ph. D., and Daniel A. Haber, M. D., Ph. D. Activating Mutations in the Epidermal Growth Factor Receptor Underlying Responsiveness of Non-Small-Cell Lung Cancer to GefitinibN Engl J Med 2004; 350:2129-2139 May 20, 2004
[244] Paez J G, Jänne P A, Lee J C, Tracy S, Greulich H, Gabriel S, Herman P, Kaye F J, Lindeman N, Boggon T J, Naoki K, Sasaki H, Fujii Y, Eck M J, Sellers W R, Johnson B E, Meyerson M. EGFR mutations in lung cancer: correlation with clinical response to gefitinib therapy. Science. 2004 Jun. 4; 304(5676):1497-500. Epub 2004 Apr. 29. [245] Pao et al. PNAS 2004, 101:13306) [243]
[246] Sherry A D, Caravan P, Lenkinski R E. Primer on gadolinium chemistry. J Magn Reson Imaging. 2009 December; 30(6):1240-8.
[247] Flacke S, Fischer S, Scott M J, Fuhrhop R J, Allen J S, McLean M, Winter P, Sicard G A, Gaffney P J, Wickline S A, Lanza G M. Novel MRI contrast agent for molecular imaging of fibrin: implications for detecting vulnerable plaques. Circulation. 2001 Sep. 11; 104(11):1280-5.
[248] Jemal A, Siegel R, Ward E, Hao Y, Xu J, Thun M J. Cancer statistics, 2010. CA Cancer J Clin. 2010 60: 277-30073.

What is claimed is:

1. A method for treating a cancer in a subject, the method comprising:
administering to the subject an effective dose of a multidomain biotag that targets one or more cancer cells;
exposing the subject to one or more rounds of radiation, the one or more rounds of radiation selectively killing the one or more cancer cells targeted by the biotag.

2. The method of claim 1, wherein the biotag comprises
one or more binding domains;
one or more internalization domain;
one or more endosomal escape domain;
one or more lysosomal escape domain; and
one or more reporter domain.

3. The method of claim 1, further comprising administering an effective dose of a cancer cell specific ROS blocker.

4. The method of claim 3, wherein the cancer-cell specific anti-ROS blocker is part of the multidomain biotag.

5. The method of claim 2, wherein at least one of the one or more target binding domains is a cancer biomarker binding domain.

6. The method of claim 5, wherein the cancer biomarker is ErbB 1-4, TfR or a mutant thereof.

7. The method of claim 2, wherein at least one of the one or more target binding domains is a cancer cell specific anti-ROS blocker.

8. The method of claim 3, wherein the molecular probe has at least two target binding domains, the at least two target binding domains comprising a cancer biomarker binding domain and a cancer cell specific anti-ROS blocker.

9. The method of claim 2, wherein the reporter domain is a metal binding domain.

10. The method of claim 9, wherein the metal binding domain is associated with a noble metal nanoparticle tag selected from Au, Pd, Pt and Ag.

11. The method of claim 9, wherein the metal binding domain is associated with a superparamagnetic, heavy, and/or fluorescent element based nanoparticle tag selected from Gd, Eu, Tb, Fe, Ni, Co, Ru, Cu, F and their stable or radioactive isotopes and products of decay.

12. The method of claim 9, wherein the metal binding domain is associated with a core shell nanoparticle comprising an inner superparamagnetic metal core and an outer noble metal shell.

13. The method of claim 1, wherein the cancer is ovarian cancer, brain cancer, breast cancer, prostate cancer, lung cancer, pancreatic cancer, or adenoma cancer.

14. The method of claim 2, wherein the one or more binding domain is a single chain variable fragment (scFv), single domain variable fragment (sdFv), complementarity determining region (CDR), or specificity determining residues (SDR)

15. The method of claim 14, wherein the scFv or sdFv is an anti-ErbB 1-4 scFv, an anti-ErbB 1-4 sdFv, an anti-TfR scFv or an anti-TfR sdFv, or a mutant thereof.

16. The method of claim 1, wherein the one or more rounds of radiation is x-ray radiation.

17. The method of claim 11, wherein the one or more rounds of radiation is AC electromagnetic radiation.

18. A method for selectively inducing apoptosis in cancer cells in a subject, the method comprising:
administering to the subject an effective dose of a multidomain biotag that targets one or more cancer cells;
exposing the subject to one or more rounds of radiation, the one or more rounds of radiation causing selective induction of apoptosis in the one or more cancer cells targeted by the biotag.

19. The method of claim 18, wherein the biotag comprises:
one or more target binding domains;
one or more internalization domain;
one or more endosomal escape domain;
one or more lysosomal escape domain; and
one or more reporter domain.

20. A method for selectively inducing apoptosis in cancer cells in a subject, the method comprising:
administering to the subject an effective dose of a multidomain biotag that targets one or more cancer cells, the biotag comprising
one or more target binding domains;
one or more internalization domain;
one or more endosomal escape domain;
one or more lysosomal escape domain; and
one or more metal-binding domain (MBD);
and a metal nanoparticle tag, wherein the metal nanoparticle tag is chelated to the MBD; and
exposing the subject to one or more rounds of radiation, the one or more rounds of radiation causing selective induction of apoptosis in the one or more cancer cells targeted by the biotag.


SEQUENCE LISTING (357 sequences)

SEQ ID NO: 1 - DNA, 51 bp, Artificial Sequence
  Synthetic primer H1-Forward
gaggaggaggaggaggaggcggggcccaggcggcccaggtgcagctggtgc

SEQ ID NO: 2 - DNA, 57 bp, Artificial Sequence
  Synthetic primer H1-Reverse
gcggacccagctcatttcataakmakmgaaakmgaaakmagaggctgcacaggagag

SEQ ID NO: 3 - DNA, 45 bp, Artificial Sequence
  Synthetic primer H2-Forward1
gaaatgagctgggtccgccaggctccaggacaasgscttgagtgg

SEQ ID NO: 4 - DNA, 45 bp, Artificial Sequence
  Synthetic primer H2-Forward2
gaaatgagctgggtccgccaggctccagggaaggccctggagtgg

SEQ ID NO: 5 - DNA, 45 bp, Artificial Sequence
  Synthetic primer H2-Forward3
gaaatgagctgggtccgccaggctccagggaagggnctrgagtgg

SEQ ID NO: 6 - DNA, 36 bp, Artificial Sequence
  Synthetic primer H2-Reverse1
attgtctctggagatggtgaccctkycctgraacty

SEQ ID NO: 7 - DNA, 36 bp, Artificial Sequence
  Synthetic primer H2-Reverse2
attgtctctggagatggtgaatcggcccttcacnga

SEQ ID NO: 8 - DNA, 36 bp, Artificial Sequence
  Synthetic primer H2-Reverse3
attgtctctggagatggtgactmgactcttgaggga

SEQ ID NO: 9 - DNA, 36 bp, Artificial Sequence
  Synthetic primer H2-Reverse4
attgtctctggagatggtgacstggccttggaagga

SEQ ID NO: 10 - DNA, 36 bp, Artificial Sequence
  Synthetic primer H2-Reverse5
attgtctctggagatggtaaaccgtcctgtgaagcc

SEQ ID NO: 11 - DNA, 36 bp, Artificial Sequence
  Synthetic primer H3-Forward1
accctgagagccgaggacacrgcyttrtattactgt

SEQ ID NO: 12 - DNA, 36 bp, Artificial Sequence
  Synthetic primer H3-Forward2
accctgagagccgaggacacagccayrtattactgt

SEQ ID NO: 13 - DNA, 36 bp, Artificial Sequence
  Synthetic primer H3-Forward3
accctgagagccgaggacacrgcygtrtattactgt

SEQ ID NO: 14 - DNA, 34 bp, Artificial Sequence
  Synthetic primer H3-Reverse
gtggccggcctggccacttgaggagacggtgacc

SEQ ID NO: 15 - DNA, 21 bp, Artificial Sequence
  Synthetic primer FR3-Forward
accatctccagagacaattcc

SEQ ID NO: 16 - DNA, 19 bp, Artificial Sequence
  Synthetic primer FR3-Reverse
gtcctcggctctcagggtg

SEQ ID NO: 17 - DNA, 13 bp, Artificial Sequence
  Synthetic oligonucleotide
ggccnnnnnggcc

SEQ ID NO: 18 - DNA, 6 bp, Artificial Sequence
  Synthetic oligonucleotide
ccgcgg

SEQ ID NO: 19 - protein, 12 aa, Artificial Sequence
  Synthetic peptide Internalization domain sequence
YHWYGYTPQNVI

SEQ ID NO: 20 - protein, 16 aa, Artificial Sequence
  Synthetic peptide Internalization domain sequence
NPVVGYIGERPQYRDL

SEQ ID NO: 21 - protein, 15 aa, Artificial Sequence
  Synthetic peptide Internalization domain sequence
ICRRARGDNPDDRCT

SEQ ID NO: 22 - protein, 26 aa, Artificial Sequence
  Synthetic peptide Endosomal escape domain
GIGAVLKVLTTGLPALISWIKRKRQQ

SEQ ID NO: 23 - protein, 13 aa, Artificial Sequence
  Synthetic peptide Endosomal escape domain
GRKKRRQRRRPPQ

SEQ ID NO: 24 - protein, 23 aa, Artificial Sequence
  Synthetic peptide Endosomal escape domain
GLFGAIAGFIENGWEGMIDGWYG

SEQ ID NO: 25 - protein, 10 aa, Artificial Sequence
  Synthetic peptide Lysosomal escape domain
CHKKKKKKHC

SEQ ID NO: 26 - protein, 8 aa, Artificial Sequence
  Synthetic peptide Lysosomal escape domain
HHHHHWYG

SEQ ID NO: 27 - protein, 2 aa, Artificial Sequence
  Synthetic peptide Metal binding domain
GC

SEQ ID NO: 28 - protein, 3 aa, Artificial Sequence
  Synthetic peptide Metal binding domain
GRC

SEQ ID NO: 29 - protein, 3 aa, Artificial Sequence
  Synthetic peptide Metal binding domain
GKC

SEQ ID NO: 30 - protein, 5 aa, Artificial Sequence
  Synthetic peptide Metal binding domain
GDGRC

SEQ ID NO: 31 - protein, 5 aa, Artificial Sequence
  Synthetic peptide Metal binding domain
GEGRC

SEQ ID NO: 32 - protein, 5 aa, Artificial Sequence
  Synthetic peptide Metal binding domain
GDGKC

SEQ ID NO: 33 - protein, 5 aa, Artificial Sequence
  Synthetic peptide Metal binding domain
GEGKC

SEQ ID NO: 34 - protein, 5 aa, Artificial Sequence
  Synthetic peptide Metal binding domain
EEEEE

SEQ ID NO: 35 - protein, 6 aa, Artificial Sequence
  Synthetic peptide Metal binding domain
EEEEEE

SEQ ID NO: 36 - protein, 5 aa, Artificial Sequence
  Synthetic peptide Metal binding domain
DDDDD

SEQ ID NO: 37 - protein, 6 aa, Artificial Sequence
  Synthetic peptide Metal binding domain
DDDDDD

SEQ ID NO: 38 - protein, 11 aa, Artificial Sequence
  Synthetic peptide Metal binding domain
FHCPYDLCHIL

SEQ ID NO: 39 - protein, 5 aa, Artificial Sequence
  Synthetic peptide Metal binding domain
GDGRH

SEQ ID NO: 40 - protein, 5 aa, Artificial Sequence
  Synthetic peptide Metal binding domain
GEGRH

SEQ ID NO: 41 - protein, 5 aa, Artificial Sequence
  Synthetic peptide Metal binding domain
GDGKH

SEQ ID NO: 42 - protein, 5 aa, Artificial Sequence
  Synthetic peptide Metal binding domain
GEGKH

SEQ ID NO: 43 - protein, 3 aa, Artificial Sequence
  Synthetic peptide Metal binding domain
GRH

SEQ ID NO: 44 - protein, 3 aa, Artificial Sequence
  Synthetic peptide Metal binding domain
GKH

SEQ ID NO: 45 - DNA, 53 bp, Artificial Sequence
  Synthetic primer CDR-H1-Forward
ctctggattcacctttagcrrttatkmtatgagctgggtccgccaggctccag

SEQ ID NO: 46 - DNA, 88 bp, Artificial Sequence
  Synthetic primer CDR-H2-Forward
gggctggagtgggtctcakbgatctmtymtrrtrrtrgtartahatattacgctgattctgtaaaaggtcggttcaccatctccagag

SEQ ID NO: 47 - DNA, 51 bp, Artificial Sequence
  Synthetic primer CDR-H3-9-Reverse
ctggccccagtagtcgaamnnmnnmnnmnntytcgcacagtaatacacggc

SEQ ID NO: 48 - DNA, 66 bp, Artificial Sequence
  Synthetic primer CDR-H3-14-Reverse
ctggccccagtagtcgaamnnmnnmnnmnnmnnmnnmnnavsayctytcgcacagtaatacacggc

SEQ ID NO: 49 - DNA, 84 bp, Artificial Sequence
  Synthetic primer CDR-H3-20-Reverse
ctggccccagacgtccatascathakmakaakamnnmnnmnnmnnmnnmnnmnnambavbanvtytcgcacagtaatacacggc

SEQ ID NO: 50 - DNA, 84 bp, Artificial Sequence
  Synthetic primer CDR-H3-20SS-Reverse
ctggccccagacgtccatascathakmakaakaacamnnmnnmnnmnnacamnnambavbanctytcgcacagtaatacacggc

SEQ ID NO: 51 - DNA, 77 bp, Artificial Sequence
  Synthetic primer CDR-L1-Forward
gagggtcaccatctcttgtastggctcttcatctaatattggcartaatdmtgtcwmctggtaccagcagctcccag

SEQ ID NO: 52 - DNA, 57 bp, Artificial Sequence
  Synthetic primer CDR-L2-Forward
cccaaactcctcatctatkmtratartmakcggccaagcggggtccctgaccgattc

SEQ ID NO: 53 - DNA, 63 bp, Artificial Sequence
  Synthetic primer CDR-L3-Reverse
gaggctgattattactgtgstdcttgggatkmtagcctgartgsttatgtcttcggcggaggc

SEQ ID NO: 54 - DNA, 54 bp, Artificial Sequence
  Synthetic primer FR-H1-Forward
gaggtgcagctgttggagtctgggggaggcttggtacagcctggggggtccctg

SEQ ID NO: 55 - DNA, 54 bp, Artificial Sequence
  Synthetic primer FR-H1-Reverse
gctaaaggtgaatccagaggctgcacaggagagtctcagggaccccccaggctg

SEQ ID NO: 56 - DNA, 42 bp, Artificial Sequence
  Synthetic primer FR-H2-Reverse
tgagacccactccagccccttccctggagcctggcggaccca

SEQ ID NO: 57 - DNA, 58 bp, Artificial Sequence
  Synthetic primer FR-H3-Reverse
ggctgttcatttgcagatacagcgtgttcttggaattgtctctggagatggtgaaccg

SEQ ID NO: 58 - DNA, 55 bp, Artificial Sequence
  Synthetic primer FR-H3-Forward
gtatctgcaaatgaacagcctgagagccgaggacacggccgtgtattactgtgcg

SEQ ID NO: 59 - DNA, 42 bp, Artificial Sequence
  Synthetic primer JH-15-Forward
ttcgactactggggccagggtacactggtcaccgtgagctca

SEQ ID NO: 60 - DNA, 42 bp, Artificial Sequence
  Synthetic primer JH-6-Forward
atggactgctggggccagggtacactggtcaccgtgagctca

SEQ ID NO: 61 - DNA, 42 bp, Artificial Sequence
  Synthetic primer FR-L1-Forward
cagtctgtgctgactcagccaccctcagcgtctgggaccccc

SEQ ID NO: 62 - DNA, 43 bp, Artificial Sequence
  Synthetic primer FR-L1_Reverse
acaagagatggtgaccctctgcccgggggtcccagacgctgag

SEQ ID NO: 63 - DNA, 46 bp, Artificial Sequence
  Synthetic primer FR-L2-Reverse
atagatgaggagtttgggggccgttcctgggagctgctggtaccag

SEQ ID NO: 64 - DNA, 57 bp, Artificial Sequence
  Synthetic primer FR-L3 -Reverse
gatggccagggaggctgaggtgccagacttggagccagagaatcggtcagggacccc

SEQ ID NO: 65 - DNA, 58 bp, Artificial Sequence
  Synthetic primer FR-L3-F
tcagcctccctggccatcagtgggctccggtccgaggatgaggctgattattactgtg

SEQ ID NO: 66 - DNA, 39 bp, Artificial Sequence
  Synthetic primer JL-Forward
tatgtcttcggcggaggcaccaagctgacggtcctaggc

SEQ ID NO: 67 - DNA, 19 bp, Artificial Sequence
  Synthetic primer FRH3-short-Reverse
cgcacagtaatacacggcc

SEQ ID NO: 68 - DNA, 18 bp, Artificial Sequence
  Synthetic primer JH15-short-Forward
ttcgactactggggccag

SEQ ID NO: 69 - DNA, 27 bp, Artificial Sequence
  Synthetic primer JH6-short-Forward
atggacgtctggggccagggtacactg

SEQ ID NO: 70 - DNA, 19 bp, Artificial Sequence
  Synthetic primer pC3X-Forward
gcacgacaggtttcccgac

SEQ ID NO: 71 - DNA, 20 bp, Artificial Sequence
  Synthetic primer pC3X-Reverse
aaccatcgatagcagcaccg

SEQ ID NO: 72 - DNA, 19 bp, Artificial Sequence
  Synthetic primer H1-Reverse
gctaaaggtgaatccagag

SEQ ID NO: 73 - DNA, 20 bp, Artificial Sequence
  Synthetic primer H2-Forward
ctgggtccgccaggctccag

SEQ ID NO: 74 - DNA, 18 bp, Artificial Sequence
  Synthetic primer H2-Reverse
tgagacccactccagccc

SEQ ID NO: 75 - DNA, 19 bp, Artificial Sequence
  Synthetic primer H3-Forward
cggttcaccatctccagag

SEQ ID NO: 76 - DNA, 18 bp, Artificial Sequence
  Synthetic primer L1-Reverse
caagagatggtgaccctc

SEQ ID NO: 77 - DNA, 20 bp, Artificial Sequence
  Synthetic primer L2-Forward
ctggtaccagcagctcccag

SEQ ID NO: 78 - DNA, 18 bp, Artificial Sequence
  Synthetic primer L2-Reverse
atagatgaggagtttggg

SEQ ID NO: 79 - DNA, 18 bp, Artificial Sequence
  Synthetic primer L3-Forward
ggggtccctgaccgattc

SEQ ID NO: 80 - DNA, 19 bp, Artificial Sequence
  Synthetic primer L3-Reverse
cacagtaataatcagcctc

SEQ ID NO: 81 - DNA, 33 bp, Homo sapiens
  misc_feature (3)..(3): n is a, c, g, or t
ggnttywsnttywsnacntayggnatgcaytrr

SEQ ID NO: 82 - DNA, 48 bp, Homo sapiens
  misc_feature (3)..(3): n is a, c, g, or t
gtnathtgggaygayggnwsntayaartayttyggngaywsngtntrr

SEQ ID NO: 83 - DNA, 48 bp, Homo sapiens; identical to SEQ ID NO: 82
  misc_feature (3)..(3): n is a, c, g, or t

SEQ ID NO: 84 - DNA, 33 bp, Homo sapiens
  misc_feature (3)..(3): n is a, c, g, or t
ggnttyacntaywsnacntayggnatgcaytrr

SEQ ID NO: 85 - DNA, 48 bp, Homo sapiens
  misc_feature (3)..(3): n is a, c, g, or t
gtnathtgggargayggnwsntayaartaytayggngaywsngtntrr

SEQ ID NO: 86 - DNA, 51 bp, Homo sapiens
  misc_feature (6)..(6): n is a, c, g, or t
gayggnathwsnatggtnmgngcngtnatgmgngaytayttygayttytrr

SEQ ID NO: 87 - DNA, 33 bp, Homo sapiens
  misc_feature (3)..(3): n is a, c, g, or t
ggnttyacnttywsnacnttygcnatgcaytrr

SEQ ID NO: 88 - DNA, 48 bp, Homo sapiens
  misc_feature (3)..(3): n is a, c, g, or t
gtnathtgggaygayggnwsntayaarttytaygcngarwsngtntrr

SEQ ID NO: 89 - DNA, 51 bp, Homo sapiens
  misc_feature (6)..(6): n is a, c, g, or t
gayggnathacnatggtnmgnggngtnatgmgngaytayttygayttytrr

SEQ ID NO: 90 - DNA, 36 bp, Homo sapiens
  misc_feature (3)..(3): n is a, c, g, or t
mgngcnwsncargayathwsnwsngcnytngtntrr

SEQ ID NO: 91 - DNA, 21 bp, Homo sapiens
  misc_feature (6)..(6): n is a, c, g, or t
gaygcnwsnwsnytngartrr

SEQ ID NO: 92 - DNA, 30 bp, Homo sapiens
  misc_feature (15)..(15): n is a, c, g, or t
carcarttyaaywsntayccnytnacntrr

SEQ ID NO: 93 - DNA, 36 bp, Homo sapiens
  misc_feature (3)..(3): n is a, c, g, or t
mgngcnwsncargarathwsnwsngcnytnytntrr

SEQ ID NO: 94 - DNA, 24 bp, Homo sapiens
  misc_feature (6)..(6): n is a, c, g, or t
gargcnwsnwsnytngaracntrr

SEQ ID NO: 95 - DNA, 30 bp, Homo sapiens
  misc_feature (15)..(15): n is a, c, g, or t
caraayttyaaywsntayccnytnwsntrr

SEQ ID NO: 96 - DNA, 36 bp, Homo sapiens
  misc_feature (3)..(3): n is a, c, g, or t
mgngcnwsncargayathacnwsngcnytnytntrr

SEQ ID NO: 97 - DNA, 21 bp, Homo sapiens
  misc_feature (6)..(6): n is a, c, g, or t
gaygcnwsnwsnytngarwsn

SEQ ID NO: 98 - DNA, 27 bp, Homo sapiens
  misc_feature (15)..(15): n is a, c, g, or t
aaycarttycarwsntayccnytnwsn

SEQ ID NO: 99 - DNA, 33 bp, Homo sapiens
  misc_feature (3)..(3): n is a, c, g, or t
ggnttywsnttymgnaarttyggnatgwsntrr

SEQ ID NO: 100 - DNA, 48 bp, Homo sapiens
  misc_feature (3)..(3): n is a, c, g, or t
wsnathwsnacnggnggntayaaywsntaytaywsngayaaygtntrr

SEQ ID NO: 101 - DNA, 36 bp, Homo sapiens
  misc_feature (3)..(3): n is a, c, g, or t
ggnttywsnwsnacnwsntaygcnatggaytaytrr

SEQ ID NO: 102 - DNA, 33 bp, Homo sapiens
  misc_feature (3)..(3): n is a, c, g, or t
ggnttyacnttyaaraarttyggnatgwsntrr

SEQ ID NO: 103 - DNA, 48 bp, Homo sapiens
  misc_feature (3)..(3): n is a, c, g, or t
wsnathwsnacnggnggnttyaayacntaytaywsngayaaygtntrr

SEQ ID NO: 104 - DNA, 36 bp, Homo sapiens
  misc_feature (3)..(3): n is a, c, g, or t
ggntaywsnwsnacnwsnttyggnatggaytaytrr

SEQ ID NO: 105 - DNA, 33 bp, Homo sapiens
  misc_feature (3)..(3): n is a, c, g, or t
ggntaywsnttymgnaarttyggnatgwsntrr

SEQ ID NO: 106 - DNA, 48 bp, Homo sapiens
  misc_feature (3)..(3): n is a, c, g, or t
wsnathwsnacnggnggntaycaracntaytaywsngayaaygtntrr

SEQ ID NO: 107 - DNA, 36 bp, Homo sapiens
  misc_feature (3)..(3): n is a, c, g, or t
ggntaywsnwsnacnwsntaygcnatggayttytrr

SEQ ID NO: 108 - DNA, 48 bp, Homo sapiens
  misc_feature (3)..(3): n is a, c, g, or t
mgngcnwsncarwsngtncaywsngayggnaayacntayatgcartrr

SEQ ID NO: 109 - DNA, 24 bp, Homo sapiens
  misc_feature (3)..(3): n is a, c, g, or t
gcngcnwsnaaymgnttywsntrr

SEQ ID NO: 110 - DNA, 30 bp, Homo sapiens
  misc_feature (9)..(9): n is a, c, g, or t
carcarggnacncarytnccnmgnacntrr

SEQ ID NO: 111 - DNA, 48 bp, Homo sapiens
  misc_feature (3)..(3): n is a, c, g, or t
mgnwsnwsncarwsngtncaywsngayggnaaywsntayytnwsntrr

SEQ ID NO: 112 - DNA, 24 bp, Homo sapiens
  misc_feature (3)..(3): n is a, c, g, or t
ggngcnwsnaayaarttywsntrr

SEQ ID NO: 113 - DNA, 30 bp, Homo sapiens; identical to SEQ ID NO: 110
  misc_feature (9)..(9): n is a, c, g, or t

SEQ ID NO: 114 - DNA, 48 bp, Homo sapiens
  misc_feature (6)..(6): n is a, c, g, or t
aarwsncarwsnytngtncaywsngayggnaaywsntayytnwsntrr

SEQ ID NO: 115 - DNA, 24 bp, Homo sapiens
  misc_feature (3)..(3): n is a, c, g, or t
mgnathwsnaaymgnttywsntrr

SEQ ID NO: 116 - DNA, 30 bp, Homo sapiens; identical to SEQ ID NO: 110
  misc_feature (9)..(9): n is a, c, g, or t

SEQ ID NO: 117 - DNA, 30 bp, Homo sapiens
  misc_feature (3)..(3): n is a, c, g, or t
ggntaywsntaywsnwsntaytggatgtrr

SEQ ID NO: 118 - DNA, 48 bp, Homo sapiens
  misc_feature (3)..(3): n is a, c, g, or t
gcnathgayccnmgnaaywsngayacnathtayaayccncarttytrr

SEQ ID NO: 119 - DNA, 21 bp, Homo sapiens
  misc_feature (3)..(3): n is a, c, g, or t
ytntaytaytaygaywsntrr

SEQ ID NO: 120 - DNA, 30 bp, Homo sapiens
  misc_feature (3)..(3): n is a, c, g, or t
ggntayacnathwsnwsntaytggatgtrr

SEQ ID NO: 121 - DNA, 48 bp, Homo sapiens
  misc_feature (3)..(3): n is a, c, g, or t
gcngcngayccnmgnaaywsngayacnathtaycarccncartaytrr

SEQ ID NO: 122 - DNA, 21 bp, Homo sapiens
  misc_feature (3)..(3): n is a, c, g, or t
ytntaytayttygaywsntrr

SEQ ID NO: 123 - DNA, 30 bp, Homo sapiens
  misc_feature (3)..(3): n is a, c, g, or t
ggntayacngcnacnacntaytggatgtrr

SEQ ID NO: 124 - DNA, 42 bp, Homo sapiens
  misc_feature (12)..(12): n is a, c, g, or t
atgathcayccnwsngaywsngargtnmgnytnaaycartrr

SEQ ID NO: 125 - DNA, 21 bp, Homo sapiens
  misc_feature (3)..(3): n is a, c, g, or t
ytntaytayttygarwsntrr

SEQ ID NO: 126 - DNA, 24 bp, Homo sapiens
  misc_feature (18)..(18): n is a, c, g, or t
gayathaayaaytaygtntgytrr

SEQ ID NO: 127 - DNA, 24 bp, Homo sapiens
  misc_feature (6)..(6): n is a, c, g, or t
aargcnaaymgnytngtngaytrr

SEQ ID NO: 128 - DNA, 30 bp, Homo sapiens
  misc_feature (3)..(3): n is a, c, g, or t
ytncartaygaygarttyccntayacntrr

SEQ ID NO: 129 - DNA, 24 bp, Homo sapiens
  misc_feature (18)..(18): n is a, c, g, or t
garathaayaaytayytntgytrr

SEQ ID NO: 130 - DNA, 24 bp, Homo sapiens
  misc_feature (3)..(3): n is a, c, g, or t
mgngcnaayaarytngtngaytrr

SEQ ID NO: 131 - DNA, 30 bp, Homo sapiens
  misc_feature (3)..(3): n is a, c, g, or t
ytncartaygaygayttyccntayacntrr

SEQ ID NO: 132 - DNA, 24 bp, Homo sapiens
  misc_feature (18)..(18): n is a, c, g, or t
gayathaaycarttyytntgytrr

SEQ ID NO: 133 - DNA, 24 bp, Homo sapiens
  misc_feature (3)..(3): n is a, c, g, or t
mgngcnaaymgnytngtngaytrr

SEQ ID NO: 134 - DNA, 30 bp, Homo sapiens
  misc_feature (3)..(3): n is a, c, g, or t
gtncartaygaygarttyccntaywsntrr

SEQ ID NO: 135 - DNA, 33 bp, Homo sapiens
ggctttagctttagcacctatggcatgcattaa

SEQ ID NO: 136 - DNA, 48 bp, Homo sapiens
gtgatttgggatgatggcagctataaatattttggcgatagcgtgtaa

SEQ ID NO: 137 - DNA, 48 bp, Homo sapiens; identical to SEQ ID NO: 136

SEQ ID NO: 138 - DNA, 33 bp, Homo sapiens
ggctttacctatagcacctatggcatgcattaa

SEQ ID NO: 139 - DNA, 48 bp, Homo sapiens
gtgatttgggaagatggcagctataaatattatggcgatagcgtgtaa

SEQ ID NO: 140 - DNA, 51 bp, Homo sapiens
gatggcattagcatggtgcgcgcggtgatgcgcgattattttgatttttaa

SEQ ID NO: 141 - DNA, 33 bp, Homo sapiens
ggctttacctttagcacctttgcgatgcattaa

SEQ ID NO: 142 - DNA, 48 bp, Homo sapiens
gtgatttgggatgatggcagctataaattttatgcggaaagcgtgtaa

SEQ ID NO: 143 - DNA, 51 bp, Homo sapiens
gatggcattaccatggtgcgcggcgtgatgcgcgattattttgatttttaa

SEQ ID NO: 144 - DNA, 36 bp, Homo sapiens
cgcgcgagccaggatattagcagcgcgctggtgtaa

SEQ ID NO: 145 - DNA, 21 bp, Homo sapiens
gatgcgagcagcctggaataa

SEQ ID NO: 146 - DNA, 30 bp, Homo sapiens
cagcagtttaacagctatccgctgacctaa

SEQ ID NO: 147 - DNA, 36 bp, Homo sapiens
cgcgcgagccaggaaattagcagcgcgctgctgtaa

SEQ ID NO: 148 - DNA, 24 bp, Homo sapiens
gaagcgagcagcctggaaacctaa

SEQ ID NO: 149 - DNA, 30 bp, Homo sapiens
cagaactttaacagctatccgctgagctaa

SEQ ID NO: 150 - DNA, 36 bp, Homo sapiens
cgcgcgagccaggatattaccagcgcgctgctgtaa

SEQ ID NO: 151 - DNA, 21 bp, Homo sapiens
gatgcgagcagcctggaaagc

SEQ ID NO: 152 - DNA, 27 bp, Homo sapiens
aaccagtttcagagctatccgctgagc

SEQ ID NO: 153 - DNA, 33 bp, Homo sapiens
ggctttagctttcgcaaatttggcatgagctaa

SEQ ID NO: 154 - DNA, 48 bp, Homo sapiens
agcattagcaccggcggctataacagctattatagcgataacgtgtaa

SEQ ID NO: 155 - DNA, 36 bp, Homo sapiens
ggctttagcagcaccagctatgcgatggattattaa

SEQ ID NO: 156 - DNA, 33 bp, Homo sapiens
ggctttacctttaaaaaatttggcatgagctaa

SEQ ID NO: 157 - DNA, 48 bp, Homo sapiens
agcattagcaccggcggctttaacacctattatagcgataacgtgtaa

SEQ ID NO: 158 - DNA, 36 bp, Homo sapiens
ggctatagcagcaccagctttggcatggattattaa

SEQ ID NO: 159 - DNA, 33 bp, Homo sapiens
ggctatagctttcgcaaatttggcatgagctaa

SEQ ID NO: 160 - DNA, 48 bp, Homo sapiens
agcattagcaccggcggctatcagacctattatagcgataacgtgtaa

SEQ ID NO: 161 - DNA, 36 bp, Homo sapiens
ggctatagcagcaccagctatgcgatggatttttaa

SEQ ID NO: 162 - DNA, 48 bp, Homo sapiens
cgcgcgagccagagcgtgcatagcgatggcaacacctatatgcagtaa

SEQ ID NO: 163 - DNA, 24 bp, Homo sapiens
gcggcgagcaaccgctttagctaa

SEQ ID NO: 164 - DNA, 30 bp, Homo sapiens
cagcagggcacccagctgccgcgcacctaa

SEQ ID NO: 165 - DNA, 48 bp, Homo sapiens
cgcagcagccagagcgtgcatagcgatggcaacagctatctgagctaa

SEQ ID NO: 166 - DNA, 24 bp, Homo sapiens
ggcgcgagcaacaaatttagctaa

SEQ ID NO: 167 - DNA, 30 bp, Homo sapiens; identical to SEQ ID NO: 164

SEQ ID NO: 168 - DNA, 48 bp, Homo sapiens
aaaagccagagcctggtgcatagcgatggcaacagctatctgagctaa

SEQ ID NO: 169 - DNA, 24 bp, Homo sapiens
cgcattagcaaccgctttagctaa

SEQ ID NO: 170 - DNA, 30 bp, Homo sapiens; identical to SEQ ID NO: 164

SEQ ID NO: 171 - DNA, 30 bp, Homo sapiens
ggctatagctatagcagctattggatgtaa

SEQ ID NO: 172 - DNA, 48 bp, Homo sapiens
gcgattgatccgcgcaacagcgataccatttataacccgcagttttaa

SEQ ID NO: 173 - DNA, 21 bp, Homo sapiens
ctgtattattatgatagctaa

SEQ ID NO: 174 - DNA, 30 bp, Homo sapiens
ggctataccattagcagctattggatgtaa

SEQ ID NO: 175 - DNA, 48 bp, Homo sapiens
gcggcggatccgcgcaacagcgataccatttatcagccgcagtattaa

SEQ ID NO: 176 - DNA, 21 bp, Homo sapiens
ctgtattattttgatagctaa

SEQ ID NO: 177 - DNA, 30 bp, Homo sapiens
ggctataccgcgaccacctattggatgtaa

SEQ ID NO: 178 - DNA, 42 bp, Homo sapiens
atgattcatccgagcgatagcgaagtgcgcctgaaccagtaa

SEQ ID NO: 179 - DNA, 21 bp, Homo sapiens
ctgtattattttgaaagctaa

SEQ ID NO: 180 - DNA, 24 bp, Homo sapiens
gatattaacaactatgtgtgctaa

SEQ ID NO: 181 - DNA, 24 bp, Homo sapiens
aaagcgaaccgcctggtggattaa

SEQ ID NO: 182 - DNA, 30 bp, Homo sapiens
ctgcagtatgatgaatttccgtatacctaa

SEQ ID NO: 183 - DNA, 24 bp, Homo sapiens
gaaattaacaactatctgtgctaa

SEQ ID NO: 184 - DNA, 24 bp, Homo sapiens
cgcgcgaacaaactggtggattaa

SEQ ID NO: 185 - DNA, 30 bp, Homo sapiens
ctgcagtatgatgattttccgtatacctaa

SEQ ID NO: 186 - DNA, 24 bp, Homo sapiens
gatattaaccagtttctgtgctaa

SEQ ID NO: 187 - DNA, 24 bp, Homo sapiens
cgcgcgaaccgcctggtggattaa

SEQ ID NO: 188 - DNA, 30 bp, Homo sapiens
gtgcagtatgatgaatttccgtatagctaa

SEQ ID NO: 189 - protein, 10 aa, Homo sapiens
GFSFSTYGMH

SEQ ID NO: 190 - protein, 15 aa, Homo sapiens
VIWDDGSYKYFGDSV

SEQ ID NO: 191 - protein, 16 aa, Homo sapiens
DAITMVRGVMKEYFDY

SEQ ID NO: 192 - protein, 10 aa, Homo sapiens
GFTYSTYGMH

SEQ ID NO: 193 - protein, 15 aa, Homo sapiens
VIWEDGSYKYYGDSV

SEQ ID NO: 194 - protein, 16 aa, Homo sapiens
DGISMVRAVMRDYFDF

SEQ ID NO: 195 - protein, 10 aa, Homo sapiens
GFTFSTFAMH

SEQ ID NO: 196 - protein, 15 aa, Homo sapiens
VIWDDGSYKFYAESV

SEQ ID NO: 197 - protein, 16 aa, Homo sapiens
DGITMVRGVMRDYFDF

SEQ ID NO: 198 - protein, 11 aa, Homo sapiens
RASQDISSALV

SEQ ID NO: 199 - protein, 6 aa, Homo sapiens
DASSLE

SEQ ID NO: 200 - protein, 9 aa, Homo sapiens
QQFNSYPLT

SEQ ID NO: 201 - protein, 11 aa, Homo sapiens
RASQEISSALL

SEQ ID NO: 202 - protein, 7 aa, Homo sapiens
EASSLET

SEQ ID NO: 203 - protein, 9 aa, Homo sapiens
QNFNSYPLS

SEQ ID NO: 204 - protein, 11 aa, Homo sapiens
RASQDITSALL

SEQ ID NO: 205 - protein, 7 aa, Homo sapiens
DASSLES

SEQ ID NO: 206 - protein, 9 aa, Homo sapiens
NQFQSYPLS

SEQ ID NO: 207 - protein, 10 aa, Homo sapiens
GFSFRKFGMS

SEQ ID NO: 208 - protein, 15 aa, Homo sapiens
SISTGGYNSYYSDNV

SEQ ID NO: 209 - protein, 11 aa, Homo sapiens
GFSSTSYAMDY

SEQ ID NO: 210 - protein, 10 aa, Homo sapiens
GFTFKKFGMS

SEQ ID NO: 211 - protein, 15 aa, Homo sapiens
SISTGGFNTYYSDNV

SEQ ID NO: 212 - protein, 11 aa, Homo sapiens
GYSSTSFGMDY

SEQ ID NO: 213 - protein, 10 aa, Homo sapiens
GYSFRKFGMS

SEQ ID NO: 214 - protein, 15 aa, Homo sapiens
SISTGGYQTYYSDNV

SEQ ID NO: 215 - protein, 11 aa, Homo sapiens
GYSSTSYAMDF

SEQ ID NO: 216 - protein, 15 aa, Homo sapiens
RASQSVHSDGNTYMQ

SEQ ID NO: 217 - protein, 7 aa, Homo sapiens
AASNRFS

SEQ ID NO: 218 - protein, 9 aa, Homo sapiens
QQGTQLPRT

SEQ ID NO: 219 - protein, 15 aa, Homo sapiens
RSSQSVHSDGNSYLS

SEQ ID NO: 220 - protein, 7 aa, Homo sapiens
GASNKFS

SEQ ID NO: 221 - protein, 9 aa, Homo sapiens
QQGTQLPRT

SEQ ID NO: 222 - protein, 15 aa, Homo sapiens
KSQSLVHSDGNSYLS

SEQ ID NO: 223 - protein, 7 aa, Homo sapiens
RISNRFS

SEQ ID NO: 224 - protein, 9 aa, Homo sapiens
QQGTQLPRT

SEQ ID NO: 225 - protein, 9 aa, Homo sapiens
GYSYSSYWM

SEQ ID NO: 226 - protein, 15 aa, Homo sapiens
AIDPRNSDTIYNPQF

SEQ ID NO: 227 - protein, 6 aa, Homo sapiens
LYYYDS

SEQ ID NO: 228 - protein, 9 aa, Homo sapiens
GYTISSYWM

SEQ ID NO: 229 - protein, 15 aa, Homo sapiens
AADPRNSDTIYQPQY

SEQ ID NO: 230 - protein, 6 aa, Homo sapiens
LYYFDS

SEQ ID NO: 231 - protein, 9 aa, Homo sapiens
GYTATTYWM

SEQ ID NO: 232 - protein, 13 aa, Homo sapiens
MIHPSDSEVRLNQ

SEQ ID NO: 233 - protein, 6 aa, Homo sapiens
LYYFES

SEQ ID NO: 234 - protein, 7 aa, Homo sapiens
DINNYVC

SEQ ID NO: 235 - protein, 7 aa, Homo sapiens
KANRLVD

SEQ ID NO: 236 - protein, 9 aa, Homo sapiens
LQYDEFPYT

SEQ ID NO: 237 - protein, 7 aa, Homo sapiens
EINNYLC

SEQ ID NO: 238 - protein, 7 aa, Homo sapiens
RANKLVD

SEQ ID NO: 239 - protein, 9 aa, Homo sapiens
LQYDDFPYT

SEQ ID NO: 240 - protein, 7 aa, Homo sapiens
DINQFLC

SEQ ID NO: 241 - protein, 7 aa, Homo sapiens
RANRLVD

SEQ ID NO: 242 - protein, 9 aa, Homo sapiens
VQYDEFPYS

SEQ ID NO: 243 - DNA, 18 bp, Artificial Sequence
  Synthetic primer JL-short-Forward
tatgtcttcggcggaggc

SEQ ID NO: 244 - DNA, 372 bp, Homo sapiens
  misc_feature (6)..(6): n is a, c, g, or t
cargtncarytngtngaywsnggngcnggngtngtncarccnggnmgnwsnytnmgngtnwsntgygcngcnwsnggnttywsnttywsnacntayggnatgcaytgggtnmgncarggnccnggnaarggnytngartgggtngcngtnathtgggaygayggnwsntayaartayttyggngaywsngtnmgnggnmgntayacnathwsnaargarcarwsnaargtnacnytnttygtncaratgaaywsnytnaargcngaygaracngcnggnttytaytgygcnmgngaygcnathacnatggtnmgnggngtnatgaargartayttygaytaytggggncarggnacnytngtnacngtntrr

SEQ ID NO: 245 - DNA, 372 bp, Homo sapiens
  misc_feature (6)..(6): n is a, c, g, or t
cargtncarytngtngaracnggngcnggngtngtncarccnggnmgnwsnytnaargtnwsntgygcngcnwsnggnttyacntaywsnacntayggnatgcaytgggtnmgncargcnccnggnmgnggnytngartgggtngcngtnathtgggargayggnwsntayaartaytayggngaywsngtnaarggnmgnttyacngcnwsnmgngayaaywsnmgnaayacnytntayytnaayatgaaywsnytnaargcngaygaywsngcngtntaytaytgygcnmgngayggnathwsnatggtnmgngcngtnatgmgngaytayttygayttytggggncarggnacnytngtnacngtntrr

SEQ ID NO: 246 - DNA, 372 bp, Homo sapiens
  misc_feature (6)..(6): n is a, c, g, or t
cargtncarytngtngaywsnggnggnggngtnytncarccnggnmgnwsnytnaarytnwsntgygcngcnwsnggnttyacnttywsnacnttygcnatgcaytgggtnmgncargcnccngcnaarggnytngartgggtngcngtnathtgggaygayggnwsntayaarttytaygcngarwsngtnmgnggnmgnttyacnggnacnmgngayaaywsnaargtnacnytnttyytncaratgcarwsnytnmgngcngargayacngcngtnttytaytgygcnmgngayggnathacnatggtnmgnggngtnatgmgngaytayttygayttytggggncarggnacnytngtnacngtntrr

SEQ ID NO: 247 - DNA, 315 bp, Homo sapiens
  misc_feature (3)..(3): n is a, c, g, or t
gcnathcarytnacnaaywsnccnwsnwsnytnwsngcnwsngtnggngaymgngtnacnathwsntgymgngcnwsncargayathwsnwsngcnytngtntggtaycarcaraarccngcnmgngcnccnaarytngtnathtaygaygcnwsnwsnytngarwsnggngtnccnacnaarttyacnggnacngaywsnggnacngayttywsnytnacnathwsnwsnytncarccngaygayttygcnacnttytaytgycarcarttyaaywsntayccnytnacnttyggnggnggnacnaargtntrr

SEQ ID NO: 248 - DNA, 315 bp, Homo sapiens
  misc_feature (3)..(3): n is a, c, g, or t
gcnathcargtnacncarwsnccnacnwsnytnwsngcnacngtnggngaymgngtnwsnathacntgymgngcnwsncargarathwsnwsngcnytnytntggtaycarcaraarccnggnaargcnccnmgnytnytnathtaygargcnwsnwsnytngaracnggngtnccnwsnaarttyacnggnwsngaracnggnwsngayttyacnmgnacnathwsnwsngtncarccngargaygcntayacntayttytgycaraayttyaaywsntayccnytnwsnttyggnggnggnacnaargtntrr

SEQ ID NO: 249 - DNA, 315 bp, Homo sapiens
  misc_feature (3)..(3): n is a, c, g, or t
gcnathcarytnacncarwsnccnwsnacnytnacngcnwsngtnggngaymgngtnacnathacntgymgngcnwsncargayathacnwsngcnytnytntggtaycarcarmgnccngcnaargcnccnaargtnytnathtaygaygcnwsnwsnytngarwsnggngtnccnwsnmgnttywsnggnwsngaywsnggnwsngartayacnytnacnathwsnwsngtnaayccngaygaytaygcnacntaytaytgyaaycarttycarwsntayccnytnwsnttyggnggnggnacnaargtntrr

SEQ ID NO: 250 - DNA, 357 bp, Homo sapiens
  misc_feature (6)..(6): n is a, c, g, or t
cargtnaarytncarcarwsnggnggnggnytnccnaargtngcnggnwsnytnaarytnwsntgygtnacnwsnggnttywsnttymgnaarttyggnatgwsntgggtnmgncaracnwsngayaarmgnytngartggathggnwsnathwsnacnggnggntayaaywsntaytaywsngayaaygtnaarggnmgnttyacnathwsnmgngaraaygcnaaraayacnytntayytnaayatgwsnwsnytnaarwsngargayacngcnytntaytaytgygcnmgnggnttywsnwsnacnwsntaygcnatggaytaytggggncarggnacnacngtnacngtntrr

SEQ ID NO: 251 - DNA, 351 bp, Homo sapiens
  misc_feature (6)..(6): n is a, c, g, or t
cargtnaargtncaraaywsnggnggnggnytngtnaarccnggngcnwsnytnaarytnwsntgygtnacnwsnggnttyacnttyaaraarttyggnatgwsntgggtnaarcaracnwsngayaaraarytngartgggtngcnwsnathwsnacnggnggnttyaayacntaytaywsngayaaygtnaarggnmgnttyacnathwsnmgngaraayggnaaraayacnytntaygtncaratgwsnwsnytnaarwsngargayacngcnytntaytaytgyacnmgnggntaywsnwsnacnwsnttyggnatggaytaytggggncarggnacnacngtntrr

SEQ ID NO: 252 - DNA, 357 bp, Homo sapiens
  misc_feature (6)..(6): n is a, c, g, or t
cargtnaarytncarcarwsnggngcnggnytngtnaarccnggngcnwsnytnaarytnwsntgygtnacnwsnggntaywsnttymgnaarttyggnatgwsntgggtnmgncarwsnacngayaarmgnytngartgggtngcnwsnathwsnacnggnggntaycaracntaytaywsngayaaygtnaarggnmgnttyacnathwsnmgngaraaygcnaaraayacnytntayytncaratgwsnwsnytnaarwsngargayacngcnytntaytaytgyacnmgnggntaywsnwsnacnwsntaygcnatggayttytggggncarggnacnacngtnacnwsntrr

SEQ ID NO: 253 - DNA, 342 bp, Homo sapiens
  misc_feature (9)..(9): n is a, c, g, or t
gayathgtnatgacncaracnccnwsnacnttywsngcnacngtnggngaraargtnacnathacntgymgngcnwsncarwsngtncaywsngayggnaayacntayatgcartggtaycarcaraarwsnggnmgnggnccnaarttyytnathtaygcngcnwsnaaymgnttywsnggngtnccngayaarwsnggnwsnggnggnggnacngayttyacnytnwsnggnathaayacnytncarwsngargayttygcnacntaytaytgycarcarggnacncarytnccnmgnacnttyggncarggnacnaargtngargcnacnmgnacntrr

SEQ ID NO: 254 - DNA, 342 bp, Homo sapiens
  misc_feature (9)..(9): n is a, c, g, or t
gayathgtnatgacncarwsnccnacnwsnttywsngcnacngtnggngaraargtnacnathwsntgymgnwsnwsncarwsngtncaywsngayggnaaywsntayytnwsntggtaycarcaraarwsnggnaarggnccnmgnttyytnathtayggngcnwsnaayaarttywsnggngtnccngayaarwsnggnwsnggngcnggnacngaytayacnytnwsnggnathaayacngtncarwsngargayttygcnacntaytaytgycarcarggnacncarytnccnmgnacnttyggncarggnacnaargtngargcnacnggngcntrr

SEQ ID NO: 255 - DNA, 345 bp, Homo sapiens
  misc_feature (9)..(9): n is a, c, g, or t
gayathgtnatgacnaaywsnccnacnwsnttyacngcnacngtnggngaraargtnacnwsnathwsntgyaarwsncarwsnytngtncaywsngayggnaaywsntayytnwsntggytncaycarmgnwsnggnmgngcnccnmgnttyytnathtaymgnathwsnaaymgnttywsnggngtnccngaygartayggnwsnggngcnggnacngaytayacnytnwsnggnathaayacnathcarwsngargayttygcnwsntaytaytgycarcarggnacncarytnccnmgnacnttyggncarggnacnaargtngargcnacnggngcntrr

SEQ ID NO: 256 - DNA, 348 bp, Homo sapiens
  misc_feature (6)..(6): n is a, c, g, or t
gargtncarytncarcarwsnggnacnytnytngcnaarccnggngcnwsngtnaaratgwsntgyaargcnwsnggntaywsntaywsnwsntaytggatgcaytggathaarcarmgnccnggncarggnytngartggathggngcnathgayccnmgnaaywsngayacnathtayaayccnaayttyaarcayaargcnaarytnwsngcngtnacnwsnacnwsnacngcntayatggargtnaaywsnytnacnaaygargaywsngcngtntaytaytgyacnccnytntaytaytaygaywsntggggncarggnacnacnytnacngtnwsnwsntrr

SEQ ID NO: 257 - DNA, 348 bp, Homo sapiens
  misc_feature (6)..(6): n is a, c, g, or t
gargtncarytncarcarwsnggnacngtnytngcnaarccngcngcnwsnatgmgnatgwsntgyaargcnwsnggntayacnathwsnwsntaytggatgcaytggathaarcarmgnccnggncarggnytngaytggathgtnggnathgayccnmgnaaywsngayacngcntayaayccncarttyaarcayaargcnaarytnacngcngtnacnwsnwsnwsnacngcntayatggarytnaaywsnytnacnaaygaygaywsngcngtntaytaytgyacnccnytntaytayttygaywsntggggncarggnacnacnytnacngtnwsnwsntrr

SEQ ID NO: 258 - DNA, 348 bp, Homo sapiens
  misc_feature (6)..(6): n is a, c, g, or t
gargtncarytncarcarwsnggnacnytnytngcnmgnccnggnathacngtnaaratgwsntgyaargcnwsnggntayacngcnacnacntaytggatgcaytggathaarcarmgnccnggncarggnytngarytnathgtngcngcngayccnmgnaaywsngayacnathtaycarccncartayaarcayaarggnaarytnacngcngtnacnwsnacnacnwsnathtayatggayytnaaywsnytnacnaaygargaywsngcngtntaytaytgyacnccnytntaytayttygarwsntggggncarggnacnacnytnacngtnwsnwsntrr

SEQ ID NO: 259 - DNA, 318 bp, Homo sapiens
  misc_feature (9)..(9): n is a, c, g, or t
gayathmgnatgwsncarwsnccnacnwsnatgtaygcnwsnytnggngarmgngtnacntayacntgymgngcnwsncargayathaayaaytaygtntgytggttycarcaraarccnggnaarwsnccnaarwsnytnathtayaargcnaaymgnytngtngayggngtnccnwsnmgntaywsnggnwsnggnwsnggncargartaywsnytnacnathwsnwsnytngartaygargayatgggnathtaytaytgyytncarttygaygarttyccntayacnttyggnggnggnacnaarytngartrr

SEQ ID NO: 260 - DNA, 324 bp, Homo sapiens
  misc_feature (15)..(15): n is a, c, g, or t
gayathaaratgacncarwsnccnwsnwsnatgtaygcnwsngtnggngaymgngtnacnttyacntgyaargcnwsncargarathaayaaytayytntgytggttycarcarmgnccnggnaaracnccnmgnacnytnathtaymgngcnaayaarytngtngayggngtnccnwsnmgnttywsnggnwsnggnwsngcncargaytaywsnytnacnathwsnwsnytngartaygargayatgggnathtaytaytgyytncartaygaygayttyccntayacnttyggnggnggnacnaarytngarathmgntrr

SEQ ID NO: 261 - DNA, 324 bp, Homo sapiens
  misc_feature (6)..(6): n is a, c, g, or t
gaygcnaaratgacnaaywsnccnwsnwsnatgtaygcnwsnytnggngarmgngtnacnttyacntgyaargcnwsncargayathaaycarttyytntgytggttycarcaraarccnggnaaracnccnaaracnytnathtaymgngcnaaymgnytngtngayggngtnccnwsnmgnttywsnggnacnggnwsnggncargaytaywsnytnacnathwsnwsnytngarttygargayatgggnathtaytaytgygtncartaygaygarttyccntaywsnttyggnggnggnacnaarytngarathaartrr

SEQ ID NO: 262 - DNA, 372 bp, Homo sapiens
caggtgcagctggtggatagcggcgcgggcgtggtgcagccgggccgcagcctgcgcgtgagctgcgcggcgagcggctttagctttagcacctatggcatgcattgggtgcgccagggcccgggcaaaggcctggaatgggtggcggtgatttgggatgatggcagctataaatattttggcgatagcgtgcgcggccgctataccattagcaaagaacagagcaaagtgaccctgtttgtgcagatgaacagcctgaaagcggatgaaaccgcgggcttttattgcgcgcgcgatgcgattaccatggtgcgcggcgtgatgaaagaatattttgattattggggccagggcaccctggtgaccgtgtaa

SEQ ID NO: 263 - DNA, 372 bp, Homo sapiens
caggtgcagctggtggaaaccggcgcgggcgtggtgcagccgggccgcagcctgaaagtgagctgcgcggcgagcggctttacctatagcacctatggcatgcattgggtgcgccaggcgccgggccgcggcctggaatgggtggcggtgatttgggaagatggcagctataaatattatggcgatagcgtgaaaggccgctttaccgcgagccgcgataacagccgcaacaccctgtatctgaacatgaacagcctgaaagcggatgatagcgcggtgtattattgcgcgcgcgatggcattagcatggtgcgcgcggtgatgcgcgattattttgatttttggggccagggcaccctggtgaccgtgtaa

SEQ ID NO: 264 - DNA, 372 bp, Homo sapiens
caggtgcagctggtggatagcggcggcggcgtgctgcagccgggccgcagcctgaaactgagctgcgcggcgagcggctttacctttagcacctttgcgatgcattgggtgcgccaggcgccggcgaaaggcctggaatgggtggcggtgatttgggatgatggcagctataaattttatgcggaaagcgtgcgcggccgctttaccggcacccgcgataacagcaaagtgaccctgtttctgcagatgcagagcctgcgcgcggaagataccgcggtgttttattgcgcgcgcgatggcattaccatggtgcgcggcgtgatgcgcgattattttgatttttggggccagggcaccctggtgaccgtgtaa

SEQ ID NO: 265 - DNA, 315 bp, Homo sapiens
gcgattcagctgaccaacagcccgagcagcctgagcgcgagcgtgggcgatcgcgtgaccattagctgccgcgcgagccaggatattagcagcgcgctggtgtggtatcagcagaaaccggcgcgcgcgccgaaactggtgatttatgatgcgagcagcctggaaagcggcgtgccgaccaaatttaccggcaccgatagcggcaccgattttagcctgaccattagcagcctgcagccggatgattttgcgaccttttattgccagcagtttaacagctatccgctgacctttggcggcggcaccaaagtgtaa

SEQ ID NO: 266 - DNA, 315 bp, Homo sapiens
gcgattcaggtgacccagagcccgaccagcctgagcgcgaccgtgggcgatcgcgtgagcattacctgccgcgcgagccaggaaattagcagcgcgctgctgtggtatcagcagaaaccgggcaaagcgccgcgcctgctgatttatgaagcgagcagcctggaaaccggcgtgccgagcaaatttaccggcagcgaaaccggcagcgattttacccgcaccattagcagcgtgcagccggaagatgcgtatacctatttttgccagaactttaacagctatccgctgagctttggcggcggcaccaaagtgtaa

SEQ ID NO: 267 - DNA, 315 bp, Homo sapiens
gcgattcagctgacccagagcccgagcaccctgaccgcgagcgtgggcgatcgcgtgaccattacctgccgcgcgagccaggatattaccagcgcgctgctgtggtatcagcagcgcccggcgaaagcgccgaaagtgctgatttatgatgcgagcagcctggaaagcggcgtgccgagccgctttagcggcagcgatagcggcagcgaatataccctgaccattagcagcgtgaacccggatgattatgcgacctattattgcaaccagtttcagagctatccgctgagctttggcggcggcaccaaagtgtaa

SEQ ID NO: 268 - DNA, 357 bp, Homo sapiens
caggtgaaactgcagcagagcggcggcggcctgccgaaagtggcgggcagcctgaaactgagctgcgtgaccagcggctttagctttcgcaaatttggcatgagctgggtgcgccagaccagcgataaacgcctggaatggattggcagcattagcaccggcggctataacagctattatagcgataacgtgaaaggccgctttaccattagccgcgaaaacgcgaaaaacaccctgtatctgaacatgagcagcctgaaaagcgaagataccgcgctgtattattgcgcgcgcggctttagcagcaccagctatgcgatggattattggggccagggcaccaccgtgaccgtgtaa

SEQ ID NO: 269 - DNA, 351 bp, Homo sapiens
caggtgaaagtgcagaacagcggcggcggcctggtgaaaccgggcgcgagcctgaaactgagctgcgtgaccagcggctttacctttaaaaaatttggcatgagctgggtgaaacagaccagcgataaaaaactggaatgggtggcgagcattagcaccggcggctttaacacctattatagcgataacgtgaaaggccgctttaccattagccgcgaaaacggcaaaaacaccctgtatgtgcagatgagcagcctgaaaagcgaagataccgcgctgtattattgcacccgcggctatagcagcaccagctttggcatggattattggggccagggcaccaccgtgtaa

SEQ ID NO: 270 - DNA, 357 bp, Homo sapiens
caggtgaaactgcagcagagcggcgcgggcctggtgaaaccgggcgcgagcctgaaactgagctgcgtgaccagcggctatagctttcgcaaatttggcatgagctgggtgcgccagagcaccgataaacgcctggaatgggtggcgagcattagcaccggcggctatcagacctattatagcgataacgtgaaaggccgctttaccattagccgcgaaaacgcgaaaaacaccctgtatctgcagatgagcagcctgaaaagcgaagataccgcgctgtattattgcacccgcggctatagcagcaccagctatgcgatggatttttggggccagggcaccaccgtgaccagctaa

SEQ ID NO: 271 - DNA, 342 bp, Homo sapiens
gatattgtgatgacccagaccccgagcacctttagcgcgaccgtgggcgaaaaagtgaccattacctgccgcgcgagccagagcgtgcatagcgatggcaacacctatatgcagtggtatcagcagaaaagcggccgcggcccgaaatttctgatttatgcggcgagcaaccgctttagcggcgtgccggataaaagcggcagcggcggcggcaccgattttaccctgagcggcattaacaccctgcagagcgaagattttgcgacctattattgccagcagggcacccagctgccgcgcacctttggccagggcaccaaagtggaagcgacccgcacctaa

SEQ ID NO: 272 - DNA, 342 bp, Homo sapiens
gatattgtgatgacccagagcccgaccagctttagcgcgaccgtgggcgaaaaagtgaccattagctgccgcagcagccagagcgtgcatagcgatggcaacagctatctgagctggtatcagcagaaaagcggcaaaggcccgcgctttctgatttatggcgcgagcaacaaatttagcggcgtgccggataaaagcggcagcggcgcgggcaccgattataccctgagcggcattaacaccgtgcagagcgaagattttgcgacctattattgccagcagggcacccagctgccgcgcacctttggccagggcaccaaagtggaagcgaccggcgcgtaa

SEQ ID NO: 273 - DNA, 345 bp, Homo sapiens
gatattgtgatgaccaacagcccgaccagctttaccgcgaccgtgggcgaaaaagtgaccagcattagctgcaaaagccagagcctggtgcatagcgatggcaacagctatctgagctggctgcatcagcgcagcggccgcgcgccgcgctttctgatttatcgcattagcaaccgctttagcggcgtgccggatgaatatggcagcggcgcgggcaccgattataccctgagcggcattaacaccattcagagcgaagattttgcgagctattattgccagcagggcacccagctgccgcgcacctttggccagggcaccaaagtggaagcgaccggcgcgtaa

SEQ ID NO: 274 - DNA, 348 bp, Homo sapiens
gaagtgcagctgcagcagagcggcaccctgctggcgaaaccgggcgcgagcgtgaaaatgagctgcaaagcgagcggctatagctatagcagctattggatgcattggattaaacagcgcccgggccagggcctggaatggattggcgcgattgatccgcgcaacagcgataccatttataacccgaactttaaacataaagcgaaactgagcgcggtgaccagcaccagcaccgcgtatatggaagtgaacagcctgaccaacgaagatagcgcggtgtattattgcaccccgctgtattattatgatagctggggccagggcaccaccctgaccgtgagcagctaa

SEQ ID NO: 275 - DNA, 348 bp, Homo sapiens
gaagtgcagctgcagcagagcggcaccgtgctggcgaaaccggcggcgagcatgcgcatgagctgcaaagcgagcggctataccattagcagctattggatgcattggattaaacagcgcccgggccagggcctggattggattgtgggcattgatccgcgcaacagcgataccgcgtataacccgcagtttaaacataaagcgaaactgaccgcggtgaccagcagcagcaccgcgtatatggaactgaacagcctgaccaacgatgatagcgcggtgtattattgcaccccgctgtattattttgatagctggggccagggcaccaccctgaccgtgagcagctaa

SEQ ID NO: 276 - DNA, 348 bp, Homo sapiens
gaagtgcagctgcagcagagcggcaccctgctggcgcgcccgggcattaccgtgaaaatgagctgcaaagcgagcggctataccgcgaccacctattggatgcattggattaaacagcgcccgggccagggcctggaactgattgtggcggcggatccgcgcaacagcgataccatttatcagccgcagtataaacataaaggcaaactgaccgcggtgaccagcaccaccagcatttatatggatctgaacagcctgaccaacgaagatagcgcggtgtattattgcaccccgctgtattattttgaaagctggggccagggcaccaccctgaccgtgagcagctaa

SEQ ID NO: 277 - DNA, 318 bp, Homo sapiens
gatattcgcatgagccagagcccgaccagcatgtatgcgagcctgggcgaacgcgtgacctatacctgccgcgcgagccaggatattaacaactatgtgtgctggtttcagcagaaaccgggcaaaagcccgaaaagcctgatttataaagcgaaccgcctggtggatggcgtgccgagccgctatagcggcagcggcagcggccaggaatatagcctgaccattagcagcctggaatatgaagatatgggcatttattattgcctgcagtttgatgaatttccgtatacctttggcggcggcaccaaactggaataa

SEQ ID NO: 278 - DNA, 324 bp, Homo sapiens
gatattaaaatgacccagagcccgagcagcatgtatgcgagcgtgggcgatcgcgtgacctttacctgcaaagcgagccaggaaattaacaactatctgtgctggtttcagcagcgcccgggcaaaaccccgcgcaccctgatttatcgcgcgaacaaactggtggatggcgtgccgagccgctttagcggcagcggcagcgcgcaggattatagcctgaccattagcagcctggaatatgaagatatgggcatttattattgcctgcagtatgatgattttccgtatacctttggcggcggcaccaaactggaaattcgctaa

SEQ ID NO: 279 - DNA, 324 bp, Homo sapiens
gatgcgaaaatgaccaacagcccgagcagcatgtatgcgagcctgggcgaacgcgtgacctttacctgcaaagcgagccaggatattaaccagtttctgtgctggtttcagcagaaaccgggcaaaaccccgaaaaccctgatttatcgcgcgaaccgcctggtggatggcgtgccgagccgctttagcggcaccggcagcggccaggattatagcctgaccattagcagcctggaatttgaagatatgggcatttattattgcgtgcagtatgatgaatttccgtatagctttggcggcggcaccaaactggaaattaaataa

SEQ ID NO: 280 - protein, 123 aa, Homo sapiens
QVQLVDSGAGVVQPGRSLRVSCAASGFSFSTYGMHWVRQGPGKGLEWVAVIWDDGSYKYFGDSVRGRYTISKEQSKVTLFVQMNSLKADETAGFYCARDAITMVRGVMKEYFDYWGQGTLVTV

SEQ ID NO: 281 - protein, 123 aa, Homo sapiens
QVQLVETGAGVVQPGRSLKVSCAASGFTYSTYGMHWVRQAPGRGLEWVAVIWEDGSYKYYGDSVKGRFTASRDNSRNTLYLNMNSLKADDSAVYYCARDGISMVRAVMRDYFDFWGQGTLVTV

SEQ ID NO: 282 - protein, 123 aa, Homo sapiens
QVQLVDSGGGVLQPGRSLKLSCAASGFTFSTFAMHWVRQAPAKGLEWVAVIWDDGSYKFYAESVRGRFTGTRDNSKVTLFLQMQSLRAEDTAVFYCARDGITMVRGVMRDYFDFWGQGTLVTV

SEQ ID NO: 283 - protein, 104 aa, Homo sapiens
AIQLTNSPSSLSASVGDRVTISCRASQDISSALVWYQQKPARAPKLVIYDASSLESGVPTKFTGTDSGTDFSLTISSLQPDDFATFYCQQFNSYPLTFGGGTKV

SEQ ID NO: 284 - protein, 104 aa, Homo sapiens
AIQVTQSPTSLSATVGDRVSITCRASQEISSALLWYQQKPGKAPRLLIYEASSLETGVPSKFTGSETGSDFTRTISSVQPEDAYTYFCQNFNSYPLSFGGGTKV

SEQ ID NO: 285 - protein, 104 aa, Homo sapiens
AIQLTQSPSTLTASVGDRVTITCRASQDITSALLWYQQRPAKAPKVLIYDASSLESGVPSRFSGSDSGSEYTLTISSVNPDDYATYYCNQFQSYPLSFGGGTKV

SEQ ID NO: 286 - protein, 118 aa, Homo sapiens
QVKLQQSGGGLPKVAGSLKLSCVTSGFSFRKFGMSWVRQTSDKRLEWIGSISTGGYNSYYSDNVKGRFTISRENAKNTLYLNMSSLKSEDTALYYCARGFSSTSYAMDYWGQGTTVTV

SEQ ID NO: 287 - protein, 116 aa, Homo sapiens
QVKVQNSGGGLVKPGASLKLSCVTSGFTFKKFGMSWVKQTSDKKLEWVASISTGGFNTYYSDNVKGRFTISRENGKNTLYVQMSSLKSEDTALYYCTRGYSSTSFGMDYWGQGTTV

SEQ ID NO: 288 - protein, 118 aa, Homo sapiens
QVKLQQSGAGLVKPGASLKLSCVTSGYSFRKFGMSWVRQSTDKRLEWVASISTGGYQTYYSDNVKGRFTISRENAKNTLYLQMSSLKSEDTALYYCTRGYSSTSYAMDFWGQGTTVTS

SEQ ID NO: 289 - protein, 113 aa, Homo sapiens
DIVMTQTPSTFSATVGEKVTITCRASQSVHSDGNTYMQWYQQKSGRGPKFLIYAASNRFSGVPDKSGSGGGTDFTLSGINTLQSEDFATYYCQQGTQLPRTFGQGTKVEATRT

SEQ ID NO: 290 - protein, 113 aa, Homo sapiens
DIVMTQSPTSFSATVGEKVTISCRSSQSVHSDGNSYLSWYQQKSGKGPRFLIYGASNKFSGVPDKSGSGAGTDYTLSGINTVQSEDFATYYCQQGTQLPRTFGQGTKVEATGA

SEQ ID NO: 291 - protein, 114 aa, Homo sapiens
DIVMTNSPTSFTATVGEKVTSISCKSQSLVHSDGNSYLSWLHQRSGRAPRFLIYRISNRFSGVPDEYGSGAGTDYTLSGINTIQSEDFASYYCQQGTQLPRTFGQGTKVEATGA

SEQ ID NO: 292 - protein, 115 aa, Homo sapiens
EVQLQQSGTLLAKPGASVKMSCKASGYSYSSYWMHWIKQRPGQGLEWIGAIDPRNSDTIYNPNFKHKAKLSAVTSTSTAYMEVNSLTNEDSAVYYCTPLYYYDSWGQGTTLTVSS

SEQ ID NO: 293 - protein, 115 aa, Homo sapiens
EVQLQQSGTVLAKPAASMRMSCKASGYTISSYWMHWIKQRPGQGLDWIVGIDPRNSDTAYNPQFKHKAKLTAVTSSSTAYMELNSLTNDDSAVYYCTPLYYFDSWGQGTTLTVSS

SEQ ID NO: 294 - protein, 115 aa, Homo sapiens
EVQLQQSGTLLARPGITVKMSCKASGYTATTYWMHWIKQRPGQGLELIVAADPRNSDTIYQPQYKHKGKLTAVTSTTSIYMDLNSLTNEDSAVYYCTPLYYFESWGQGTTLTVSS

SEQ ID NO: 295 - protein, 107 aa, Homo sapiens
DIRMSQSPTSMYASLGERVTYTCRASQDINNYVCWFQQKPGKSPKSLIYKANRLVDGVPSRYSGSGSGQEYSLTISSLEYEDMGIYYCLQFDEFPYTFGGGTKLEIK

SEQ ID NO: 296 - protein, 107 aa, Homo sapiens
DIKMTQSPSSMYASVGDRVTFTCKASQEINNYLCWFQQRPGKTPRTLIYRANKLVDGVPSRFSGSGSAQDYSLTISSLEYEDMGIYYCLQYDDFPYTFGGGTKLEIR

SEQ ID NO: 297 - protein, 107 aa, Homo sapiens
DAKMTNSPSSMYASLGERVTFTCKASQDINQFLCWFQQKPGKTPKTLIYRANRLVDGVPSRFSGTGSGQDYSLTISSLEFEDMGIYYCVQYDEFPYSFGGGTKLEIK

SEQ ID NO: 298 - DNA, 20 bp, Artificial Sequence
  Synthetic oligonucleotide
cagcgctaccttgtcattca

SEQ ID NO: 299 - DNA, 20 bp, Artificial Sequence
  Synthetic oligonucleotide
gaatgcatttgccaagtcct

SEQ ID NO: 300 - DNA, 20 bp, Artificial Sequence
  Synthetic oligonucleotide
gggctcacagcaaacttctc

SEQ ID NO: 301 - DNA, 20 bp, Artificial Sequence
  Synthetic oligonucleotide
aagccagactcgctcatgtt

SEQ ID NO: 302 - DNA, 20 bp, Artificial Sequence
  Synthetic oligonucleotide
acacacacacacacacaccg

SEQ ID NO: 303 - DNA, 20 bp, Artificial Sequence
  Synthetic oligonucleotide
ggctcacagcaaacttctcc

SEQ ID NO: 304 - DNA, 20 bp, Artificial Sequence
  Synthetic oligonucleotide
acttgacaggggaaacatgc

SEQ ID NO: 305 - DNA, 20 bp, Artificial Sequence
  Synthetic oligonucleotide
caaggtctgggaaccactgt

SEQ ID NO: 306 - DNA, 20 bp, Artificial Sequence
  Synthetic oligonucleotide
ttgcacaattccaaccttga

SEQ ID NO: 307 - DNA, 20 bp, Artificial Sequence
  Synthetic oligonucleotide
gcatgtttcccctgtcaagt

SEQ ID NO: 308 - DNA, 20 bp, Artificial Sequence
  Synthetic oligonucleotide
ccataacacccacctctgct

SEQ ID NO: 309 - DNA, 20 bp, Artificial Sequence
  Synthetic oligonucleotide
actggctgcagttgacacac

SEQ ID NO: 310 - DNA, 20 bp, Artificial Sequence
  Synthetic oligonucleotide
accaagctctgctccacact

SEQ ID NO: 311 - DNA, 20 bp, Artificial Sequence
  Synthetic oligonucleotide
acacagcggtgtgagaagtg

SEQ ID NO: 312 - DNA, 20 bp, Artificial Sequence
  Synthetic oligonucleotide
aggccaggggtagagagtag

SEQ ID NO: 313 - DNA, 20 bp, Artificial Sequence
  Synthetic oligonucleotide
tcagaccctcttgggaccta

SEQ ID NO: 314 - DNA, 20 bp, Artificial Sequence
  Synthetic oligonucleotide
gcctccacttcaaccacagt

SEQ ID NO: 315 - DNA, 20 bp, Artificial Sequence
  Synthetic oligonucleotide
cccacgtccgtagaaaggta

SEQ ID NO: 316 - DNA, 20 bp, Artificial Sequence
  Synthetic oligonucleotide
tgtgactgcctgtccctaca

SEQ ID NO: 317 - DNA, 20 bp, Artificial Sequence
  Synthetic oligonucleotide
cccagctctttgaggacaac

SEQ ID NO: 318 - DNA, 20 bp, Artificial Sequence
  Synthetic oligonucleotide
agccagctggttgttcttgt

SEQ ID NO: 319 - DNA, 20 bp, Artificial Sequence
  Synthetic oligonucleotide
agcttcgaagcctcacagag

SEQ ID NO: 320 - DNA, 20 bp, Artificial Sequence
  Synthetic oligonucleotide
tggggagagagttctgagga

SEQ ID NO: 321 - DNA, 20 bp, Artificial Sequence
  Synthetic oligonucleotide
acagatgccactgtggttga

SEQ ID NO: 322 - DNA, 19 bp, Artificial Sequence
  Synthetic oligonucleotide
gactgctgccatgagcagt

SEQ ID NO: 323 - DNA, 20 bp, Artificial Sequence
  Synthetic oligonucleotide
ggatcaagacccctcctttc

SEQ ID NO: 324 - DNA, 20 bp, Artificial Sequence
  Synthetic oligonucleotide
ccatctgcaccattgatgtc

SEQ ID NO: 325 - DNA, 20 bp, Artificial Sequence
  Synthetic oligonucleotide
gagcggtagaaggtgctgtc

SEQ ID NO: 326 - DNA, 20 bp, Artificial Sequence
  Synthetic oligonucleotide
cgggagttggtgtctgaatt

SEQ ID NO: 327 - DNA, 20 bp, Artificial Sequence
  Synthetic oligonucleotide
ccctcatccaccataacacc

SEQ ID NO: 328 - DNA, 20 bp, Artificial Sequence
  Synthetic oligonucleotide
cgcttttggcacagtctaca

SEQ ID NO: 329 - DNA, 20 bp, Artificial Sequence
  Synthetic oligonucleotide
tcccggacatggtctaagag

SEQ ID NO: 330 - DNA, 20 bp, Artificial Sequence
  Synthetic oligonucleotide
aattccagtggccatcaaag

SEQ ID NO: 331 - DNA, 20 bp, Artificial Sequence
  Synthetic oligonucleotide
ggtgacacagcttatgccct

SEQ ID NO: 332 - DNA, 20 bp, Artificial Sequence
  Synthetic oligonucleotide
tttcccggacatggtctaag

SEQ ID NO: 333 - DNA, 19 bp, Artificial Sequence
  Synthetic oligonucleotide
gagcccagaggagaagact

SEQ ID NO: 334 - DNA, 20 bp, Artificial Sequence
  Synthetic oligonucleotide
tctgatgcgacagacactcc

SEQ ID NO: 335 - DNA, 20 bp, Artificial Sequence
  Synthetic oligonucleotide
gagtctgagtgttcggaggg

SEQ ID NO: 336 - DNA, 20 bp, Artificial Sequence
  Synthetic oligonucleotide
aattgactggagggacatcg

SEQ ID NO: 337 - DNA, 20 bp, Artificial Sequence
  Synthetic oligonucleotide
ggagcacagatggtcttggt

SEQ ID NO: 338 - DNA, 20 bp, Artificial Sequence
  Synthetic oligonucleotide
aggacaatggcagaagctgt

SEQ ID NO: 339 - DNA, 20 bp, Artificial Sequence
  Synthetic oligonucleotide
aggagcacagatggtcttgg

SEQ ID NO: 340 - DNA, 20 bp, Artificial Sequence
  Synthetic oligonucleotide
cgaggtacacaggctccact

SEQ ID NO: 341 - DNA, 20 bp, Artificial Sequence
  Synthetic oligonucleotide
accaagaccatctgtgctcc

SEQ ID NO: 342 - DNA, 20 bp, Artificial Sequence
  Synthetic oligonucleotide
ggaagtttgccatcttcgtc

SEQ ID NO: 343 - DNA, 20 bp, Artificial Sequence
  Synthetic oligonucleotide
acagcttctgccattgtcct

SEQ ID NO: 344 - DNA, 20 bp, Artificial Sequence
  Synthetic oligonucleotide
gagggacccaggtctacgat

SEQ ID NO: 345 - DNA, 20 bp, Artificial Sequence
  Synthetic oligonucleotide
tttcgggagtttgagaatgg

SEQ ID NO: 346 - DNA, 20 bp, Artificial Sequence
  Synthetic oligonucleotide
gaaactgtttgccccctgta

SEQ ID NO: 347 - DNA, 20 bp, Artificial Sequence
  Synthetic oligonucleotide
aagatggaagatggcctcct

SEQ ID NO: 348 - DNA, 20 bp, Artificial Sequence
  Synthetic oligonucleotide
ggtgaatttcgggagtttga

SEQ ID NO: 349 - DNA, 20 bp, Artificial Sequence
  Synthetic oligonucleotide
ggtgcttttggaacggttta

SEQ ID NO: 350 - DNA, 20 bp, Artificial Sequence
  Synthetic oligonucleotide
aaccggactaggtgtggatg

SEQ ID NO: 351 - DNA, 20 bp, Artificial Sequence
  Synthetic oligonucleotide
caaggcaaatgtggagttca

SEQ ID NO: 352 - DNA, 20 bp, Artificial Sequence
  Synthetic oligonucleotide
caaccggactaggtgtggat

SEQ ID NO: 353 - DNA, 20 bp, Artificial Sequence
  Synthetic oligonucleotide
ccagaccaatgtctgtcgtg

SEQ ID NO: 354 - DNA, 20 bp, Artificial Sequence
  Synthetic oligonucleotide
aggaggccatcttccatctt

SEQ ID NO: 355 - DNA, 20 bp, Artificial Sequence
  Synthetic oligonucleotide
tgcactcagagagctcagga

SEQ ID NO: 356 - protein, 7 aa, Artificial Sequence
  Synthetic peptide
PKKKRKV

SEQ ID NO: 357 - protein, 16 aa, Artificial Sequence
  Synthetic peptide
KRPAATKKAGQAKKKK